(19) United States
(12) Patent Application Publication
(10) Pub. No.: US 2017/0304336 A1
(43) Pub. Date: Oct. 26, 2017

(54) COMBINATION

(71) Applicant: Aleksander Vladimirovich DIKOVSKIY, Moscow (RU)

(72) Inventors: Alexander Vladimirovich DIKOVSKIY, Moscow (RU); Svetlana Anatoleva ZAKIROVA, Moscow (RU); Aliaksandr Viktorovich MATSIUSHONAK, Moscow (BY)

(21) Appl. No.: 15/527,080

(22) PCT Filed: Oct. 30, 2015
(86) PCT No.: PCT/EP2015/075357

§ 371 Date: May 16, 2017

(30) Foreign Application Priority Data
Nov. 10, 2014 (RU) 2014145102

(51) Int. Cl. A61K 31/704; A61K 31/405; A61K 31/40; A61K 31/366; A61K 31/215; A61K 31/505; A61K 9/10

(52) U.S. Cl. CPC A61K 31/704; A61K 31/505; A61K 31/405; A61K 9/10; A61K 31/366; A61K 31/215; A61K 31/40

(57) ABSTRACT

A combination comprising: (a) a glycyrrhizin derivative; and (b) a hypolipidemic drug; is disclosed. Pharmaceutical compositions, kits, methods of treatment and medical uses of the combination are also disclosed.

### CROSS REFERENCE TO RELATED APPLICATIONS

The present patent application is National stage application from PCT application PCT/EP2015/075357 filed on Oct. 30, 2015 which claims priority to Russian patent applications RU2014145102 filed Nov. 10, 2014, all of which incorporated herein by reference entirely.

### FIELD OF THE INVENTION

This invention relates to a pharmaceutical combination. It also relates to pharmaceutical compositions, kits, methods of treatment and medical uses of the combination.

### BACKGROUND TO THE INVENTION

Hypercholesterolemia is one of the most important lipid metabolism disorders in development of cardiovascular diseases. In recent decades, the majority of developed countries have adopted national cardiovascular disease (CVD) prevention and treatment programs, which helped to reduce CVD pathology mortality by more than 50%. An important part of these programs is the widespread use of statins in clinical practice.

HMG-CoA reductase inhibitors, commonly known as statins, are a class of drugs used to lower blood lipid (especially blood cholesterol) levels by inhibiting the enzyme HMG-CoA reductase. This enzyme plays a central role in the production of cholesterol in the liver, which produces about 70 percent of total cholesterol in the body. Increased cholesterol levels have been associated with cardiovascular disease. The cholesterol-lowering properties of statins make them useful in the treatment and prevention of cardiovascular disease.

In particular, simvastatin is one of the most commonly prescribed effective, relatively safe and available statins. Extensive multi-center clinical studies conducted in accordance with strict modern standards have proven efficacy of simvastatin not only in secondary prevention of CVD, but also in diabetes mellitus patients and dyslipidemia patients. There is strong evidence of the significant impact of statins on the pathogenesis of certain rheumatic diseases. Although simvastatin was not the first statin to be synthesized, the most high-profile evidence of efficacy of this drug group, as clinically significant cardiovascular disease outcomes are concerned, were obtained with this drug. As published, the results of virtually all studies of simvastatin radically changed the modern cardiology practice and earnestly claimed indications for use of this drug in new diseases and syndromes—see Karpov and Sorokin E. V. Russian Medical Journal: independent edition for practitioners. 2008, 16 (21), 1435-1438.

However, a number of adverse effects are known to be associated with statins, particularly when they are administered at high daily doses (20-80 mg/day). Examples of adverse effects include raised levels of liver enzymes such as alanine-aminotransferase and aspartate-aminotransferase, myalgia (muscle pain), myopathy (muscle disease), and rhabdomyolysis (muscle breakdown). In addition, long-term administration of statins can be costly to the patient and healthcare provider.

In particular, in June 2011, the US FDA recommended to limit the use of simvastatin in doses of 80 mg per day because of the risk of damaging muscle tissues. It was noted that patients taking simvastatin in the dose of 80 mg per day have a higher risk of myopathy than patients who received it or other statin drugs in smaller doses. It has been observed that the likelihood of such adverse reaction is especially high during the first year of statin therapy and may be associated both with drug interactions and a genetic predisposition to development of simvastatin-dependent myopathy. Therefore, 40 mg of simvastatin was recognized as the maximum safe daily dose. At the same time, FDA proportionally reduced (by 50% or more) the maximum safe dose of simvastatin when used in combination with other drugs that are capable to increase concentrations of statin in serum (due to drug interaction).

In addition to rhabdomyolysis, statins can contribute to development of myorenal syndrome (due to blocked renal tubules with myoglobin and uromodulin aggregates) and acute renal failure.

Glycyrrhizic acid (also known as glycyrrhizin or glycyrrhizinic acid) is the main sweet-tasting constituent of Glycyrrhiza glabra (liquorice) root. Glycyrrhizic acid exhibits antiatherosclerotic activity: it is believed that its mechanism of action comprises inhibition of activity of phospholipase A2 and accelerated bile acid synthesis. Glycyrrhizin is also known to inhibit liver cell injury and is approved for intravenous administration in Japan for the treatment of chronic viral hepatitis and cirrhosis (Inoue H., Saito H., Koshihara Y., Murota S. // Chem. Pharm. Bull. 1986. V. 34(2). P. 897-901).

Glycyrrhizins are also known to exhibit hypolipidemic and antiatherosclerotic properties. For example, Fuhrman et al. Nutrition. 2002, 18(3), 268-273, describes the antiatherosclerotic effects of an ethanolic extract of Glycyrrhiza glabra L. When this extract was administered to the patients with high cholesterol, decrease of cholesterol content and triglyceride level in plasma was observed as well as increase of resistance of low-molecular lipoproteins to oxidation and reduction of systolic blood pressure.

Furthermore, Vasilenko et al. Chemical-Pharmaceutical Journal, 1981, [symbol] o 5, 50-53 and Skulipe et al. Biol. sciences. 1952. [symbol] o 10. 56-60, describe that Glycyram, glycyrrhetic acid and sodium salt of glycyrrhetic acid (sodium glycyrrhizinate) (10 mg/kg) also display hypolipidemic and antiatherosclerotic activity, decrease content of cholesterol, β-lipoproteins and triglycerides in blood of rabbits with experimental atherosclerosis, reduce cholesterol level in liver tissues, increase blood coagulation.

In other experiment increase of time of blood plasma recalcification and decrease of tolerance of blood to heparin are observed in animals used in experiments (Baran J. S., Langford D. D., Chi-Dean Liang B. S., Pitzele B. S. // J. Med. Chem. 1974. V. 17. P. 184-191). Powerful hypolipidemic properties have been revealed in acetates of glycyrrhizic acid, glycyrrhetic acid and 3-amino-glycyrrhetic acid in animals with experimental atherosclerosis (see Fuhrman et al. cited above; Vasilenko et al. Abs. Sev.-kavk. learn. (centers higher school of Natural science) 1984. [symbol] o 4, 83-87; and Vasilenko et al. Pharmacology and Toxicology. 1952. [symbol] o 5, 66-70). When glycyrrhizinate was administered (10 mg/kg), decrease of level of cholesterol and β-lipoproteins in aorta and cholesterol content in liver tissues were observed (Vasilenko et al., // Chemical-Pharmaceutical, journal, 1981. [symbol] o 5, 50-53).

The hypolipidemic and antiatherosclerotic activity of derived glycyrrhizic acid and glycyrrhetic acid is higher than in official preparations such as polysponine and Miscleron. For example, 18-dehydro-glycyrrhetic acid is considerably superior to the antisclerotic drug polysponine in hypolipidemic and anticoagulant properties in case of experimental atherosclerosis and is of interest as potential antiatherosclerotic preparation (Abdullaev et al. Analysis, synthesis and pharmaceutical activity of physiological substances Tashkent: Tashk. Goss. med. in-t, 1991. p. 3).

The ammonium salt of glycyrrhetic acid and 18-dehydro-glycyrrhetic acid reduce concentration of general cholesterol, triglycerides, lipoproteins in blood plasma of rabbits with model cholesterol atherosclerosis. Owing to their powerful hypolipidemic and antioxidant effect, these compounds substantially decrease surface of atherosclerotic changes of the aorta and exceed notably Polysponine as for activity (Zakirov et al, Experim. and din. pharmacology. 1996. T. J9(5)). However, glycyrrhizic acid does not influence synthesis of cholesterol (Novikov et al Biochemistry. 1992. T. 57 (6). p. 897-903).

The mechanism of antiatherosclerotic activity of glycyrrhizic acid is explained by inhibition of phospholipase A2 activity (Inoue H., Saito H., Koshihara Y., Murota S. // Chem. Pharm. Bull. 1986. V. 34(2). P. 897-901; Yano Sh., Harada M., Watanabe K., Nakamura K., Hatakeyama Y., Shibata Sh., Takahashi K, Mori T., Hirabayashi K., TakedaM., Nagata N. // Chem. Pharm. Bull. 1989. V. 37(9). P. 2500-2504; Farina C, PinzaM., Pifferi G. // IL Farmaco. 1998. V. 53. P. 22-32). In 1964 it was shown that triterpene saponosides had specific affinity to cholesterol, destroyed cholesterol complexes with proteins and other lipid complex compounds of blood serum (Turova et al., Pharmacology and Toxicology. 1964. T. 27(2) P. 242-249). Therefore, saponosides, including glycyrrhizic acid, can be preparations for treatment of atherosclerosis.

In the experiments in vitro it has been shown that glycyrrhizic acid inhibits formation and release of lipoproteins from [14C]-glucose and binding of [14C]-cholesterol with low-molecular lipoproteins in concentration of 25-50 μm (Shiraiv et al., Chem. Abs. 1986. V. 104. 28680). The survey of antisclerotic activity of glycyrrhizic acid and its derivants has been made in the paper (Kumagai et al., Chiryogaku. 1985. V. 14 (1). P. 127-134 (Chem. Abs. 1985. V. 103. 47653).

Pharmaceutical compositions including a pharmaceutically active ingredient and optionally indicating a statin as a possible second pharmaceutically active ingredient are known in the art. Some of these publications indicate that the composition may include a sweetener, and list glycyrrhizic acid or a salt thereof as one of a list of possible sweeteners. Examples of such publications include WO2005/041962, EP2295406A, EP2172200A, WO2012/104654, WO2004/084865, EP2359812A, EP1304121A, EP2597095A, and US2007/116829. However, none of these publications disclose a specific example of a combination product containing both a statin and a glycyrrhizin. Moreover, none of the documents disclose that the glycyrrhizic acid (or salt thereof) may exhibit any pharmacological properties when administered in combination with a statin, since in all of the documents the glycyrrhizic acid component is used for taste improvement of the finished pharmaceutical form.

RU 2308947 describes a composition which is a molecular complex of simvastatin with β-glycyrrhizic acid at the molar ratio simvastatin:β-glycyrrhizic acid of between 1:1 and 1:4, and the preparation of this complex by mixing the two components in solution in a solvent such as water, ethanol or acetone.

Preparation of simvastatin with glycyrrhizic acid in the ratio 1:4 was achieved by dissolving 3.48 g of 95% of glycyrrhizic acid in 30 ml of 70% aqueous ethanol and adding to the resulting solution a solution of 0.41 g of simvastatin in 1 mL of acetone. The mixture was refluxed for 2 h, the solvents were evaporated on a rotary evaporator to precipitate evacuate (3 hours, room temperature, a residual pressure of 1 mm Hg).

Similarly, RU 2396079 describes a composition which is a molecular complex of atorvastatin with β-glycyrrhizic acid at the molar ratio atorvastatin:β-glycyrrhizic acid of between 1:1 and 1:4, and the preparation of this complex by mixing the two components in solution in a solvent such as water, ethanol or acetone. Without wishing to be bound by theory, it is believed that, in both cases, the molecular complex is formed by non-covalent interactions, such as van der Waals forces, between the two components of the combination: it is possible that the statin molecule may be a guest molecule in a micelle of 4 molecules of glycyrrhizic acid.

Stability of these complexes was determined on the basis of quantitative content of glycyrrhizic acid (in percentage form). The interval between the testing points was 7 months. The testing methodology was taken from European Pharmacopoeia 7.0. The data obtained show that both glycyrrhizic acid itself and the complex formed when glycyrrhizic acid is mixed with statin in liquid phase are unstable with regard to the factor of time because of the rapid decline of glycyrrhizic acid content. This decline is related both to the conditions of synthesis (maximum decline observed for liquid-phase synthesis) and to the properties of the active ingredient itself.

Consequently, the molecular complexes described in both of the above documents are both unstable to long-term storage (as characterized, for example, by the % content of glycyrrhizic acid reducing over time). Such reduction may be associated either with the synthesis conditions, the properties of the active substance itself, or both. In addition, the water solubility of the simvastatin/glycyrrhizic acid molecular complex described in RU 2308947 also declines with time. The instability of these molecular complexes renders them industrially unsuitable for pharmaceutical production.

### SUMMARY OF THE INVENTION

In one aspect of the invention, there is provided a combination comprising:

(a) a glycyrrhizin derivative; and
(b) a hypolipidemic drug;
provided that: wherein the hypolipidemic drug is atorvastatin, the combination does not contain a molecular complex of atorvastatin and glycyrrhizic acid; and
wherein the hypolipidemic drug is simvastatin, the combination does not contain a molecular complex of simvastatin and glycyrrhizic acid.

In one embodiment of the invention, there is provided a combination wherein the glycyrrhizin derivative and the hypolipidemic drug are present in a ratio by mass (glycyrrhizin derivative:hypolipidemic drug) of from 1:0.03 to 1:5.

In another aspect of the invention, there is provided a pharmaceutical composition comprising:
(a) a glycyrrhizin derivative; and
(b) a hypolipidemic drug;
provided that: wherein the hypolipidemic drug is atorvastatin, the composition does not contain a molecular complex of atorvastatin and glycyrrhizic acid; and wherein the hypolipidemic drug is simvastatin, the composition does not contain a molecular complex of simvastatin and glycyrrhizic acid.

In another embodiment of the invention, there is provided a pharmaceutical composition, wherein the glycyrrhizin derivative and the hypolipidemic drug are present in a ratio by mass (glycyrrhizin derivative:hypolipidemic drug) of from 1:0.03 to 1:5.

Preferably, the pharmaceutical composition is a solid pharmaceutical composition. Therefore, in one aspect of the invention, there is provided a solid pharmaceutical composition comprising:

(a) a glycyrrhizin derivative; and
(b) a hypolipidemic drug.

In another embodiment of the invention, there is provided a solid pharmaceutical composition wherein the glycyrrhizin derivative and the hypolipidemic drug are present in a ratio by mass (glycyrrhizin derivative:hypolipidemic drug) of from 1:0.03 to 1:5.

In a further aspect of the invention, there is provided the above solid pharmaceutical composition, which is a solid mixture of the glycyrrhizin derivative and the hypolipidemic drug.

More preferably, the solid pharmaceutical composition is a solid oral pharmaceutical composition. Therefore, in one embodiment, there is provided a solid oral pharmaceutical composition comprising:

(a) a glycyrrhizin derivative; and
(b) a hypolipidemic drug;
wherein the glycyrrhizin derivative and the hypolipidemic drug are present in a ratio by mass (glycyrrhizin derivative:hypolipidemic drug) of from 0.03:1 to 5:1.

In a further aspect of the invention, there is provided a kit comprising:

(a) a therapeutically effective amount of a glycyrrhizin derivative, and optionally a pharmaceutically acceptable carrier or diluent in a first unit dosage form;
(b) a therapeutically effective amount of a hypolipidemic drug, and optionally a pharmaceutically acceptable carrier or diluent in a second unit dosage form; and
(c) container means for containing said first and second dosage forms;
wherein the glycyrrhizin derivative and the hypolipidemic drug are present in a ratio by mass of from 1:0.03 to 1:5.

In a further aspect of the invention, there is provided a method of preparing the above solid pharmaceutical composition, the method comprising mixing a solid form of the glycyrrhizin and a solid form of the hypolipidemic drug.

In some embodiments, the hypolipidemic drug is a statin. Therefore, in one embodiment of the invention, there is provided a combination comprising:

(a) a glycyrrhizin derivative; and
(b) a statin;
provided that: wherein the statin is atorvastatin, the combination does not contain a molecular complex of atorvastatin and glycyrrhizic acid; and wherein the statin is simvastatin, the combination does not contain a molecular complex of simvastatin and glycyrrhizic acid.

In another embodiment of the invention, the glycyrrhizin derivative and the statin are present in a ratio by mass (glycyrrhizin derivative:statin) of from 1:0.03 to 1:5.

In another embodiment, there is provided a pharmaceutical composition comprising:

(a) a glycyrrhizin derivative; and
(b) a statin;
wherein the statin is atorvastatin, the composition does not contain a molecular complex of atorvastatin and glycyrrhizic acid; and wherein the statin is simvastatin, the composition does not contain a molecular complex of simvastatin and glycyrrhizic acid.

In another embodiment of the invention, the glycyrrhizin derivative and the statin are present in a ratio by mass (glycyrrhizin derivative:statin) of from 1:0.03 to 1:5.

Preferably, the pharmaceutical composition containing the glycyrrhizin derivative is a solid pharmaceutical composition. Therefore, in one aspect of the invention, there is provided a solid pharmaceutical composition comprising:

(a) a glycyrrhizin derivative; and
(b) a statin;
wherein the glycyrrhizin derivative and the statin are present in a ratio by mass (glycyrrhizin derivative:statin) of from 1:0.03 to 1:5.

In a further aspect of the invention, there is provided the above solid pharmaceutical composition, which is a solid mixture of the glycyrrhizin derivative and the statin.

In a further aspect of the invention, there is provided a kit comprising:

(a) a therapeutically effective amount of a glycyrrhizin derivative, and optionally a pharmaceutically acceptable carrier or diluent in a first unit dosage form;
(b) a therapeutically effective amount of a statin, and optionally a pharmaceutically acceptable carrier or diluent in a second unit dosage form; and
(c) container means for containing said first and second dosage forms;
wherein the glycyrrhizin derivative and the statin are present in a ratio by mass (glycyrrhizin derivative:statin) of from 1:0.03 to 1:5.

In a further aspect of the invention, there is provided a method of preparing the above solid pharmaceutical composition, the method comprising mixing a solid form of the glycyrrhizin and a solid form of the statin.

In some embodiments, the combination does not include combinations of glycyrrhizic acid and atorvastatin in ratios disclosed in RU 2396079, or combinations of glycyrrhizic acid and simvastatin in ratios disclosed in RU 2308947, at which these documents describe molecular complexes are formed. Therefore, in some embodiments, there is provided a combination comprising:

(a) a glycyrrhizin derivative; and
(b) a hypolipidemic drug;
wherein the glycyrrhizin derivative and the hypolipidemic drug are present in a ratio by mass (glycyrrhizin derivative:hypolipidemic drug) of from 1:0.03 to 1:5; and excluding the following combinations:
(i) glycyrrhizic acid and atorvastatin in a ratio by mass (glycyrrhizic acid:atorvastatin) of from 1:0.17 to 1:0.182; (ii) glycyrrhizic acid and atorvastatin in a ratio by mass (glycyrrhizic acid:atorvastatin) of from 1:0.45 to 1:0.5; (iii) glycyrrhizic acid and simvastatin in a ratio by mass (glycyrrhizic acid:simvastatin) of from 1:0.1 to 1:0.14; and (iv) glycyrrhizic acid and simvastatin in a ratio by mass (glycyrrhizic acid:simvastatin) of from 1:0.45 to 1:0.5.

In one embodiment, there is provided a combination comprising: (a) a glycyrrhizin derivative; and (b) a statin;

wherein the glycyrrhizin derivative and the statin are present in a ratio by mass (glycyrrhizin derivative:statin) of from 1:0.03 to 1:5; and excluding the following combinations:
(i) glycyrrhizic acid and atorvastatin in a ratio by mass (glycyrrhizic acid:atorvastatin) of from 1:0.17 to 1:0.182; (ii) glycyrrhizic acid and atorvastatin in a ratio by mass (glycyrrhizic acid:atorvastatin) of from 1:0.45 to 1:0.5; (iii) glycyrrhizic acid and simvastatin in a ratio by mass (glycyrrhizic acid:simvastatin) of from 1:0.1 to 1:0.14; and (iv) glycyrrhizic acid and simvastatin in a ratio by mass (glycyrrhizic acid:simvastatin) of from 1:0.45 to 1:0.5.

In one embodiment, there is provided a pharmaceutical composition comprising:

(a) a glycyrrhizin derivative; and a statin;

wherein the glycyrrhizin derivative and the statin are present in a ratio by mass (glycyrrhizin derivative:statin) of from 1:0.03 to 1:5; and excluding the following compositions:

(i) glycyrrhizic acid and atorvastatin in a ratio by mass (glycyrrhizic acid:atorvastatin) of from 1:0.17 to 1:0.182; (ii) glycyrrhizic acid and atorvastatin in a ratio by mass (glycyrrhizic acid:atorvastatin) of from 1:0.45 to 1:0.5; (iii) glycyrrhizic acid and simvastatin in a ratio by mass (glycyrrhizic acid:simvastatin) of from 1:0.1 to 1:0.14; and (iv) glycyrrhizic acid and simvastatin in a ratio by mass (glycyrrhizic acid:simvastatin) of from 1:0.45 to 1:0.5.

In one aspect of the invention, there is provided a combination comprising:

(a) a glycyrrhizin derivative; and a hypolipidemic drug; wherein the glycyrrhizin derivative and the hypolipidemic drug are present in a ratio by mass (glycyrrhizin derivative:hypolipidemic drug) of from 1:0.03 to 1:5.

In another aspect of the invention, there is provided a pharmaceutical composition comprising: (a) a glycyrrhizin derivative; and (b) a hypolipidemic drug; wherein the glycyrrhizin derivative and the hypolipidemic drug are present in a ratio by mass (glycyrrhizin derivative:hypolipidemic drug) of from 1:0.03 to 1:5.

In one aspect of the invention, there is provided a combination comprising:

(a) a glycyrrhizin derivative; and a statin; wherein the glycyrrhizin derivative and the statin are present in a ratio by mass (glycyrrhizin derivative:statin) of from 1:0.03 to 1:5.

In another aspect of the invention, there is provided a pharmaceutical composition comprising: (a) a glycyrrhizin derivative; and (b) a statin; wherein the glycyrrhizin derivative and the statin are present in a ratio by mass (glycyrrhizin derivative:statin) of from 1:0.03 to 1:5.

In another embodiment, the invention provides combinations, pharmaceutical compositions and kits in which the hypolipidemic drug is other than a statin. Examples include hypolipidemic drugs of the fibrate class, such as clofibrate, gemfibrozil and fenofibrate, bile acid sequestrants such as cholestipol, cholestryamine and cholesevelam, and other hypolipidemic drugs such as nicotinic acid.

Therefore, in one embodiment, there is provided a combination comprising:

(a) a glycyrrhizin derivative; and (b) a fibrate (preferably selected from the group consisting of clofibrate, gemfibrozil and fenofibrate); wherein the glycyrrhizin derivative and the fibrate are present in a ratio by mass (glycyrrhizin derivative:fibrate) of from 1:0.03 to 1:5.

In another embodiment, there is provided a pharmaceutical composition (preferably a solid pharmaceutical composition) comprising:

(a) a glycyrrhizin derivative; and
(b) a fibrate (preferably selected from the group consisting of clofibrate, gemfibrozil and fenofibrate); wherein the glycyrrhizin derivative and the fibrate are present in a ratio by mass (glycyrrhizin derivative:fibrate) of from 1:0.03 to 1:5.

Therefore, in one embodiment, there is provided a combination comprising:

(a) a glycyrrhizin derivative; and (b) a bile acid sequestrant (preferably selected from the group consisting of cholestipol, cholestryamine and cholesevelam); wherein the glycyrrhizin derivative and the bile acid sequestrant are present in a ratio by mass (glycyrrhizin derivative:bile acid sequestrant) of from 1:0.05 to 1:5.

In another embodiment, there is provided a pharmaceutical composition (preferably a solid pharmaceutical composition) comprising:

(a) a glycyrrhizin derivative; and
(b) a bile acid sequestrant (preferably selected from the group consisting of clofibrate, gemfibrozil and fenofibrate); wherein the glycyrrhizin derivative and the bile acid sequestrant are present in a ratio by mass (glycyrrhizin derivative:bile acid sequestrant) of from 1:0.05 to 1:5.

Therefore, in one embodiment, there is provided a combination comprising:

(a) a glycyrrhizin derivative; and
(b) nicotinic acid; wherein the glycyrrhizin derivative and the nicotinic acid are present in a ratio by mass (glycyrrhizin derivative:nicotinic acid) of from 1:0.05 to 1:4.

In another embodiment, there is provided a pharmaceutical composition (preferably a solid pharmaceutical composition) comprising:

(a) a glycyrrhizin derivative; and
(b) nicotinic acid; wherein the glycyrrhizin derivative and the nicotinic acid are present in a ratio by mass (glycyrrhizin derivative:nicotinic acid) of from 1:0.05 to 1:4.

In yet another aspect of the invention, there is provided any of the above combinations or pharmaceutical compositions, for use as a medicament.

In still another aspect of the invention, there is provided the combination or pharmaceutical composition, for use in treating hyperlipidemia. In one embodiment, the hyperlipidemia is selected from hypercholesterolemia (also known as hyperlipoproteinemia), hypertriglyceridemia or a co-morbidity thereof.

In yet another aspect of the invention, there is provided the above combination or pharmaceutical composition, for use in treating cardiovascular disease (including but not limited to ischemic heart disease, myocardial infarction, angina, stroke, atherosclerotic vascular disease, coronary heart disease, coronary artery disease, peripheral vascular disease, peripheral arterial disease, and intermittent claudication).

In still another aspect of the invention, there is provided use of the combination or pharmaceutical composition, in the manufacture of a medicament for treating hyperlipidemia. In one embodiment, the hyperlipidemia is selected from hypercholesterolemia (also known as hyperlipoproteinemia), hypertriglyceridemia or a co-morbidity thereof.

In yet another aspect of the invention, there is provided use of the above combination or composition, in the manufacture of a medicament for treating cardiovascular disease (including but not limited to ischemic heart disease, myocardial infarction, angina, stroke, atherosclerotic vascular disease, coronary heart disease, coronary artery disease, peripheral vascular disease, peripheral arterial disease, and intermittent claudication).

In still another aspect of the invention, there is provided a method of treating hyperlipidemia. the method comprising administering to the patient the above combination or pharmaceutical composition. In one embodiment, the hyperlipidemia is selected from hypercholesterolemia (also known as hyperlipoproteinemia), hypertriglyceridemia or a co-morbidity thereof.

In yet another aspect of the invention, there is provided a method of treating cardiovascular disease (including but not limited to ischemic heart disease, myocardial infarction, angina, stroke, atherosclerotic vascular disease, coronary heart disease, coronary artery disease, peripheral vascular disease, peripheral arterial disease, and intermittent claudication) in a patient, the method comprising administering to the patient the above combination or pharmaceutical composition.

### Advantages and Surprising Findings

The present inventors have surprisingly observed advantageous and synergistic effects when a statin is combined with a glycyrrhizin derivative, thus conferring the potential for improved properties in the treatment of diseases such as hyperlipidemia, hypercholesterolemia, hypertriglyceridemia, and cardiovascular diseases. In particular, the use of the combination has the potential to improve safety of long-term therapy with statins, since the risk of the above-described adverse effects characteristic for statins is significantly reduced.

The issue of adverse effects of statins has been solved by reducing the dose of statins by combining them with glycyrrhizin derivatives while preserving hypolipidemic effect typical for the maximum statin dose. This obviates the need to prescribe high doses of statins in long-term therapy. This could not have been predicted or expected from the prior art.

In particular, it has been unexpectedly found by the present inventors that administration of a statin (particularly but not exclusively simvastatin) in combination with a glycyrrhizin derivative (particularly but not exclusively glycyrrhizic acid or a salt thereof, such as ammonium glycyrrhizinate) resulted in an improved reduction of total blood cholesterol compared with the same dose of statin when dosed alone, and reduction of total blood cholesterol similar to the effect of higher doses of statin (such as twice the dose) when dosed alone. This favourable hypocholesterolemic efficacy of the combination therefore provides the potential to reduce the dose of statin in the composition, with the potential to reduce or eliminate some of the adverse side effects associated with statins.

In particular, it is demonstrated herein that combinations of statins and glycyrrhizinates according to the present invention (such as combinations of statins with ammonium glycyrrhizinate, glycyrrhizic acid, sodium glycyrrhizinate and glycyrrhetic acid) possess a synergistic hypocholesterolemic activity.

Furthermore, it has been unexpectedly found by the present inventors that administration of a statin (particularly but not exclusively simvastatin) in combination with a glycyrrhizin derivative (particularly but not exclusively glycyrrhizic acid or a salt thereof, such as ammonium glycyrrhizinate), while having a therapeutic effect similar to the effect of higher doses of statin (such as twice the dose) when dosed alone, exhibits much reduced adverse side effects such as hepatotoxicity and mytotoxicity when compared with this higher dose of statin. This confers the potential for statin compositions with an improved safety profile than was known in the prior art.

A solid pharmaceutical composition containing a statin and glycyrrhizin derivative in the specified mass ratio has not previously been disclosed in the art. In addition, it has been unexpectedly found by the present inventors that mixing a solid form of a statin (particularly but not exclusively simvastatin or atorvastatin) with a solid form of a glycyrrhizin derivative (particularly but not exclusively glycyrrhizic acid), in the absence of solvents, avoids the formation of the unstable molecular complexes generated when these components are mixed together in solution. This increased stability confers the potential for these mixtures to be suitable as commercial pharmaceutical products.

### BRIEF DESCRIPTION OF THE FIGURES

FIG. 1 shows the morphometry of the aorta, coloration with red of the area of arterial sclerotic disease as tested in Example 3;

FIG. 2 shows a section of rabbit's aorta, after 100× magnification, haematoxylin and eosin stained (HE stained) as described in Example 3 of the present invention;

FIG. 3 shows a section of rabbit's aorta after 100× magnification, toluidine blue stained (TB stained) as described in Example 3 of the present invention;

FIG. 4 shows a section of rabbit's aorta after 200× magnification Sudan stained as described in Example 3 of the present invention;

FIG. 5 shows a section of rabbit's aorta after 100× magnification (HE stained) as described in Example 3 of the present invention;

FIG. 6 shows a section of rabbit's aorta, the arrow showing conjunctive tissue disorganization, after 100× magnification (TB stained) as described in Example 3 of the present invention;

FIG. 7 shows a section of rabbit's aorta after 200× magnification (oil red stained) as described in Example 3 of the present invention;

FIG. 8 shows a section of rabbit's aorta (rabbit from subgroup 6A) after 100× magnification (HE stained), as described in Example 3 of the present invention, the arrow showing large atherosclerotic plaque with lipid vacuoles and foam cells;

FIG. 9 shows a section of rabbit's aorta (rabbit from subgroup 6A) after 100× magnification (TB stained) as described in Example 3 of the present invention the arrow showing conjunctive tissue disorganization (lilac staining);

FIG. 10 shows a section of rabbit's aorta (rabbit from subgroup 6A) after 200× magnification (red oil stained), as described in Example 3 of the present invention, the arrow showing lipid vacuoles and xanthome cells in aortal intima;

FIG. 11 shows a section of rabbit's aorta (rabbit from subgroup 2B) after 100× magnification (HE stained) as described in Example 3 of the present invention, the arrows showing a large atherosclerotic plaque with nuclear calcination and sclerosis;

FIG. 12 shows a section of rabbit's aorta (rabbit from subgroup 2B) after 100× magnification (TB stained), as described in Example 3 of the present invention, the arrows showing calcinosis, peripheral conjunctive tissue disorganization (lilac staining);

FIG. 13 shows a section of rabbit's aorta (rabbit from subgroup 2B) after 200× magnification (oil red stained), as described in Example 3 of the present invention, the arrows showing lipids concretions (orange staining) and nuclear calcination;

FIG. 14 shows the hepar section of intact group rabbits after 100× magnification (HE stained) as described in Example 3 of the present invention;

FIG. 15 shows the hepar section of a subgroup 7A rabbit after 100× magnification (HE stained) as described in Example 3 of the present invention, showing mostly granulose dystrophy of hepatocytes (dark arrows) and drop-size steatosis (light arrows);

FIG. 16 shows the hepar section of a subgroup 3B rabbit after 100× magnification (HE stained) as described in Example 3 of the present invention, showing mostly balloon dystrophy of hepatocytes (dark arrows) and drop-size steatosis (light arrows);

FIG. 17 shows the hepar section of a subgroup 2B rabbit after 50× magnification (HE stained) as described in Example 3 of the present invention, showing mostly balloon dystrophy of hepatocytes (dark arrows) and small-large drop steatosis (light arrows).

FIG. 18 shows the hepar section of a sub group 10A rabbit after 50× magnification (HE stained) as described in Example 3 of the present invention, showing mostly balloon dystrophy of hepatocytes (dark arrows) and small-large drop steatosis (light arrows);

FIG. 19 shows the hepar section of a sub group 11A rabbit after 100× magnification (HE stained) as described in Example 3 of the present invention, showing mostly balloon dystrophy of hepatocytes (dark arrows) and small-large drop steatosis (light arrows);

FIG. 20 shows the hepar section of a subgroup 6A rabbit after 200× magnification (HE stained) as described in Example 3 of the present invention, showing severe balloon dystrophy of hepatocytes (dark arrows) and small-large drop steatosis (light arrows);

FIG. 21 shows the hepar section of a sub group 6B rabbit after 100× magnification (HE stained) as described in Example 3 of the present invention, showing severe balloon dystrophy of hepatocytes (large with prominent vacuoles, with reduced pycnotic nuclei);

FIG. 22 shows the hepar section of a sub group 10B rabbit after 100× magnification (HE stained) as described in Example 3 of the present invention, showing expansion of conjunctive tissue in periportal zone along with severe balloon dystrophy of hepatocytes;

FIG. 23 shows the pancreas section of a subgroup 6B rabbit after 100× magnification (HE stained) as described in Example 3 of the present invention;

FIG. 24 shows the pancreas section of a subgroup 6B rabbit after 200× magnification (HE stained) as described in Example 3 of the present invention;

FIG. 25 shows the pancreas section of a subgroup 6B rabbit after 200× magnification (HE stained) as described in Example 3 of the present invention, the arrow showing pancreatic vessel wall hyalinosis;

FIG. 26 shows a section of rabbits heart valve after 100× magnification (HE stained) as described in Example 3 of the present invention;

FIG. 27 shows a section of rabbits aorta after 100× magnification (HE stained) as described in Example 3 of the present invention;

FIG. 28 shows a section of rabbits heart valve (rabbit from subgroup 9B) after 100× magnification (HE stained) as described in Example 3 of the present invention, the arrow showing foam cells and lipids deposits under valve endothelium;

FIG. 29 shows a section of rabbits heart valve (rabbit from subgroup 6A), after 100× magnification (HE stained) as described in Example 3 of the present invention, the arrow showing foam cells and lipids deposits under valve endothelium;

FIG. 30 shows a section of rabbits heart valve (rabbit from subgroup 11A), after 100× magnification (HE stained) as described in Example 3 of the present invention, the arrow showing calcification at the valve basis;

FIG. 31 shows a section of rabbit s heart valve (rabbit from subgroup 6A) after 100× magnification (HE stained) as described in Example 3 of the present invention, the arrow showing small calcified focus at the valve basis;

FIG. 32 shows the total efficacy score of studied drugs combinations as described in Example 3 of the present invention relative to monotherapy for scheme A; and

FIG. 33 shows the total efficacy score of studied drugs combinations as described in Example 3 of the present invention relative to monotherapy for scheme B.

### DETAILED DESCRIPTION

### General Definitions

Unless otherwise stated, the following terms used in this specification shall have the following meanings for the purposes of this application.

In this specification, the singular forms “a,” “an” and “the” include the plural unless the context clearly dictates otherwise.

Definitions of standard chemistry terms may be found in reference works, including Carey and Sundberg “Advanced Organic Chemistry” 4^{th} Ed. Vols. A (2000) and B (2001), Plenum Press, New York. Some specific definitions are set out below.

In this specification, unless otherwise specified, the term “combination of the present invention” refers generally to all of the aspects of the present invention (combination, pharmaceutical composition, method of preparation, method of use/treatment, kit).

“Alkyl” means a straight or branched, saturated, aliphatic radical having a chain of carbon atoms. (C_{X})alkyl and (C_{X-Y})alkyl are typically used where X and Y indicate the number of carbon atoms in the chain. For example, (C₁₋₆)alkyl includes alkyls that have a chain of between 1 and 6 carbons. Examples of alkyl include methyl, ethyl, propyl, isopropyl, butyl, sec-butyl, isobutyl, tert-butyl, pentyl and hexyl. In one embodiment alkyl is (C₁₋₁₀)alkyl. In one embodiment alkyl is (C₁₋₆)alkyl. In one embodiment alkyl is (C₁₋₄)alkyl.

“Alkoxy” means “—O-alkyl”, wherein “alkyl” is as defined above. Examples of alkoxy include methoxy, ethoxy, propoxy, isopropoxy, butyloxy, sec-butyloxy, isobutyloxy, tert-butyloxy, pentyloxy and hexyloxy. In one embodiment alkoxy is (C₁₋₁₀)alkoxy. In one embodiment alkoxy is (C₁₋₆)alkoxy. In one embodiment alkoxy is (C₁₋₄)alkoxy.

“Aryl” means a monocyclic or polycyclic ring assembly wherein each ring is aromatic (i.e. having a total number of pi electrons is equal to 4n+2, wherein n is an integer, preferably 1 or 2) or when fused with one or more rings forms an aromatic ring assembly. Examples of aryl include phenyl and naphthyl.

“Acyl” means the group R′C(═O)—, wherein R′ is a further substituent such as an alkyl group (as defined and exemplified above), an aryl group (as defined and exemplified above), or a benzyl group.

“Acyloxy” means the group R′C(═O)—O—, wherein R′ is a further substituent such as an alkyl group (as defined and exemplified above)., an aryl group (as defined and exemplified above), or a benzyl group. “Carboxyl” means the group —C(═O)—OH. “Halo” means fluorine, chlorine, bromine, or iodine. “Hydroxy” means the group —OH. “Cyano” means the group —CN. “Nitro” means the group —NO₂. “Amino” means the group —NR₂, wherein each R is independently hydrogen or alkyl (as defined and exemplified above).

Unless specified otherwise, the alkyl, alkoxy and aryl groups may be substituted by one or more substituents. The number of substituents is limited only by the number of substitutable positions, but is preferably 1, 2, 3, 4 or 5. Examples of substituents include alkyl, alkoxy, carboxy, halo, hydroxy, cyano, nitro, amino (—NR₂, wherein each R is independently hydrogen or alkyl).

“Monosaccharide” means a carbohydrate (sugar) moiety that cannot be hydrolyzed into a simpler sugar. The term “monosaccharide” is intended to cover both free monosaccharides and monosaccharide moieties which form part of a larger molecule (particularly although not exclusively monosaccharides bonded via an oxygen atom, in particular a glycoside bond), to the rest of the molecule (typically at the anomeric position). The monosaccharide may have the D- or L-configuration, and may be an aldose or ketose. In one embodiment, the monosaccharide is a hexose, examples of which include aldohexoses such as glucose, galactose, allose, altrose, mannose, gulose, idose and talose and ketohexoses such as fructose, tagatose, psicose and sorbose. In another embodiment, the monosaccharide is a pentose, examples of which include aldopentoses such as ribose, arabinose, xylose and lyxose and ketopentoses such as ribulose and xylulose. The term “monosaccharide” is also intended to cover oxidised monosaccharide moieties (where one or more primary alcohol groups are oxidised to carboxyl groups, in particular uronic acids wherein the terminal primary alcohol group of the monosaccharide is oxidised to a carboxyl group), reduced monosaccharide moieties (where one or more carbonyl groups are reduced to hydroxy groups), deoxy monosaccharide moieties (where one or more hydroxy groups are replaced with hydrogen), etherified monosaccharide moieties (where one or more free hydroxyl groups are converted to ether groups, such as alkoxy or benzyloxy groups) and esterified monosaccharide moieties (where one or more free hydroxyl groups are converted to acyloxy groups).

“Disaccharide” means a moiety having two monosaccharide moieties as defined and exemplified above, joined together by a glycoside bond. The term “disaccharide” is intended to cover both free disaccharides and disaccharide moieties which form part of a larger molecule (particularly although not exclusively disaccharides bonded via an oxygen atom, in particular a glycoside bond, on the free anomeric position), to the rest of the molecule. When the monosaccharide moieties are hexose moieties, the glycoside bonds may be 1,4′-glycoside bonds (which may be 1,4′-α- or 1,4′-β-glycoside bonds), 1,6′-glycoside bonds (which may be 1,6′-α- or 1,6′-β-glycoside bonds), 1,2′-glycoside bonds (which may be 1,2′-α- or 1,2′-βglycoside bonds), or 1,3′-glycoside bonds (which may be 1,3′-α- or 1,3′-β-glycoside bonds). Examples of suitable disaccharides include lactose, maltose, cellobiose, sucrose, trehalose, isomaltulose and trehalulose. Each of the monosaccharide moieties of the disaccharide moiety may be optionally oxidised, reduced, deoxy, etherified and/or esterified.

“Oligosaccharide” means a moiety having 3 to 10 monosaccharide moieties (as defined and exemplified above) joined together by glycoside bonds (as defined and exemplified above in a branched or unbranched chain or a ring (optionally having a saccharide side chain). In one embodiment, the monosaccharide units may be in a chain (‘chain oligosaccharides’), examples of which include maltotriose, maltotetraose, maltopentaose, maltohexaose, maltoheptaose, cellobiose, cellotriose, cellotetraose, cellopentaose, cellohexaose and celloheptaose, fructo-oligosaccharides (FOS) consist of short chains of fructose molecules; mannanoligosaccharides, isomaltooligosaccharides, galactooligosaccharides and xylooligosaccharides. In another embodiment, the monosaccharide units may form a ring ‘cyclic oligosaccharides’, typically, the ring consists of 5 to 8 monosaccharide units, preferably 6 to 8, and more preferably 6 monosaccharide units; examples of such cyclic oligosaccharides include cyclodextrins such as α-cyclodextrin (6-membered sugar ring molecule), β-cyclodextrin (7-membered sugar ring molecule) and γ-cyclodextrin (8-membered sugar ring molecule).

### Statins and Other Hypolipidemic Drugs

One element of the combination of the present invention is a hypolipidemic drug. In this specification the term “antihyperlipidemic drug” is synonymous with “hypolipidemic drug” and covers any drug which is effective in lowering blood lipid levels in a subject. The term “lipids” typically includes, for example, triglycerides, monoglycerides, diglycerides, free fatty acids, phospholipids, glycerolipids, glycerophospholipids, sphingolipids, lipoprotein (low density lipoprotein, high density lipoprotein), sterol lipids (in particular cholesterol and derivatives thereof such as cholesteryl esters), prenol lipds, saccahrolipids and polyketides.

The hypolipidemic effect of the antihyperlipidemic drug may comprise a hypocholesterolemic effect (i.e. lowering blood cholesterol levels in a subject), a hypotriglyceridemic effect (i.e. lowering blood triglyceride levels in a subject) or both. In one embodiment the antihyperlipidemic drug is a hypocholesterolemic drug (or antihypercholesterolemic drug). In one embodiment, the hypocholesterolemic effect of the drug comprises a reduction of the ratio of LDL cholesterol to HDL cholesterol. In one embodiment, the antihypercholesterolemic effect comprises a reduction of the ratio of a reduction of the ratio of total cholesterol to HDL cholesterol.

In one embodiment, one element of the combination of the present invention is a statin. In this specification the term “statin” is synonymous with “HMG-CoA reductase inhibitor” and means a compound which is capable of inhibiting HMG-CoA reductase. HMG-CoA reductase (also known as 3-hydroxy-3-methyl-glutaryl-CoA reductase) is the rate-controlling enzyme of the mevalonate pathway, the metabolic pathway that produces cholesterol as the first committed enzyme of the HMG-CoA reductase pathway. Statins take the place of HMG-CoA in the enzyme and reduce the rate by which it is able to produce mevalonate, the next molecule in the cascade that eventually produces cholesterol.

In another embodiment the combination of the present invention includes a single statin. In another embodiment the combination of the present invention includes a mixture of two or more (such as two, three or four) statins.

In one embodiment the statin is selected from the group consisting of atorvastatin, lovastatin, pravastatin, rosuvastatin, simvastatin and fluvastatin, and mixtures of any thereof.

The statin may be in its free form (i.e. not ionized) or in the form of a pharmaceutically acceptable salt (as defined and exemplified below).

In one embodiment the statin is atorvastatin. Atorvastatin is sold (as a calcium salt) by Pfizer under the trade mark Lipitor® and by a number of generic pharmaceutical manufacturers. It has the systematic name (3R,5R)-7-[2-(4-fluorophenyl)-3-phenyl-4-(phenylcarbamoyl)-5-propan-2-ylpyrrol-1-yl]-3,5-dihydroxyheptanoic acid and the structure below:

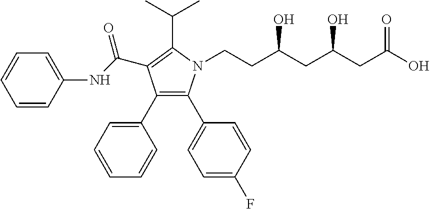 [SMILES: CC(C)c1c(C(=O)Nc2ccccc2)c(-c2ccccc2)c(-c2ccc(F)cc2)n1CC[C@@H](O)C[C@@H](O)CC(=O)O]

The synthesis of atorvastatin is described in U.S. Pat. No. 4,681,893.

In one embodiment the statin is lovastatin. Lovastatin is sold by Merck under the trade mark Mevacor®. It has the systematic name (1S,3R,7S,8S,8aR)-8-{2-[(2R,4R)-4-hydroxy-6-oxooxan-2-yl]ethyl}-3, 7-dimethyl-1,2,3,7,8,8a-hexahydronaphthalen-1-yl (2S)-2-methylbutanoate and the following structure:

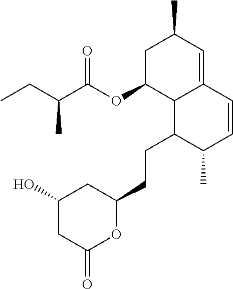 [SMILES: CC[C@H](C)C(=O)O[C@H]1C[C@@H](C)C=C2C=C[C@H](C)C(CC[C@@H]3C[C@@H](O)CC(=O)O3)C21]

Lovastatin is a naturally occurring compound found in oyster mushrooms and red yeast rice. The synthesis/isolation of lovastatin is described in patent number EP022478B and U.S. Pat. No. 4,231,983.

In one embodiment the statin is pravastatin. Pravastatin is sold by Bristol-Myers Squibb and Daiichi Sankyo under trade marks including Pravachol® and Selektine®, and by a number of generic pharmaceutical manufacturers. It has the systematic name (3R,5R)-3,5-dihydroxy-7-((1R,2S,6S,8R,8aR)-6-hydroxy-2-methyl-8-{[(2S)-2-methylbutanoyl]oxy}-1,2,6,7,8,8a-hexahydronaphthalen-1-yl)-heptanoic acid and the following structure:

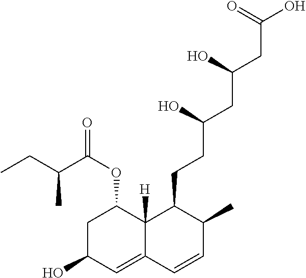 [SMILES: CC[C@H](C)C(=O)O[C@H]1C[C@H](O)C=C2C=C[C@H](C)[C@H](CC[C@@H](O)C[C@@H](O)CC(=O)O)[C@H]21]

Pravastatin can be obtained as a fermentation product of the bacterium Nocardia autotrophica. The synthesis/isolation of pravastatin is described in patent number GB2111052B.

In one embodiment the statin is rosuvastatin. Rosuvastatin is sold (as a calcium salt) by AstraZeneca under the trade mark Crestor®. It has the systematic name (3R,5S,6E)-7-[4-(4-fluorophenyl)-2-(N-methylmethanesulfonamido)-6-(propan-2-yl)pyrimidin-5-yl]-3,5-dihydroxyhept-6-enoic acid and the following structure: uvastatin is described in patent publication EP 521471A.

In one embodiment the statin is simvastatin. Simvastatin is marketed by a number of generic pharmaceutical manufacturers, and under the trade mark Zocor®. Its systematic name is (1S,3R,7S,8S,8aR)-8-{2-[(2R,4R)-4-hydroxy-6-oxotetrahydro-2H-pyran-2-yl]ethyl}-3,7-dimethyl-1,2,3,7,8,8a-hexahydronaphthalen-1-yl 2,2-dimethylbutanoate and it has the following structure:

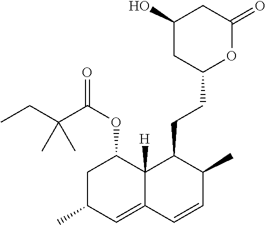 [SMILES: CCC(C)(C)C(=O)O[C@H]1C[C@@H](C)C=C2C=C[C@H](C)[C@H](CC[C@@H]3C[C@@H](O)CC(=O)O3)[C@H]21]

Simvastatin is a synthetic derivative of a fermentation product of the fungus Aspergillus terreus. The synthesis of simvastatin is described in patent number EP033538B.

In one embodiment the statin is fluvastatin. Fluvastatin is marketed by a number of generic pharmaceutical manufacturers, and under the trade mark Lescol®. It has the systematic name (3R,5S,6E)-7-[3-(4-fluorophenyl)-1-(propan-2-yl)-1H-indol-2-yl]-3,5-dihydroxyhept-6-enoic acid and the following structure:

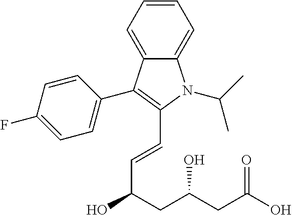 [SMILES: CC(C)n1c(/C=C/[C@H](O)C[C@H](O)CC(=O)O)c(-c2ccc(F)cc2)c2ccccc21]

The synthesis of fluvastatin is described in patent publication EP0114027B.

In one embodiment the statin is pitavastatin. Pitavastatin is sold under the trade mark Livalo®. It has the systematic name (3R,5S,6E)-7-[2-cyclopropyl-4-(4-fluorophenyl)quinolin-3-yl]-3,5-dihydroxyhept-6-enoic acid and the following structure:

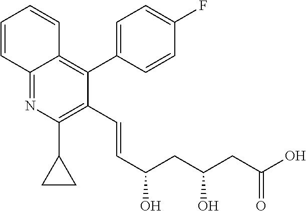 [SMILES: O=C(O)C[C@H](O)C[C@H](O)/C=C/c1c(C2CC2)nc2ccccc2c1-c1ccc(F)cc1]

The synthesis of pitavastatin is described in patent number U.S. Pat. No. 5,753,675.

In another embodiment, the combination of the present invention may comprise an antihyperlipidemic drug other than a statin. Examples of other such drugs include those of the fibrate class, such as bezafibrate (Bezalip®, ciprofibrate (Modalim®), clofibrate, gemfibrozil (Lopid®) and fenofibrate (TriCor®), bile acid sequestrants such as cholestipol, cholestryamine and cholesevelam, and other hypolipidemic drugs such as nicotinic acid.

In one embodiment, the hypolipidemic drug is not a glycyrrhizin derivative (as defined and exemplified below). In one embodiment, the hypolipidemic drug is not glycyrrhizic acid or a salt thereof. In one embodiment, the hypolipidemic drug is not glycyrrhetic acid or a salt thereof.

### Glycyrrhizin Derivative

Another element of the combination of the present invention is a glycyrrhizin derivative. In one embodiment, the glycyrrhizin derivative is a compound of the formula:

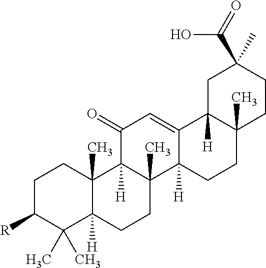

wherein R is selected from the group consisting of: hydrogen; a monosaccharide, disaccharide or oligosaccharide moiety, said moiety being optionally oxidised, reduced, deoxy, etherified and/or esterified; hydroxy; halo; amino; C₁₋₁₀ alkoxy optionally substituted by one or more substituents selected from halo, hydroxy, C₁₋₁₀ alkoxy, carboxy, alkoxy)carbonyl, or a monosaccharide, disaccharide or oligosaccharide moiety, said moiety being optionally oxidised, reduced, deoxy, etherified and/or esterified; C₁₋₁₀ alkyl optionally substituted by one or more substituents selected from halo, hydroxy, C₁₋₁₀ alkoxy, carboxy, (C₁₋₁₀ alkoxy)carbonyl, or a monosaccharide, disaccharide or oligosaccharide moiety; said moiety being optionally oxidised, reduced, deoxy, etherified and/or esterified; or a dehydro derivative thereof.

In one embodiment, R is a monosaccharide moiety. Typically, this monosaccharide moiety is attached to the rest of the molecule via a glycoside bond at its anomeric position, such that an oxygen atom links the monosaccharide moiety to the glycyrrhetic acid portion of the molecule. Examples of suitable monosaccharide moieties include aldohexoses such as glucose, galactose, allose, altrose, mannose, gulose, idose and talose; ketohexoses such as fructose, tagatose, psicose and sorbose; aldopentoses such as ribose, arabinose, xylose and lyxose; and ketopentoses such as ribulose and xylulose. A preferred monosaccharide moiety is a glucose moiety.

The monosaccharide moiety may be unmodified (i.e. having all the functional groups of the natural monosaccharide moiety) or modified. In one embodiment, the monosaccharide moiety is unmodified. In one embodiment, the monosaccharide moiety is modified. Examples of modifications include oxidation (where one or more primary alcohol groups are oxidised to carboxyl groups), reduction (where one or more carbonyl groups are reduced to hydroxy groups), deoxy (where one or more hydroxy groups are replaced with hydrogen), etherification (where one or more free hydroxyl groups are converted to ether groups, such as alkoxy or benzyloxy groups) and esterification (where one or more free hydroxyl groups are converted to acyloxy groups).

In one embodiment, the monosaccharide moiety is oxidised, typically by oxidising one or more primary alcohol groups on the moiety to carboxy groups. In one embodiment, the monosaccharide moiety is a uronic acid moiety, in which the terminal primary alcohol group on the monosaccharide moiety is oxidised to a carboxy group. Examples of such uronic acid moieties include glucuronic acid and galacturonic acid. A preferred example is a glucuronic acid moiety.

In one embodiment, R is a disaccharide moiety. Typically, this comprises a first monosaccharide moiety (as defined and exemplified above) attached to the rest of the molecule via a glycoside bond, and having a second monosaccharide moiety (as defined and exemplified above) attached to the first monosaccharide moiety via a further glycoside bond. When the monosaccharide moieties are hexose moieties, the glycoside bonds may be 1,4′-glycoside bonds (which may be 1,4′-α- or 1,4′-β-glycoside bonds), 1,6′-glycoside bonds (which may be 1,6′-α- or 1,6′-β-glycoside bonds), 1,2′-glycoside bonds (which may be 1,2′-α- or 1,2′-βglycoside bonds), or 1,3′-glycoside bonds (which may be 1,3′-α- or 1,3′-β-glycoside bonds). The first and second monosaccharide moieties may be the same or different, and are preferably selected from those monosaccharide moieties exemplified above. In one embodiment, at least one of the monosaccharide moieties of the disaccharide moiety is a glucose moiety. In one embodiment, both of the monosaccharide moieties of the disaccharide moiety are glucose moieties. In one embodiment, the disaccharide comprises two monosaccharide moieties (preferably glucose moieties) linked by a 1,2′-glycoside bond (preferably a 1,2′-β-glycoside bond).

In one embodiment, both of the two monosaccharide moieties of the disaccharide moiety are unmodified. In another embodiment, either or both (preferably both) of the two monosaccharide moieties of the disaccharide are modified, by any of the modifications exemplified above for monosaccharide groups. In one embodiment, either or both (preferably both) of the monosaccharide moieties of the disaccharide moiety are oxidised, typically by oxidising one or more primary alcohol groups on the moiety to carboxy groups. In one embodiment, either or both (preferably both) of the monosaccharide moieties of the disaccharide moiety are uronic acid moieties. In one embodiment, either or both (preferably both) of the monosaccharide moieties of the disaccharide moiety are glucuronic acid moieties. In one embodiment, the two uronic acid moieties (preferably glucuronic acid moieties) are linked by a 1,2′-glycoside bond (preferably a 1,2′-βglycoside bond).

In one embodiment, R is an acyl group R′C(═O)— (wherein R′ is a C₁₋₃₀ alkyl group optionally substituted with a carboxyl group, an aryl group such as a phenyl or naphthyl group, or a benzyl group) In this embodiment, preferably R′ is a C₁₋₆ alkyl group optionally substituted with a carboxyl group, and more preferably methyl, ethyl, propyl or 2-carboxyethyl.

In one embodiment, R is hydroxy or a monosaccharide, disaccharide or oligosaccharide moiety, said moiety being optionally oxidised, reduced, deoxy, etherified and/or esterified.

In one embodiment, R is hydroxy or a monosaccharide or disaccharide moiety, said moiety being optionally oxidised, reduced, deoxy, etherified and/or esterified.

In one embodiment, R is hydroxy or a monosaccharide or disaccharide moiety, said moiety being optionally oxidised.

In one embodiment, R is hydroxy or a disaccharide moiety, said moiety being optionally oxidised.

In one embodiment, R is hydroxy.

In one embodiment, R is a disaccharide moiety wherein both of the monosaccharide moieties are uronic acid moieties.

In one embodiment, R is a disaccharide moiety wherein both of the monosaccharide moieties are gluconic acid moieties.

In one embodiment the glycyrrhizin derivative is glycyrrhizic acid (also known as glycyrrhizin or glycyrrhizinic acid) or a pharmaceutically acceptable salt, solvate or hydrate thereof. This compound has two glucuronic acid moieties linked to one another by a 1,2′-β-glycoside bond, and linked to the glycyrrhetic acid portion of the molecule by a further glycoside bond. Glycyrrhizic acid has the systematic name (3β,18α)-30-hydroxy-11,30-dioxoolean-12-en-3-yl 2-O-β-D-glucopyranuronosyl-β-D-glucopyranosiduronic acid and the following structure:

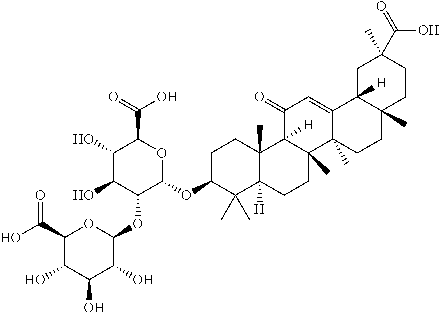

In one embodiment the glycyrrhizin derivative is glycyrrhizic acid. In another embodiment the glycyrrhizin derivative is a pharmaceutically acceptable salt of glycyrrhizic acid. Examples of pharmaceutically acceptable salts include those generally listed and exemplified below. Particularly preferred examples include salts of glycyrrhizic acid with alkali metals (such as sodium glycyrrhizinate and potassium glycyrrhizinate) and salts of glycyrrhizin with ammonia or organic amines (such as ammonium glycyrrhizinate).

In one embodiment (wherein R is hydroxy) the glycyrrhizin derivative is glycyrrhetic acid or a pharmaceutically acceptable salt, solvate or hydrate thereof. Glycyrrhetic acid (also known as glycyrrhetinic acid or enoxolone) is obtained from the hydrolysis of glycyrrhizic acid. It has the systematic name (2S,4aS,6aS,6bR,8aR,10S,12aS,12bR,14bR)-10-hydroxy-2,4a,6a,6b,9,9,12a-heptamethyl-13-oxo-1,2,3,4,4a,5,6,6a,6b,7,8,8a,9,10,11,12,12a,12b,13,14b-icosahydropicene-2-carboxylic acid and the following structure:

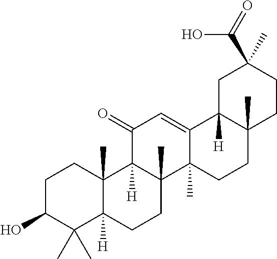

In another embodiment the glycyrrhizin derivative is a pharmaceutically acceptable salt of glycyrrhetic acid. Examples of pharmaceutically acceptable salts include those generally listed and exemplified below. Particularly preferred examples include salts of glycyrrhetic acid with alkali metals (such as sodium and potassium), and salts of glycyrrhetic acid with ammonia or organic amines (such as ammonia).

In another embodiment the glycyrrhizin derivative is a pharmaceutically acceptable derivative of glycyrrhetic acid. Various derivatives of glycyrrhetic acid can be prepared by converting the hydroxyl group into another functional group R as defined above.

Specific examples of glycyrrhetic acid derivatives include acetoxolone (wherein R is CH₃C(═O)—O—) and carbenoxolone (wherein R is HO₂C(CH₂)₂C(═O)—O—).

In another embodiment the glycyrrhizin derivative is a dehydro derivative of glycyrrhetic acid or glycyrrhizic acid. Such a derivative has a double bond between carbons 18 and 19, and is of the following general structure:

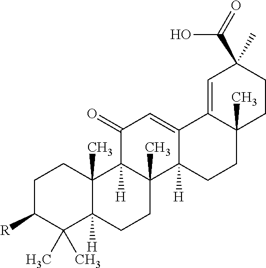

wherein R is as defined and exemplified above with respect to glycyrrhizic acid derivatives.

In one embodiment, the glycyrrhizin derivative may be present in pure form. In another embodiment, the glycyrrhizin derivative may be present in the combination in the form of a mixture with other ingredients, typically those other ingredients found in the natural sources, such as liquorice (Glycyrrhiza glabra) from which the compound is usually derived. Typically, these other ingredients comprise other terpenoid glycosides. The other ingredients may be pharmaceutically inactive or may also have a pharmacological effect comparable to the glycyrrhizin derivatives referred to above. In one embodiment, the glycyrrhizin derivative comprises at least 10%, such as at least 15%, such as at least 20%, such as at least 25%, such as at least 30%, such as at least 35%, such as at least 40%, such as at least 45%, such as at least 50%, such as at least 55%, such as at least 60%, such as at least 65%, such as at least 70%, such as at least 75%, such as at least 80%, such as at least 85%, such as at least 90%, such as at least 95%, such as at least 97%, such as at least 98%, such as at least 99%, such as at least 99.5%, by weight of the mixture.

### Preferred Combinations

In one embodiment, the statin is simvastatin or a pharmaceutically acceptable salt or solvate thereof, and the glycyrrhizin derivative is glycyrrhizic acid or a pharmaceutically acceptable salt or solvate thereof. In one embodiment, the statin is simvastatin and the glycyrrhizin derivative is monoammonium glycyrrhizinate.

In one embodiment, the statin is atorvastatin or a pharmaceutically acceptable salt or solvate thereof, and the glycyrrhizin derivative is glycyrrhizic acid or a pharmaceutically acceptable salt or solvate thereof. In one embodiment, the statin is atorvastatin and the glycyrrhizin derivative is glycyrrhizic acid. In one embodiment, the statin is atorvastatin and the glycyrrhizin derivative is monoammonium glycyrrhizinate.

In one embodiment, the statin is lovastatin or a pharmaceutically acceptable salt or solvate thereof, and the glycyrrhizin derivative is glycyrrhizic acid or a pharmaceutically acceptable salt or solvate thereof. In one embodiment, the statin is lovastatin and the glycyrrhizin derivative is mono-, di- or trisodium glycyrrhizinate.

In one embodiment, the statin is pravastatin or a pharmaceutically acceptable salt or solvate thereof, and the glycyrrhizin derivative is glycyrrhetic acid or a pharmaceutically acceptable salt or solvate thereof. In one embodiment, the statin is pravastatin and the glycyrrhizin derivative is glycyrrhetic acid.

In one embodiment, the statin is rosuvastatin or a pharmaceutically acceptable salt or solvate thereof, and the glycyrrhizin derivative is glycyrrhizic acid or a pharmaceutically acceptable salt or solvate thereof. In one embodiment, the statin is rosuvastatin and the glycyrrhizin derivative is monoammonium glycyrrhizinate.

In one embodiment, the statin is fluvastatin or a pharmaceutically acceptable salt or solvate thereof, and the glycyrrhizin derivative is glycyrrhizic acid or a pharmaceutically acceptable salt or solvate thereof. In one embodiment, the statin is fluvastatin and the glycyrrhizin derivative is glycyrrhizic acid.

In one embodiment, the composition does not contain a molecular complex of the statin and the glycyrrhizin derivative. In this specification the term “molecular complex” means a complex wherein the statin and the glycyrrhizin derivative are bonded together. In one embodiment the term “molecular complex” means a complex wherein the statin and the glycyrrhizin derivative are bonded by a covalent bond. In one embodiment the term “molecular complex” means a complex wherein the statin and the glycyrrhizin derivative are bonded by non-covalent interactions, for example electrostatic interactions such as van der Waals forces, dipole-dipole interactions or hydrogen bonding. Wherein the statin is atorvastatin, in one embodiment, the composition does not contain a molecular complex of atorvastatin and glycyrrhizic acid. Wherein the statin is simvastatin, in one embodiment, the composition does not contain a molecular complex of simvastatin and glycyrrhizic acid.

In some embodiments, the combinations prepared by liquid-phase synthesis as described in RU 2308947 and RU 2396079 (particularly Examples 1 and 2 of each document) are excluded from the present invention. As described herein, combinations containing such molecular complexes are unstable with regard to the factor of time because of the rapid decline of glycyrrhizic acid content. In addition, the water solubility of the simvastatin/glycyrrhizic acid molecular complex described in RU 2308947 also declines with time. The instability of these molecular complexes renders them industrially unsuitable for pharmaceutical production.

Therefore, in particular embodiments, the following combinations are excluded:

(i) a combination (especially a composition prepared by liquid-phase synthesis, such as using solvents, e.g. those selected from ethanol, acetone or any mixtures thereof) containing glycyrrhizic acid and atorvastatin in a ratio by mass (glycyrrhizic acid:atorvastatin) of from 1:0.17 to 1:0.182, such as 1:0.171 to 1:0.18, such as 1:0.171 to 1:0.173 or 1:0.18 to 1:0.182;
(ii) a combination (especially a composition prepared by liquid-phase synthesis, such as using solvents, e.g. those selected from ethanol, acetone or any mixtures thereof) containing glycyrrhizic acid and atorvastatin in a ratio by mass (glycyrrhizic acid:atorvastatin) of from 1:0.45 to 1:0.5; such as 1:0.47 to 1.0.5, such as 1:0.47 to 1:0.472 or 1:0.495 to 1:0.497;
(iii) a combination (especially a composition prepared by liquid-phase synthesis, such as using solvents, e.g. those selected from ethanol, acetone or any mixtures thereof) containing glycyrrhizic acid and simvastatin in a ratio by mass (glycyrrhizic acid:simvastatin) of from 1:0.1 to 1:0.14, such as 1:0.115 to 1:0.125, such as 1:0.116 to 1:0.118 or 1:0.123 to 0:0.125; and (iv) a combination (especially a composition prepared by liquid-phase synthesis, such as using solvents, e.g. those selected from ethanol, acetone or any mixtures thereof) glycyrrhizic acid and simvastatin in a ratio by mass (glycyrrhizic acid:simvastatin) of from 1:0.45 to 1:0.5 such as 1:0.47 to 1.0.5, such as 1:0.47 to 1:0.472 or 1:0.495 to 1:0.497.

### Preferred Ratios of Glycyrrhizin Derivative to Statin

In one embodiment of the combination of the present invention, the glycyrrhizin derivative and the hypolipidemic drug (preferably statin) are present in a ratio by mass (glycyrrhizin derivative:hypolipidemic drug) of from 1:0.03 to 1:5. Preferred mass ratios of the combination of the present invention are expressed below. In this specification, the mass ratios are calculated by the mass of each active ingredient of the component (i.e. excluding the contribution of a counter-ion when the active ingredient is in a salt form). In particular, when the active ingredient possesses a free acid group, the mass is calculated based on the free acid form, excluding any counter-ions. Similarly, when the active ingredient possesses a free base group, the mass is calculated based on the free base form, excluding any counter-ions.

In one embodiment of the combination of the present invention, the glycyrrhizin derivative and the hypolipidemic drug (preferably statin) are present in a ratio by mass (glycyrrhizin derivative:statin) of from 1:0.03 to 1:2. In one embodiment of the combination of the present invention, the glycyrrhizin derivative and the hypolipidemic drug (preferably statin) are present in a ratio by mass (glycyrrhizin derivative:statin) of from 1:0.05 to 1:2. In one embodiment of the combination of the present invention, the glycyrrhizin derivative and the hypolipidemic drug (preferably statin) are present in a ratio by mass (glycyrrhizin derivative:statin) of from 1:0.05 to 1:1.

In one embodiment of the combination of the present invention, the glycyrrhizin derivative and the hypolipidemic drug (preferably statin) are present in a ratio by mass (glycyrrhizin derivative:statin) of from 1:0.03 to 1:0.5. In one embodiment of the combination of the present invention, the glycyrrhizin derivative and the hypolipidemic drug (preferably statin) are present in a ratio by mass of from 1:0.1 to 1:0.45. In one embodiment of the combination of the present invention, the glycyrrhizin derivative and the hypolipidemic drug (preferably statin) are present in a ratio by mass of from 1:0.1 to 1:0.3. In one embodiment of the combination of the present invention, the glycyrrhizin derivative and the hypolipidemic drug (preferably statin) are present in a ratio by mass of from 1:0.15 to 1:0.45. In one embodiment of the combination of the present invention, the glycyrrhizin derivative and the hypolipidemic drug (preferably statin) are present in a ratio by mass of from 1:0.2 to 1:0.4. In one embodiment of the combination of the present invention, the glycyrrhizin derivative and the hypolipidemic drug (preferably statin) are present in a ratio by mass of from 1:0.2 to 1:0.35. In one embodiment of the combination of the present invention, the glycyrrhizin derivative and the hypolipidemic drug (preferably statin) are present in a ratio by mass of from 1:1 to 1:0.8.

In one embodiment, the statin is simvastatin or a pharmaceutically acceptable salt or solvate thereof, the glycyrrhizin derivative is glycyrrhizic acid or a pharmaceutically acceptable salt or solvate thereof, and the glycyrrhizic acid or pharmaceutically acceptable salt or solvate thereof and the simvastatin or pharmaceutically acceptable salt or solvate thereof are present in a ratio by mass of from 1:0.1 to 1:0.3, preferably 1:0.15 to 1:0.25, more preferably 1:0.17 to 1:0.21, even more preferably 1:0.18 to 1:0.2, still more preferably 1:0.182 to 1:0.2, even more preferably 1:0.184 to 1:0.195, and most preferably 1:0.185 to 1:0.19.

In one embodiment, the statin is simvastatin, the glycyrrhizin derivative is ammonium glycyrrhizinate, and the ammonium glycyrrhizinate and the simvastatin are present in a ratio by mass of from 1:0.1 to 1:0.3, preferably 1:0.15 to 1:0.25, more preferably 1:0.17 to 1:0.21, and most preferably 1:0.18 to 1:0.2.

In one embodiment, the statin is atorvastatin or a pharmaceutically acceptable salt or solvate thereof, the glycyrrhizin derivative is glycyrrhizic acid or a pharmaceutically acceptable salt or solvate thereof, and the glycyrrhizic acid or pharmaceutically acceptable salt or solvate thereof and the atorvastatin or pharmaceutically acceptable salt or solvate thereof are present in a ratio by mass of from 1:0.01 to 1:0.2, preferably 1:0.05 to 1:0.15, more preferably 1:0.07 to 1:0.11, and most preferably 1:0.09 to 1:0.13.

In one embodiment, the statin is atorvastatin calcium salt, the glycyrrhizin derivative is glycyrrhizic acid, and the glycyrrhizic acid and the atorvastatin are present in a ratio by mass of from 1:0.01 to 1:0.2, preferably 1:0.05 to 1:0.15, more preferably 1:0.07 to 1:0.11, and most preferably 1:0.09 to 1:0.13.

In one embodiment, the statin is lovastatin or a pharmaceutically acceptable salt or solvate thereof, the glycyrrhizin derivative is glycyrrhizic acid or a pharmaceutically acceptable salt or solvate thereof, and the glycyrrhizic acid or pharmaceutically acceptable salt or solvate thereof and the lovastatin or pharmaceutically acceptable salt or solvate thereof are present in a ratio by mass of from 1:0.1 to 1:0.3, preferably 1:0.15 to 1:0.25, more preferably 1:0.17 to 1:0.21, and most preferably 1:0.18 to 1:0.2.

In one embodiment, the statin is lovastatin, the glycyrrhizin derivative is sodium glycyrrhizinate, and the sodium glycyrrhizinate and the lovastatin are present in a ratio by mass of from 1:0.1 to 1:0.3, preferably 1:0.15 to 1:0.25, more preferably 1:0.17 to 1:0.21, and most preferably 1:0.18 to 1:0.2.

In one embodiment, the statin is pravastatin or a pharmaceutically acceptable salt or solvate thereof, the glycyrrhizin derivative is glycyrrhetic acid or a pharmaceutically acceptable salt or solvate thereof, and the glycyrrhetic acid or pharmaceutically acceptable salt or solvate thereof and the pravastatin or pharmaceutically acceptable salt or solvate thereof are present in a ratio by mass of from 1:0.3 to 1:0.5, preferably 1:0.35 to 1:0.45, more preferably 1:0.35 to 1:0.4, and most preferably 1:0.36 to 1:0.38.

In one embodiment, the statin is pravastatin, the glycyrrhizin derivative is glycyrrhetic acid, and the glycyrrhetic acid and the pravastatin are present in a ratio by mass of from 1:0.3 to 1:0.5, preferably 1:0.35 to 1:0.45, more preferably 1:0.35 to 1:0.4, and most preferably 1:0.36 to 1:0.38.

In one embodiment, the statin is rosuvastatin or a pharmaceutically acceptable salt or solvate thereof, the glycyrrhizin derivative is glycyrrhizic acid or a pharmaceutically acceptable salt or solvate thereof, and the glycyrrhizic acid or pharmaceutically acceptable salt or solvate thereof and the rosuvastatin or pharmaceutically acceptable salt or solvate thereof are present in a ratio by mass of from 1:0.01 to 1:0.2, preferably 1:0.05 to 1:0.15, more preferably 1:0.07 to 1:0.11, and most preferably 1:0.08 to 1:0.12.

In one embodiment, the statin is rosuvastatin calcium salt, the glycyrrhizin derivative is ammonium glycyrrhizinate, and the ammonium glycyrrhizinate and the rosuvastatin are present in a ratio by mass of from 1:0.01 to 1:0.2, preferably 1:0.05 to 1:0.15, more preferably 1:0.07 to 1:0.11, and most preferably 1:0.08 to 1:0.12.

In one embodiment, the statin is fluvastatin or a pharmaceutically acceptable salt or solvate thereof, the glycyrrhizin derivative is glycyrrhizic acid or a pharmaceutically acceptable salt or solvate thereof, and the glycyrrhizic acid or pharmaceutically acceptable salt or solvate thereof and the fluvastatin or pharmaceutically acceptable salt or solvate thereof are present in a ratio by mass of from 1:1 to 1:0.8, preferably 1:0.95 to 1:0.85, more preferably 1:0.925 to 1:0.875, and most preferably 1:0.9 to 1:0.88.

In one embodiment, the statin is fluvastatin, the glycyrrhizin derivative is glycyrrhizic acid, and the glycyrrhizic acid and the fluvastatin are present in a ratio by mass of from 1:1 to 1:0.8, preferably 1:0.95 to 1:0.85, more preferably 1:0.925 to 1:0.875, and most preferably 1:0.9 to 1:0.88.

In one embodiment, the statin is pitavastatin or a pharmaceutically acceptable salt or solvate thereof, the glycyrrhizin derivative is glycyrrhetic acid or a pharmaceutically acceptable salt or solvate thereof, and the glycyrrhetic acid or pharmaceutically acceptable salt or solvate thereof and the pitavastatin or pharmaceutically acceptable salt or solvate thereof are present in a ratio by mass of from 1:0.2 to 1:0.5, preferably 1:0.35 to 1:0.45, more preferably 1:0.35 to 1:0.4, and most preferably 1:0.36 to 1:0.48.

In one embodiment, the statin is pitavastatin, the glycyrrhizin derivative is glycyrrhetic acid, and the glycyrrhetic acid and the pitavastatin are present in a ratio by mass of from 1:0.2 to 1:0.5, preferably 1:0.25 to 1:0.45, more preferably 1:0.35 to 1:0.4, and most preferably 1:0.36 to 1:0.48.

### Salts, Solvates, Hydrates, and Prodrugs

It should be recognized that the compounds used in the combination of the present invention may be present and optionally administered in the form of salts, solvates hydrates and prodrugs that are converted in vivo into the compounds used in the combination of the present invention. For example, it is within the scope of the present invention to use the compounds of the present invention in the form of their pharmaceutically acceptable salts derived from various organic and inorganic acids and bases in accordance with procedures well known in the art. In this specification the term “pharmaceutically acceptable salt”, is intended to encompass any compound used in the combination of the present invention the form of a salt thereof.

When the compounds used in the combination of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids and their corresponding salts such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptonate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, isobutyrate, lactate, lactobionate, malate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds used in the combination of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

Compounds of the present invention may also be conveniently prepared, or formed during the process of the invention, as solvates (e.g., hydrates). Hydrates of compounds of the present invention may be conveniently prepared by recrystallization from an aqueous/organic solvent mixture, using organic solvents such as dioxane, tetrahydrofuran or methanol.

Prodrug derivatives of compounds used in the combination of the present invention can be prepared by modifying substituents of compounds of the present invention that are then converted in vivo to a different substituent. For example, prodrugs can be prepared by reacting a compound with a carbamylating agent (e.g., 1,1-acyloxy-alkylcarbonochloridate, para-nitrophenyl carbonate, or the like) or an acylating agent. Further examples of methods of making prodrugs are described in Saulnier et al., Bioorganic and Medicinal Chemistry Letters, 1994, 4, 1985).

### Pharmaceutical Compositions

A wide variety of compositions may be used to deliver the combination of the present invention. Such compositions may include, in addition to the statin and glycyrrhizin derivative used in the combination of the present invention, conventional pharmaceutical excipients, and other conventional, pharmaceutically inactive agents. Additionally, the compositions may include active agents in addition to the statin and glycyrrhizin derivative used in the combination of the present invention. These additional active agents may include additional statin and/or glycyrrhizin derivatives according to the invention, and/or one or more other pharmaceutically active agents.

Compositions comprising the combination of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. Preferably, the combination of the present invention may be administered or coadministered orally. The compounds and/or compositions according to the invention may also be administered or coadministered in slow release dosage forms.

Oral pharmaceutical dosage forms may be as a solid, gel or liquid. Examples of solid dosage forms include, but are not limited to tablets, capsules, granules, and bulk powders. More specific examples of oral tablets include compressed, chewable lozenges and tablets that may be enteric-coated, sugar-coated or film-coated. Examples of capsules include hard or soft gelatin capsules. Granules and powders may be provided in non-effervescent or effervescent forms. Each may be combined with other ingredients known to those skilled in the art.

In addition to the statin and glycyrrhizin derivative used in the combination according to the present invention, the composition may comprise: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyvinylpyrrolidine, celluloses and derivatives thereof, crospovidones and other such binders known to those of skill in the art.

Liquid pharmaceutically administrable compositions can, for example, be prepared by dissolving, dispersing, or otherwise mixing the active compounds as defined above and optional pharmaceutical adjuvants in a carrier, such as, for example, water, saline, aqueous dextrose, glycerol, glycols, ethanol, and the like, to form a solution or suspension. If desired, the pharmaceutical composition to be administered may also contain minor amounts of auxiliary substances such as wetting agents, emulsifying agents, or solubilizing agents, pH buffering agents and the like, for example, acetate, sodium citrate, cyclodextrin derivatives, sorbitan monolaurate, triethanolamine sodium acetate, triethanolamine oleate, and other such agents. Actual methods of preparing such dosage forms are known in the art, or will be apparent, to those skilled in this art; for example, see Remington's Pharmaceutical Sciences, Mack Publishing Company, Easton, Pa., 15th Edition, 1975.

### Dosage

Preferred doses by mass of each ingredient of the combination of the present invention are expressed below. In one embodiment, the mass is expressed as the total mass of each ingredient of the component (i.e. excluding the mass contribution of a counter-ion when the active ingredient is in a salt form). In one embodiment, the mass is expressed as the mass of active ingredient of the component (i.e. excluding the contribution of a counter-ion when the active ingredient is in a salt form). Unless otherwise specified, the mass of the ingredient may be expressed as either of the above quantities.

In one embodiment, the statin is simvastatin and the simvastatin is dosed at 0.1 to 200 mg/day, preferably 1 to 100 mg/day, more preferably 5 to 50 mg/day, even more preferably 10 to 30 mg/day, still more preferably 15 to 25 mg/day, and most preferably 20 mg/day.

In one embodiment, the statin is atorvastatin and the atorvastatin is dosed at 0.05 to 100 mg/day, preferably 0.5 to 50 mg/day, more preferably 1 to 40 mg/day, even more preferably 2 to 20 mg/day, still more preferably 5 to 15 mg/day, and most preferably 10 mg/day.

In one embodiment, the statin is lovastatin and the lovastatin is dosed at 0.1 to 200 mg/day, preferably 1 to 100 mg/day, more preferably 5 to 50 mg/day, even more preferably 10 to 30 mg/day, still more preferably 15 to 25 mg/day, and most preferably 20 mg/day.

In one embodiment, the statin is pravastatin and the pravastatin is dosed at 0.02 to 400 mg/day, preferably 1 to 200 mg/day, more preferably 2 to 100 mg/day, even more preferably 5 to 50 mg/day, still more preferably 20 to 30 mg/day, and most preferably 40 mg/day.

In one embodiment, the statin is rosuvastatin and the rosuvastatin is dosed at 0.05 to 100 mg/day, preferably 0.5 to 50 mg/day, more preferably 1 to 40 mg/day, even more preferably 2 to 20 mg/day, still more preferably 5 to 15 mg/day, and most preferably 10 mg/day.

In one embodiment, the statin is fluvastatin and the fluvastatin is dosed at 0.1 to 800 mg/day, preferably 1 to 400 mg/day, more preferably 20 to 200 mg/day, even more preferably 40 to 120 mg/day, still more preferably 60 to 100 mg/day, and most preferably 80 mg/day.

In one embodiment, the statin is pitavastatin and the pitavastatin is dosed at 0.2 to 800 mg/day, preferably 0.5 to 200 mg/day, more preferably 2 to 100 mg/day, even more preferably 5 to 50 mg/day, still more preferably 10 to 30 mg/day, and most preferably 40 mg/day.

In one embodiment, the hypolipidemic drug is a fibrate and the fibrate is dosed at 9 to 9300 mg/day, preferably 45 to 600 mg/day.

In one embodiment, the hypolipidemic drug is a bile acid sequestrant and the bile acid sequestrant is dosed at 9 to 9300 mg/day, preferably 625 to 4000 mg/day.

In one embodiment, the hypolipidemic drug is nicotinic acid and the nicotinic acid is dosed at 22.5 to 9300 mg/day, preferably 50 to 1000 mg/day.

In one embodiment, the glycyrrhizin derivative is glycyrrhizic acid or a pharmaceutically acceptable salt, solvate or hydrate thereof and the glycyrrhizic acid or pharmaceutically acceptable salt, solvate or hydrate thereof is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day further more preferably 80 to 120 mg/day, yet further more preferably 85 to 110 mg/day and most preferably 90 or 108 mg/day.

In one embodiment, the glycyrrhizin derivative is glycyrrhizic acid and the glycyrrhizic acid is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day, further more preferably 80 to 120 mg/day, yet further more preferably 85 to 100 mg/day and most preferably 90 mg/day.

In one embodiment, the glycyrrhizin derivative is glycyrrhetic acid or a pharmaceutically acceptable salt, solvate or hydrate thereof and the glycyrrhetic acid or pharmaceutically acceptable salt, solvate or hydrate thereof is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day further more preferably 80 to 120 mg/day, yet further more preferably 100 to 110 mg/day and most preferably 108 mg/day.

In one embodiment, the glycyrrhizin derivative is glycyrrhetic acid and the glycyrrhetic acid is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day, further more preferably 80 to 120 mg/day, yet further more preferably 100 to 110 mg/day and most preferably 108 mg/day.

In one embodiment, the glycyrrhizin derivative is ammonium glycyrrhizinate and the ammonium glycyrrhizinate is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day, further more preferably 80 to 120 mg/day, yet further more preferably 100 to 110 mg/day and most preferably 108 mg/day.

In one embodiment, the glycyrrhizin derivative is sodium glycyrrhizinate and the sodium glycyrrhizinate is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day further more preferably 80 to 120 mg/day, yet further more preferably 100 to 110 mg/day and most preferably 108 mg/day.

In one embodiment, the statin is simvastatin or a pharmaceutically acceptable salt, solvate or hydrate thereof, the glycyrrhizin derivative is glycyrrhizic acid or a pharmaceutically acceptable salt, solvate or hydrate thereof, the simvastatin or pharmaceutically acceptable salt, solvate or hydrate thereof is dosed at 0.1 to 200 mg/day, preferably 1 to 100 mg/day, more preferably 5 to 50 mg/day, even more preferably 10 to 30 mg/day, still more preferably 15 to 25 mg/day, and most preferably 20 mg/day, and the glycyrrhizic acid or pharmaceutically acceptable salt, solvate or hydrate thereof is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day and most preferably 80 to 120 mg/day. In one embodiment, the statin is simvastatin, the glycyrrhizin derivative is ammonium glycyrrhizinate, the simvastatin is dosed at 0.1 to 200 mg/day, preferably 1 to 100 mg/day, more preferably 5 to 50 mg/day, even more preferably 10 to 30 mg/day, still more preferably 15 to 25 mg/day, and most preferably 20 mg/day, and the ammonium glycyrrhizinate is dosed at 5 to 1000 mg/day, preferably 5 to 500 mg/day, more preferably 5 to 400 mg/day, even more preferably 10 to 300 mg/day, still more preferably 20 to 250 mg/day, yet more preferably 50 to 200 mg/day, further more preferably 80 to 120 mg/day, yet further more preferably 100 to 110 mg/day and most preferably 108 mg/day.

In one embodiment, the statin is atorvastatin or a pharmaceutically acceptable salt, solvate or hydrate thereof, the glycyrrhizin derivative is glycyrrhizic acid or a pharmaceutically acceptable salt, solvate or hydrate thereof, the atorvastatin or pharmaceutically acceptable salt, solvate or hydrate thereof is dosed at 0.05 to 100 mg/day, preferably 0.5 to 50 mg/day, more preferably 1 to 40 mg/day, even more preferably 2 to 20 mg/day, still more preferably 5 to 15 mg/day, and most preferably 10 mg/day, and the glycyrrhizic acid or pharmaceutically acceptable salt, solvate or hydrate thereof is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day and most preferably 80 to 120 mg/day In one embodiment, the statin is atorvastatin, the glycyrrhizin derivative is glycyrrhizic acid, the atorvastatin is dosed at 0.05 to 100 mg/day, preferably 0.5 to 50 mg/day, more preferably 1 to 40 mg/day, even more preferably 2 to 20 mg/day, still more preferably 5 to 15 mg/day, and most preferably 10 mg/day, and the glycyrrhizic acid is dosed at 0.5 to 1000 mg/day, preferably 5 to 500 mg/day, more preferably 5 to 400 mg/day, even more preferably 10 to 300 mg/day, still more preferably 20 to 250 mg/day, yet more preferably 50 to 200 mg/day, further more preferably 80 to 120 mg/day, yet further more preferably 85 to 100 mg/day and most preferably 90 mg/day.

In one embodiment, the statin is lovastatin or a pharmaceutically acceptable salt, solvate or hydrate thereof, the glycyrrhizin derivative is glycyrrhizic acid or a pharmaceutically acceptable salt, solvate or hydrate thereof, the lovastatin or a pharmaceutically acceptable salt, solvate or hydrate thereof is dosed at 0.1 to 200 mg/day, preferably 1 to 100 mg/day, more preferably 5 to 50 mg/day, even more preferably 10 to 30 mg/day, still more preferably 15 to 25 mg/day, and most preferably 20 mg/day, and the glycyrrhizic acid or a pharmaceutically acceptable salt, solvate or hydrate thereof is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day and most preferably 80 to 120 mg/day. In one embodiment, the statin is lovastatin, the glycyrrhizin derivative is sodium glycyrrhizinate, the lovastatin is dosed at 0.1 to 200 mg/day, preferably 1 to 100 mg/day, more preferably 5 to 50 mg/day, even more preferably 10 to 30 mg/day, still more preferably 15 to 25 mg/day, and most preferably 20 mg/day, and the sodium glycyrrhizinate is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day further more preferably 80 to 120 mg/day, yet further more preferably 100 to 110 mg/day and most preferably 108 mg/day.

In one embodiment, the statin is pravastatin or a pharmaceutically acceptable salt, solvate or hydrate thereof, the glycyrrhizin derivative is glycyrrhetic acid or a pharmaceutically acceptable salt or solvate thereof, the pravastatin or pharmaceutically acceptable salt, solvate or hydrate thereof is dosed at 0.02 to 400 mg/day, preferably 1 to 200 mg/day, more preferably 2 to 100 mg/day, even more preferably 5 to 50 mg/day, still more preferably 20 to 30 mg/day, and most preferably 40 mg/day, and the glycyrrhetic acid or pharmaceutically acceptable salt, solvate or hydrate thereof is dosed at 0.5 to 1000 mg/day, preferably 5 to 500 mg/day, more preferably 5 to 400 mg/day, even more preferably 10 to 300 mg/day, still more preferably 20 to 250 mg/day, yet more preferably 50 to 200 mg/day and most preferably 80 to 120 mg/day. In one embodiment, the statin is pravastatin, the glycyrrhizin derivative is glycyrrhetic acid, the pravastatin is dosed at 0.02 to 400 mg/day, preferably 1 to 200 mg/day, more preferably 2 to 100 mg/day, even more preferably 5 to 50 mg/day, still more preferably 20 to 30 mg/day, and most preferably 40 mg/day, and the glycyrrhetic acid is dosed at 0.5 to 1000 mg/day, preferably 5 to 500 mg/day, more preferably 5 to 400 mg/day, even more preferably 10 to 300 mg/day, still more preferably 20 to 250 mg/day, yet more preferably 50 to 200 mg/day further more preferably 80 to 120 mg/day, yet further more preferably 100 to 110 mg/day and most preferably 108 mg/day.

In one embodiment, the statin is rosuvastatin or a pharmaceutically acceptable salt, solvate or hydrate thereof, and the glycyrrhizin derivative is glycyrrhizic acid or a pharmaceutically acceptable salt, solvate or hydrate thereof, the rosuvastatin is dosed at 0.05 to 100 mg/day, preferably 0.5 to 50 mg/day, more preferably 1 to 40 mg/day, even more preferably 2 to 20 mg/day, still more preferably 5 to 15 mg/day, and most preferably 10 mg/day, and the glycyrrhizic acid or a pharmaceutically acceptable salt, solvate or hydrate thereof is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day and most preferably 80 to 120 mg/day. In one embodiment, the statin is rosuvastatin, the glycyrrhizin derivative is ammonium glycyrrhizinate, the rosuvastatin is dosed at 0.05 to 100 mg/day, preferably 0.5 to 50 mg/day, more preferably 1 to 40 mg/day, even more preferably 2 to 20 mg/day, still more preferably 5 to 15 mg/day, and most preferably 10 mg/day, and the ammonium glycyrrhizinate is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day, further more preferably 80 to 120 mg/day, yet further more preferably 100 to 110 mg/day and most preferably 108 mg/day.

In one embodiment, the statin is fluvastatin or a pharmaceutically acceptable salt, solvate or hydrate thereof, and the glycyrrhizin derivative is glycyrrhizic acid or a pharmaceutically acceptable salt, solvate or hydrate thereof, the fluvastatin or pharmaceutically acceptable salt, solvate or hydrate thereof is dosed at 0.04 to 800 mg/day, preferably 1 to 400 mg/day, more preferably 20 to 200 mg/day, even more preferably 40 to 120 mg/day, still more preferably 60 to 100 mg/day, and most preferably 80 mg/day and the glycyrrhizic acid or a pharmaceutically acceptable salt, solvate or hydrate thereof is dosed at 5 to 1000 mg/day, preferably 5 to 500 mg/day, more preferably 5 to 400 mg/day, even more preferably 10 to 300 mg/day, still more preferably 20 to 250 mg/day, yet more preferably 50 to 200 mg/day and most preferably 80 to 120 mg/day. In one embodiment, the statin is fluvastatin, the glycyrrhizin derivative is glycyrrhizic acid, the fluvastatin is dosed at 0.04 to 800 mg/day, preferably 1 to 400 mg/day, more preferably 20 to 200 mg/day, even more preferably 40 to 120 mg/day, still more preferably 60 to 100 mg/day, and most preferably 80 mg/day, and the glycyrrhizic acid is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day, further more preferably 80 to 120 mg/day, yet further more preferably 85 to 100 mg/day and most preferably 90 mg/day.

In one embodiment, the statin is pitavastatin or a pharmaceutically acceptable salt, solvate or hydrate thereof, and the glycyrrhizin derivative is glycyrrhizic acid or a pharmaceutically acceptable salt, solvate or hydrate thereof, the pitavastatin or pharmaceutically acceptable salt, solvate or hydrate thereof is dosed at 0.05 to 800 mg/day, preferably 1 to 400 mg/day, more preferably 20 to 200 mg/day, even more preferably 40 to 120 mg/day, still more preferably 50 to 100 mg/day, and most preferably 80 mg/day and the glycyrrhizic acid or a pharmaceutically acceptable salt, solvate or hydrate thereof is dosed at 0.5 to 1000 mg/day, preferably 1 to 500 mg/day, more preferably 5 to 400 mg/day, even more preferably 10 to 300 mg/day, still more preferably 15 to 250 mg/day, yet more preferably 50 to 200 mg/day and most preferably 70 to 120 mg/day. In one embodiment, the statin is pitavastatin, the glycyrrhizin derivative is glycyrrhizic acid, the pitavastatin is dosed at 0.05 to 800 mg/day, preferably 1 to 400 mg/day, more preferably 20 to 200 mg/day, even more preferably 40 to 120 mg/day, still more preferably 60 to 100 mg/day, and most preferably 80 mg/day, and the glycyrrhizic acid is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day, further more preferably 70 to 120 mg/day, yet further more preferably 85 to 100 mg/day and most preferably 90 mg/day.

### Methods of Preparation

The combination and pharmaceutical composition of the present invention may typically be prepared by mixing the glycyrrhizin derivative and the statin, together with any required excipients. This mixing can be carried out using a number of methods well known to those skilled in the art.

The solid pharmaceutical composition of the present invention may typically be prepared by mixing a solid form of the glycyrrhizin derivative and a solid form of the statin, together with any required excipients. This mixing can be carried out using a number of methods well known to those skilled in the art.

Advantageously, the above method is carried out in the absence of solvents. In contrast to the methods disclosed in the prior art (in particular, those described in RU 2308947 and RU 2396079), mixing the components of the combination in the absence of solvents avoids the formation of the unstable molecular complexes of statin and glycyrrhizic acid disclosed in these documents and enables the preparation of a pharmaceutical composition which is more stable (especially to long-term storage) and retains its water solubility over time.

### Kits

The present invention also encompasses kits for administering the combination of the present invention, wherein the hypolipidemic drug (preferably statin) and glycyrrhizin components of the combination are supplied as separate preparations in the same or different containers. Without wishing to be bound by theory, it is understood that the two components of the combination may be administered simultaneously, separately or sequentially and still achieve the advantageous effects described herein.

Therefore, in yet another aspect of the invention, there is provided a kit comprising:

(a) a therapeutically effective amount of a glycyrrhizin derivative, and optionally a pharmaceutically acceptable carrier or diluent in a first unit dosage form;
(b) a therapeutically effective amount of a hypolipidemic drug (preferably statin), and optionally a pharmaceutically acceptable carrier or diluent in a second unit dosage form; and (c) container means for containing said first and second dosage forms.

The combination kit can comprise a glycyrrhizin derivative, and the hypolipidemic drug (preferably statin) or a pharmaceutically acceptable salt or solvate thereof, in separate pharmaceutical compositions in a single container or in separate pharmaceutical compositions in separate containers.

In one embodiment, the kit comprises:

(a) a glycyrrhizin derivative, in association with a pharmaceutically acceptable carrier; and
(b) a hypolipidemic drug (preferably statin), in association with a pharmaceutically acceptable carrier, wherein the components (a) and (b) are provided in a form which is suitable for sequential, separate and/or simultaneous administration.

In one embodiment the kit comprises:

(a) a first container containing a glycyrrhizin derivative, in association with a pharmaceutically acceptable carrier;
(b) a second container comprising a hypolipidemic drug (preferably statin), in association with a pharmaceutically acceptable carrier; and
(c) a container means for containing said first and second containers.

The kit may also comprise instructions, such as dosage and administration instructions. Such dosage and administration instructions can be of the kind that is provided to a doctor, for example by a drug product label, or they can be of the kind that is provided by a doctor, such as instructions to a patient.

### Medical Uses and Methods of Treatment

The combination, such as pharmaceutical compositions and kits, according to the present invention, are suitable for use as medicaments for the treatment of disease. In this specification the term “treatment”, “treating” or “treat” means any administration of the combination of the present invention and includes: (1) preventing the disease from occurring in a subject which may be predisposed to the disease but does not yet experience or display the pathology or symptomatology of the disease, including the lowering of risk factors to the disease; (2) inhibiting the disease in a subject that is experiencing or displaying the pathology or symptomatology of the diseased (i.e., arresting further development of the pathology and/or symptomatology), or (3) ameliorating the disease in a subject that is experiencing or displaying the pathology or symptomatology of the diseased (i.e., reversing the pathology and/or symptomatology) and includes all processes providing slowing, interrupting, arresting, controlling, or stopping of the progression of the conditions described herein, but does not necessarily indicate a total elimination of all symptoms or a cure of the disease.

The combination of the present invention may be used in a human or non-human subject. Non-human subjects include companion animals such as dogs, cats, rabbits and horses, and livestock such as cows, sheep, pigs and goats. Preferably the subject is a human subject.

The combination of the present invention may be used to treat any of the diseases and conditions for which hypolipidemic drugs (such as statins) are known to be useful, particularly in diseases treatable by the lowering of cholesterol and other lipids in blood. Therefore, in another aspect of the invention, there is provided the combination or pharmaceutical composition, for use in treating hyperlipidemia. In one embodiment, the hyperlipidemia is selected from hypercholesterolemia, (also known as hyperlipoproteinemia), hypertriglyceridemia or a co-morbidity thereof.

In one embodiment, the hyperlipidemia is a primary (or familial) hyperlipidemia. Familial hyperlipidemias are classified according to the Fredrickson classification which is based on the pattern of lipoproteins on electrophoresis or ultracentrifugation. Examples of primary hyperlipidemias include Type Ia, Type Ib, Type Ic, Type IIa, Type IIb (familial combined hyperlipidemia), Type III (familial disbetalipoproteinemia), Type IV (familial hypertriglyceridemia) and Type V. Primary hyperlipidemias particularly responsive to treatment with statins include Type IIa, Type IIb, Type III and Type IV.

In another embodiment, the hyperlipidemia is a secondary (or acquired) hyperlipidemia. Acquire hyperlipidemia is typically secondary to other diseases, such as diabetes mellitus, hypothyroidism; renal failure, nephrotic syndrome; alcohol consumption; or the use of drugs such as diuretics, beta blockers, and estrogens.

Lowering blood cholesterol and other lipid levels is well known to be effective in the treatment of prevention of cardiovascular disease. Therefore, in another aspect of the invention, there is provided the combination or pharmaceutical composition, for use in treating cardiovascular disease. In one embodiment, the cardiovascular disease is selected from ischemic heart disease, myocardial infarction, angina, stroke, atherosclerosis, atherosclerotic vascular disease, coronary heart disease, coronary artery disease, peripheral vascular disease, peripheral arterial disease, and intermittent claudication.

Lowering blood cholesterol and other blood lipid levels is also known to be effective in the treatment of prevention of diseases and conditions other than cardiovascular disease. Therefore, in another aspect of the invention, there is provided the combination or pharmaceutical composition, for use in treating a disease or condition selected from liver disease, fatty liver, chronic viral hepatitis, cirrhosis, apoplectic attack, pathology of cerebral and peripheral vascular, arterial hypertension, and diabetes mellitus.

In particular, the combination of the present invention may be used to treat or prevent, and/or lower the risk of contracting, atherosclerosis (whether or not the patients have hyperlipidemia). Therefore, in another aspect of the invention, there is provided the combination or pharmaceutical composition, for use in treating atherosclerosis.

In some embodiments, the combination of the present invention may be used to treat or prevent, and/or lower the risk of contracting, coronary heart disease (CHD) and or ischemic heart disease (whether or not the patients have hyperlipidemia). For example, the combination of the present invention may be used in patients with diabetes, patients with stroke or other cerebrovascular disease, patients with a history of peripheral vascular disease, or in patients with coronary heart disease or a predisposition thereto. The combination may reduce the risk of total mortality by reducing mortality from ischemic heart disease; reduce the risk of serious cardiovascular and coronary events (such as but not limited to non-fatal myocardial infarction; revascularization; and/or apoplectic attack).

The combination may also reduce the risk of the need for operations to restore coronary blood flow (such as coronary artery bypass grafting and percutaneous transluminal coronary angioplasty); reduce the risk of surgical intervention necessary to restore the peripheral blood flow and non-coronary revascularization of other species; and reduce the risk of hospitalization due to angina.

In patients with diabetes, the combination may also reduce the risk of peripheral vascular complications (holding revascularization, amputation of lower limbs of trophic ulcers).

In patients with coronary artery disease and hypercholesterolemia the combination may also slow the development of coronary atherosclerosis, including the reduction in the incidence of new complications.

In one embodiment, the combination may be used to treat hypercholesterolemia. In one embodiment, treatment of hypercholesterolemia comprises the reduction of total blood cholesterol from above baseline levels. In one embodiment, treatment of hypercholesterolemia comprises the reduction of LDL cholesterol from above baseline levels. In one embodiment, treatment of hypercholesterolemia comprises the reduction of triglycerides from above baseline levels. In one embodiment, treatment of hypercholesterolemia comprises the reduction of apolipoprotein B (apo B) from above baseline levels. Normal levels of cholesterol: 3.2-5.6 mmol/l. Normal levels of LDL cholesterol: 1.71-3.5 mmol/l. Normal levels of HDL cholesterol>0.9 mmol/l. Normal levels of triglycerides: 0.41-1.8 mmol/l. Normal values of atherogenic factor: <3.5.

In one embodiment, the antihypercholesterolemic effect of the combination of the present invention comprises a reduction of the ratio of LDL cholesterol (low density lipoprotein; “bad” cholesterol) to HDL cholesterol (high density lipoprotein; “good” cholesterol). In one embodiment, the antihypercholesterolemic effect is a reduction of the ratio of a reduction of the ratio of total cholesterol to HDL cholesterol.

In yet another aspect of the invention, there is provided use of the above combination or composition, in the manufacture of a medicament for treating hyperlipidemia (particularly hypercholesterolemia and/or hypertriglyceridemia). In a further aspect of the invention, there is provided use of the above combination or composition, in the manufacture of a medicament for treating a cardiovascular disease (such as a disease selected from the group consisting of ischemic heart disease, myocardial infarction, angina, stroke, atherosclerotic vascular disease, coronary heart disease, coronary artery disease, peripheral vascular disease, peripheral arterial disease, and intermittent claudication).

In yet another aspect of the invention, there is provided a method of treating hyperlipidemia (particularly hypercholesterolemia and/or, hypertriglyceridemia), the method comprising administering to the patient the above combination or pharmaceutical composition. In yet another aspect of the invention, there is provided a method of treating a cardiovascular disease (such as a disease selected from the group consisting of ischemic heart disease, myocardial infarction, angina, stroke, atherosclerotic vascular disease, coronary heart disease, coronary artery disease, peripheral vascular disease, peripheral arterial disease, and intermittent claudication), the method comprising administering to the patient the above combination or pharmaceutical composition.

### EXAMPLES

The present invention is now described in more detail with references to the Examples below. However, the present invention is not limited to these Examples.

In the Examples, the following abbreviations are used:

AGA Monoammonium glycyrrhizinate; ALT Alanine-aminotransferase;
AST Aspartate transaminase; GA Glycyrrhizic acid;
CPK Creatinphosphokinase; HDL Cholesterol of lipoproteins of high density;
LDL Cholesterol of lipoproteins of low density; CVD Cardiovascular diseases;

### TG Triglycerides; CH Cholesterol;

### IFCC International Federation of Clinical Chemistry and Laboratory Medicine;

M average; m average error; n number of supervision;
TB Total bilirubin; DB Direct bilirubin; Bind Indirect bilirubin.

The objectives of the non-clinical studies were as follows:

To determine an effective and safe dose of glycyrrhizin derivatives and statins in fixed combinations according to the present invention.

To determine the breadth of therapeutic action of glycyrrhizin derivatives and statins in fixed combinations according to the present invention with the prospect of selection of the possible range of dosage for usage in clinical practice.

To study the cholesterol-lowering effect of glycyrrhizin derivatives and statins in fixed combinations according to the present invention.

To examine the safety profile of glycyrrhizin derivatives and statins in fixed combinations in comparison with monotherapy by statins (hepatoprotective and mitoprotective activity).

To study the level of possible steroid-like effect of glycyrrhizin derivatives and statins in fixed combinations.

To study the impact of fluctuations in glucose levels during therapy.

Draw a conclusion of comparative effectiveness and safety profile of fixed combinations according to the result of change in biometric, biochemical and pathological parameters.

Estimate the antiatherosclerotic efficiency of glycyrrhizin derivatives and statins in fixed combinations according to the present invention (in Example 3).

### Example 1—Non-Clinical Study

### Materials and Methods

### Animals:

The animals used were male rats of Wistar line. The weight of animals prior to beginning of the study ranged from 200-220 g. The proposed model using rats as the experimental animals is a reproducible standard model for evaluation of hypocholesterolemic effect. The number of animals used in the study is sufficient for full statistically significant registration of the studied effects and is minimally rational from the point of ethical principles.

Laboratory animals before the start of the study were kept groupwise in cages for 14 days for adaptation purposes. During this period, every day the clinical condition of the animals was visually monitored.

The number of animals in each group was 12 male rats. The animals were randomly divided into groups, using as a criterion the body weight, so that the individual weight of the animals did not vary by more than 20% from the average weight of animals of same sex.

### Dose Calculation:

According to the instructions on medical use of each component as the active substance of the relevant drug, and in view of the potentiating effect of the second component, presumably effective daily therapeutic dose of drug combinations were selected.

The doses tested are shown in Table 1.

TABLE 1
The tested combinations

| | Daily therapeutic |
|---|---|
| Composition | dose (mg) |
| simvastatin (SV) | 20 |
| ammonium glycyrrhizinate (AGA) | 108 |
| atorvastatin (AV) | 10 |
| glycyrrhizic acid (GA) | 90 |
| lovastatin (LV) | 20 |
| sodium glycyrrhizinate (SGA) | 108 |
| pravastatin (PV) | 40 |
| glycyrrhetic acid (GtA) | 108 |
| rosuvastatin (RV) | 10 |
| ammonium glycyrrhizinate (AGA) | 108 |
| fluvastatin (FV) | 80 |
| glycyrrhizic acid (GA) | 90 |

In each case, the masses expressed above are the mass of the active ingredient (including any counter-ions when the active ingredient is in a salt form). The doses were calculated based on the weight of dry substance (according to pharmaceutical standards).

These combinations were prepared as physical mixtures of the solid ingredients in 0.5% (weight/volume) methylcellulose solution in water, with no other excipients. The total sample of component in the required amount was placed into the mortar. 0.5% methylcellulose solution was added to the components in the mortar and suspended until a uniform stable suspension was formed. The suspension was transferred to a vial and washed three times in a mortar and pestle, brought up to volume 56 ml and mixed well to obtain a homogeneous suspension.

In the tests below, the masses of the statin and glycyrrhizin derivative are expressed as the equivalent dose in humans (based on the known effective therapeutic doses shown in Table 1). According to the dose conversion formula, an equi-therapeutic dose was calculated for each component, taking into account the 250 g body weight of rat.

(X mg/kg*39)/7.0 wherein X is the therapeutic dose for humans; 39 is the conversion factor, in view of the average human body weight (70 kg) and 7.0 is the conversion factor, taking into account the body weight of rat (250 g).

The placebos used as a control contained the same a 0.5% (weight/volume) methylcellulose solution in water which was used to suspend the compositions of the invention, with no further excipients.

### Methodology

### Study Design

Evaluation of hypocholesterolemic efficacy and safety of pharmaceutical combination was performed in vivo in experimental rat hypercholesterolemia model.

After pathology formulation for 30 days, the study groups were formed.

Manipulations during the study needed to assess the efficacy of the test objects are shown in Table 2.

TABLE 2
Manipulations during the study

| | Day of the study | | | |
|---|---|---|---|---|
| Evaluation | Baseline | 30 | 60 | 90 |
| Evaluation of general condition | daily | | | |
| Biochemistry, lipid spectrum | + | + | + | + |
| parametrs | | | | |
| Weight of organs | | | | + |

### Induction of Hypercholesterolemia

For 90 days the studied animals received hypercholesterol diet for pathology induction. The diet included Cholesterol+Cholic acid+Overheated fats (deep fat).

The daily food was a standard ration, enriched with overheated (for 5 hours) unrefined sunflower oil and 82.5% butter in the ratio of 4:1, with addition of cholesterol (3%) and cholic acid (0.5%).

For 90 days the animals received the said ration ad libitum (30 g feed per animal). Development of hypercholesterolemia in animals was evaluated based on changes in lipid spectrum parameters on Day 30 of the study.

### Administration of Test Objects and Sample Preparation

Drugs were introduced intragastrically with an esophageal bougie daily on Day 31-90 of the study at the same time of day.

### Clinical Examination of Animals

Examination of animals in cages was performed daily. Appearance and behavior of the animals were monitored, with any deviations recorded in the log.

Body weight was recorded just before the introduction, and then once per week throughout the study in order to calculate body weight gain, volume and concentration of the drugs studied.

The animals were deprived of food 14 hours before blood sampling and euthanasia. In this case, access to water was not limited.

### Biochemical Blood Tests

The following parameters of animal blood were examined:

Parameters for evaluation of efficacy of drugs:

Biochemical parameters and activity of blood serum enzymes (total cholesterol (CHS), LDL-CHS, HDL-CHS, triglycerides.

Parameters for evaluation of drug toxicity:

Biochemical parameters and activity of serum enzymes (aspartate- and alanine aminotransferase, creatine phosphokinase, urea, serum amylase, total bilirubin, direct bilirubin, indirect bilirubin, serum glucose, cations K⁺, Na⁺).

1.0-1.84 ml blood samples for the study of biochemical parameters were taken from tail vein of rats. Blood samples were taken from the animals after 14-15-hour's fasting at same time of day (9.00-11.00). For biochemical parameters tests a Biochemical Analyzer A-25 (Biosystems, Spain) was used.

Standard kinetic methods of spectrophotometry described in recommendations were used (“IFCC methods for the measurement of catalytic concentration of enzymes” Part 7: IFCC method for creatine kinase // JIFCC. 1989. Vol. 1. pp. 130-139.) and publication were used (Quim Clin. 1987. Vol. 6. pp. 241-244; J. Clin Chem Clin Biochem. 1986. Vol. 24. pp. 481-495; and Gella et al. Clin Chim Acta. 1985. Vol. 153. pp. 241-247; Talke H. and, Schubert G. E. Klinische Wochenschrift. 1965. Vol. 43. pp. 174-175; Tanase H et al Jpn Circ J. 1970. Vol. 34(12). pp. 1197-1212; Gutmann I., Bergmeyer H. U. Methods of enzymatic Analysis, ed Bergmeyer H. U., Academic Press, N Y. 1974. Vol. 4. pp. 1794-1798; Pearlman F C and Lee R T Y. Clin Chem 1974, 20, 447-453; Zoppi F et al, Peracino A., Fenili D., Marcovina S. and Ramella C. Giorn It Chim Cl. 1976; 1:343-359; Allain C. C. et al Clin Chem. 1974. Vol. 20. pp. 470-475; and in Meiattini F., et al, Clin Chem. 1978. Vol. 24. pp. 2161-2165 (total cholesterol); Warnick G R et al. Clin Chem 2001, 47, 1579-96 (HDL/LDL); Bucolo G and David H. Clin Chem. 1973.-Vol. 19. pp. 476-482; and in Fossati P. and, Prencipe L., Clin Chem. 1982. Vol. 28. pp. 2077-2080 (triglycerides); Trinder P. Ann Clin Biochem. 1969. Vol. 6. pp. 24-27).

### Determination of Mass Coefficients of Organs

To calculate mass coefficients the pancreas, liver and kidneys of the animals were extracted. The organs were weighed and the mass coefficients were calculated by the following formula:

Mass coefficient=(organ weight/animal body weight)×100%

### Data Analysis

Records and data from primary cards, were transferred into a Statistica 6.0 package (StatSoft, Russia). For all quantitative data, group arithmetic mean (M) and standard error of the mean (SEM) were calculated. The results obtained were processed on an IBM PC/AT with Statistica 6.0 application package (StatSoft, Russia). The probability of differences between M values in the groups was determined using the Student or Mann-Whitney test. The differences were assumed true at confidence level p<0.05.

### Results

### Monitoring of Pathology Development

The animals were on hypercholesterolemic diet for 30 days prior to the start of the treatment. During this period, the researchers monitored development of pathologic changes according to results of analysis of blood lipid spectrum of the studied animals on Days 0 and 30 of diet.

There is not a statistically significant difference in the groups of animals the results of measurements of lipid spectrum prior to the diet beginning and on Day 30 of the diet as the mean for all experimental groups were provided in table 3.

TABLE 3
Lipid spectrum prior and on Day 30 of the diet

| | Lipid spectrum, M ± m | | | |
|---|---|---|---|---|
| | Total | | | |
| | CHS, | HDL CHS, | LDL CHS, | TG, |
| | mmol/l | mmol/l | mmol/l | mmol/l |
| Lipid spectrum | 2.2 ± 0.1 | 0.77 ± 0.03 | 0.96 ± 0.12 | 1.01 ± 0.04 |
| prior to the diet | | | | |
| beginning | | | | |
| Lipid spectrum on | 6.1 ± 0.2* | 0.58 ± 0.01* | 4.98 ± 0.20* | 1.20 ± 0.03 |
| Day 30 of the diet | | | | |

Note—
*differences are statistically significant as compared to Lipid spectrum prior to the diet beginning, t-test for independent variables at p < 0.05

Values of lipid spectrum parameters in experimental animals in this age group are consistent with the literature data (Physiological, biochemical and biometrics standard values for experimental animals Ed. by Makarov V. G., Makarova M. N. Authors: Abrashova T. V., Gushchin Ya. A., Kovaleva M. A., Rybakova A. V., Selezneva A. I., Sokolova A. P., Khodko S. V.-St. Petersburg: Lema Publishers. 2013. pp. 17-33).

On Day 30 of the diet, pronounced changes in all indicators of lipid spectrum were observed in the studied animals. It was observed that the level of CHS increased on average by 3 times, LDL—by 5 times, TG—by 1.2 times, while concentration of HDL fell by 1.5 times, all as compared to baseline level in blood of animals.

Despite the fact that in rats pronounced shifts of lipid spectrum values are much less rarely observed, by Day 30 critical values of each parameter were achieved in experimental animals. Thus, the pattern of blood lipid spectrum in the studied animals on Day 30 of the diet was sufficient to determine existing pathology and to start the treatment.

### Treatment Results

### Parameters of Lipid Spectrum of Blood

By Day 30 of the treatment development of the pathology continued in the background of the diet, which manifested itself in further increases in total cholesterol (CHS), LDL cholesterol, and triglycerides (TG), and reduced level of HDL cholesterol in animals of the control group (Tables 4-10).

On Day 30 of the treatment, stable hypocholesterolemic effect both from the use of the studied mixture and the reference drug was shown (Tables 4-10).

TABLE 4
Lipid spectrum on Day 30 day of the treatment in simvastatin +
monoammonium glycyrrhizinate groups (Day 60 of the study)

| | | | | Lipid spectrum, M ± m | | | |
|---|---|---|---|---|---|---|---|
| | | | | Total CHS, | HDL CHS, | LDL CHS, | TG, |
| Pos. | Group | Dose, mg | No. | mmol/l | mmol/l | mmol/l | mmol/l |
| 1 | SV + AGA | 20 + 108 | 12 | 4.0 ± 0.3* | 0.75 ± 0.05* | 3.20 ± 0.30* | 1.28 ± 0.12* |
| 2 | SV | 20 | 12 | 5.2 ± 0.2* | 0.82 ± 0.06* | 3.75 ± 0.25* | 1.41 ± 0.11 |
| 3 | SV | 40 | 12 | 4.2 ± 0.2* | 0.58 ± 0.02* | 3.54 ± 0.20* | 1.27 ± 0.04* |
| 4 | AGA | 108 | 12 | 5.8 ± 0.5* | 0.53 ± 0.05* | 4.40 ± 0.4* | 1.5 ± 0.11 |
| 5 | Placebo | — | 12 | 7.0 ± 0.5 | 0.44 ± 0.04 | 5.78 ± 0.50 | 1.71 ± 0.12 |

Note—
*differences are statistically significant as compared to Group No. 5, t-test for independent variables at p < 0.05

Thus, during administration of simvastatin (SV) 20 mg as monotherapy (as reference drug), one noted a decrease of the concentration of CHS by 26%, LDL decrease by 35%, TG decrease by 18%, with the increase in concentration of HDL by 86% as compared to the control group of animals.

In contrast, treatment with the mixture SV+AGA for 30 days led to the decrease in the level of CHS by 43%, LDL decrease by 44%, TG decrease by 25%, with the increase in the concentration of HDL by 70%.

Therefore, one should conclude that by Day 30 of the treatment the use of AGA made a significant contribution to the development of the hypocholesterolemic effect of the SV+AGA mixture.

Efficacy of the SV20 mg+AGA mixtures on Day 30 of the treatment relative to lipid spectrum was higher than that of the reference drug statin (20 mg) and compare to simvastatin with 40 mg daily therapeutic dose.

In group of AGA the hypocholesterolemic activity were detected too. But efficacy in combination with SV was higher, than effect of each component, suggesting a synergistic effect

Similar hypolipidemic effect for other statin+glycyrrhizinate combinations was observed (Tables 5-9). This evidences favourable hypocholesterolemic efficacy of pharmaceutical compositions and potential to reduce effective drug dose, with the view of statin proportion in the composition.

TABLE 5
Lipid spectrum on Day 30 day of the treatment in atorvastatin (AV) +
glycyrrhizic acid (GA) groups (Day 60 of the study)

| | | | | Lipid spectrum, M ± m | | | |
|---|---|---|---|---|---|---|---|
| | | | | Total CHS, | HDL CHS, | LDL CHS, | TG, |
| Pos. | Group | Dose, mg | No. | mmol/l | mmol/l | mmol/l | mmol/l |
| 1 | +GA | 10 + 90 | 12 | 4.0 ± 0.4* | 0.79 ± 0.04* | 3.20 ± 0.40* | 1.28 ± 0.11* |
| 2 | AV | 10 | 12 | 4.7 ± 0.3* | 0.75 ± 0.05* | 3.61 ± 0.25* | 1.44 ± 0.10 |
| 3 | AV | 20 | 12 | 3.8 ± 0.2* | 0.60 ± 0.01* | 3.31 ± 0.10* | 1.20 ± 0.03* |
| 4 | GA | 90 | 12 | 5.2 ± 0.5* | 0.63 ± 0.04* | 4.00 ± 0.3* | 1.6 ± 0.10 |
| 5 | Placebo | — | 12 | 7.0 ± 0.5 | 0.44 ± 0.04 | 5.78 ± 0.50 | 1.71 ± 0.12 |

Note—
*differences are statistically significant as compared to Group No. 5, t-test for independent variables at p < 0.05

TABLE 6
Lipid spectrum on Day 30 day of the treatment in lovastatin (LV) +
sodium glycyrrhizinate (SGA) groups (Day 60 of the study)

| | | | | Lipid spectrum, M ± m | | | |
|---|---|---|---|---|---|---|---|
| | | | | Total CHS, | HDL CHS, | LDL CHS, | TG, |
| Pos. | Group | Dose, mg | No. | mmol/l | mmol/l | mmol/l | mmol/l |
| 1 | LV + SGA | 20 + 108 | 12 | 4.2 ± 0.4* | 0.75 ± 0.05* | 3.51 ± 0.30* | 1.39 ± 0.11* |
| 2 | LV | 20 | 12 | 5.6 ± 0.1* | 0.64 ± 0.05* | 4.25 ± 0.44* | 1.31 ± 0.12* |
| 3 | LV | 40 | 12 | 4.5 ± 0.2* | 0.89 ± 0.01* | 3.82 ± 0.20* | 1.22 ± 0.03* |
| 4 | SGA | 108 | 12 | 5.0 ± 0.4* | 0.53 ± 0.04* | 4.30 ± 0.41* | 1.3 ± 0.11* |
| 5 | Placebo | — | 12 | 7.0 ± 0.5 | 0.44 ± 0.04 | 5.78 ± 0.50 | 1.71 ± 0.12 |

Note—
*differences are statistically significant as compared to Group No. 5, t-test for independent variables at p < 0.05

TABLE 7
Lipid spectrum on Day 30 day of the treatment in pravastatin (PV) +
glycyrrhetic acid (GtA) groups (Day 60 of the study)

| | | | | Lipid spectrum, M ± m | | | |
|---|---|---|---|---|---|---|---|
| | | | | Total CHS, | HDL CHS, | LDL CHS, | TG, |
| Pos. | Group | Dose, mg | No. | mmol/l | mmol/l | mmol/l | mmol/l |
| 1 | PV + GtA | 40 + 108 | 12 | 4.0 ± 0.2* | 0.72 ± 0.03* | 3.60 ± 0.20* | 1.48 ± 0.12 |
| 2 | PV | 40 | 12 | 4.4 ± 0.3* | 0.65 ± 0.04* | 4.12 ± 0.35* | 1.33 ± 0.12* |
| 3 | GtA | 108 | 12 | 4.9 ± 0.5* | 0.51 ± 0.04* | 4.40 ± 0.4* | 1.6 ± 0.11 |
| 4 | Placebo | — | 12 | 7.0 ± 0.5 | 0.44 ± 0.04 | 5.78 ± 0.50 | 1.71 ± 0.12 |

Note—
*differences are statistically significant as compared to Group No. 4, t-test for independent variables at p < 0.05

TABLE 8
Lipid spectrum on Day 30 day of the treatment in rosuvastatin (RV) +
monoammonium glycyrrhizinate (AGA) groups (Day 60 of the study)

| | | | | Lipid spectrum, M ± m | | | |
|---|---|---|---|---|---|---|---|
| | | | | Total CHS, | HDL CHS, | LDL CHS, | TG, |
| Pos. | Group | Dose, mg | No. | mmol/l | mmol/l | mmol/l | mmol/l |
| 1 | RV + AGA | 10 + 108 | 12 | 3.2 ± 0.2* | 0.88 ± 0.04* | 2.01 ± 0.20* | 1.18 ± 0.11* |
| 2 | RV | 10 | 12 | 4.0 ± 0.2* | 0.81 ± 0.05* | 3.23 ± 0.21* | 1.21 ± 0.10* |
| 3 | RV | 20 | 12 | 3.6 ± 0.1* | 0.9 ± 0.03* | 3.00 ± 0.10* | 1.27 ± 0.03* |
| 4 | AGA | 108 | 12 | 5.8 ± 0.5* | 0.53 ± 0.05* | 4.40 ± 0.4* | 1.5 ± 0.11 |
| 5 | Placebo | — | 12 | 7.0 ± 0.5 | 0.44 ± 0.04 | 5.78 ± 0.50 | 1.71 ± 0.12 |

Note—
*differences are statistically significant as compared to Group No. 5, t-test for independent variables at p < 0.05

TABLE 9
Lipid spectrum on Day 30 of the treatment fluvastatin (FV) +
glycyrrhizic acid (GA) groups (Day 60 of the study)

| | | | | Lipid spectrum, M ± m | | | |
|---|---|---|---|---|---|---|---|
| | | Dose, | | Total CHS, | HDL CHS, | LDL CHS, | TG, |
| Pos. | Group | mg | No. | mmol/l | mmol/l | mmol/l | mmol/l |
| 1 | FV + GA | 80 + 108 | 12 | 4.5 ± 0.4* | 0.70 ± 0.03* | 3.00 ± 0.20* | 1.33 ± 0.11* |
| 2 | FV | 80 | 12 | 5.0 ± 0.3* | 0.61 ± 0.04* | 3.31 ± 0.44 | 1.39 ± 0.14* |
| 4 | GA | 90 | 12 | 5.2 ± 0.5* | 0.63 ± 0.04* | 4.00 ± 0.3* | 1.6 ± 0.10 |
| 5 | Placebo | — | 12 | 7.0 ± 0.5 | 0.44 ± 0.04 | 5.78 ± 0.50 | 1.71 ± 0.12 |

Note—
*differences are statistically significant as compared to Group No. 4, t-test for independent variables at p < 0.05

60 days:

On Day 60 of the treatment, pronounced changes in lipid spectrum values were observed during the treatment with the mixture and reference drug (Tables 10-15).

For example, in the group which received the studied mixture simvastatin+monoammonium glycyrrhizinate, on Day 60 of the treatment concentration of CHS reduced by 49%, TG fell by 28%, LDL dropped by 64%, with the increase in the concentration of HDL by 136% as compared to that in the control group.

TABLE 10
Lipid spectrum on Day 60 of the treatment in simvastatin (SV) +
monoammonium glycyrrhizinate (AGA) groups (Day 90 of the study)

| | | | | Lipid spectrum, M ± m | | | |
|---|---|---|---|---|---|---|---|
| | | Dose, | | Total CHS, | HDL CHS, | LDL CHS, | TG, |
| Pos. | Group | mg | No. | mmol/l | mmol/l | mmol/l | mmol/l |
| 1 | SV + AGA | 20 + 108 | 12 | 3.7 ± 0.3* | 0.97 ± 0.06* | 2.27 ± 0.31* | 1.25 ± 0.10* |
| 2 | SV | 20 | 12 | 4.9 ± 0.3* | 0.77 ± 0.05* | 3.50 ± 0.05* | 1.43 ± 0.11 |
| 3 | SV | 40 | 12 | 3.8 ± 0.1* | 0.91 ± 0.02* | 3.20 ± 0.1* | 1.09 ± 0.05* |
| 4 | AGA | 108 | 12 | 4.5 ± 0.4* | 0.63 ± 0.05* | 3.80 ± 0.3* | 1.30 ± 0.12* |
| 5 | Placebo | 0 | 12 | 7.4 ± 0.6 | 0.41 ± 0.04 | 6.21 ± 0.60 | 1.73 ± 0.15 |

Note—
*differences are statistically significant as compared to Group No. 5, t-test for independent variables at p < 0.05

It should be noted that by Day 60 of the treatment the efficacy of SV+AGA mixture much more exceeded than in 30 days of the treatment.

The above total cholesterol results demonstrated that pharmaceutical composition of simvastatin 20 mg+ammonium glycyrrhizinate 108 mg according to the invention after 60-day therapy resulted in reduction of total blood cholesterol by 50% compared with control group. The hypolipidemic effect of simvastatin 20 mg+ammonium glycyrrhizinate 108 mg treatment according to the invention was similar to effect of simvastatin 40 mg as monotherapy and exceeded simvastatin 20 mg as monotherapy.

Similar hypolipidemic effect for other statin+glycyrrhizinate combinations according to the invention was observed, as demonstrated by the results set out below (Tables 11-15). In particular, the hypolipidemic effects of the atorvastatin (10 mg)+glycyrrhizic acid group, the lovastatin (20 mg)+glycyrrhizic acid group, the rosuvastatin (10 mg)+monoammonium glycyrrhizinate group exceeded the effects of statin monotherapy in the same doses and was comparable with use of double the statin doses for atorvastatin (20 mg), lovastatin (40 mg), and rosuvastatin (20 mg) as monotherapy. In addition, the hypolipidemic effects of pravastatin (40 mg)+glycyrrhetic acid group and fluvastatin+glycyrrhizic acid group exceeded the effects of statin monotherapy in the same doses.

The hypocholesterolemic activity for all glycyrrhizin derivatives were detected and were lower than in other study groups. But, summarizing, the effect of the two components statin+glycyrrhizin derivative in each combination according to the present invention is substantially greater than the effect of each individual component in the free form. The evidences favourable hypocholesterolemic efficacy of pharmaceutical compositions and potential to reduce effective drug dose, with the view of statin proportion in the composition were demonstrated.

TABLE 11
Lipid spectrum on Day 60 of the treatment in atorvastatin (AV) +
glycyrrhizic acid (GA) groups (Day 90 of the study)

| | | | | Lipid spectrum, M ± m | | | |
|---|---|---|---|---|---|---|---|
| | | Dose, | | Total CHS, | HDL CHS, | LDL CHS, | TG, |
| Pos. | Group | mg | No. | mmol/l | mmol/l | mmol/l | mmol/l |
| 1 | +GA | 10 + 90 | 12 | 3.2 ± 0.3* | 0.99 ± 0.05* | 2.11 ± 0.40* | 1.20 ± 0.11* |
| 2 | AV | 10 | 12 | 4.0 ± 0.3* | 0.82 ± 0.04* | 3.23 ± 0.29* | 1.43 ± 0.10 |
| 3 | AV | 20 | 12 | 3.5 ± 0.2* | 0.98 ± 0.02* | 2.94 ± 0.20* | 1.10 ± 0.02* |
| 4 | GA | 90 | 12 | 4.5 ± 0.5* | 0.73 ± 0.04* | 3.90 ± 0.3* | 1.6 ± 0.10 |
| 5 | Placebo | 0 | 12 | 7.4 ± 0.6 | 0.41 ± 0.04 | 6.21 ± 0.60 | 1.73 ± 0.15 |

Note—
*differences are statistically significant as compared to Group No. 5, t-test for independent variables at p < 0.05

TABLE 12
Lipid spectrum on Day 60 of the treatment in lovastatin (LV) +
sodium glycyrrhizinate groups (Day 90 of the study)

| | | | | Lipid spectrum, M ± m | | | |
|---|---|---|---|---|---|---|---|
| | | Dose, | | Total CHS, | HDL CHS, | LDL CHS, | TG, |
| Pos. | Group | mg | No. | mmol/l | mmol/l | mmol/l | mmol/l |
| 1 | LV + SGA | 20 + 108 | 12 | 4.0 ± 0.3* | 0.91 ± 0.04* | 3.00 ± 0.35* | 1.29 ± 0.11* |
| 2 | LV | 20 | 12 | 4.8 ± 0.2* | 0.74 ± 0.04* | 3.92 ± 0.43* | 1.21 ± 0.11 |
| 3 | LV | 40 | 12 | 4.1 ± 0.3* | 0.90 ± 0.01* | 3.43 ± 0.23* | 1.20 ± 0.03* |
| 4 | SGA | 108 | 12 | 5.0 ± 0.4* | 0.63 ± 0.03* | 4.00 ± 0.3* | 1.6 ± 0.11 |
| 5 | Placebo | 0 | 12 | 7.4 ± 0.6 | 0.41 ± 0.04 | 6.21 ± 0.60 | 1.73 ± 0.15 |

Note—
*differences are statistically significant as compared to Group No. 5, t-test for independent variables at p < 0.05

TABLE 13
Lipid spectrum on Day 60 of the treatment in pravastatin (PV) +
glycyrrhetic acid (GtA) groups (Day 90 of the study)

| | | | | Lipid spectrum, M ± m | | | |
|---|---|---|---|---|---|---|---|
| | | Dose, | | Total CHS, | HDL CHS, | LDL CHS, | TG, |
| Pos. | Group | mg | No. | mmol/l | mmol/l | mmol/l | mmol/l |
| 1 | PV + GtA | 40 + 108 | 12 | 3.8 ± 0.3 | 0.89 ± 0.02* | 3.23 ± 0.20* | 1.28 ± 0.15* |
| 2 | PV | 40 | 12 | 4.0 ± 0.3* | 0.75 ± 0.04* | 3.70 ± 0.35* | 1.33 ± 0.11* |
| 3 | GtA | 108 | 12 | 4.5 ± 0.5* | 0.61 ± 0.03* | 4.24 ± 0.4* | 1.5 ± 0.11 |
| 4 | Placebo | 0 | 12 | 7.4 ± 0.6 | 0.41 ± 0.04 | 6.21 ± 0.60 | 1.73 ± 0.15 |

Note—
*differences are statistically significant as compared to Group No. 4, t-test for independent variables at p < 0.05

TABLE 14
Lipid spectrum on Day 60 of the treatment in rosuvastatin (RV) +
monoammonium glycyrrhizinate (AGA) groups (Day 90 of the study)

| | | | | Lipid spectrum, M ± m | | | |
|---|---|---|---|---|---|---|---|
| | | Dose, | | Total CHS, | HDL CHS, | LDL CHS, | TG, |
| Pos. | Group | mg | No. | mmol/l | mmol/l | mmol/l | mmol/l |
| 1 | RV + AGA | 10 + 108 | 12 | 3.0 ± 0.2* | 0.99 ± 0.03* | 2.00 ± 0.10* | 1.28 ± 0.11* |
| 2 | RV | 10 | 12 | 3.7 ± 0.2* | 0.81 ± 0.05* | 2.90 ± 0.21* | 1.21 ± 0.10* |
| 3 | RV | 20 | 12 | 3.3 ± 0.1* | 0.91 ± 0.03* | 2.72 ± 0.20* | 1.29 ± 0.03* |
| 4 | AGA | 108 | 12 | 4.5 ± 0.4* | 0.63 ± 0.05* | 3.80 ± 0.3* | 1.30 ± 0.12* |
| 5 | Placebo | 0 | 12 | 7.4 ± 0.6 | 0.41 ± 0.04 | 6.21 ± 0.60 | 1.73 ± 0.15 |

Note—
*differences are statistically significant as compared to Group No. 5, t-test for independent variables at p < 0.05

TABLE 15
Lipid spectrum on Day 60 of the treatment in fluvastatin (FV) +
glycyrrhizic acid groups (GA) (Day 90 of the study)

| | | | | Lipid spectrum, M ± m | | | |
|---|---|---|---|---|---|---|---|
| | | Dose, | | Total CHS, | HDL CHS, | LDL CHS, | TG, |
| Pos. | Group | mg | No. | mmol/l | mmol/l | mmol/l | mmol/l |
| 1 | FV + GA | 80 + 90 | 12 | 4.1 ± 0.4* | 0.80 ± 0.03* | 3.00 ± 0.30* | 1.49 ± 0.11 |
| 2 | FV | 80 | 12 | 4.9 ± 0.4* | 0.72 ± 0.03* | 3.52 ± 0.44* | 1.45 ± 0.14* |
| 3 | GA | 90 | 12 | 4.5 ± 0.5* | 0.73 ± 0.04* | 3.90 ± 0.3* | 1.6 ± 0.10 |
| 4 | Placebo | 0 | 12 | 7.4 ± 0.6 | 0.41 ± 0.04 | 6.21 ± 0.60 | 1.73 ± 0.15 |

Note—
*differences are statistically significant as compared to Group No. 4, t-test for independent variables at p < 0.05

Given the data obtained within 60 days of the treatment, it can be concluded that the efficacy of statins is enhanced if used together with glycyrrhizin derivatives according to the present invention, which in some embodiments indicates a synergistic effect of the combination.

### Biochemical Blood Analysis

Biochemistry data prior to the diet, and in 30 days of its use, are presented in Table 16. There was no statistically significant difference in the groups of animals the results of measurements of biochemical factors of blood at the start of the study and on Day 30 of the diet as the mean for all experimental groups were provided.

On Day 30 of the diet the shift in biochemical parameters occurred relative to baseline points in all the groups studied. The changed observed reflect the process of impairment of all kinds of metabolism during the use of fat-rich diet. AST activity concentration increased as compared to initial values (Table 16). The availability of the source substrate for the synthesis of cholesterol (acetyl-CoA) increases as a result of food intake containing carbohydrates and fats, as acetyl-CoA is formed in the oxidation of glucose and fatty acids.

In patients with atherosclerosis, in some cases a hidden form of carbohydrate metabolism is observed, i.e. of glucose fixation in tissues and organs. It often goes unnoticed, but is a very serious risk factor.

Tables 17-22 and 23-28 present biochemical factors of blood on Days 30 and 60 of the treatment, respectively (Days 60 and 90 of the study).

TABLE 16
Biochemical factors of blood prior to and on Day 30 of the diet

| | | | | | | | | Bili- | Bili- | Bili- | | | |
|---|---|---|---|---|---|---|---|---|---|---|---|---|---|
| | | | | | | | | rubin | rubin | rubin | | Potas- | |
| | | | AST, | ALT, | Urea, | CPK, | Amylase, | total, | direct, | indirect, | Glucose, | sium, | Sodium, |
| Pos. | Group | No. | U/l | U/l | mmol/l | U/l | U/l | mg/dl | mg/dl | mg/dl | mg/dl | mmol | mmol |
| 1 | Lipid spectrum prior | 12 | 125 ± | 81 ± | 6.9 ± | 425 ± | 731 ± | 2.9 ± | 0.9 ± | 2.0 ± | 112 ± | 2.9 ± | 125 ± |
| | to the diet beginning | | 2 | 2 | 0.2 | 16 | 9 | 0.2 | 0.1 | 0.2 | 6 | 0.3 | 4 |
| 2 | Lipid spectrum on | 12 | 165 ± | 85 ± | 6.7 ± | 435 ± | 799 ± | 3.9 ± | 1.4 ± | 2.3 ± | 125 ± | 3.1 ± | 129 ± |
| | Day 30 of the diet | | 5* | 5 | 0.2 | 12 | 10 | 0.1 | 0.1 | 0.1 | 2 | 0.2 | 6 |

Note—
*differences are statistically significant as compared to Lipid spectrum prior to the diet beginning, t-test for independent variables at p < 0.05

TABLE 17
Biochemical factors of blood on Day 30 of the treatment in simvastatin (SV) +
monoammonium glycyrrhizinate (AGA) groups (Day 60 of the study)

| | | | | | | | | | Bili- | Bili- | Bili- | | | |
|---|---|---|---|---|---|---|---|---|---|---|---|---|---|---|
| | | | | | | | | | rubin | rubin | rubin | | Potas- | |
| | | Dose, | | AST, | ALT, | Urea, | CPK, | Amylase, | total, | direct, | indirect, | Glucose, | sium, | Sodium, |
| Pos. | Group | mg | No. | U/l | U/l | mmol/l | U/l | U/l | mg/dl | mg/dl | mg/dl | mg/dl | mmol | mmol |
| 1 | SV + AGA | 20 + | 12 | 135 ± | 117 ± | 5.4 ± | 305 ± | 625 ± | 4.2 ± | 2.8 ± | 1.9 ± | 117 ± | 3.2 ± | 125 ± |
| | | 108 | | 11* | 12*¹ | 0.5* | 24*¹ | 22 | 0.3 | 0.2 | 0.4 | 11¹ | 0.3 | 11 |
| 2 | SV | 20 | 12 | 172 ± | 169 ± | 7.3 ± | 611 ± | 643 ± | 4.6 ± | 2.5 ± | 2.0 ± | 139 ± | 2.3 ± | 128 ± |
| | | | | 10* | 14 | 0.2 | 10* | 28 | 0.5 | 0.2 | 0.5 | 7* | 0.6 | 12 |
| 3 | AGA | 108 | 12 | 130 ± | 98 ± | 6.8 ± | 310 ± | 620 ± | 4.1 ± | 2.1 ± | 1.8 ± | 112 ± | 2.7 ± | 120 ± |
| | | | | 7*¹ | 19*¹ | 0.7 | 18*¹ | 33 | 0.3 | 04* | 0.4 | 6¹ | 0.5 | 8 |
| 4 | placebo | — | 12 | 221 ± | 173 ± | 7.8 ± | 715 ± | 621 ± | 4.8 ± | 3.4 ± | 1.4 ± | 110 ± | 2.9 ± | 130 ± |
| | | | | 9 | 19 | 0.7 | 14 | 35 | 0.4 | 0.4 | 0.4 | 5¹ | 0.5 | 8 |

*differences are statistically significant as compared to Group No. 4,
¹differences are statistically significant as compared to groups No. 2. t-test for independent variables at p < 0.05

TABLE 18
Biochemical factors of blood on Day 30 of the treatment in atorvastatin (AV) +
glycyrrhizic acid (AGA) groups (Day 60 of the study)

| | | | | | | | | | Bili- | Bili- | Bili- | | | |
|---|---|---|---|---|---|---|---|---|---|---|---|---|---|---|
| | | | | | | | | | rubin | rubin | rubin | | Potas- | |
| | | Dose, | | AST, | ALT, | Urea, | CPK, | Amylase, | total, | direct, | indirect, | Glucose, | sium, | Sodium, |
| Pos. | Group | mg | No. | U/l | U/l | mmol/l | U/l | U/l | mg/dl | mg/dl | mg/dl | mg/dl | mmol | mmol |
| 1 | AV + GA | 10 + | 12 | 130 ± | 118 ± | 5.0 ± | 400 ± | 621 ± | 4.0 ± | 2.7 ± | 1.8 ± | 115 ± | 3.1 ± | 126 ± |
| | | 90 | | 10* | 11*¹ | 0.5* | 23*¹ | 21 | 0.3 | 0.2 | 0.4 | 11¹ | 0.3 | 11 |
| 2 | AV | 10 | 12 | 171 ± | 140 ± | 7.4 ± | 620 ± | 640 ± | 4.7 ± | 2.6 ± | 2.2 ± | 140 ± | 3.0 ± | 120 ± |
| | | | | 10* | 13 | 0.2 | 11* | 29 | 0.5 | 0.2 | 0.5 | 7* | 0.6 | 12 |
| 3 | GA | 90 | 12 | 125 ± | 100 ± | 5.7 ± | 320 ± | 618 ± | 4.5 ± | 2.2 ± | 1.4 ± | 111 ± | 2.2 ± | 135 ± |
| | | | | 7*¹ | 18*¹ | 0.3 | 15*¹ | 32 | 0.4 | 04* | 0.5 | 7¹ | 0.4 | 7 |
| 4 | placebo | — | 12 | 221 ± | 173 ± | 7.8 ± | 715 ± | 621 ± | 4.8 ± | 3.4 ± | 1.4 ± | 110 ± | 2.9 ± | 130 ± |
| | | | | 9 | 19 | 0.7 | 14 | 35 | 0.4 | 0.4 | 0.4 | 5¹ | 0.5 | 8 |

*differences are statistically significant as compared to Group No. 4,
¹differences are statistically significant as compared to groups No. 2. t-test for independent variables at p < 0.05

TABLE 19
Biochemical factors of blood on Day 30 of the treatment in lovastatin (LV) +
sodium glycyrrhizinate (SGA) groups (Day 60 of the study)

| | | | | | | | | | Bili- | Bili- | Bili- | | | |
|---|---|---|---|---|---|---|---|---|---|---|---|---|---|---|
| | | | | | | | | | rubin | rubin | rubin | | Potas- | |
| | | Dose, | | AST, | ALT, | Urea, | CPK, | Amylase, | total, | direct, | indirect, | Glucose, | sium, | Sodium, |
| Pos. | Group | mg | No. | U/l | U/l | mmol/l | U/l | U/l | mg/dl | mg/dl | mg/dl | mg/dl | mmol | mmol |
| 1 | LV + SGA | 20 + | 12 | 151 ± | 110 ± | 5.2 ± | 420 ± | 623 ± | 3.5 ± | 1.8 ± | 1.7 ± | 117 ± | 3.2 ± | 130 ± |
| | | 108 | | 11*¹ | 10*¹ | 0.5* | 23*¹ | 21 | 0.3 | 0.2 | 0.4 | 11¹ | 0.3 | 11 |
| 2 | LV | 20 | 12 | 183 ± | 160 ± | 7.3 ± | 510 ± | 646 ± | 4.6 ± | 2.1 ± | 2.1 ± | 139 ± | 2.1 ± | 128 ± |
| | | | | 9* | 13 | 0.2 | 11* | 27 | 0.5 | 0.2 | 0.5 | 7* | 0.4 | 12 |
| 3 | SGA | 108 | 12 | 125 ± | 91 ± | 6.8 ± | 308 ± | 600 ± | 4.3 ± | 3.0 ± | 1.2 ± | 110 ± | 2.5 ± | 132 ± |
| | | | | 8*¹ | 18*¹ | 0.5 | 18*¹ | 32 | 0.3 | 05* | 0.5 | 6¹ | 0.4 | 8 |
| 4 | placebo | — | 12 | 221 ± | 173 ± | 7.8 ± | 715 ± | 621 ± | 4.8 ± | 3.4 ± | 1.4 ± | 110 ± | 2.9 ± | 130 ± |
| | | | | 9 | 19 | 0.7 | 14 | 35 | 0.4 | 0.4 | 0.4 | 5¹ | 0.5 | 8 |

*differences are statistically significant as compared to Group No. 4,
¹differences are statistically significant as compared to groups No. 2. t-test for independent variables at p < 0.05

TABLE 20
Biochemical factors of blood on Day 30 of the treatment in pravastatin (PV) +
glycyrrhetic acid (GtA) groups (Day 60 of the study)

| | | | | | | | | | Bili- | Bili- | Bili- | | | |
|---|---|---|---|---|---|---|---|---|---|---|---|---|---|---|
| | | | | | | | | | rubin | rubin | rubin | | Potas- | |
| | | Dose, | | AST, | ALT, | Urea, | CPK, | Amylase, | total, | direct, | indirect, | Glucose, | sium, | Sodium, |
| Pos. | Group | mg | No. | U/l | U/l | mmol/l | U/l | U/l | mg/dl | mg/dl | mg/dl | mg/dl | mmol | mmol |
| 1 | PV + GtA | 40 + | 12 | 127 ± | 118 ± | 5.1 ± | 420 ± | 620 ± | 3.2 ± | 2.0 ± | 1.6 ± | 126 ± | 3.1 ± | 128 ± |
| | | 108 | | 10*¹ | 12*¹ | 0.6* | 25*¹ | 22 | 0.2 | 0.2 | 0.3 | 10¹ | 0.2 | 10 |
| 2 | PV | 40 | 12 | 173 ± | 177 ± | 7.0 ± | 604 ± | 641 ± | 4.7 ± | 2.8 ± | 2.2 ± | 150 ± | 3.4 ± | 127 ± |
| | | | | 12* | 17 | 0.3 | 13* | 26 | 0.2 | 0.2 | 0.5 | 7* | 0.7 | 10 |
| 3 | GtA | 108 | 12 | 130 ± | 97 ± | 6.5 ± | 390 ± | 622 ± | 3.2 ± | 1.1 ± | 1.7 ± | 119 ± | 2.8 ± | 113 ± |
| | | | | 9*¹ | 17*¹ | 0.4 | 17*¹ | 30 | 0.3 | 03* | 0.6 | 7¹ | 0.6 | 9 |
| 4 | placebo | — | 12 | 221 ± | 173 ± | 7.8 ± | 715 ± | 621 ± | 4.8 ± | 3.4 ± | 1.4 ± | 110 ± | 2.9 ± | 130 ± |
| | | | | 9 | 19 | 0.7 | 14 | 35 | 0.4 | 0.4 | 0.4 | 5¹ | 0.5 | 8 |

*differences are statistically significant as compared to Group No. 4,
¹differences are statistically significant as compared to groups No. 2. t-test for independent variables at p < 0.05

TABLE 21
Biochemical factors of blood on Day 30 of the treatment in rosuvastatin (RV) +
monoammonium glycyrrhizinate (AGA) groups (Day 60 of the study)

| | | | | | | | | | Bili- | Bili- | Bili- | | | |
|---|---|---|---|---|---|---|---|---|---|---|---|---|---|---|
| | | | | | | | | | rubin | rubin | rubin | | Potas- | |
| | | Dose, | | AST, | ALT, | Urea, | CPK, | Amylase, | total, | direct, | indirect, | Glucose, | sium, | Sodium, |
| Pos. | Group | mg | No. | U/l | U/l | mmol/l | U/l | U/l | mg/dl | mg/dl | mg/dl | mg/dl | mmol | mmol |
| 1 | RV + AGA | 10 + | 12 | 136 ± | 115 ± | 5.0 ± | 450 ± | 621 ± | 4.1 ± | 2.8 ± | 1.5 ± | 115 ± | 3.2 ± | 117 ± |
| | | 108 | | 12*¹ | 11*¹ | 0.5* | 24*¹ | 21 | 0.2 | 0.3 | 0.2 | 11¹ | 0.1 | 11 |
| 2 | RV | 10 | 12 | 182 ± | 179 ± | 7.2 ± | 702 ± | 641 ± | 4.6 ± | 2.7 ± | 2.1 ± | 158 ± | 4.3 ± | 128 ± |
| | | | | 13* | 18 | 0.3 | 12* | 25 | 0.3 | 0.3 | 0.4 | 6* | 0.6 | 11 |
| 3 | AGA | 108 | 12 | 130 ± | 98 ± | 6.8 ± | 312 ± | 620 ± | 4.1 ± | 2.1 ± | 1.8 ± | 112 ± | 2.7 ± | 120 ± |
| | | | | 7*¹ | 19*¹ | 0.7 | 18*¹ | 33 | 0.3 | 04* | 0.4 | 6¹ | 0.5 | 8 |
| 4 | Placebo | — | 12 | 221 ± | 173 ± | 7.8 ± | 715 ± | 621 ± | 4.8 ± | 3.4 ± | 1.4 ± | 110 ± | 2.9 ± | 130 ± |
| | | | | 9 | 19 | 0.7 | 14 | 35 | 0.4 | 0.4 | 0.4 | 5¹ | 0.5 | 8 |

*differences are statistically significant as compared to Group No. 4,
¹differences are statistically significant as compared to groups No. 2. t-test for independent variables at p < 0.05

TABLE 22
Biochemical factors of blood on Day 30 of the treatment in fluvastatin (FV) +
glycyrrhizic acid (GA) groups (Day 60 of the study)

| | | | | | | | | | Bili- | Bili- | Bili- | | | |
|---|---|---|---|---|---|---|---|---|---|---|---|---|---|---|
| | | | | | | | | | rubin | rubin | rubin | | Potas- | |
| | | Dose, | | AST, | ALT, | Urea, | CPK, | Amylase, | total, | direct, | indirect, | Glucose, | sium, | Sodium, |
| Pos. | Group | mg | No. | U/l | U/l | mmol/l | U/l | U/l | mg/dl | mg/dl | mg/dl | mg/dl | mmol | mmol |
| 1 | FV + GA | 80 + | 12 | 138 ± | 110 ± | 4.9 ± | 400 ± | 590 ± | 4.0 ± | 2.7 ± | 1.9 ± | 100 ± | 3.1 ± | 129 ± |
| | | 90 | | 10*¹ | 12*¹ | 0.6* | 22*¹ | 20 | 0.3 | 0.2 | 0.3 | 12¹ | 0.2 | 12 |
| 2 | FV | 80 | 12 | 191 ± | 170 ± | 7.3 ± | 690 ± | 637 ± | 4.0 ± | 2.6 ± | 2.2 ± | 150 ± | 3.0 ± | 125 ± |
| | | | | 12* | 13 | 0.2 | 11* | 26 | 0.4 | 0.4 | 0.5 | 7* | 0.5 | 10 |
| 3 | GA | 90 | 12 | 132 ± | 100 ± | 5.7 ± | 320 ± | 618 ± | 4.5 ± | 2.2 ± | 1.4 ± | 111 ± | 2.2 ± | 135 ± |
| | | | | 7*¹ | 10*¹ | 0.3 | 15*¹ | 32 | 0.4 | 04* | 0.5 | 7¹ | 0.4 | 7 |
| 4 | placebo | — | 12 | 221 ± | 173 ± | 7.8 ± | 715 ± | 621 ± | 4.8 ± | 3.4 ± | 1.4 ± | 110 ± | 2.9 ± | 130 ± |
| | | | | 9 | 19 | 0.7 | 14 | 35 | 0.4 | 0.4 | 0.4 | 5¹ | 0.5 | 8 |

*differences are statistically significant as compared to Group No. 4,
¹differences are statistically significant as compared to groups No. 2 t-test for independent variables at p < 0.05

In the control group, on Day 60 of the study the increasing of AST and AST activity of while investigation has been detected. While De Rytis factor (AST/ALT) was equal to 2.2, which may indicate the development of pathological processes in myocardium during the diet. The concentration of glucose in blood of animals of statin groups was high. Glucose intolerance with dislipoproteinemia could be the cause of the increase in level of glucose in blood of animals on Day 60 of the diet. During 30-days treatment by SV+AGA mixture, a statistically significant decrease of activity of AST and ALT, as well as the tendency of reduction in glucose concentration were detected. Contribution of AGA in reduced activity of transaminases and stabilization of glucose levels was observed in the study. Similar effects for other statin and glycyrrhizin derivative combinations according to the present invention were observed.

Without wishing to be bound by theory, it is believed that the decrease in concentration of glucose in the reference group of statins may be caused with dysfunction of liver enzyme system involved in carbohydrate metabolism, while the use of the studied mixtures of statin and glycyrrhizin derivative according to the present invention prevented a sharp decline in the concentration of glucose in blood of animals.

By Day 60 day of the treatment pronounced hepatoprotective effect of the mixtures statin+glycyrrhizin derivative that manifested in pronounced decline in activity of transaminases. In addition, reduction in mytotoxicity which manifested itself in reduced activity of CPK in the group of animals which received the mixture statin+glycyrrhizin derivative was detected. Values of AST, ALT and CPK parameters were significantly lower than those during the use of the reference drug statins in the same doses. In groups of glycyrrhizin derivatives the values of AST, ALT and CPK was compared to baseline parameters.

In addition to the impact on the activity of enzymes, the use of the studied mixture statin+glycyrrhizin derivative effected carbohydrate metabolism, i.e. glucose concentration values in the group which received the studied mixture had not been critically changed compare to baseline, as was the case in the group which received monotherapy of statins. Results of the analysis of the biochemical profile by Day 60 of the treatment (Day 90 of the study) are given in Tables 23-28.

TABLE 23
Biochemical factors of blood on Day 60 of the treatment in simvastatin (SV) +
monoammonium glycyrrhizinate groups (AGA) (Day 90 of the study)

| | | | | | | | | | Bili- | Bili- | Bili- | | | |
|---|---|---|---|---|---|---|---|---|---|---|---|---|---|---|
| | | | | | | | | | rubin | rubin | rubin | | Potas- | |
| | | Dose, | | AST, | ALT, | Urea, | CPK, | Amylase, | total, | direct, | indirect, | Glucose, | sium, | Sodium, |
| Pos. | Group | mg | No. | U/l | U/l | mmol/l | U/l | U/l | mg/dl | mg/dl | mg/dl | mg/dl | mmol | mmol |
| 1 | SV + AGA | 20 + | 12 | 137 ± | 107 ± | 5.7 ± | 300 ± | 579 ± | 3.1 ± | 2.4 ± | 1.8 ± | 119 ± | 3.1 ± | 131 ± |
| | | 108 | | 8*¹ | 7*¹ | 0.4 | 11*¹ | 38 | 0.3* | 0.3* | 0.2 | 10¹ | 0.4 | 10 |
| 2 | SV | 20 | 12 | 189 ± | 134 ± | 6.0 ± | 599 ± | 587 ± | 4.8 ± | 2.9 ± | 1.9 ± | 160 ± | 3.2 ± | 128 ± |
| | | | | 15 | 11 | 0.6 | 47 | 25 | 0.4 | 0.3* | 0.3 | 7* | 0.3 | 7 |
| 3 | AGA | 108 | 12 | 131 ± | 100 ± | 5.5 ± | 310 ± | 600 ± | 2.9 ± | 2.3 ± | 1.9 ± | 110 ± | 3.2 ± | 122 ± |
| | | | | 10*¹ | 9*¹ | 0.4 | 12*¹ | 38 | 0.3* | 03* | 0.2 | 4¹ | 0.4 | 10 |
| 4 | placebo | — | 12 | 207 ± | 139 ± | 6.4 ± | 611 ± | 593 ± | 5.7 ± | 4.1 ± | 1.6 ± | 109 ± | 3.0 ± | 132 ± |
| | | | | 14 | 16 | 0.4 | 43 | 55 | 0.5 | 0.3 | 0.3 | 8¹ | 0.5 | 12 |

*differences are statistically significant as compared to Group No. 4, t-test for independent variables at p < 0.05,
¹differences are statistically significant as compared to groups No. 2 t-test for independent variables at p < 0.05

TABLE 24
Biochemical factors of blood on Day 60 of the treatment in
atorvastatin (AV) + glycyrrhizic acid (GA) groups (Day 90 of the study)

| | | | | | | | | | Bili- | Bili- | Bili- | | | |
|---|---|---|---|---|---|---|---|---|---|---|---|---|---|---|
| | | | | | | | | | rubin | rubin | rubin | | Potas- | |
| | | Dose, | | AST, | ALT, | Urea, | CPK, | Amylase, | total, | direct, | indirect, | Glucose, | sium, | Sodium, |
| | Group | mg | No. | U/l | U/l | mmol/l | U/l | U/l | mg/dl | mg/dl | mg/dl | mg/dl | mmol | mmol |
| 1 | AV + GA | 10 + | 12 | 124 ± | 105 ± | 5.5 ± | 332 ± | 581 ± | 3.0 ± | 1.3 ± | 1.7 ± | 100 ± | 3.4 ± | 130 ± |
| | | 90 | | 7*¹ | 6*¹ | 0.3 | 11*¹ | 34 | 0.2* | 0.3* | 0.2 | 10*¹ | 0.2 | 11 |
| 2 | AV | 10 | 12 | 191 ± | 160 ± | 6.1 ± | 587 ± | 577 ± | 4.5 ± | 3.0 ± | 2.0 ± | 150 ± | 3.4 ± | 129 ± |
| | | | | 14 | 9 | 0.5 | 45 | 20 | 0.4 | 0.3* | 0.3 | 7* | 0.3 | 6 |
| 3 | GA | 90 | 12 | 115 ± | 103 ± | 5.0 ± | 233 ± | 600 ± | 4.3 ± | 2.2 ± | 1.8 ± | 116 ± | 3.1 ± | 123 ± |
| | | | | 8*¹ | 10*¹ | 0.4 | 10*¹ | 35 | 0.3* | 0.3* | 0.2 | 9 | 0.3 | 10 |
| 4 | placebo | = | 12 | 207 ± | 139 ± | 6.4 ± | 611 ± | 593 ± | 5.7 ± | 4.1 ± | 1.6 ± | 109 ± | 3.0 ± | 132 ± |
| | | | | 14 | 16 | 0.4 | 43 | 55 | 0.5 | 0.3 | 0.3 | 8¹ | 0.5 | 12 |

*differences are statisically sigmficant as compared to Group No. 4, t-test for independent variables at p < 0.05,
¹differences are statistically significant as compared to groups No. 2 t-test for independent variables at p < 0.05

TABLE 25
Biochemical factors of blood on Day 60 of the treatment in
lovastatin (LV) + sodium glycyrrhizinate (SGA) groups (Day 90 of the study)

| | | | | | | | | | Bili- | Bili- | Bili- | | | |
|---|---|---|---|---|---|---|---|---|---|---|---|---|---|---|
| | | | | | | | | | rubin | rubin | rubin | | Potas- | |
| | | Dose, | | AST, | ALT, | Urea, | CPK, | Amylase, | total, | direct, | indirect, | Glucose, | sium, | Sodium, |
| | Group | mg | No. | U/l | U/l | mmol/l | U/l | U/l | mg/dl | mg/dl | mg/dl | mg/dl | mmol | mmol |
| 1 | LV + SGA | 20 + | 12 | 121 ± | 106 ± | 5.8 ± | 380 ± | 580 ± | 3.2 ± | 2.0 ± | 1.8 ± | 100 ± | 3.5 ± | 131 ± |
| | | 108 | | 6*¹ | 6*¹ | 0.2 | 11*¹ | 33 | 0.2* | 0.3* | 0.3 | 8*¹ | 0.2 | 11 |
| 2 | LV | 20 | 12 | 190 ± | 141 ± | 6.0 ± | 597 ± | 597 ± | 4.4 ± | 4.4 ± | 2.3 ± | 148 ± | 3.3 ± | 128 ± |
| | | | | 10 | 8 | 0.4 | 45 | 21 | 0.4 | 0.3* | 0.2 | 5* | 0.3 | 6 |
| 3 | SGA | 108 | 12 | 123 ± | 102 ± | 5.2 ± | 331 ± | 603 ± | 2.9 ± | 2.0 ± | 1.9 ± | 120 ± | 3.2 ± | 121 ± |
| | | | | 8*¹ | 6*¹ | 0.2 | 10*¹ | 32 | 0.5* | 0.3* | 0.2 | 4¹ | 0.4 | 9 |
| 4 | placebo | — | 12 | 207 ± | 139 ± | 6.4 ± | 611 ± | 593 ± | 5.7 ± | 4.1 ± | 1.6 ± | 109 ± | 3.0 ± | 132 ± |
| | | | | 14 | 16 | 0.4 | 43 | 55 | 0.5 | 0.3 | 0.3 | 8¹ | 0.5 | 12 |

*differences are statisically sigmficant as compared to Group No. 4, t-test for independent variables at p < 0.05,
¹differences are statistically significant as compared to groups No. 2 t-test for independent variables t p < 0.05

TABLE 26
Biochemical factors of blood on Day 60 of the treatment in
pravastatin (PV) + glycyrrhetic acid (GtA) groups (Day 90 of the study)

| | | | | | | | | | Bili- | Bili- | Bili- | | | |
|---|---|---|---|---|---|---|---|---|---|---|---|---|---|---|
| | | | | | | | | | rubin | rubin | rubin | | Potas- | |
| | | Dose, | | AST, | ALT, | Urea, | CPK, | Amylase, | total, | direct, | indirect, | Glucose, | sium, | Sodium, |
| | Group | mg | No. | U/l | U/l | mmol/l | U/l | U/l | mg/dl | mg/dl | mg/dl | mg/dl | mmol | mmol |
| 1 | PV + GtA | 40 + | 12 | 128 ± | 108 ± | 5.7 ± | 327 ± | 579 ± | 3.0 ± | 2.4 ± | 1.8 ± | 107 ± | 3.0 ± | 131 ± |
| | | 108 | | 8*¹ | 7*¹ | 0.4 | 11*¹ | 38 | 0.3* | 0.3* | 0.2 | 9*¹ | 0.4 | 10 |
| 2 | PV | 20 | 12 | 188 ± | 135 ± | 6.0 ± | 598 ± | 586 ± | 4.9 ± | 2.7 ± | 1.9 ± | 160 ± | 4.3 ± | 129 ± |
| | | | | 15 | 11 | 0.6 | 47 | 23 | 0.4 | 0.3* | 0.3 | 5* | 0.2 | 6 |
| 3 | GtA | 108 | 12 | 120 ± | 101 ± | 5.5 ± | 300 ± | 599 ± | 2.7 ± | 1.2 ± | 1.8 ± | 112 ± | 3.1 ± | 123 ± |
| | | | | 10*¹ | 9*¹ | 0.4 | 12*¹ | 37 | 0.2* | 0.3* | 0.2 | 5 | 0.3 | 11 |
| 4 | placebo | — | 12 | 207 ± | 139 ± | 6.4 ± | 611 ± | 593 ± | 5.7 ± | 4.1 ± | 1.6 ± | 109 ± | 4.0 ± | 132 ± |
| | | | | 14 | 16 | 0.4 | 43 | 55 | 0.5 | 0.3 | 0.3 | 8¹ | 0.5 | 12 |

*differences are statisically sigmficant as compared to Group No. 4, t-test for independent variables at p < 0.05,
¹differences are statistically significant as compared to groups No. 2 t-test for independent variables at p < 0.05

TABLE 27
Biochemical factors of blood on Day 60 of the treatment in
rosuvastatin (RV) + monoammonium glycyrrhizinate (AGA) groups (Day 90 of the study)

| | | | | | | | | | Bili- | Bili- | Bili- | | | |
|---|---|---|---|---|---|---|---|---|---|---|---|---|---|---|
| | | | | | | | | | rubin | rubin | rubin | | Potas- | |
| | | Dose, | | AST, | ALT, | Urea, | CPK, | Amylase, | total, | direct, | indirect, | Glucose, | sium, | Sodium, |
| | Group | mg | No. | U/l | U/l | mmol/l | U/l | U/l | mg/dl | mg/dl | mg/dl | mg/dl | mmol | mmol |
| 1 | RV + AGA | 10 + | 12 | 124 ± | 107 ± | 5.6 ± | 318 ± | 578 ± | 3.8 ± | 2.5 ± | 1.9 ± | 122 ± | 3.0 ± | 132 ± |
| | | 108 | | 8*¹ | 6*¹ | 0.4 | 10*¹ | 38 | 0.3* | 0.2* | 0.2 | 9*¹ | 0.4 | 10 |
| 2 | RV | 10 | 12 | 190 ± | 139 ± | 6.4 ± | 597 ± | 584 ± | 4.8 ± | 2.9 ± | 1.7 ± | 130 ± | 2.2 ± | 130 ± |
| | | | | 15 | 11 | 0.6 | 44 | 23 | 0.4 | 0.4* | 0.3 | 5* | 0.2 | 6 |
| 3 | AGA | 108 | 12 | 131 ± | 100 ± | 5.5 ± | 310 ± | 600 ± | 2.9 ± | 2.3 ± | 1.9 ± | 110 ± | 3.2 ± | 122 ± |
| | | | | 10*¹ | 9*¹ | 0.4 | 12*¹ | 38 | 0.3* | 0.3* | 0.2 | 4¹ | 0.4 | 10 |
| 4 | placebo | — | 12 | 207 ± | 139 ± | 6.4 ± | 611 ± | 593 ± | 5.7 ± | 4.1 ± | 1.6 ± | 109 ± | 3.0 ± | 132 ± |
| | | | | 14 | 16 | 0.4 | 43 | 55 | 0.5 | 0.3 | 0.3 | 8¹ | 0.5 | 12 |

*differences are statisically sigmficant as compared to Group No. 4, t-test for independent variables at p < 0.05,
¹differences are statistically significant as compared to groups No. 2 t-test for independent variables at p < 0.05

TABLE 28
Biochemical factors of blood on Day 60 of the treatment in
fluvastatin (FV) + glycyrrhizic acid (GA) groups (Day 90 of the study)

| | | | | | | | | | Bili- | Bili- | Bili- | | | |
|---|---|---|---|---|---|---|---|---|---|---|---|---|---|---|
| | | | | | | | | | rubin | rubin | rubin | | Potas- | |
| | | Dose, | | AST, | ALT, | Urea, | CPK, | Amylase, | total, | direct, | indirect, | Glucose, | sium, | Sodium, |
| | Group | mg | No. | U/l | U/l | mmol/l | U/l | U/l | mg/dl | mg/dl | mg/dl | mg/dl | mmol | mmol |
| 1 | FV + GA | 80 + | 12 | 120 ± | 108 ± | 5.3 ± | 310 ± | 560 ± | 3.1 ± | 1.6 ± | 1.8 ± | 112 ± | 2.1 ± | 133 ± |
| | | 90 | | 9*¹ | 5*¹ | 0.4 | 10*¹ | 38 | 0.3* | 0.2* | 0.2 | 9 | 0.5 | 10 |
| 2 | FV | 80 | 12 | 200 ± | 148 ± | 6.3 ± | 680 ± | 590 ± | 4.2 ± | 1.4 ± | 1.6 ± | 127 ± | 2.3 ± | 124 ± |
| | | | | 17 | 17 | 0.5 | 54 | 24 | 0.2 | 0.4* | 0.2 | 7 | 0.2 | 7 |
| 3 | GA | 90 | 12 | 121 ± | 103 ± | 5.0 ± | 233 ± | 600 ± | 4.3 ± | 2.2 ± | 1.8 ± | 116 ± | 3.1 ± | 123 ± |
| | | | | 8*¹ | 10*¹ | 0.4 | 10*¹ | 35 | 0.3* | 0.3* | 0.2 | 9 | 0.3 | 10 |
| 4 | placebo | — | 12 | 207 ± | 139 ± | 6.4 ± | 611 ± | 593 ± | 5.7 ± | 4.1 ± | 1.6 ± | | 3.0 ± | 132 ± |
| | | | | 14 | 16 | 0.4 | 43 | 55 | 0.5 | 0.3 | 0.3 | | 0.5 | 12 |

*differences are statisically sigmficant as compared to Group No. 4, t-test for independent variables at p < 0.05,
¹differences are statistically significant as compared to groups No. 2 t-test for independent variables at p < 0.05

As seen from Table 23, on day 60 of the treatment, there was observed a pronounced reduction of activity of AST by 33% and that of CPK by 51% as compared to the control group when the mixture SV+AGA was used.

The use of the studied mixture was also effective against bilirubinemia, as the concentration of total bilirubin decreased by 45%, while that of direct bilirubin fell by 41% as compared to the control group. However, the efficacy of the studied mixture against bilirubinemia exceeded that of the reference drug. Such biochemical profile dynamics may indicate a hepatoprotectory action of the studied mixture and antimyotoxic effect manifested mainly through the contribution of AGA efficacy.

By Day 60 day of the treatment a pronounced hepatoprotective effect of the mixtures statin+glycyrrhizin derivative was observed, that manifested in pronounced decline in activity of transaminases. Significantly, a reduction of activity of CPK in all groups of animals which received the combination of statin+glycyrrhizin according to the present invention compared with those groups that received statin as monotherapy was detected.

Values of AST, ALT parameters were significantly lower than those during the use of the reference drug statins in the same doses. In groups of glycyrrhizin derivatives the values of AST, ALT and CPK was comparable to baseline parameters.

In addition, the studied mixtures of statin+glycyrrhizin derivative according to the present invention had a pronounced effect on glucose concentration: the tendency to reduction of glucose concentration compared with to statin as monotherapy was observed. In groups of glycyrrhizin derivatives the value of AST, ALT and CPK was compare to baseline data.

Summarising, it has been shown that mytotoxicity and hepatotoxicity of the studied combinations of the present invention was reduced compared with statin as monotherapy due to the glycyrrhizin component of the combination. Similar protective effects for other statin+glycyrrhizinate combinations were observed.

### Mass Coefficients of Organs

Due to the fact that in the animals biochemical profile shifts were observed in terms of transaminases, CPK, organs of the studied animals were posthumously weighed, with mass coefficients calculated for pancreas, liver and kidney (Table 29-34).

TABLE 29
The weight coefficients for pancreas, liver and kidneys of the studied animals in
simvastatin (SV) + monoammonium glycyrrhizinate (AGA) groups, M ± m

| Pos. | Group | Dose, mg | No. | Pancreas | Liver | Kidneys |
|---|---|---|---|---|---|---|
| 1 | SV + AGA | 20 + 108 | 12 | 0.22 ± 0.01² | 4.24 ± 0.10 | 0.78 ± 0.03 |
| 2 | SV (20 mg) | 20 | 12 | 0.35 ± 0.03¹ | 4.42 ± 0.12 | 0.76 ± 0.03 |
| 3 | SV | 40 | 12 | 0.48 ± 0.04¹ | 4.49 ± 0.14 | 0.79 ± 0.01 |
| 4 | AGA | 108 | 12 | 0.21 ± 0.01² | 4.40 ± 0.09 | 0.81 ± 0.02 |
| 5 | placebo | — | 12 | 0.26 ± 0.01 | 4.44 ± 0.09 | 0.80 ± 0.02 |

Notes:
¹differences are statistically significant as compared to Group No. 5, t-test for independent variables at p < 0.05;
²differences are statistically significant as compared to Group No. 3, t-test for independent variables at p < 0.05.

TABLE 30
The weight coefficients for pancreas, liver and kidneys of the studied animals in
atorvastatin (AV) + glycyrrhizic acid (GA) groups, M ± m

| Pos. | Group | Dose, mg | No. | Pancreas | Liver | Kidneys |
|---|---|---|---|---|---|---|
| 1 | AV + GA | 10 + 90 | 12 | 0.21 ± 0.01² | 4.22 ± 0.10 | 0.79 ± 0.02 |
| 2 | AV | 20 | 12 | 0.34 ± 0.03 | 4.43 ± 0.12 | 0.75 ± 0.03 |
| 3 | AV | 40 | 12 | 0.49 ± 0.04¹ | 4.48 ± 0.14 | 0.78 ± 0.01 |
| 4 | GA | 90 | 12 | 0.20 ± 0.01² | 4.41 ± 0.09 | 0.80 ± 0.01 |
| 5 | placebo | — | 12 | 0.27 ± 0.01 | 4.45 ± 0.09 | 0.81 ± 0.02 |

Notes:
¹differences are statistically significant as compared to Group No. 5, t-test for independent variables at p < 0.05;
²differences are statistically significant as compared to Group No. 3, t-test for independent variables at p < 0.05.

TABLE 31
The weight coefficients for pancreas, liver and kidneys of the studied animals in
lovastatin (LV) + sodium glycyrrhizinate (SGA) groups, M ± m

| Pos. | Group | Dose, mg | No. | Pancreas | Liver | Kidneys |
|---|---|---|---|---|---|---|
| 1 | LV + SGA | 20 + 108 | 12 | 0.23 ± 0.01² | 4.23 ± 0.09 | 0.80 ± 0.03 |
| 2 | LV | 20 | 12 | 0.35 ± 0.04 | 4.45 ± 0.11 | 0.74 ± 0.02 |
| 3 | LV | 40 | 12 | 0.48 ± 0.03¹ | 4.46 ± 0.15 | 0.77 ± 0.01 |
| 4 | SGA | 108 | 12 | 0.22 ± 0.01² | 4.42 ± 0.10 | 0.81 ± 0.02 |
| 5 | placebo | — | 12 | 0.29 ± 0.01 | 4.44 ± 0.09 | 0.79 ± 0.01 |

¹differences are statistically significant as compared to Group No. 5, t-test for independent variables at p < 0.05;
²differences are statistically significant as compared to Group No. 3, t-test for independent variables at p < 0.05.

TABLE 32
The weight coefficients for pancreas, liver and kidneys of the studied animals in
pravastatin (PV) + glycyrrhetic acid (GtA) groups, M ± m

| Pos. | Group | Dose, mg | No. | Pancreas | Liver | Kidneys |
|---|---|---|---|---|---|---|
| 1 | PV + GtA | 40 + 108 | 12 | 0.20 ± 0.01² | 4.25 ± 0.09 | 0.77 ± 0.02 |
| 2 | PV | 40 | 12 | 0.47 ± 0.03¹ | 4.47 ± 0.17 | 0.76 ± 0.01 |
| 3 | GtA | 108 | 12 | 0.21 ± 0.01² | 4.43 ± 0.10 | 0.80 ± 0.02 |
| 4 | Placebo | — | 12 | 0.26 ± 0.01 | 4.47 ± 0.10 | 0.78 ± 0.02 |

¹differences are statistically significant as compared to Group No. 4, t-test for independent variables at p < 0.05;
²differences are statistically significant as compared to Group No. 2, t-test for independent variables at p < 0.05.

TABLE 33
The weight coefficients for pancreas, liver and kidneys of the studied animals in
rosuvastatin + monoammonium glycyrrhizinate groups, M ± m

| Pos. | Group | Dose, mg | No. | Pancreas | Liver | Kidneys |
|---|---|---|---|---|---|---|
| 1 | RV + AGA | 10 + 108 | 12 | 0.23 ± 0.01² | 4.23 ± 0.10 | 0.79 ± 0.03 |
| 2 | RV | 10 | 12 | 0.35 ± 0.04 | 4.42 ± 0.11 | 0.75 ± 0.03 |
| 3 | PV | 20 | 12 | 0.49 ± 0.03¹ | 4.49 ± 0.15 | 0.78 ± 0.02 |
| 4 | AGA | 108 | 12 | 0.22 ± 0.01² | 4.41 ± 0.08 | 0.81 ± 0.01 |
| 5 | Placebo | = | 12 | 0.27 ± 0.01 | 4.43 ± 0.09 | 0.80 ± 0.01 |

¹differences are statistically significant as compared to Group No. 5, t-test for independent variables at p < 0.05;
²differences are statistically significant as compared to Group No. 3, t-test for independent variables at p < 0.05.

TABLE 33a
The weight coefficients for pancreas, liver and kidneys of the studied animals in
fluvastatin (FV) + glycyrrhizic acid (GA) groups, M ± m

| Pos. | Group | Dose, mg | No. | Pancreas | Liver | Kidneys |
|---|---|---|---|---|---|---|
| 1 | FV + GA | 80 + 90 | 12 | 0.25 ± 0.02² | 4.24 ± 0.10 | 0.77 ± 0.02 |
| 2 | FV | 80 | 12 | 0.37 ± 0.03¹ | 4.41 ± 0.12 | 0.75 ± 0.03 |
| 3 | GA | 90 | 12 | 0.23 ± 0.01² | 4.42 ± 0.09 | 0.80 ± 0.03 |
| 4 | placebo | — | 12 | 0.25 ± 0.01 | 4.44 ± 0.08 | 0.79 ± 0.02 |

¹differences are statistically significant as compared to Group No. 4, t-test for independent variables at p < 0.05;
²differences are statistically significant as compared to Group No. 2, t-test for independent variables at p < 0.05.

It follows from the results shown in Tables 29-33a that the reference drugs when used as monotherapy increase the mass coefficients of pancreas. Such changes can be due to adverse reactions described for statins among which one of the most significant is aggravation of inflammatory changes in pancreas. In addition, it should be noted that in some cases simvastatin and atorvastatin mono therapy increase the risk of development of diabetes mellitus.

It was noted that statins are able to provide inhibitive effect on the processes of intracellular signal transduction of insulin, leading to a decrease in the expression of GLUT4 and deregulating GLUT1 in adipose tissues (Takaguri et al J Pharmacol Sci. 2008 Vol. 107, No. 1. P. 80-89). This helps to reduce insulin-dependent transport of glucose to cells and insulin sensitivity, which can induce intolerance to glucose. It is also possible that insulin resistance associated with statins, could lead to suppression of biosynthesis of isoprenoid, an intermediate product in formation of cholesterol. In addition, statins may directly influence the secretion of insulin, by influencing β-cells of pancreas by inhibiting glucose-stimulated increase of free cytoplasmic calcium and L-channels for this ion. The properties of the statins to intensify the processes of inflammation and oxidation in pancreatic islets may cause the development of diabetes in patients with impaired carbohydrate metabolism or susceptibility and risk factors of this disease (Otocka-Kmiecik et al Postepy Biochem. 2012. Vol. 58, No. 2. pp. 195-203).

The use of the studied combinations of the present invention, in particular SV+AGA and AV+GA, resulted in a statistically significant reduction of mass coefficients of pancreas as compared to those in the group which received monotherapy of statins. The use of the studied mixture and the reference drug did not influence mass coefficients of liver and kidneys.

### Conclusions

The use of the physical mixtures of statin and glycyrrhizin derivative according to the present invention had an effect onto the parameters of lipid spectrum in rat hypercholesterolemic model. The use of the physical mixture led to a decrease in the level of CHS, TG and LDL throughout the treatment course.

The efficacy of the combinations relative to lipid spectrum was higher than that of the reference drug statin as monotherapy in the same doses and close to double the therapeutic dose of statin as monotherapy.

The hypolipidemic effects of the combinations of statin and glycyrrhizin derivative according to the present invention, as exemplified by atorvastatin (10 mg)+glycyrrhizic acid, lovastatin (20 mg)+glycyrrhizic acid, and rosuvastatin (10 mg)+monoammonium glycyrrhizinate exceeded the effects of statin monotherapy in the same doses and was comparable with that achieved using of double the corresponding statin doses, atorvastatin (20 mg), lovastatin (40 mg), rosuvastatin (20 mg) as monotherapy. The hypolipidemic effects of pravastatin (40 mg)+glycyrrhetic acid group and fluvastatin+glycyrrhizic acid group exceeded the effects of statin monotherapy in the same doses. Therefore, the efficacy of a representative selection of combinations of statin and glycyrrhizin derivative according to the present invention the present invention relative to lipid spectrum was higher than that of the reference drug statin in the same doses and close to double therapeutic dose.

The hypocholesterolemic activity for all glycyrrhizin derivatives were detected and were lower than in other study groups. However, the effect of the combination of statin and glycyrrhizin derivative according to the present invention derivative in each of the tested combinations was is substantially greater than the effect of each individual component when used alone, indicating a synergistic effect for the tested combinations.

Evidence of the favourable hypocholesterolemic efficacy of pharmaceutical compositions and potential to reduce effective drug dose, with the view of statin proportion in the composition has therefore been demonstrated.

It can therefore be concluded that combinations of statins and glycyrrhizin derivatives (exemplified by ammonium glycyrrhizinate, glycyrrhizic acid, sodium glycyrrhizinate and glycyrrhetic acid) according to the present invention possess a synergistic hypocholesterolemic activity.

Thus, as the combinations of statin and glycyrrhizin derivative according to the present invention was administered for 30 and 60 days, the pronounced contribution of glycyrrhizin derivative to hypocholesterolemic effect of the mixture was noted—as in these days of the treatment more pronounced decrease of the concentration of CHS, LDL, TG, and increase in HDL than in the group of animals which received statin.

Statins did not prevent toxic effect of the diet on liver tissue of experimental animals. Alanine-aminotransferase and aspartate-aminotransferase activities were a little lower compared with the control group but exceeded the normal values. The treatment of animals with statins and glycyrrhizinates combination produced effect on transaminase activity, particularly alanine-aminotransferase, reducing it. Similar effects were reported for other study groups using the combinations of statin and glycyrrhizin derivative according to the present invention. These effects confirm hepatoprotective properties of the combinations of statin and glycyrrhizin derivative according to the present invention compared with the components when used alone.

A marked reduction of creatine phosphokinase activity at using the combinations of statin and glycyrrhizin derivative according to the present invention compared with the group receiving reference drug with a therapeutic dose was observed. The obtained data afford an opportunity to conclude undoubted role of glycyrrhizinates in reduced mytotoxicity being adverse effect of statins.

The obtained data afford an opportunity to conclude the undoubted role of glycyrrhizinates in reduced mytotoxicity, a known adverse effect of statins.

The use of the studied mixture of according to the present invention according to the present invention contributed to the reduction of mass coefficients of pancreas as compared to those in the group which received the reference drug statins as monotherapy.

The studied combinations of statin and glycyrrhizin derivative according to the present invention had pronounced effect onto glucose concentration, the tendency to reduction of glucose concentration compared with statin as monotherapy was observed.

The obtained data allow us to conclude that combinations of statin and glycyrrhizin derivatives (exemplified by ammonium glycyrrhizinate, glycyrrhizic acid, sodium glycyrrhizinate and glycyrrhetic acid) according to the present invention possess a synergistic effect not characteristic for each of these components individually.

Thus, the use of the combinations of statin and glycyrrhizin derivative according to the present invention led to less pronounced manifestations of hypercholesterolemia and protection of target organs in the simulated pathology.

Pharmaceutical compositions containing combinations of glycyrrhizinates with statins according to the present invention, with indicated daily dose demonstrated both hypolipidemic effect compared to double therapeutic dose of statin as monotherapy. Safety profile was associated with reduced of adverse effects such as hepatotoxicity and mytotoxicity. Similar effects were observed for all tested combinations of statin and glycyrrhizinates according to the present invention.

### Example 2—Chromatographic Analysis

The solid pharmaceutical compositions of simvastatin and ammonium glycyrrhizinate (SV+AGA), atorvastatin and glycyrrhizic acid (AV+GA) and rosuvastatin and, ammonium glycyrrhizinate (RV+AGA) were tested by chromatography to investigate whether they contained a molecular complex of the two ingredients. In all cases, the doses were the same as those set out in Table 1 of Example 1.

The following reagents were used:

SV+AGA: HPLC grade acetonitrile and analytical grade orthophosphoric acid, acetic acid, sodium phosphate dihydrate, sodium hydroxide. AV+GA: The HPLC grade acetonitrile and analytical grade orthophosphoric acid, ammonium citrate, tetrahydrofuran, sodium hydroxide. RV+AGA: HPLC grade acetonitrile and analytical grade orthophosphoric acid, trifluoroacetic acid.

These materials were purchased from Merck, Darmstadt, Germany. Water was prepared using Millipore Milli.Q Plus water purification system, Bedford, Mass., USA.

The following chromatographic conditions and equipment was used:

A UV detector was employed. The output signal was monitored and processed using empowers software.

The column conditions are set out in Table 34 below.

TABLE 34
The column condition description of HPLC method

| Condition | SV + AGA | AV + GA | RV + GA |
|---|---|---|---|
| chromatographic | C18, 5 μm, | C18, 5 μm, | C18, 5 μm, |
| column | 250 mm, | 250 mm, | 250 mm, |
| | Ø 4.6 mm | Ø 4.6 mm | Ø 3.2 mm |
| separation | isocratic | Isocratic | gradient |
| method | | | |
| solvent | The solvent | The solvent | The solvent A |
| | contains a | contains a | contains a |
| | mixture of | mixture of | mixture of 1.0% |
| | 0.025M sodium | 0.05M | trifluoroacetic |
| | phosphate | ammonium | acid in water, |
| | dihydrate buffer | citrate buffer, | Acetonitrile in |
| | and acetonitrile | tetrahydrofuran | the ratio |
| | in the ratio | and acetonitrile | 63:37 (v/v); |
| | 35:65 (v/v) | in the ratio | and the solvent |
| | | 27:20:53 (v/v) | B contains a |
| | | | mixture of 1.0% |
| | | | trifluoroacetic |
| | | | acid in water |
| | | | and acetonitrile |
| | | | in the ratio |
| | | | 10:90 (v/v), |
| | | | respectively. |
| flow rate of | 1.5 ml/min | 15 ml/min | 0.75 ml/min |
| mobile phase | | | |
| column | 45° C. | 25° C. | 40° C. |
| temperature | | | |
| wavelength | 238 nm | 244 nm | 242 nm |
| detection | | | |
| injection volume | 10.0 μl | 20.0 μl | 10.0 μl |

The results are shown in Table 35 below. In all cases, the tests did not detect the presence of a molecular complex of the statin and the glycyrrhizinate derivative.

TABLE 35
The results of chemical stability study

| | | | | starting point | 6 months | 12 months | 24 months |
|---|---|---|---|---|---|---|---|
| | | | | mix | mix | mix | mix |
| | Daily | | Specification | Test result | Test result | Test result | Test result |
| | therapeutic | | Limit, not | not more | not more | not more | not more |
| | dose | | more than | than | than | than | than |
| Composition | (mg) | Impurity | (NMT), % | (NMT), % | (NMT), % | (NMT), % | (NMT), % |
| simvastatin (SV) | 20, 108 | Hydroxyacid | 1.00 | 1.00 | 1.00 | 1.00 | 1.00 |
| ammonium | | simvastatin | | | | | |
| glycyrrhizinate | | any other relative | 0.25 | 0.25 | 0.25 | 0.25 | 0.25 |
| (AGA) | | Impurity | | | | | |
| | | any other Impurity | 0.25 | 0.25 | 0.25 | 0.25 | 0.25 |
| | | sum Impurity | 1.50 | 1.50 | 1.50 | 1.50 | 1.50 |
| atorvastatin (AV), | 10, 90 | ImpurityATN1 | 0.20 | 0.20 | 0.20 | 0.20 | 0.20 |
| glycyrrhizic | | Impurity ATN2 | 0.20 | 0.20 | 0.20 | 0.20 | 0.20 |
| acid (GA) | | Impurity ATN14 | 0.30 | 0.30 | 0.30 | 0.30 | 0.30 |
| | | Impurity ATN4,5 | 0.40 | 0.40 | 0.40 | 0.40 | 0.40 |
| | | Impurity ATN12 | 0.50 | 0.50 | 0.50 | 0.50 | 0.50 |
| | | Impurity ATN13 | 0.50 | 0.50 | 0.50 | 0.50 | 0.50 |
| | | Impurity ATN15 | 0.50 | 0.50 | 0.50 | 0.50 | 0.50 |
| | | Impurity ATN16 | 0.50 | 0.50 | 0.50 | 0.50 | 0.50 |
| | | Impurity ATL1 | 0.50 | 0.50 | 0.50 | 0.50 | 0.50 |
| | | unknown Impurity | 0.20 | 0.20 | 0.20 | 0.20 | 0.20 |
| | | sum Impurity | 2.00 | 2.00 | 2.00 | 2.00 | 2.00 |
| rosuvastatin (RV), | 10, 108 | 5-ketoacid | 0.50 | 0.50 | 0.50 | 0.50 | 0.50 |
| ammonium | | rosuvastatin lactone | 0.50 | 0.50 | 0.50 | 0.50 | 0.50 |
| glycyrrhizinate | | rosuvastatin | 0.50 | 0.50 | 0.50 | 0.50 | 0.50 |
| (AGA) | | antiisomer | | | | | |
| | | any other not detective | 0.40 | 0.40 | 0.40 | 0.40 | 0.40 |
| | | Impurity | | | | | |
| | | sum Impurity | 2.00 | 2.00 | 2.00 | 2.00 | 2.00 |

### Example 3—Further Non-Clinical Study

The present study was aimed at determining the specific pharmacological activity of the compounds on the model of hypercholesterolemia and atherosclerosis caused by impact of atherogenic factors, identifying the type of dependence dose-effect and determination of the optimal therapeutic dose for extrapolation to the clinic in rabbit model. The model described herein using rabbits as the experimental animals is a best reproducible standard model for confirmation of hypocholesterolemic effect.

### Materials and Methods

### Animals:

The animals used in this experiment were involved reproductive male rabbits of Californian breed. 144 rabbits were used. The weight of the animals at the beginning of the trial ranged from 2.5 to 3 kg; the animals were 8 weeks old. Before the study the laboratory animals were contained for 27 and for 37 days in the separate coops for adaptation. During this period, clinical condition of animals was controlled every day by visual inspection. The criteria of the inclusion of animals in the experiment were health and body weight.

The division of animals in groups was carried out randomly. Animals were selected into the experimental groups, using a random number generator in the statistical program Statistica 6.0. The animals were kept under standard conditions in accordance with the “Guidelines for the Care and Use of Laboratory Animals” National Academy Press, Washington, D C 1996, and regulations approved by the USSR Ministry of Health on 6 Jul. 1973, on arrangement, equipment and maintenance of experimental biological clinics (vivariums).

### Administration and Selection of Doses

Intragastric administration was used during the study as the upper route is planned for administration to humans in clinical practice.

Based on the results of Example 1 to determine an effective and safe dose of glycyrrhizinates in fixed combinations, the selected dose of monoammonium glycyrrhizinate was 50 mg, 100 mg and 200 mg. This is the amount by weight of glycyrrhizic acid excluding the contribution of the ammonium counter ion. The selection of doses of rosuvastatin and atorvastatin is based on average daily dose of these medicines for human according to Basic Prescribing Information.

The dose of the combinations of statin and glycyrrhizin derivative according to the present invention tested in this Example are shown in Table 36 below. Atorvastatin and rosuvastatin were both administered as calcium salts. For both the statin and the glycyrrhizinate, the amounts by weight are expressed as weight of the free acid equivalent, excluding the contribution of the counter-ion.

TABLE 36

| | Daily therapeutic |
|---|---|
| Composition | dose (mg) |
| atorvastatin (AV) | 20 |
| ammonium glycyrrhizinate (AGA) | 50 |
| atorvastatin (AV) | 20 |
| ammonium glycyrrhizinate (AGA) | 100 |
| atorvastatin (AV) | 20 |
| ammonium glycyrrhizinate (AGA) | 200 |
| rosuvastatin (RV) | 20 |
| ammonium glycyrrhizinate (AGA) | 50 |
| rosuvastatin (RV) | 20 |
| ammonium glycyrrhizinate (AGA) | 100 |
| rosuvastatin (RV) | 20 |
| ammonium glycyrrhizinate (AGA) | 200 |

These combinations were prepared as physical mixtures of the solid ingredients in 0.5% (weight/volume) methylcellulose solution in water, with no other excipients.

### Methodology

### Study Design

The introduction of drugs under two schemes was used to determine the mechanism of action of the studied drugs and the impact on the different stages of atherosclerosis. Each group was divided into subgroup A, receiving medicines from 31 to 90 days and subgroup B, receiving medicines from 61 to 120 days During the treatment from 31 to 90 days the efficacy of medicines indicated the hypoglycemic activity evaluated among other things besides lipid metabolism changes prevention or slowing of all stages of development of atherocalcinosis and atheromatosis. In the treatment from 61 to 120 days efficacy indicated its ability to induce regression of atherosclerotic plaques (antiatherosclerotic efficiency). Characteristics of the study groups are presented in Table 37.

TABLE 37
Characteristics of the studied groups of animals

| | | | Dose of | | | |
|---|---|---|---|---|---|---|
| Number | Number | Number | active | | | |
| of | of | of | substance | | Days of | Day of |
| group | subgroup | animals | (mg) | Group Description | treatment | euthanasia |
| 1 | | 6 ♂ | 0 | Intact (no abnormality, no | — | On 121 day study |
| | | | | treatment) | | |
| 2 | | 6 ♂ | 0 | Control—with pathology, no | — | 91 day study |
| | | | | treatment | | |
| 3 | 2 B | 6 ♂ | 0 | Control—pathology, no | — | 61 day study |
| | | | | treatment | | (control of plaque) |
| 4 | 2 B1 | 6 ♂ | 0 | Control—pathology, no | — | 91 or 121 days of |
| | | | | treatment | | research, |
| 5 | 3 A | 6 ♂ | 20 + 200 | Experimental—with | 31-90 | respectively, |
| 6 | 3 B | 6 ♂ | | pathology + treatment by | 61-120 | |
| 7 | 4 A | 6 ♂ | 20 + 100 | tested object | 31-90 | |
| 8 | 4 B | 6 ♂ | | (rosuvastatin + AGA) | 61-120 | |
| 9 | 5 A | 6 ♂ | 20 + 50 | | 31-90 | |
| 10 | 5 B | 6 ♂ | | | 61-120 | |
| 11 | 6 A | 6 ♂ | 20 | Animals with pathology + | 31-90 | |
| 12 | 6 B1 | 6 ♂ | 20 | rosuvastatin treatment | 61-120 | |
| 13 | 6 A2 | 6 ♂ | 40 | | 31-90 | |
| 14 | 7 A | 6 ♂ | 20 + 200 | Experimental—with | 31-90 | |
| 15 | 7 B | 6 ♂ | | pathology + treatment by | 61-120 | |
| 16 | 8 A | 6 ♂ | 20 + 100 | tested object | 31-90 | |
| 17 | 8 B | 6 ♂ | | (atorvastatin + AGA) | 61-120 | |
| 18 | 9 A | 6 ♂ | 20 + 50 | | 31-90 | |
| 19 | 9 B | 6 ♂ | | | 61-120 | |
| 20 | 10 A | 6 ♂ | 20 | Animals with pathology + | 31-90 | |
| 21 | 10 B1 | 6 ♂ | 20 | atorvastatin treatment | 61-120 | |
| 22 | 0 A2 | 6 ♂ | 40 | | 31-90 | |
| 23 | 11 A | 6 ♂ | 100 | Animals with pathology + | 31-90 | |
| 24 | 11 B | 6 ♂ | 100 | treatment AGA | 61-120 | |

Research Design

Research design for the subgroup A and B is shown in Tables 38 and 39.

TABLE 38
Research Design—Group A

| | Day of experiment | | | | | | | |
|---|---|---|---|---|---|---|---|---|
| Evaluation | | | | 45 | 60 | 75 | 90 | 91 |
| Cholesterol administration | Every day from day 1 till 90 | | | | | | | |
| of vitamin D3 and adrenaline | — | — | Every day since 31 | | | — | — | — |
| | | | till 60 days | | | | | |
| Administration of drugs | — | — | Daily since day 31 till 90 | | | | | — |
| Assessment of the general condition | Daily | | | | | | | |
| Weighing | Weekly | | | | | | | |
| Lipid profile | | | | | | | | — |
| Parameters of the coagulation system | | | | | * | | | — |
| Toxicological data | | | | | * | | * | — |
| Euthanasia (macroscopic of aorta, heart, | | | | | | | | |
| morphometry of the aorta, the aorta and | | | | | | | | |
| liver histology) | | | | | | | | |
| Mass coefficients (liver, pancreas) | | | | | | | | * |

TABLE 39
Design of research group B

| | Day of experiment | | | | | | | | |
|---|---|---|---|---|---|---|---|---|---|
| Evaluation | | 30 | 45 | 60 | 75 | 90 | 105 | 120 | 121 |
| Administration of cholesterol | Daily from day 1 to 120 | | | | | | — | — | — |
| of vitamin D3 and adrenaline | | Daily from 31 to 60 days | | | | | | | |
| Administration of drugs | | | | Daily from 61 to 120 days | | | | | |
| Assessment of the general | Daily | | | | | | | | |
| condition | | | | | | | | | |
| Weighing | Weekly | | | | | | | | |
| Lipid profile | | | | | | | | | — |
| Parameters of the coagulation | | | | * | | | | | — |
| system | | | | | | | | | |
| Toxicological data | | | | | | | | | — |
| Euthanasia (macroscopic of aorta, | | | | | | | | | |
| heart, morphometry of the aorta, | | | | | | | | | |
| the aorta and liver histology) | | | | | | | | | |
| Mass coefficients (liver, pancreas) | | | | | | | | | |

### Induction of Hypercholesterolemia and Atherosclerosis

The offered model of hypercholesterolemia and atherosclerosis includes the impact of all major pathogenetic factors of atherosclerosis:

1. Cholesterol administered daily in an oil solution at 0.3 g/kg of animal body weight, one time, 4 hours after drug administration. Cholesterol was dissolved in hot sunflower oil and administered to the animals through noninvasive probe into the stomach. The duration of administration amounted cholesterol from 1 to 90 days in subgroup A and 1 to 120 days in subgroup B.
2. After 30 days from the initiation of administration of cholesterol vitamin D3 (cholecalciferol) in a dose of 0.256 ml/kg was added to the diet of animals to enhance aortic lipidosis. Cholecalciferol was administered intragastrically via a probe from 31 till 60 days (Scheme A and B).
3. In order to gain atherosclerotic lesions in aorta and atherosclerosis induction time reduction the animals were administered a dose of epinephrineequal to 0.04 mg/kg intravenously since 31 till 60 every five days (Scheme A and B).

Animal body weight was measured in accordance with standard procedures prior to the study, then—weekly (for calculating the volume of administration of the test compound and the reference drug) and immediately before euthanasia (on days 60 and 120)—to calculate the weight gain.

### Administration of Test Objects and Sample Preparation

The administration was carried out intragastrically in accordance with standard procedures using atraumatic probe according to two treatment regimens every day at the same time.

### Biochemical Blood Tests

Parameters for evaluation of efficacy of drugs: Biochemical parameters and activity of blood serum enzymes (lipid spectrum; coagulation system).

Parameters for evaluation of drug toxicity (biochemical parameters and activity of serum enzymes (AST, ALT, CPK, total bilirubin, direct and indirect bilirubin, glucose, potassium ions, sodium).

Blood sampling was carried out in test animals in vivo from the marginal ear vein of the rabbit. Preparation of blood for research was carried out in accordance with standard procedures for preparation of blood for biochemical and coagulometric studies). Venous blood was sampled in a sterile plastic tubes containing anticoagulant heparin. Tubes of blood were centrifuged at 1000-3000 rev/min for 10-15 minutes. Plasma samples were used without hemolysis.

Lipid profile parameters were evaluated with biochemical analyzer of open type A-25 Random Access (Spain) using reagents of firm BioSystems (Spain) in accordance with standard procedures for its use.

Standard kinetic methods of spectrophotometry described in recommendations were used (indicated in Example 1), as follows:

### Determination of the Concentration of Total Cholesterol (TC)

The total cholesterol in the test material was determined photometrically at a wavelength of 490-520 nm, as described in Allain C. C., et al. Clin Chem. 1974, 20, 470-475 and. Meiattini F et al. Clin Chem. 1978, 24, 2161-2165.

### Determination of the Concentrations of HDL Cholesterol and Low-Density Lipoproteins (HDL, LDL)

HDL Cholesterol is measured spectrophotometrically at 600 nm as described in Warnick G R et al. Clin Chem 2001; 47: 1579-96. Content of LDL cholesterol was then determined by calculation as described in J. Marshall. Clinical chemistry/Per.s Eng.-M-Petersburg.: “Publisher Bean”-“Nevsky Dialect”, Pub. 1999. 368.

Determination of the concentration of triglycerides (TG)

The content of triglycerides was measured spectrophotometrically at 365 or 405 nm as described in Bucolo G., et al. Clin Chem.-1973.-Vol. 19.-P. 476-482, and in Fossati P., Prencipe L. Clin Chem. 1982, vol. 28-P. 2077-2080.

### Analysis of the Blood Coagulation System Profile:

Coagulation system parameters were evaluated on a coagulometer APG2-02P (Russia) according to standard procedures (Biggs R: Thromb Diath Haem Supl 17: 303 (1965). Proctor R & Rapaport S: J Clin Path 36: 212 (1961). Hardisty R M & Ingram G I C: Bleeding disorders investigation and management. Blackwell Scientific Publications, Oxford, 1965.)

The development of atherosclerosis in the clinic in most cases accompanied by atherothrombosis, which is the leading cause of death due to myocardial and brain ischemia. In case of hypercholesterolemia and atherosclerosis simulation by means of impact of atherogenic factors as well as in clinic we can observe expressed changes of blood coagulation parameters in rabbits which contribute to the thrombus formation and plaque abraption. Experimental evaluation of the parameters of the blood coagulation system allows determination of the possible impact of drugs on the dynamics of atherothrombosis.

Animals were euthanized on 61, 91 and 121 day of research.

### Morphometry of Aorta

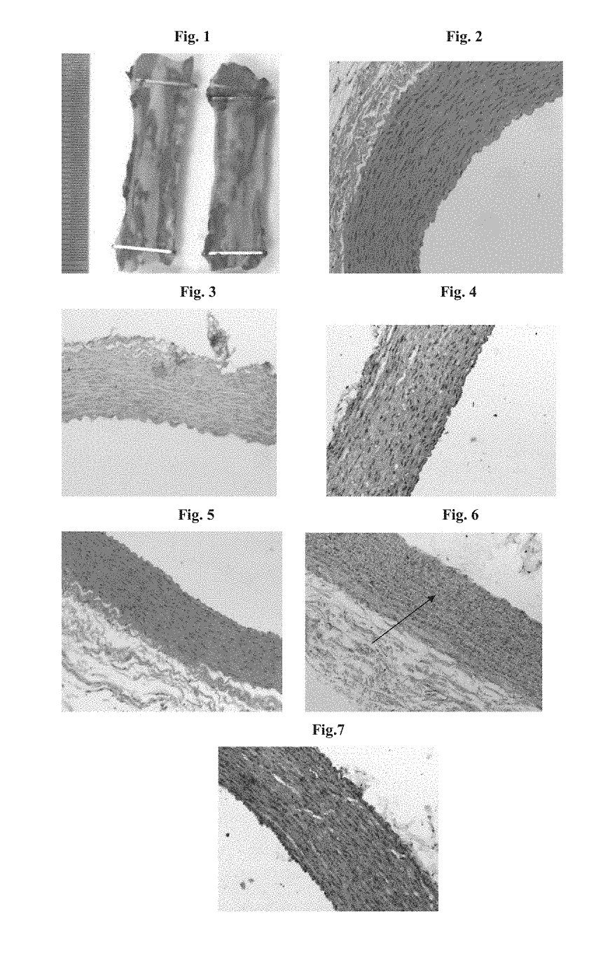

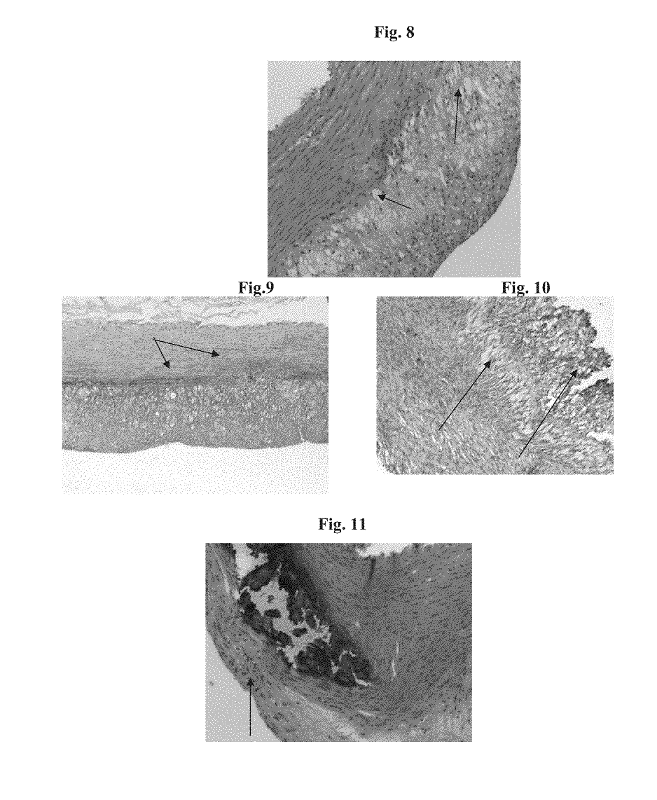

Section of the aorta was stained with fatty red dye (Oil Red O) for 30 minutes, then washed with 70% alcohol for differentiation. In 15 minutes it was washed with water and photographed. The square of the “red area” was calculated (FIG. 1)—the interior surface of the spots was measured with the usage of morphometry computer system VideoTest Size 5.0 (Russia). The percentage of plaque area was expressed as a percentage of the total surface area of the taken portion of the aorta.

### Histological Analysis

Tissues of liver, aorta, left heart valve and pancreas were subjected to histological analysis. Part of the aorta, left heart valve and liver fragments were fixed in 10% neutral buffered formalin for 1 day and poured in paraffin. Aortic sections were coloured with hematoxylin and toluidine blue and examined by light microscopy. Additionally for histochemical studies and for the purpose of identifying of lipids from fixed biopsies of aorta were arranged sections of aorta 7-10 μm in thickness, the sections were coloured with Sudan-3 on the neutral fats and with fat red dye (Oil Red O). Sections of the liver of 5-7 μm in thickness were coloured with hematoxylin and eosin and examined by light microscopy.

### Determination of the Mass Ratios of the Organs

Determination of the mass ratios of the liver were conducted according method described in Example 1

### Data Analysis:

Data analysis was conducted according to the methods described in Example 1.

### Results of the Study of Specific Pharmacological Activity of Combinations

### Biometrics

### Dynamics of Body Weight of Experimental Animals

Tables 40 and 41 show data on the dynamics of body weight in experimental animals prior to the study, on the background of disease without treatment and under the treatment.

TABLE 40
Dynamics of body weight - Scheme A

| | | | | | Body weight, g, M ± SEM | | |
|---|---|---|---|---|---|---|---|
| | | | | | | Day 28 (in the | |
| | | Dose of | | | | setting of | Day 90 (in the |
| | | active | | | | pathology | setting of |
| | | substance | Group | | Day 0 | without | pathology with |
| | subgroup | (mg) | Description | | (Before the study) | treatment) | treatment) |
| 2 | 2A | 0 | Control | 6 | 2748 ± 100 | 3184 ± 92 | 3877 ± 41 |
| 5 | 3A | 20 + 200 | Treatment with | 6 | 2763 ± 97 | 3315 ± 99 | 3971 ± 54 |
| 7 | 4A | 20 + 100 | rosuvastatin + AGA | 6 | 2950 ± 198 | 3678 ± 131 | 4139 ± 36 |
| 9 | 5A | 20 + 50 | (the present invention) | 6 | 2951 ± 123 | 3396 ± 102 | 4035 ± 36 |
| 11 | 6A | 20 | Comparison - treatment | 6 | 2874 ± 165 | 3350 ± 157 | 4340 ± 53 |
| 13 | 6A2 | 40 | with rosuvastatin | 6 | 2787 ± 141 | 3428 ± 146 | 4348 ± 66 |
| 14 | 7A | 20 + 200 | Treatment with | 6 | 3010 ± 183 | 3447 ± 131 | 4022 ± 75 |
| 16 | 8A | 20 + 100 | atorvastatin + AGA (the | 6 | 2832 ± 116 | 3228 ± 112 | 3979 ± 95 |
| 18 | 9A | 20 + 50 | present invention) | 6 | 2887 ± 86 | 3211 ± 68 | 3937 ± 85 |
| | | | Comparison- treatment | 6 | 3017 ± 132 | 3334 ± 160 | 4111 ± 148 |
| | | | with atorvastatin | 6 | 2913 ± 157 | 3416 ± 128 | 4266 ± 101 |
| | | | Comparison- treatment | 6 | 2936 ± 150 | 3369 ± 168 | 3955 ± 119 |
| | | | with AGA | | | | |

Dispersive analysis with repeated measures prior to treatment (on day 0 and day 28 of the study) revealed that the combined effect of the time factor and the group is characterized by values F 11; 60=2.07, p=0.04. However, on day 0 and day 28, a statistically significant difference between study groups were absent (according to criteria Newman-Keuls test). Thus, prior to the start of treatment the body weight gain in all treatment groups were equally expressed.

Dispersive analysis with repeated measures on the 28 day and 90 day of study revealed that the combined influence of the time factor and the use of drugs is characterized by values F 11, 60=3.23, p=0.002. In this case also statistically significant differences between treatment groups during treatment (on 90 day of study) were absent according to Newman-Keuls test criterion.

Thus, the use of study drugs had no effect on the dynamics of body weight of animals in the treatment according to Scheme A.

TABLE 41
Dynamics of body weight - Scheme B

| | | | | | Body weight, g, M ± SEM | | |
|---|---|---|---|---|---|---|---|
| | | | | | | Day 56 (in the | Day 120 ((in |
| | | Dose of | | | | setting of | the setting of |
| | | active | | | Day 0 | pathology | pathology |
| | | substance | Group | | (Before the | without | with |
| | | (mg) | Description | N | study) | treatment) | treatment)) |
| 1 | 1 | 0 | Intact | 6 | 2744 ± 76 | 3272 ± 140 | 4066 ± 47 |
| 3 | 2B | 0 | Control | 6 | 2641 ± 71 | 3432 ± 122 | —* |
| 4 | 2 B 1 | 0 | Control | 6 | 2684 ± 72 | 3481 ± 64 | 4726 ± 120¹ |
| 6 | 3 B | 20 + 200 | Treatment with rosuvastatin + | 6 | 2680 ± 162 | 3444 ± 109 | 4049 ± 62² |
| 8 | 4 B | 20 + 100 | AGA (the present invention) | 6 | 2734 ± 66 | 3501 ± 84 | 4288 ± 145 |
| 10 | 5 B | 20 + 50 | | 6 | 2620 ± 73 | 3600 ± 87 | 4603 ± 80¹ |
| 12 | 6 B | 20 | Comparison- treatment with | 6 | 2665 ± 85 | 3479 ± 106 | 4699 ± 131¹ |
| | | | rosuvastatin | | | | |
| 15 | 7 B | 20 + 200 | Treatment with atorvastatin + | 6 | 2674 ± 76 | 3513 ± 95 | 4273 ± 59 |
| 17 | 8 B | 20 + 100 | AGA (the present invention) | 6 | 2773 ± 126 | 3539 ± 118 | 4225 ± 97² |
| 19 | 9 B | 20 + 50 | | 6 | 2627 ± 85 | 3514 ± 140 | 4594 ± 116¹ |
| 21 | 10 B | 20 | Comparison- treatment with | 6 | 2804 ± 170 | 3564 ± 100 | 4664 ± 126¹ |
| | | | atorvastatin | | | | |
| 24 | 11 B | 100 | Comparison- treatment with | 6 | 2809 ± 145 | 3569 ± 164 | 4150 ± 144² |
| | | | AGA | | | | |

Notes:
¹p < 0.05 - a significant difference from the intact group (Newman-Keuls test);
²p < 0.05 - a significant difference from the control group (Newman-Keuls test);
*Control group 2B was euthanized in order to control the development of plaques on 61 day of the study.

Dispersive analysis with repeated measures prior to treatment (on day 0 and day 60 of study) showed that the combined effect of the time factor and the group is characterized by values F 11, 60=1.00; p=0.45. According to the Newman-Keuls test differences between the groups on 0 and 60 days of research were absent. However, body weight values on day 60 for all groups exceeded those on day 0, indicating uniform growth of body weight in all groups.

Dispersive analysis with repeated measures on the 60 day and 120 day study revealed that the combined effect of the time factor and the use of drugs is characterized by values of F 10, 55=3.39, p=0.002.

In the analysis of intergroup differences on day 120 it was revealed by Newman-Keuls test that the body weight in the control group was significantly higher than in the intact group, indicating the development of experimental pathology. Herewith the mass of the animal body receiving rosuvastatin and atorvastatin as monotherapy and combinations with the lowest AGA (rosuvastatin+AGA 20 mg+50 mg and atorvastatin+AGA 20 mg+50 mg) also differed significantly from body weight in intact group.

The animals' body weight was significantly lower than in control animals in case of administration of the combination of rosuvastatin+AGA in dose 20+200 mg, combination of atorvastatin+AGA in dose 20+100 mg and monotherapy with AGA in dose 100 mg.

### Data of Pathomorphological Study

Table 42 shows mass coefficients of liver and pancreas of animals of Scheme A.

TABLE 42
Mass coefficients of organs - Scheme A

| | | Dose of | | | | |
|---|---|---|---|---|---|---|
| | | active | | | | |
| | | substance | | | Mass ratios, M ± SEM | |
| | | (mg) | Group Description | N | Liver | Pancreas |
| 2 | 2A | 0 | Control | 6 | 3.5 ± 0.2 | 0.07 ± 0.005 |
| 5 | 3A | 20 + 200 | Treatment with rosuvastatin + | 6 | 3.2 ± 0.1 | 0.06 ± 0.003 |
| 7 | 4A | 20 + 100 | AGA (the present invention) | 6 | 3.1 ± 0.2 | 0.06 ± 0.004 |
| 9 | 5A | 20 + 50 | | 6 | 3.4 ± 0.3 | 0.07 ± 0.005 |
| 11 | 6A | 20 | Comparison - treatment with | 6 | 3.8 ± 0.2 | 0.07 ± 0.006 |
| 13 | 6A2 | 40 | rosuvastatin | 6 | 4.0 ± 0.1 | 0.09 ± 0.004* |
| 14 | 7A | 20 + 200 | Treatment with atorvastatin + AGA | 6 | 3.0 ± 0.3 | 0.05 ± 0.004 |
| 16 | 8A | 20 + 100 | (the present invention) | 6 | 3.1 ± 0.2 | 0.06 ± 0.004 |
| 18 | 9A | 20 + 50 | | 6 | 3.6 ± 0.3 | 0.07 ± 0.005 |
| 20 | 10A | 20 | Comparison- treatment with - | 6 | 3.7 ± 0.2 | 0.07 ± 0.005 |
| 22 | 10A2 | 40 | atorvastatin | 6 | 3.8 ± 0.3 | 0.08 ± 0.04 |
| 23 | 11A | 100 | Comparison- treatment with AGA | 6 | 3.3 ± 0.1 | 0.05 ± 0.003* |

Notes:
-.
*P < 0.05 - a significant difference from the control group (Newman-Keuls test).

Dispersive analysis showed that the influence of the factor of the use of drugs on the mass ratios of the liver characterized by the values F 11, 60=2.16, p=0.03. According to Newman-Keuls test differences of liver mass coefficients between the two groups were absent.

However, it is noteworthy that we can observe the tendency of mass coefficients increase relative to the normal values of the control group, as well as the tendency of liver mass coefficients increase in case of monotherapies with rosuvastatin and atorvastatin. In the setting of administration of statins and AGA combinations according to the present invention, the liver mass coefficients were lower than in the control group and significantly lower than in the groups receiving atorvastatin and rosuvastatin, which may bear indirect evidence of a possible hepatoprotective effect of the combination of the present invention. It should be noted that, despite the lack of statistical significance, the liver mass coefficients in the group of AGA monotherapy, were less than in the control group.

Dispersive analysis of pancreas mass coefficients data showed the influence of the factor of drug administration on that parameter, which were characterized with the values F 11, 60=6.74, p<0.00001. Statistically expressed significant increase in pancreatic mass coefficients relative to the control group according to the Newman-Keuls test was observed in the group of monotherapy with rosuvastatin 40 mg. Statistically significant decrease in pancreatic mass coefficients relative to the control group was observed during the treatment with AGA monotherapy in a dose of 100 mg. The observed trends are consistent with the studies described in Example 1.

The mass ratios of the liver and pancreas of animals from scheme B are shown in table 43.

TABLE 43
Mass coefficients of organs - Scheme B

| | | Dose of | | | | |
|---|---|---|---|---|---|---|
| | | active | | | | |
| | | substance | | | Mass ratios, M ± SEM | |
| | | (mg) | Group Description | N | Liver | Pancreas |
| 1 | 1 | 0 | Intact | 6 | 2.8 ± 0.2 | 0.05 ± 0.005 |
| 3 | 2 B | 0 | Control * | 6 | 3.4 ± 0.3 | 0.06 ± 0.003 |
| 4 | 2 B 1 | 0 | Control | 6 | 3.8 ± 0.2 | 0.07 ± 0.005¹ |
| 6 | 3 B | 20 + 200 | Treatment with - | 6 | 3.3 ± 0.3 | 0.06 ± 0.004 |
| 8 | 4 B | 20 + 100 | rosuvastatin + AGA (the | 6 | 3.6 ± 0.2 | 0.07 ± 0.002¹ |
| 10 | 5 B | 20 + 50 | present invention) | 6 | 3.8 ± 0.3 | 0.07 ± 0.004¹ |
| 12 | 6 B | 20 | Comparison- treatment | 6 | 3.8 ± 0.2 | 0.09 ± 0.004¹² |
| | | | with - rosuvastatin | | | |
| 15 | 7 B | 20 + 200 | Treatment with - | 6 | 2.9 ± 0.1 | 0.05 ± 0.002² |
| 17 | 8 B | 20 + 100 | atorvastatin + AGA (the | 6 | 3.0 ± 0.3 | 0.06 ± 0.004 |
| 19 | 9 B | 20 + 50 | present invention) | 6 | 3.5 ± 0.2 | 0.07 ± 0.002¹ |
| 21 | 10 B | 20 | Comparison- treatment | 6 | 3.8 ± 0.1 | 0.08 ± 0.006¹ |
| | | | with - atorvastatin | | | |
| 24 | 11 B | 100 | Comparison- treatment | 6 | 3.0 ± 0.2 | 0.06 ± 0.004 |
| | | | with AGA | | | |

Notes:
¹p < 0.05 - a significant difference from the intact group (Newman-Keuls test);
²p < 0.05 - a significant difference from the control group 2B1 (Newman-Keuls test);
* Control group 2B was euthanized in order to control the development of plaques on day 61 of the study.

Dispersive analysis showed that the influence of the factor of drugs administration on the liver mass coefficients characterized by the values F 11, 60=2.87, p=0.004. According to the Newman-Keuls test liver mass coefficients differences between the groups were absent. However, it is noteworthy that we can observe the tendency of the mass coefficients increase in the control group with respect to normal values, as well as the tendency of liver mass coefficients increase in case of rosuvastatin and atorvastatin monotherapies and combinations with AGA according to the present invention at a dose of 50 mg.

The dispersive analysis of data of pancreas mass coefficients shows influence of factor of drugs administration on the parameters characterizing the values F 11, 60=8.62, p<0.00001. Pancreas mass coefficients of control animals under Newman-Keuls test were statistically significantly greater than those in the intact group. The expressed statistically significant increase in pancreatic weight ratios relative to the control group according to the Newman-Keuls test was observed in the group of monotherapy with rosuvastatin 40 mg. A statistically significant decrease in pancreatic mass coefficient relative to the control group was observed during the treatment with AGA monotherapy in a dose of 100 mg.

The observed trends are consistent with the studies described in Example 1 above.

The most effective against pancreatic mass coefficients was the combination of atorvastatin+AGA according to the present invention in a dose 20+200 mg. In the group treated with this combination, the pancreas mass coefficients values were identical with corresponding of the intact group.

### Results of Changes of the Lipid Profile

### Scheme A

A treatment Scheme A was accomplished from 31 to 90 days of research in the setting of the modeling of pathology and showed hypocholesteremic action which was evaluated by prevention or slowing of all stages of the development of atheromatosis and atherocalcinosis besides changes in lipid metabolism.

Results of statistical processing of lipid spectrum prior to the diet for Scheme A (ANOVA, F-criterion value, the value of p) were in line with the normal distribution. All lipid profile values were within the physiological range. There was no difference in average values of all parameters, that allowed to start the research.

Increasing concentration of cholesterol, LDL and triglycerides was observed in animals in comparison with the data prior to the diet. Atherogenic index values in the setting of 30 days lasting pathology increased in comparison with corresponding values on the 0 day.

The data of lipid profile in animals on 30 days of Scheme A are shown in Table 44.

TABLE 44
The lipid profile on day 30 (before treatment) - Scheme A

| | | | | | Lipid profile, M ± SEM | | | | |
|---|---|---|---|---|---|---|---|---|---|
| | | | | | Total | HDL | LDL- | | |
| | | Dose | Group | | Cholesterol | Cholesterol, | Cholesterol, | | Index of |
| | | (mg) | Description | N | mmol/L | mmol/L | mmol/L | TG mmol/l | atherogenicity |
| 2 | 2A | 0 | Control | 6 | 6.78 ± 0.23 | 0.44 ± 0.04 | 6.06 ± 0.23 | 0.62 ± 0.05 | 15.16 ± 1.72 |
| 5 | 3A | 20 + 200 | Treatment with | 6 | 6.85 ± 0.21 | 0.47 ± 0.03 | 6.10 ± 0.20 | 0.61 ± 0.04 | 13.76 ± 0.84 |
| 7 | 4A | 20 + 100 | rosuvastatin + | 6 | 6.72 ± 0.11 | 0.45 ± 0.04 | 5.97 ± 0.11 | 0.65 ± 0.05 | 14.68 ± 1.59 |
| 9 | 5A | 20 + 50 | AGA (the present | 6 | 6.84 ± 0.23 | 0.46 ± 0.04 | 6.10 ± 0.25 | 0.62 ± 0.05 | 14.50 ± 1.51 |
| | | | invention) | | | | | | |
| 11 | 6A | 20 | Comparison- | 6 | 6.59 ± 0.44 | 0.44 ± 0.04 | 5.86 ± 0.45 | 0.64 ± 0.06 | 14.50 ± 1.51 |
| 13 | 6A2 | 40 | treatment with - | 6 | 6.88 ± 0.22 | 0.45 ± 0.04 | 6.13 ± 0.23 | 0.65 ± 0.05 | 15.02 ± 1.60 |
| | | | rosuvastatin | | | | | | |
| 14 | 7A | 20 + 200 | Treatment with - | 6 | 6.76 ± 0.29 | 0.47 ± 0.05 | 6.00 ± 0.28 | 0.63 ± 0.05 | 14.38 ± 1.93 |
| 16 | 8A | 20 + 100 | rosuvastatin + | 6 | 6.84 ± 0.32 | 0.46 ± 0.04 | 6.10 ± 0.33 | 0.62 ± 0.05 | 14.69 ± 2.05 |
| 18 | 9A | 20 + 50 | AGA (the present | 6 | 6.75 ± 0.34 | 0.45 ± 0.05 | 6.01 ± 0.35 | 0.64 ± 0.05 | 15.43 ± 2.77 |
| | | | invention) | | | | | | |
| 20 | 10A | 20 | Comparison- | 6 | 6.49 ± 0.51 | 0.44 ± 0.04 | 5.77 ± 0.53 | 0.62 ± 0.04 | 14.67 ± 2.25 |
| 22 | 10A2 | 40 | treatment with - | 6 | 6.84 ± 0.32 | 0.42 ± 0.03 | 6.12 ± 0.29 | 0.65 ± 0.05 | 15.50 ± 0.78 |
| | | | atorvastatin | | | | | | |
| 23 | 11A | 100 | Comparison- | 6 | 6.45 ± 0.21 | 0.43 ± 0.04 | 5.73 ± 0.22 | 0.64 ± 0.04 | 14.79 ± 1.67 |
| | | | treatment with | | | | | | |
| | | | AGA | | | | | | |

Results of statistical data processing biochemical parameters of rabbits blood of Scheme A in the setting of 30-days lasing pathology (ANOVA, F-criterion value, the value of p) were in line with the normal distribution. According to the results of dispersive analysis and evaluation under the Newman-Keuls method average values of all parameters did not differ between groups.

### The Parameters of Lipid Profile During Treatment—Scheme A

The results of the analysis of lipid profile during treatment scheme A are shown in Table 45.

TABLE 45
Lipid spectrum in the setting of 15 days of treatment (45 days of research) - Scheme A

| | | | | | Lipid profile, M ± SEM | | | | |
|---|---|---|---|---|---|---|---|---|---|
| | | | | | | HDL | LDL- | | |
| | | Dose | Group | | Cholesterol | Cholesterol, | Cholesterol, | | Index of |
| | | (mg) | Description | | mmol/L | mmol/L | mmol/L | TG mmol/l | atherogenicity |
| 2 | 2A | 0 | Control | 6 | 8.39 ± 0.81 | 0.41 ± 0.04 | 7.61 ± 0.78 | 0.81 ± 0.07 | 20.17 ± 2.61 |
| 5 | 3A | 20 + 200 | Treatment with | 6 | 6.98 ± 0.32 | 0.53 ± 0.04 | 6.00 ± 0.34 | 0.73 ± 0.02 | 12.64 ± 1.51 |
| 7 | 4A | 20 + 100 | rosuvastatin + AGA | 6 | 7.00 ± 0.56 | 0.49 ± 0.05 | 6.16 ± 0.55 | 0.75 ± 0.03 | 13.97 ± 1.68 |
| 9 | 5A | 20 + 50 | (the present | 6 | 7.19 ± 0.54 | 0.45 ± 0.04 | 6.40 ± 0.57 | 0.74 ± 0.05 | 16.09 ± 2.73 |
| | | | invention) | | | | | | |
| 11 | 6A | 20 | Cmparison- treatment | 6 | 7.25 ± 0.62 | 0.44 ± 0.03 | 6.47 ± 0.59 | 0.75 ± 0.04 | 15.49 ± 1.06 |
| 13 | 6A2 | 40 | with rosuvastatin | 6 | 6.49 ± 0.23 | 0.49 ± 0.04 | 5.67 ± 0.26 | 0.72 ± 0.01 | 12.97 ± 1.70 |
| 14 | 7A | 20 + 200 | Treatment with | 6 | 6.85 ± 0.04 | 0.55 ± 0.02 | 5.98 ± 0.06 | 0.71 ± 0.07 | 11.50 ± 0.48* |
| 16 | 8A | 20 + 100 | atorvastatin + AGA | 6 | 6.90 ± 0.05 | 0.51 ± 0.04 | 6.15 ± 0.06 | 0.71 ± 0.05 | 13.13 ± 1.00 |
| 18 | 9A | 20 + 50 | (the present | 6 | 7.06 ± 0.06 | 0.49 ± 0.02 | 6.24 ± 0.07 | 0.72 ± 0.04 | 13.56 ± 0.55 |
| | | | invention) | | | | | | |
| 20 | 10A | 20 | Comparison treatment | 6 | 7.10 ± 0.70 | 0.47 ± 0.03 | 6.30 ± 0.73 | 0.73 ± 0.05 | 14.63 ± 2.28 |
| 22 | 10A2 | 40 | with atorvastatin | 6 | 6.35 ± 0.52 | 0.51 ± 0.04 | 5.52 ± 0.55 | 0.71 ± 0.04 | 12.07 ± 1.82 |
| 23 | 11A | 100 | Comparison- | 6 | 8.08 ± 0.52 | 0.45 ± 0.03 | 7.28 ± 0.53 | 0.78 ± 0.06 | 17.48 ± 1.91 |
| | | | treatment with AGA | | | | | | |

Note -
*p < 0.05 - a significant difference from the control group (Newman-Keuls test criterion).

As shown in Table 45, the concentrations of cholesterol, LDL cholesterol and triglycerides in the groups treated with the investigated combinations of drugs and drugs of comparisons were significantly lower than in the control group. Despite the lack of statistically significant differences between the groups in the parameters of cholesterol, LDL HDL and TG, in 15 days of treatment under the scheme A, a statistically significant decrease in atherogenic index was discovered in the group receiving the combination of atorvastatin and AGA in dose 20+200 mg according to the present invention (approximately 40% decrease in comparison with the control group).

It should be noted that with respect to all lipid parameters according to the point system of estimation, administration of combination of atorvastatin+investigated AGA at a dose 20+200 mg according to the present invention was the most effective. Effectiveness of this combination in relation to the normalization of lipid profile parameters, was substantiated by a tendency of exceeding the effectiveness of atorvastatin monotherapy in a equivalent dose equal to 20 mg, and it was equitable to the effectiveness of atorvastatin monotherapy in double dose (40 mg). On day 15 of treatment according to Scheme A efficacy of the combination of atorvastatin+AGA at all exceeded corresponding combination of rosuvastatin+AGA.

The primary data were in line with the normal distribution. The statistically significant difference of atherogenic index in the group receiving atorvastatin+AGA in a dose of 20+200 mg according to the present invention was discovered according to the dispersive analysis results and evaluation under the Newman-Keuls method.

The results of the analysis of lipid profile after 30 days of treatment are shown in Table 46.

TABLE 46
lipid spectrum in setting of 30 days of treatment (on day 60 of research): Scheme A

| | | | | | Lipid profile, M ± SEM | | | | |
|---|---|---|---|---|---|---|---|---|---|
| | | | | | | HDL | LDL- | | |
| | | Dose | | | Cholesterol | Cholesterol, | Cholesterol, | | Index of |
| | | (mg) | Group Description | | mmol/L | mmol/L | mmol/L | TG mmol/l | atherogenicity |
| 2 | 2A | 0 | Control | 6 | 11.45 ± 0.89 | 0.53 ± 0.05 | 10.42 ± 0.92 | 1.11 ± 0.07 | 21.94 ± 3.48 |
| 5 | 3A | 20 + 200 | Treatment with | 6 | 7.64 ± 0.32* | 0.70 ± 0.05 | 6.24 ± 0.29* | 0.89 ± 0.05 | 10.13 ± 0.71* |
| 7 | 4A | 20 + 100 | rosuvastatin + AGA | 6 | 7.90 ± 0.67* | 0.69 ± 0.03 | 6.50 ± 0.66* | 0.89 ± 0.01 | 10.63 ± 1.11* |
| 9 | 5A | 20 + 50 | (the present invention) | 6 | 8.06 ± 0.52* | 0.67 ± 0.05 | 6.98 ± 0.51* | 0.90 ± 0.07 | 11.32 ± 1.17* |
| 11 | 6A | 20 | Comparison- | 6 | 8.14 ± 0.75* | 0.63 ± 0.04 | 7.10 ± 0.76* | 0.91 ± 0.08 | 12.36 ± 1.83* |
| 13 | 6A2 | 40 | treatment with | 6 | 7.21 ± 0.62* | 0.72 ± 0.03 | 6.09 ± 0.64* | 0.89 ± 0.07 | 9.16 ± 0.98* |
| | | | rosuvastatin | | | | | | |
| 14 | 7A | 20 + 200 | Treatment with | 6 | 7.61 ± 0.04* | 0.73 ± 0.06 | 6.56 ± 0.07* | 0.85 ± 0.09 | 9.90 ± 1.00* |
| 16 | 8A | 20 + 100 | atorvastatin + AGA | 6 | 7.80 ± 0.05* | 0.71 ± 0.03 | 6.70 ± 0.05* | 0.87 ± 0.08 | 10.19 ± 0.54* |
| 18 | 9A | 20 + 50 | (the present invention) | 6 | 7.95 ± 0.08* | 0.68 ± 0.06 | 6.87 ± 0.12* | 0.87 ± 0.05 | 11.13 ± 1.03* |
| 20 | 10A | 20 | Comparison- | 6 | 8.02 ± 0.07* | 0.65 ± 0.05 | 6.96 ± 0.09* | 0.90 ± 0.09 | 11.90 ± 1.30* |
| 22 | 10A2 | 40 | treatment with | 6 | 6.92 ± 0.05* | 0.78 ± 0.05 | 5.76 ± 0.09* | 0.84 ± 0.07 | 8.09 ± 0.63* |
| | | | atorvastatin | | | | | | |
| 23 | 11A | 100 | Comparison- | 6 | 10.02 ± 0.89 | 0.62 ± 0.04 | 8.94 ± 0.90 | 1.02 ± 0.08 | 15.78 ± 2.21* |
| | | | treatment with AGA | | | | | | |

Note -
*p < 0.05 - a significant difference from the control group (Newman-Keuls test method).

As can be seen from Table 46, aggravation of simulated pathology continued on 60 day of research, that can be confirmed by expressed increase in the concentration of Cholesterol, LDL, TG and atherogenic index in animals of the control group relative to the value on 45 day of research.

Herewith the administration of the combinations of the present invention and drugs of comparison prevented the development of the pathology, which is confirmed by statistically significant differences of cholesterol, LDL cholesterol and atherogenic index among the treatment groups in comparison with corresponding values of the control group of animals (under criterion of Newman-Keuls method). It should be noted that the effectiveness of investigational combinations regarding the parameters of lipid profile characterized by a direct dependence on the AGA dose as part of the combination, which allows to suggest the existence of AGA contribution to antiatherosclerotic efficacy of combinations.

According to the results of dispersive analysis a statistically significant impact of the group factor was discovered on cholesterol, LDL and atherogenic index. The evaluating under the Newman-Keuls method showed statistically significant differences of cholesterol, LDL in animals of all groups except the group receiving AGA. However, the atherogenic index in the group receiving monotherapy with AGA was significantly different from corresponding index in the control group, which confirms the assumption of AGA efficacy in the prevention of the process of atherosclerosis.

According to the score of evaluation study of the effectiveness of combinations according to Scheme A, the most effective treatment on 30 day of treatment the administration of the combinations of rosuvastatin+AGA and atorvastatin+AGA according to the present invention was equally effective in relation to all lipid parameters. The tendency of the anti-atherosclerotic effect increase was definitive in case of including of AGA in combination according to the present invention in a dose of 200 mg. Among the groups treated with both investigated combinations of the present invention in doses 20+200 mg, the efficacy in relation to all lipid parameters including a key parameter-LDL exceeded corresponding efficacy of the comparison drug at equivalent doses of 20 mg. However the use of drugs of comparison in doses of 40 mg has shown the efficacy advantage of atorvastatin in relation to rosuvastatin in relation to all parameters of lipid profile on day 30 of treatment, that allows to suggest that combination of atorvastatin+AGA according to the present invention is the most promising.

The results of a study of lipid profile after 45 days of treatment under the scheme A are shown in Table 47.

TABLE 47
lipid spectrum in the setting of 45 days of treatment (day 75 of research) - Scheme A

| | | | | | Lipid profile, M ± SEM | | | | |
|---|---|---|---|---|---|---|---|---|---|
| | | | | | Total | HDL | LDL- | | |
| | | Dose | | | Cholesterol | Cholesterol, | Cholesterol, | | Index of |
| | | (mg) | Group Description | | mmol/L | mmol/L | mmol/L | TG mmol/L | atherogenicity |
| 2 | 2A | 0 | Control | 6 | 11.75 ± 1.02 | 0.47 ± 0.04 | 10.8 ± 1.0 | 1.12 ± 0.11 | 25.13 ± 3.49 |
| 5 | 3A | 20 + 200 | Treatment with | 6 | 5.54 ± 0.45* | 0.57 ± 0.04 | 4.63 ± 0.46* | 0.74 ± 0.08* | 8.93 ± 0.93* |
| 7 | 4A | 20 + 100 | rosuvastatin + AGA | 6 | 5.90 ± 0.23* | 0.62 ± 0.05 | 4.99 ± 0.19* | 0.76 ± 0.07* | 8.80 ± 068* |
| 9 | 5A | 20 + 50 | (the present invention) | 6 | 6.06 ± 0.22* | 0.65 ± 0.04 | 5.05 ± 0.24* | 0.79 ± 0.04* | 8.66 ± 1.04* |
| 11 | 6A | 20 | | 6 | 6.23 ± 0.22* | 0.64 ± 0.05 | 5.22 ± 0.23* | 0.81 ± 0.04* | 9.03 ± 0.85* |
| 13 | 6A2 | 40 | Comparison- | 6 | 5.27 ± 0.51* | 0.54 ± 0.04 | 4.40 ± 0.47* | 0.72 ± 0.05* | 8.81 ± 0.81* |
| | | | treatment with | | | | | | |
| | | | rosuvastatin | | | | | | |
| 14 | 7A | 20 + 200 | Treatment with - | 6 | 5.10 ± 0.02* | 0.52 ± 0.04 | 4.14 ± 0.04* | 0.71 ± 0.04* | 9.23 ± 0.83* |
| 16 | 8A | 20 + 100 | atorvastatin + AGA | 6 | 5.80 ± 0.04* | 0.57 ± 0.04 | 4.78 ± 0.05* | 0.72 ± 0.05* | 9.55 ± 0.60* |
| 18 | 9A | 20 + 50 | (the present invention) | 6 | 5.93 ± 0.05* | 0.61 ± 0.03 | 4.97 ± 0.03* | 0.77 ± 0.06* | 8.83 ± 0.47* |
| 20 | 10A | 20 | Comparison- | 6 | 6.02 ± 0.06* | 0.62 ± 0.04 | 5.04 ± 0.07* | 0.78 ± 0.05* | 8.89 ± 0.50* |
| 22 | 10A2 | 40 | treatment with | 6 | 4.99 ± 0.03* | 0.51 ± 0.05 | 4.16 ± 0.06* | 0.70 ± 0.06* | 9.14 ± 0.82* |
| | | | atorvastatin | | | | | | |
| 23 | 11A | 100 | Comparison- | 6 | 7.89 ± 0.05* | 0.72 ± 0.05* | 6.78 ± 0.04* | 0.85 ± 0.04* | 10.24 ± 0.74* |
| | | | treatment with AGA | | | | | | |

Note -
*p < 0.05 - a significant difference from the control group (Newman-Keuls test criterion).

As shown in Table 47, in case of administration of investigated drugs statistically significant differences were observed between the values of Cholesterol, HDL, LDL, triglycerides and atherogenic index according to the Newman-Keuls method in the setting of 75 days of simulated pathology.

The most expressed efficacy in relation to lipid parameters was observed in the use of a combination of atorvastatin+AGA according to the present invention at a dose of 20 mg+200. The effectiveness of this combination exceeded corresponding efficacy of atorvastatin monotherapy in a dose of 20 mg and was comparable with efficacy of atorvastatin in a twice dose−40 mg.

According to the results of dispersive analysis statistically significant effect of the group factor has been discovered for all lipid parameters. The effectiveness of the test combinations and statin monotherapies characterized by a direct dose-dependent manner.

The results on 60 days of treatment against scheme A are shown in Table 48.

TABLE 48
the lipid spectrum in the setting of 60 days lasting of treatment (90 day of research) - Scheme A

| | | | | | Lipid profile, M ± SEM | | | | |
|---|---|---|---|---|---|---|---|---|---|
| | Sub- | | | | Total | HDL | LDL- | | Index of |
| Group | group | Dose | Group | | Cholesterol | Cholesterol, | Cholesterol, | | athero- |
| [symbol] o | [symbol] o | (mg) | Description | N | mmol/L | mmol/L | mmol/L | TG mmol/1 | genicity |
| 2 | 2A | 0 | Control | 6 | 12.42 ± 1.05 | 0.54 ± 0.03 | 10.95 ± 1.03 | 2.04 ± 0.12 | 22.87 ± 2.56 |
| 5 | 3A | 20 + 200 | Treatment with | 6 | 4.10 ± 0.34* | 1.17 ± 0.05* | 2.51 ± 0.37* | 1.03 ± 0.03* | 2.61 ± 0.40* |
| 7 | 4A | 20 + 100 | rosuvastatin + | 6 | 4.50 ± 0.19* | 1.05 ± 0.06* | 3.08 ± 0.19* | 1.01 ± 0.05* | 3.40 ± 0.20* |
| 9 | 5A | 20 + 50 | AGA (the | 6 | 4.73 ± 0.24* | 1.04 ± 0.04* | 3.22 ± 0.25* | 1.03 ± 0.06* | 3.59 ± 0.30* |
| | | | present | | | | | | |
| | | | invention) | | | | | | |
| 11 | 6A | 20 | Comparison- | 6 | 4.80 ± 0.11* | 1.07 ± 0.05* | 3.26 ± 0.12* | 1.04 ± 0.04* | 3.54 ± 0.24* |
| 13 | 6A2 | 40 | treatment with | 6 | 3.42 ± 0.21* | 1.05 ± 0.04* | 1.96 ± 0.24* | 0.91 ± 0.05* | 2.30 ± 0.30* |
| | | | rosuvastatin | | | | | | |
| 14 | 7A | 20 + 200 | Treatment with | 6 | 3.38 ± 0.25* | 1.18 ± 0.05* | 2.22 ± 0.28* | 0.85 ± 0.04* | 2.42 ± 0.31* |
| 16 | 8A | 20 + 100 | atorvastatin + | 6 | 4.09 ± 0.15* | 1.11 ± 0.04* | 2.70 ± 0.18* | 0.86 ± 0.02* | 2.84 ± 0.25* |
| 18 | 9A | 20 + 50 | AGA (the | 6 | 4.35 ± 0.21* | 1.04 ± 0.02* | 2.91 ± 0.22* | 0.88 ± 0.03* | 3.20 ± 0.23* |
| | | | present | | | | | | |
| | | | invention) | | | | | | |
| 20 | 10A | 20 | Comparison- | 6 | 4.41 ± 0.12* | 1.03 ± 0.05* | 2.98 ± 0.14* | 0.87 ± 0.05* | 3.32 ± 0.22* |
| 22 | 10A2 | 40 | treatment with - | 6 | 3.29 ± 0.37* | 1.10 ± 0.06* | 1.83 ± 0.44* | 0.80 ± 0.06* | 2.12 ± 0.48* |
| | | | atorvastatin | | | | | | |
| 23 | 11A | 100 | Comparison- | 6 | 7.36 ± 0.49* | 0.74 ± 0.02* | 6.12 ± 0.48* | 1.11 ± 0.07* | 8.96 ± 0.68* |
| | | | treatment with | | | | | | |
| | | | AGA | | | | | | |

Note -
*p < 0.05 - a significant difference from the control group (Newman-Keuls test criterion).

According to Scheme A high efficacy of all studied combinations has been discovered in 60 days of administration of all investigated combinations and drugs of comparison in relation of Cholesterol, HDL, LDL, triglycerides atherogenic index. The effectiveness is confirmed by the statistically significant differences of these parameters in groups receiving drugs from corresponding values of the control group according to the Newman-Keuls method. According to the evaluation points of all lipid parameters the highest efficiency with respect to all lipid parameters has shown the administration of the combination of atorvastatin and AGA according to the present invention in a dose 20+200 mg within 60 days under Scheme A. The effectiveness of this combination in specified dose exceeded the corresponding efficacy of atorvastatin monotherapy in a dose of 20 mg and was comparable with efficacy of atorvastatin in a twice dose−40 mg.

According to the results of dispersive analysis the statistically significant effect of the group factor for all lipid parameters has been observed. The effectiveness of the test combinations and statin monotherapies was characterized by a direct dose-dependent manner.

Thus the expressed increase in cholesterol and LDL level has been observed among the animals of the Scheme A within 90 days of the study. In the setting of the administration of the investigated drugs and drugs of comparison, the increase of these parameters was characterized by a much lower degree. The expressed differences were observed by the last day of the study under Scheme A (90 day of pathology, 60 day of treatment): the concentrations of cholesterol in the groups treated with the fixed combination, were lower than in the control group by a factor of 2.6-3.1, LDL by a factor of 3 4-4.5.

By the 30th day of treatment the administration of the combination of AGA with rosuvastatin according to the present invention was the most effective and comparable to the efficacy of the combination of AGA with atorvastatin according to the present invention. By the end of treatment the efficacy of combination of AGA with atorvastatin according to the present invention in relation to LDL-Cholesterol lowering was greater than the combination of AGA with rosuvastatin according to the present invention. The effectiveness of the test combinations was characterized by a direct dose-dependent manner.

The decrease in LDL cholesterol level has also been observed on the 60 day of treatment in the group of animals treated with AGA, which shows the contribution of its activity in the lipid-lowering effect of the combination.

Efficacy of the combinations with the highest content of AGA (200 mg) was greater than corresponding efficacy of the drugs of comparison. In case of administration of atorvastatin, the efficiency of combination with AGA (200 mg) according to the present invention was comparable with the efficacy of atorvastatin monotherapy in a dose 40 mg. In the case of administration of rosuvastatin, the efficacy of the combination of with AGA (200 mg) according to the present invention was comparable with the efficacy of rosuvastatin monotherapy in a dose 40 mg.

### Scheme B

Treatment scheme B was conducted from 61 to 120 day of the study. The concept of the administration of this scheme was to achieve regression of atherosclerotic plaque (antiatherosclerotic efficiency).

Statistically significant differences between the study groups according to the Newman-Keuls method are absent in all lipid parameters on 0 day. According to the results of dispersive analysis the statistically significant difference was not discovered. There was no influence of the group factor in relation to lipid parameters. All lipid profile values were within the physiological range before the pathology development. There was no difference in average values of all parameters, that allowed to start the research.

The estimation data of lipid parameters in animals under Scheme B in 15 days of pathology simulating are shown in the Table 49.

TABLE 49
Lipid parameters on 15 day (in the setting of pathology before treatment) Scheme B

| | | | | | Lipid profile, M ± SEM | | | | |
|---|---|---|---|---|---|---|---|---|---|
| | Sub- | | | | Total | HDL | LDL- | | Index of |
| Group | group | Dose | Group | | Cholesterol | Cholesterol, | Cholesterol, | | athero- |
| [symbol] o | [symbol] o | (mg) | Description | N | mmol/L | mmol/L | mmol/L | TG mmol/l | genicity |
| 1 | 1 | 0 | Intact | 6 | 0.83 ± 0.06 | 0.38 ± 0.03 | 0.19 ± 0.06 | 0.57 ± 0.06 | 1.26 ± 0.27 |
| 3 | 2B | 0 | Control | 6 | 2.67 ± 0.25* | 0.41 ± 0.04 | 1.99 ± 0.28* | 0.60 ± 0.05 | 5.89 ± 0.92* |
| 4 | 2B1 | 0 | Control | 6 | 2.78 ± 0.21* | 0.40 ± 0.03 | 2.11 ± 0.20* | 0.59 ± 0.04 | 6.13 ± 0.67* |
| 6 | 3B | 20 + 200 | Treatment with | 6 | 2.71 ± 0.18* | 0.39 ± 0.03 | 2.04 ± 0.18* | 0.62 ± 0.05 | 6.14 ± 0.81* |
| 8 | 4B | 20 + 100 | rosuvastatin + | 6 | 2.69 ± 0.22* | 0.41 ± 0.04 | 2.00 ± 0.26* | 0.61 ± 0.04 | 6.18 ± 1.27* |
| 10 | 5B | 20 + 50 | AGA (the | 6 | 2.74 ± 0.25* | 0.42 ± 0.03 | 2.02 ± 0.27* | 0.65 ± 0.05 | 5.80 ± 0.93* |
| | | | present | | | | | | |
| | | | invention) | | | | | | |
| 12 | 6B | 20 | Comparison- | 6 | 2.65 ± 0.24* | 0.39 ± 0.03 | 1.98 ± 0.25* | 0.61 ± 0.06 | 5.92 ± 0.69* |
| | | | treatment with | | | | | | |
| | | | rosuvastatin | | | | | | |
| 15 | 7B | 20 + 200 | Treatment with | 6 | 2.69 ± 0.21* | 0.42 ± 0.04 | 2.00 ± 0.19* | 0.60 ± 0.05 | 5.57 ± 0.44* |
| 17 | 8B | 20 + 100 | atorvastatin + | 6 | 2.70 ± 0.20* | 0.39 ± 0.03 | 2.01 ± 0.19* | 0.65 ± 0.04 | 6.14 ± 0.89* |
| 19 | 9B | 20 + 50 | AGA (the | 6 | 2.73 ± 0.25* | 0.40 ± 0.04 | 2.04 ± 0.25* | 0.64 ± 0.05 | 6.15 ± 0.94* |
| | | | present | | | | | | |
| | | | invention) | | | | | | |
| 21 | 10B | 20 | Comparison- | 6 | 2.68 ± 0.14* | 0.41 ± 0.03 | 1.99 ± 0.13* | 0.62 ± 0.06 | 5.63 ± 0.34* |
| | | | treatment with | | | | | | |
| | | | atorvastatin | | | | | | |
| 24 | 11B | 108 | Experimental- | 6 | 2.75 ± 0.24* | 0.39 ± 0.03 | 2.06 ± 0.25* | 0.66 ± 0.05 | 6.39 ± 1.05* |
| | | | treatment with | | | | | | |
| | | | AGA | | | | | | |

Note -
*p < 0.05 - evidential difference from the intact group (Newman-Keuls test criterion).

The primary data corresponded to a normal distribution.

The result of dispersive analysis revealed that on 15 day of study the increase in the concentration of cholesterol, LDL atherogenic index occurred in animals with pathology in comparison with the intact group, indicating the development of dyslipidemia with high risk of atherosclerosis. In addition, statistically significant differences between the parameters of lipid groups with pathology were absent. The dispersive analysis discovered the influence of the group factor on Cholesterol, LDL Cholesterol and atherogenic index.

Table 50 presents the lipid profile of animals of Scheme B in the setting of 60 days pathology.

TABLE 50
lipid profile 60 day (before treatment) - Scheme B

| | | | | | Lipid profile, M ± SEM | | | | |
|---|---|---|---|---|---|---|---|---|---|
| | Sub- | | | | Total | HDL | LDL- | | Index of |
| Group | group | Dose | Group | | Cholesterol | Cholesterol, | Cholesterol, | | athero- |
| [symbol] o | [symbol] o | (mg) | Description | N | mmol/L | mmol/L | mmol/L | TG mmol/L | genicity |
| 1 | 1 | 0 | Intact | 6 | 0.80 ± 0.02 | 0.36 ± 0.02 | 0.21 ± 0.03 | 0.49 ± 0.04 | 1.22 ± 0.13 |
| 3 | 2B | 0 | Control | 6 | 11.62 ± 0.92* | 0.51 ± 0.03 | 10.63 ± 0.94* | 1.05 ± 0.09* | 22.24 ± 2.32* |
| 4 | 2B1 | 0 | Control | 6 | 11.50 ± 1.05* | 0.50 ± 0.05 | 10.55 ± 1.03* | 0.98 ± 0.07* | 23.06 ± 3.15* |
| 6 | 3B | 20 + 200 | Treatment with | 6 | 11.34 ± 0.99* | 0.49 ± 0.03 | 10.42 ± 0.99* | 0.95 ± 0.04* | 22.59 ± 2.49* |
| 8 | 4B | 20 + 100 | rosuvastatin + | 6 | 11.48 ± 0.65* | 0.44 ± 0.04 | 10.60 ± 0.64* | 0.97 ± 0.02* | 22.57 ± 1.85* |
| 10 | 5B | 20 + 50 | AGA (the | 6 | 11.52 ± 0.67* | 0.47 ± 0.05 | 10.60 ± 0.66* | 0.99 ± 0.05* | 25.27 ± 3.51* |
| | | | present | | | | | | |
| | | | invention) | | | | | | |
| 12 | 6B | 20 | Comparison- | 6 | 11.44 ± 0.85* | 0.48 ± 0.04 | 10.52 ± 0.87* | 0.97 ± 0.05* | 23.73 ± 3.02* |
| | | | treatment with | | | | | | |
| | | | rozuvastatin | | | | | | |
| 15 | 7B | 20 + 200 | Treatment with | 6 | 11.09 ± 0.92* | 0.51 ± 0.05 | 10.15 ± 0.91* | 0.95 ± 0.04* | 21.70 ± 2.65* |
| 17 | 8B | 20 + 100 | atorvastatin + | 6 | 11.54 ± 0.89* | 0.49 ± 0.03 | 10.56 ± 0.85* | 1.08 ± 0.08* | 22.47 ± 1.19* |
| 19 | 9B | 20 + 50 | AGA (the | 6 | 11.23 ± 0.76* | 0.50 ± 0.04 | 10.28 ± 0.77* | 0.98 ± 0.05* | 22.29 ± 2.67* |
| | | | present | | | | | | |
| | | | invention) | | | | | | |
| 21 | 10B | 20 | Experimental- | 6 | 11.45 ± 0.59* | 0.52 ± 0.05 | 10.48 ± 0.59* | 0.99 ± 0.04* | 22.00 ± 2.28* |
| | | | treatment with | | | | | | |
| | | | atorvastatin | | | | | | |
| 24 | 11B | 108 | Experimental- | 6 | 11.68 ± 0.92* | 0.51 ± 0.04 | 10.73 ± 0.89* | 0.97 ± 0.08* | 22.29 ± 1.80* |
| | | | treatment with | | | | | | |
| | | | AGA | | | | | | |

Note -
*p < 0.05 - evidential difference from the intact group (Newman-Keuls test criterion).

On 60 day of pathology simulation a statistically significant increase in Cholesterol, LDL, triglycerides and atherogenic index was observed in animals in comparison with the intact group, as well as an increase in the values of these parameters in relation to 45 day of the study.

The dispersive analysis discovered the influence of the group factor on the parameters of cholesterol, LDL cholesterol, triglycerides and atherogenic index on 60 day of research. There were no statistically significant differences according to the Newman-Keuls method between groups with simulated pathology.

Table 51 shows the lipid profile of animals in the setting of 75 days of pathology and 15 days of treatment.

TABLE 51
lipid profile in the setting of 15 days of treatment (day 75 of research) - Scheme B

| | | | | | Lipid profile, M ± SEM | | | | |
|---|---|---|---|---|---|---|---|---|---|
| | Sub- | | | | Total | HDL | LDL- | | Index of |
| Group | group | Dose | Group | | Cholesterol | Cholesterol, | Cholesterol, | | athero- |
| [symbol] o | [symbol] o | (mg) | Description | N | mmol/L | mmol/L | mmol/L | TG mmol/L | genicity |
| 1 | 1 | 0 | Intact | 6 | 0.85 ± 0.04 | 0.37 ± 0.03 | 0.23 ± 0.03 | 0.51 ± 0.02 | 1.37 ± 0.16 |
| 4 | 2B | 0 | Control | 6 | 11.75 ± 1.02¹ | 0.47 ± 0.04¹ | 10.77 ± 0.97¹ | 1.12 ± 0.11¹ | 23.90 ± 0.43¹ |
| 6 | 2B1 | 20 + 200 | Treatment with | 6 | 8.08 ± 0.72^{1,2} | 0.52 ± 0.04¹ | 7.17 ± 0.74^{1,2} | 0.85 ± 0.06¹ | 15.10 ± 2.01^{1,2} |
| 8 | 3B | 20 + 100 | rosuvastatin + | 6 | 8.11 ± 0.65^{1,2} | 0.50 ± 0.02¹ | 7.21 ± 0.65^{1,2} | 0.88 ± 0.07¹ | 15.28 ± 1.29^{1,2} |
| 10 | 4B | 20 + 50 | AGA (the present | 6 | 8.09 ± 0.52^{1,2} | 0.53 ± 0.04¹ | 7.16 ± 0.53^{1,2} | 0.89 ± 0.06¹ | 14.67 ± 1.61^{1,2} |
| | | | invention) | | | | | | |
| 12 | 5B | 20 | Ttreatment with | 6 | 8.25 ± 0.52^{1,2} | 0.54 ± 0.04¹ | 7.31 ± 0.52^{1,2} | 0.89 ± 0.05¹ | 14.66 ± 1.33^{1,2} |
| | | | rosuvastatin | | | | | | |
| 15 | 6B | 20 + 200 | Treatment with | 6 | 7.89 ± 0.77^{1,2} | 0.52 ± 0.03¹ | 6.97 ± 0.74^{1,2} | 0.89 ± 0.07^{1,2} | 14.15 ± 1.29^{1,2} |
| 17 | 7B | 20 + 100 | atorvastatin + | 6 | 7.98 ± 0.75^{1,2} | 0.55 ± 0.04¹ | 7.03 ± 0.73^{1,2} | 0.89 ± 0.05¹ | 13.46 ± 0.83^{1,2} |
| 19 | 8B | 20 + 50 | AGA (the present | 6 | 7.92 ± 0.69^{1,2} | 0.55 ± 0.02¹ | 7.00 ± 0.67^{1,2} | 0.81 ± 0.06¹ | 13.34 ± 1.04^{1,2} |
| | | | invention) | | | | | | |
| 21 | 9B | 20 | Comparison- | 6 | 7.95 ± 0.81^{1,2} | 0.54 ± 0.05¹ | 7.04 ± 0.84^{1,2} | 0.82 ± 0.08¹ | 14.87 ± 2.61^{1,2} |
| | | | treatment with | | | | | | |
| | | | atorvastatin | | | | | | |
| 24 | 10B | 108 | Comparison- | 6 | 10.89 ± 1.05^{1,2} | 0.52 ± 0.03¹ | 9.93 ± 1.04^{1,2} | 0.98 ± 0.07¹ | 20.26 ± 2.29¹ |
| | | | treatment with | | | | | | |
| | | | AGA | | | | | | |

Notes:
¹p < 0.05 - evidential difference from the intact group (Newman-Keuls test criterion).
²Note -
* - p < 0.05 - evidential difference from the control group (Newman-Keuls test criterion).

The dispersive analysis discovered the influence of the group factor on the parameters of cholesterol, LDL cholesterol, triglycerides and atherogenic index.

The primary data corresponded to a normal distribution. According to Newman-Keuls test criteria were established statistically significant differences between the intact group and groups with pathology in the parameters of cholesterol, triglycerides, LDL cholesterol and atherogenic index, which indicates the development of the simulated disease.

The expressed statistically significant reduction in the concentration of cholesterol and LDL cholesterol, as well as a decrease in atherogenic index were observed in case of administration of the combination of the present invention.

Table 52 shows the lipid profile of animals in the setting of 90 days of pathology and 30 days of treatment.

TABLE 52
lipid profile in the setting of 30 days of treatment (day 90 of research) - Scheme B

| | | | | | Lipid profile, M ± SEM | | | | |
|---|---|---|---|---|---|---|---|---|---|
| | Sub- | | | | Total | HDL | LDL- | | Index of |
| Group | group | Dose | Group | | Cholesterol | Cholesterol, | Cholesterol, | | athero- |
| [symbol] o | [symbol] o | (mg) | Description | N | mmol/L | mmol/L | mmol/L | TG mmol/L | genicity |
| 1 | 1 | 0 | Intact | 6 | 0.96 ± 0.06 | 0.44 ± 0.02 | 0.27 ± 0.07 | 0.53 ± 0.04 | 1.13 ± 0.21 |
| 4 | 2B | 0 | Control | 6 | 12.67 ± 1.21¹ | 0.52 ± 0.05¹ | 11.57 ± 1.23¹ | 1.28 ± 0.09¹ | 25.45 ± 4.45¹ |
| 6 | 2B1 | 20 + 200 | Treatment with | 6 | 6.03 ± 0.41^{1,2} | 0.79 ± 0.06^{1,2} | 4.81 ± 0.45^{1,2} | 0.72 ± 0.03² | 6.98 ± 1.00² |
| 8 | 3B | 20 + 100 | rosuvastatin + | 6 | 6.11 ± 0.45^{1,2} | 0.78 ± 0.05^{1,2} | 5.03 ± 0.49^{1,2} | 0.74 ± 0.06² | 7.17 ± 1.02² |
| 10 | 4B | 20 + 50 | AGA (the present | 6 | 6.29 ± 0.32^{1,2} | 0.82 ± 0.05^{1,2} | 5.14 ± 0.30^{1,2} | 0.72 ± 0.03² | 6.78 ± 0.50² |
| | | | invention) | | | | | | |
| 12 | 5B | 20 | Comparison | 6 | 6.32 ± 0.62^{1,2} | 0.81 ± 0.08^{1,2} | 5.19 ± 0.59^{1,2} | 0.71 ± 0.05² | 7.03 ± 0.85² |
| | | | treatment with | | | | | | |
| | | | rosuvastatin | | | | | | |
| 15 | 6B | 20 + 200 | Treatment with | 6 | 5.08 ± 0.32^{1,2} | 0.91 ± 0.08^{1,2} | 3.86 ± 0.34^{1,2} | 0.69 ± 0.03¹² | 4.91 ± 0.63² |
| 17 | 7B | 20 + 100 | atorvastatin + AGA | 6 | 5.45 ± 0.49^{1,2} | 0.88 ± 0.01^{1,2} | 4.36 ± 0.47^{1,2} | 0.69 ± 0.05² | 5.29 ± 0.52² |
| 19 | 8B | 20 + 50 | (the present | 6 | 5.68 ± 0.41^{1,2} | 0.85 ± 0.04^{1,2} | 4.51 ± 0.40^{1,2} | 0.71 ± 0.06² | 5.76 ± 0.62² |
| | | | invention) | | | | | | |
| 21 | 9B | 20 | Experimental- | 6 | 5.87 ± 0.39^{1,2} | 0.84 ± 0.07^{1,2} | 4.71 ± 0.38^{1,2} | 0.70 ± 0.05² | 6.19 ± 0.74² |
| | | | treatment with | | | | | | |
| | | | atorvastatin | | | | | | |
| 24 | 10B | 100 | Experimental- | 6 | 8.49 ± 0.75^{1,2} | 0.68 ± 0.05^{1,2} | 7.42 ± 0.78^{1,2} | 0.85 ± 0.06¹² | 12.01 ± 1.64¹² |
| | | | treatment with | | | | | | |
| | | | AGA | | | | | | |

Notes:
¹p < 0.05 - evidential difference from the intact group (Newman-Keuls test criterion).
²Note -
* - p < 0.05 - evidential difference from the control group (Newman-Keuls test criterion).

The primary data corresponded to a normal distribution.

The statistically significant decrease in the parameters of cholesterol, triglycerides, LDL cholesterol and atherogenic index were observed by the 30th day of treatment among animals receiving the combinations of the present invention and drugs of comparison. The efficacy of investigated combinations in the setting of pathology on 90 day of research directly depended on the AGA dose. In the case of treatment with the combination of rosuvastatin and AGA according to the present invention and in case of treatment with the combination of atorvastatin and AGA according to the present invention, the efficacy of combinations containing the greater dose of AGA (200 mg) exceeded the efficacy of monotherapy with statins in equivalent doses. The dispersive analysis discovered the influence of the group factor on all of the parameters of lipid profile.

Table 53 shows lipid profile in animals in the setting of 105 days of pathology and 45 days of treatment.

TABLE 53
Lipid profile in the setting of 45 days of treatment (105 day of research) - Scheme B

| | | | | | Lipid profile, M ± SEM | | | | |
|---|---|---|---|---|---|---|---|---|---|
| | Sub- | | | | Total | HDL | LDL- | | Index of |
| Group | group | Dose | | | Cholesterol | Cholesterol, | Cholesterol, | | athero- |
| [symbol] o | [symbol] o | (mg) | Group Description | N | mmol/L | mmol/L | mmol/L | TG mmol/L | genicity |
| 1 | 1 | 0 | Intact | 6 | 0.91 ± 0.02 | 0.45 ± 0.02 | 0.25 ± 0.03 | 0.47 ± 0.04 | 1.03 ± 0.12 |
| 4 | 2B | 0 | Contol | 6 | 12.09 ± 1.14¹ | 0.57 ± 0.06 | 10.93 ± 1.10¹ | 1.29 ± 0.11¹ | 21.26 ± 3.17^{1,2} |
| 6 | 2B1 | 20 + 200 | Ttreatment with | 6 | 3.83 ± 0.41^{1,2} | 0.96 ± 0.04^{1,2} | 2.70 ± 0.42^{1,2} | 0.74 ± 0.03^{1,2} | 3.25 ± 0.51² |
| 8 | 3B | 20 + 100 | rosuvastatin + | 6 | 4.05 ± 0.42^{1,2} | 0.92 ± 0.07^{1,2} | 2.85 ± 0.41¹² | 0.72 ± 0.05² | 3.63 ± 0.50² |
| 10 | 4B | 20 + 50 | AGA (the present | 6 | 4.21 ± 0.31^{1,2} | 0.94 ± 0.08¹² | 2.95 ± 0.36^{1,2} | 0.71 ± 0.05¹² | 3.74 ± 0.66² |
| | | | invention) | | | | | | |
| 12 | 5B | 20 | Comparison- | 6 | 4.39 ± 0.41^{1,2} | 0.95 ± 0.07^{1,2} | 3.10 ± 0.42^{1,2} | 0.74 ± 0.04² | 3.80 ± 0.63² |
| | | | treatment with | | | | | | |
| | | | rosuvastatin | | | | | | |
| 15 | 6B | 20 + 200 | Treatment with | 6 | 3.89 ± 0.18^{1,2} | 0.99 ± 0.05^{1,2} | 2.50 ± 0.18¹'² | 0.71 ± 0.05² | 3.08 ± 0.27² |
| 17 | 7B | 20 + 100 | atorvastatin + | 6 | 4.00 ± 0.37^{1,2} | 0.96 ± 0.03^{1,2} | 2.78 ± 0.37^{1,2} | 0.71 ± 0.08^{1,2} | 3.40 ± 0.47² |
| 19 | 8B | 20 + 50 | AGA (the present | 6 | 4.12 ± 0.41^{1,2} | 0.98 ± 0.09^{1,2} | 2.90 ± 0.48^{1,2} | 0.70 ± 0.04^{1,2} | 3.59 ± 0.70² |
| | | | invention) | | | | | | |
| 21 | 9B | 20 | Comparison- | 6 | 4.25 ± 0.32^{1,2} | 0.97 ± 0.04^{1,2} | 2.96 ± 0.32^{1,2} | 0.71 ± 0.03^{1,2} | 3.38 ± 0.31² |
| | | | treatment with | | | | | | |
| | | | atorvastatin | | | | | | |
| 24 | 10B | 108 | Comparison- | 6 | 8.12 ± 0.06^{1,2} | 0.81 ± 0.03^{1,2} | 6.92 ± 0.32^{1,2} | 0.85 ± 0.08^{1,2} | 9.09 ± 0.48¹² |
| | | | treatment with | | | | | | |
| | | | AGA | | | | | | |

Notes:
¹p < 0.05 - evidential difference from the intact group (Newman-Keuls test criterion).
²Note -
* - p < 0.05 - evidential difference from the control group (Newman-Keuls test criterion).

The primary data corresponded to a normal distribution.

Tendency of efficacy of the investigated drugs were kept on 105 day of research.

The statistically significant decrease in the parameters of cholesterol, triglycerides, LDL cholesterol and atherogenic index as well as HDL increase were observed in comparison with control group. It was noted the efficacy of the combinations of the present invention expressly exceeded the efficacy of drugs of comparison on the 30th day of treatment. The efficacy of the combinations of the present invention and drugs of comparison (statins as monotherapy) on the 45th day of treatment under Scheme B had an expressed tendency of superiority of combination therapy with AGA over monotherapy. Retention of expressed statistically significant efficacy of monotherapy AGA in relation to lipid profile on day 45 of treatment according to Scheme B shall also be noted. The dispersive analysis discovered the influence of the group factor on all of the parameters of lipid profile.

Table 54 shows data on lipid profile of animals in the setting of 120 days of pathology and 60 days of treatment.

TABLE 54
lipid profile in the setting of 60 days of treatment (120 day of research) - Scheme B

| | | | | | Lipid profile, M ± SEM | | | | |
|---|---|---|---|---|---|---|---|---|---|
| | Sub- | | | | Total | HDL | LDL- | | Index of |
| Group | group | Dose | Group | | Cholesterol | Cholesterol, | Cholesterol, | | athero- |
| [symbol] o | [symbol] o | (mg) | Description | N | mmol/L | mmol/L | mmol/L | TG mmol/L | genicity |
| 1 | 1 | 0 | Intact | 6 | 1.04 ± 0.08 | 0.48 ± 0.02 | 0.38 ± 0.08 | 0.46 ± 0.03 | 1.18 ± 0.20 |
| 4 | 2B | 0 | Control | 6 | 13.45 ± 1.28¹ | 0.59 ± 0.06 | 11.87 ± 1.26¹ | 2.18 ± 0.20¹ | 22.95 ± 3.65¹ |
| 6 | 2B1 | 20 + 200 | Treatment with | 6 | 3.17 ± 0.34^{1,2}2 | 0.85 ± 0.07^{1,2} | 1.93 ± 0.33^{1,2} | 0.72 ± 0.05² | 2.93 ± 0.47² |
| 8 | 3B | 20 + 100 | rosuvastatin + | 6 | 3.35 ± 0.28^{1,2} | 0.87 ± 0.08^{1,2} | 2.11 ± 0.30^{1,2} | 0.80 ± 0.07² | 3.13 ± 0.45² |
| 10 | 4B | 20 + 50 | AGA (the | 6 | 3.52 ± 0.22^{1,2} | 0.90 ± 0.07^{1,2} | 2.25 ± 0.18^{1,2} | 0.82 ± 0.04² | 3.06 ± 0.13² |
| | | | present | | | | | | |
| | | | invention) | | | | | | |
| 12 | 5B | 20 | Comparison- | 6 | 3.94 ± 0.15^{1,2} | 0.91 ± 0.08^{1,2} | 2.64 ± 0.13^{1,2} | 0.85 ± 0.07^{1,2} | 3.49 ± 0.39² |
| | | | treatment with | | | | | | |
| | | | rosuvastatin | | | | | | |
| 15 | 6B | 20 + 200 | Treatment with | 6 | 3.00 ± 0.12^{1,2} | 0.85 ± 0.02^{1,2} | 1.60 ± 0.12^{1,2} | 0.70 ± 0.05² | 2.63 ± 0.11² |
| 17 | 7B | 20 + 100 | atorvastatin + | 6 | 3.10 ± 0.22^{1,2} | 0.88 ± 0.04^{1,2} | 1.85 ± 0.22^{1,2} | 0.74 ± 0.07² | 2.61 ± 0.25² |
| 19 | 8B | 20 + 50 | AGA (the | 6 | 3.35 ± 0.21^{1,2} | 0.86 ± 0.03^{1,2} | 2.10 ± 0.21^{1,2} | 0.76 ± 0.05² | 2.92 ± 0.25² |
| | | | present | | | | | | |
| | | | invention) | | | | | | |
| 21 | 9B | 20 | Experimental- | 6 | 3.52 ± 0.12^{1,2} | 0.89 ± 0.06^{1,2} | 2.26 ± 0.13^{1,2} | 0.81 ± 0.04² | 3.04 ± 0.29² |
| | | | treatment with | | | | | | |
| | | | atorvastatin | | | | | | |
| 24 | 10B | 100 | Experimental- | 6 | 7.45 ± 0.05^{1,2} | 0.82 ± 0.06^{1,2} | 6.27 ± 0.09^{1,2} | 0.80 ± 0.071² | 8.37 ± 0.77^{1,2} |
| | | | treatment with | | | | | | |
| | | | AGA | | | | | | |

Notes:
¹p < 0.05 - evidential difference from the intact group (Newman-Keuls test criterion).
²Note -
* - p < 0.05 - evidential difference from the control group (Newman-Keuls test criterion).

The primary data corresponded to a normal distribution.

The efficacy of the combinations of the present invention is increased in comparison with monotherapies 45 day of treatment. The tendency of the efficiency of the combinations to depend on the AGA dose was also observed on 30 and 45 days of treatment. The influence of a group factor on all parameters of a lipidic range was established under the dispersive analysis.

By 60th day of treatment efficiency of the studied combinations concerning an atherogenic index especially should be noted. In groups with treatment, this parameter was statistically significantly lower, than in control group by 3 times (at monotherapy with AGA) and by 9 times (at treatment with combinations and monotherapies with a statins). Thus, all studied drugs, including monotherapy with AGA had the expressed anti-atherogenous effect in case of administration within 60 days.

In case of administration within 60 days all investigated drugs, including monotherapy with AGA, had the expressed anti-atherogenous effect. However, anti-atherogenous action was more expressed in the combined therapy with AGA according to the present invention. It should be noted that the tendency of efficiency of combination increased with the AGA dose.

Treatment with atorvastatin in combination with AGA according to the present invention in a dose of 20+200 mg was the most effective in case of administration according to the scheme A and scheme B. AGA contribution to anti-atherosclerotic activity of combinations is expressed that is confirmed not only by the tendency observed at treatment according to the scheme B, but also statistically significant results received at an assessment of efficiency of therapy according to the scheme A.

Treatment with the combination of atorvastatin with AGA in a dose of 20+200 mg according to the present invention was the most effective both at application according to the scheme A, and at treatment according to the scheme B. It should be noted that in case of research according to the scheme B by the time of an initiation of treatment (by 60th day) observed heavier dyslipidemia, than by the time of initiation of treatment according to the scheme A inasmuch as the treatment according to the scheme A begun earlier for the purpose of an assessment of treatment-and-prophylactic efficiency. Therefore administration of the combinations containing AGA according to the treatment-and-prophylactic scheme will be the most effective.

The above results show that the increase in markers of a dyslipidemia and atherosclerosis was registered in animals of the scheme B during 120 days of research.

Aspiration of lipidic range parameters to normalization was observed in the setting of treatment with the fixed combinations. By 120th day of research (the 60th day of treatment) the Cholesterol level decreased by 3.3-4.5 times, LDL—by 5-6 times in comparison with control group.

The efficacy of administration of the studied combinations of the present invention was also characterized by a direct dose-dependence and exceeded the efficiency of drugs of comparison in case of inclusion of AGA in combination in a dose of 200 mg. The efficacy of administration of statin combinations according to the present invention was comparable by the end of therapy, but by the end of treatment it was observed that the combination with atorvastatin and AGA of the present invention was most efficacious. Decrease in Cholesterol and LDL level was observed on the 60th day of treatment according to the scheme B in group of the animals receiving AGA. Anti-atherogenous action was more expressed in case of the combined therapy with AGA according to the present invention.

### Changes of Parameters of Biochemical Blood Test Before Treatment

### Scheme A

### Toxicological Indicators Before Treatment

Data showing the range of toxicological parameters for animals of the scheme A before the pathology (background values) are presented in Table 55. Primary data corresponded to normal distribution. According to Newman-Keuls test statistically significant differences between groups were absent in all studied parameters.

Data showing the range of toxicological parameters of animals of the scheme A for the 30th day of pathology are presented in Table 56. Against 30 days of modeling of pathology animals had a further development of pathology that was confirmed by the increase in activity of transaminase, concentration of bilirubin mainly at the expense of direct bilirubin, increase in concentration of glucose concerning values of these parameters for the 15th day testified.

### Toxicological Indicators Under the Treatment

Data showing the range of toxicological parameters of animals of the scheme A for the 45th day of pathology, in 15 days of treatment are presented in Table 57. Primary data corresponded to normal distribution. According to Newman-Keuls test statistically significant differences between groups were absent in all studied parameters.

On the 45th day of research animals had a further development of violations of exchange processes accompanying the modelled pathology. There was an increase in activity of transaminase, CPK, concentration of bilirubin and glucose in comparison with the 30th day of research. Against promptly developing pathology the studied medicines didn't affect biochemical markers of an experimental dyslipidemia and atherosclerosis on the 15th day of administration.

Data showing the range of toxicological parameters of animals on 60 day of pathology, after 30 days of treatment, is represented in table 58. Primary data corresponded to normal distribution. Under Newman-Keuls test, there was no statistically significant difference between groups across all experimental variables. The progression of experimental pathology and increase of biochemical markers' values continued on 60 day of pathology. Despite the absence of statistical significance, there was a tendency to decreasing the transaminase activity and CPK while using the combination of statins and AGA in dose 20+200 mg according to the present invention.

Results of dispersion analysis demonstrated no influence of group factor on analyzed biochemical parameters of scheme A animals' blood after 60 days of pathology and 30 days of treatment with pathology, except for ALT parameter. Despite the absence (under Newman-Keuls test) of statistically significant difference of ALT activity and CPK between groups with treatment and control groups, the results of dispersion analysis probably confirm the tendency of effectiveness of combination of statins with AGA in dose 20+200 mg according to the present invention concerning transaminase and CPK.

Data showing the range of toxicological parameters of animals on 75 day of pathology, after 45 days of treatment, is represented in table 59. Under Newman-Keuls test, statistically significant difference was determined from control group on AST activity parameter in all groups with treatment except for group of AGA monotherapy, and on CPK in groups with 200 mg AGA dose in case of rosuvastatin, and with 200 mg dose, 100 mg in atorvastatin case. In combination with atorvastatin 20+200 and 20+100 mg doses a significant decrease of CPK activity was noted as compared to control group, while using rosuvastatin combination effectiveness in point of CPK was observed only in applying combination in 20+200 mg dose.

A Significant tendency of studied combinations' effectiveness on ALT was observed on scheme A 45 day of treatment. The most significant effectiveness as for these parameters on 45 day of treatment was observed with inclusion in both combinations of AGA according to the present invention in 200 mg dose. It was noted that the effectiveness of combination atorvastatin+AGA according to the present invention was characterized by tendency to exceed the effectiveness of the combination rosuvastatin+AGA according to the present invention on 45 day of scheme A treatment.

Statistically significant differences on AST activity parameter in all groups with treatment except for group of AGA monotherapy was set from control group under Newman-Keuls test.

It should be noted that on 75 day of study intensive development of pathology was going on, and the increase of transaminase activity is associated with the severity and intensity of developed alterations. In such case side effects of statins were not able to develop yet and their therapeutic effect on modeled pathology came on the first place, resulted in decrease of AST activity in compare to control group. Despite no statistical difference was found, AGA administration occurred to have tendency to decrease AST activity on 75 day of pathology development.

Data showing the range of toxicological parameters of scheme A animals on 90 day of pathology, after 60 days of treatment, is represented in table 60.

Primary data corresponded to normal distribution. Under Newman-Keuls test, statistically significant difference was determined from control group on AST activity parameter in all groups with treatment, on CPK activity parameter in groups with AGA in a dose of 200 mg and 100 mg in combination with atorvastatin and rosuvastatin according to the present invention. Modeling of pathology during 90 days also resulted in an increase of glucose concentration above background level. In drug intake groups statistically significant difference was determined in glucose concentration from control group. Statistically significant difference in groups with treatment was determined only on ALT activity. For example, animals received atorvastatin and rosuvastatin during 60 days on scheme A, demonstrated tendency of increasing ALT activity while applying these drugs in double dose of 40 mg. In groups treated with combinations of statins and AGA in 20+200 mg dose according to the present invention, ALT activity was statistically much lower than in groups with 40 mg monotherapy.

The administration of combinations of statins and AGA in 20+200 mg dose was most effective concerning biochemical parameters on 60 day of treatment on scheme A. The effectiveness of both combinations on all studied doses on 60 day of treatment on scheme A was the same concerning studied biochemical parameters.

Results of dispersion analysis demonstrated the influence of group factor on AST and ALT activity, CPK and glucose level. But under Newman-Keuls test, there was statistically significant difference between treatment and control groups only on AST, CPK and Glucose parameters.

As a result of pathology modeling during 90 days, animals demonstrated the elevation of activity of AST, ALT, glucose concentration, the tendency to increase of direct bilirubin concentration. Long-term administration of statins, especially in double 40 mg dose, made additional contribution to increase of these parameters. For example, during 60 days of treatment tendencies of different intensity to increase of activity of AST, ALT and CPK were observed. The inclusion of AGA into the combination counter-balanced side effects of long-term therapy. According to data received, the inclusion of AGA in dose 200 mg into the combination of the present invention was most perspective as for biochemical parameters. In case of such pathology and treatment on scheme A both combinations in such dose were equipotent.

### Scheme B

### Toxicity Parameters Before Treatment

Data showing the range of toxicological parameters of scheme B animals before modeling of pathology and drug intake is represented in table 61. Primary data corresponded to normal distribution. Under Newman-Keuls test, there was no statistically significant difference between groups across all experimental variables.

Data showing the range of toxicological parameters of scheme B animals on 60 day of pathology, before treatment, is presented in table 62. Results of dispersion analysis demonstrated the influence of group factor on direct bilirubin parameter value. However, the presence of statistically significant difference (under Newman-Keuls test) between groups with pathology and intact group on AST, CPK and direct bilirubin parameters shows significantly compromised liver function and disorganization of metabolism affected by pathology modeling at 60 day of study.

### Toxicity Parameters Under the Treatment

Data showing the range of toxicological parameters of scheme B animals on 75 day of pathology (15 day of treatment), is represented in table 63. Primary data corresponded to normal distribution. Under Newman-Keuls test, there was statistically significant difference between pathology groups and intact group only on activity of AST, ALT, CPK and bilirubin concentration parameters values, showing pathology progression. Meanwhile, in groups receiving combination of rosuvastatin+AGA and atorvastatin+AGA in a dose of 20+200 mg according to the present invention, a statistically significant difference from control group on AST and ALT parameters was demonstrated also, and direct bilirubin concentrations in those groups did not differ from ones in intact group. Statistically significant difference in ALT concentrations should also be noted—in group received the combination of the present invention with AGA concentration of 200 mg, the ALT activity was statistically much lower than in groups of rosuvastatin and atorvastatin monotherapy.

Data showing the range of toxicological parameters of scheme B animals on 90 day of pathology (30 day of treatment), is represented in table 64. Primary data corresponded to normal distribution. Under Newman-Keuls test, there was statistically significant difference between pathology groups and intact group only on activity of AST, ALT, bilirubin and glucose concentration parameters values, showing pathology progression. Meanwhile, groups receiving study drugs demonstrated statistically significant difference from control group on AST and direct bilirubin concentrations parameters, and glucose concentration values did not differ from those in intact group and were significantly lower than in control group. According to effectiveness score, on the 30 day of treatment on scheme B most effective concerning biochemical parameters was the administration of combinations with AGA 200 mg dose. The administration of combinations during 30 days on scheme B demonstrated significant decrease of activity of AST, ALT and CPK, decrease direct bilirubin and glucose concentration compare to control group of animals. The effectiveness of combinations in a dose of 20+200 mg exceeded such of reference drug in equivalent dose of 20 mg. There was a tendency for the combination's effect to depend on the AGA dose.

Compared to monotherapy, the most effective was administration of the combination atorvastatin+AGA according to the present invention in a dose of 20+200 mg according to scheme B during 30 days. There is also a tendency for the drugs to be more effective compare to statins monotherapy.

Results of dispersion analysis demonstrated the influence of group factor on AST, ALT, CPK, direct and indirect bilirubin parameters values, approving the presence of pathology as well as the influence of administration of study drugs on blood biochemical parameters.

Data showing the range of toxicological parameters of scheme B animals on 105 day of pathology (45 day of treatment), is presented in table 65. Primary data corresponded to normal distribution. Under Newman-Keuls test, there was statistically significant difference between pathology groups and intact group on activity of AST, ALT, bilirubin and glucose concentrations parameters values. Meanwhile, in groups receiving study drugs, statistically significant difference from control group on AST and glucose concentrations parameters was demonstrated also, and activity of ALT in some groups did not differ from ones in intact group. Administration of both combinations of the present invention in a dose of 20+200 mg during 45 days by scheme B showed to be most effective according to estimation of drugs activity on all parameters of biochemical analysis. Studied combinations possessed equipotency on 45 day of treatment. All groups demonstrated direct dose-dependence of AGA concentration.

Results of dispersion analysis demonstrated the influence of group factor on AST, ALT, direct bilirubin and glucose parameters values, approving the presence of pathology as well as the influence of administration of study drugs on blood biochemical parameters.

Data showing the range of toxicological parameters of scheme B animals on 120 day of pathology (60 day of treatment), is presented in table 66. Primary data corresponded to normal distribution. Under Newman-Keuls test, there was statistically significant difference between pathology groups and intact group on activity of AST, ALT, CPK bilirubin and glucose concentration parameters values, showing pathology progression.

Results of dispersion analysis demonstrated the influence of group factor on AST, ALT, CPK, direct bilirubin and glucose parameters values, approving the presence of pathology as well as the influence of administration of study drugs on blood biochemical parameters.

The administration of the combinations of rosuvastatin+AGA and atorvastatin+AGA according to the present invention showed effectiveness in decreasing toxicological markers of modeled pathology. During 60 days of administration of studied combinations, significant decrease of activity of AST, ALT and CPK, bilirubin and glucose concentrations was demonstrated. Dynamics of CPK activity should be especially mentioned. At 120 day of study the activity of this enzyme in monotherapy groups of rosuvastatin and atorvastatin exceeded such of control group, meaning the development of one of the main side effect of statins—myopathy. Combinations with AGA demonstrated much lower activity of CPK than in control group. In AGA monotherapy group the decrease of CPK activity compared to control group was also demonstrated, approving the protective effect of AGA. The administration of studied rosuvastatin+AGA and atorvastatin+AGA in a dose of 20+200 mg combinations by scheme B during 60 days was most effective according to total score.

The study results show that the administration of studied drugs during 60 days by scheme B was characterized by high effectiveness on the intensity of changing of biochemical markers of modeled pathology. Long administration of statins also was accompanied by an increase of ALT and CPK activity. Including in combinations AGA according to the present invention in a dose of 200 and 100 mg statistically significant reduced the activity of these enzymes, demonstrating the possible role of AGA in decreasing of mytotoxicity and side effects of statins according to hepatic function.

The administration of the combination of atorvastatin+AGA according to the present invention in a dose of 20+200 mg showed in according to total score to be most perspective concerning biochemical markers.

TABLE 55
Toxicological Indicators Day 0 - Background (Before Treatment, To Pathology) - Scheme A

| | Sub- | | | | Toxicologocal parametrs M ± SEM | | |
|---|---|---|---|---|---|---|---|
| Group | group | Dose of active | | | AST, | ALT, | CPK, |
| [symbol] o | [symbol] o | ingredient mg | Group description | N | U/l | U/l | U/l |
| 2 | 2A | 0 | Control | 6 | 85 ± 5 | 71 ± 5 | 425 ± 16 |
| 5 | 3A | 20 + 200 | Treatment with | 6 | 81 ± 7 | 72 ± 6 | 422 ± 15 |
| 7 | 4A | 20 + 100 | rosuvastatin + AGA | 6 | 83 ± 8 | 74 ± 5 | 412 ± 11 |
| 9 | 5A | 20 + 50 | (the present invention) | 6 | 80 ± 5 | 75 ± 4 | 410 ± 12 |
| 11 | 6A | 20 | Comparison- treatment | 6 | 82 ± 9 | 73 ± 5 | 415 ± 14 |
| 13 | 6A2 | 40 | with rosuvastatin | 6 | 89 ± 9 | 75 ± 5 | 399 ± 29 |
| 14 | 7A | 20 + 200 | Treatment with | 6 | 90 ± 5 | 73 ± 2 | 428 ± 32 |
| 16 | 8A | 20 + 100 | atorvastatin + AGA | 6 | 91 ± 8 | 75 ± 4 | 432 ± 42 |
| 18 | 9A | 20 + 50 | (the present invention) | 6 | 89 ± 9 | 77 ± 5 | 425 ± 35 |
| 20 | 10A | 20 | Comparison - treatment | 6 | 91 ± 8 | 78 ± 7 | 411 ± 41 |
| 22 | 10A2 | 40 | with atorvastatin | 6 | 90 ± 9 | 79 ± 8 | 415 ± 39 |
| 23 | 11A | 108 | Comparison - treatment | 6 | 89 ± 8 | 72 ± 5 | 412 ± 32 |
| | | | with AGA | | | | |

| | Toxicologocal parametrs M ± SEM | | | | | |
|---|---|---|---|---|---|---|
| Group | TB, | DB, | B.ind., | Glucose, | K⁺, | Na⁺, |
| [symbol] o | μmol/l | μmol/l | μmol/l | mmol/l | mmol/l | mmol/l |
| 2 | 2.9 ± 0.2 | 0.9 ± 0.1 | 2.0 ± 0.2 | 88 ± 6 | 3.9 ± 0.3 | 125 ± 4 |
| 5 | 3.0 ± 0.1 | 0.9 ± 0.1 | 2.1 ± 0.2 | 89 ± 7 | 3.8 ± 0.2 | 127 ± 11 |
| 7 | 2.8 ± 0.2 | 1.0 ± 0.1 | 1.8 ± 0.3 | 87 ± 6 | 3.7 ± 0.3 | 125 ± 12 |
| 9 | 2.9 ± 0.3 | 0.9 ± 0.1 | 1.8 ± 0.3 | 88 ± 9 | 3.9 ± 0.3 | 123 ± 11 |
| 11 | 3.1 ± 0.2 | 0.9 ± 0.1 | 2.2 ± 0.3 | 90 ± 8 | 3.8 ± 0.3 | 129 ± 10 |
| 13 | 2.9 ± 0.1 | 1.0 ± 0.1 | 1.9 ± 0.1 | 90 ± 7 | 3.9 ± 0.2 | 132 ± 14 |
| 14 | 3.0 ± 0.2 | 0.9 ± 0.1 | 2.1 ± 0.2 | 92 ± 5 | 3.8 ± 0.3 | 127 ± 12 |
| 16 | 2.9 ± 0.3 | 1.0 ± 0.1 | 1.9 ± 0.3 | 90 ± 8 | 3.9 ± 0.3 | 128 ± 13 |
| 18 | 3.1 ± 0.3 | 0.9 ± 0.1 | 2.2 ± 0.4 | 91 ± 9 | 3.8 ± 0.3 | 129 ± 11 |
| 20 | 3.0 ± 0.3 | 0.9 ± 0.1 | 2.1 ± 0.4 | 92 ± 9 | 3.9 ± 0.3 | 130 ± 12 |
| 22 | 2.9 ± 0.2 | 1.0 ± 0.1 | 1.9 ± 0.2 | 90 ± 8 | 3.8 ± 0.3 | 128 ± 13 |
| 23 | 3.0 ± 0.3 | 0.9 ± 0.1 | 2.1 ± 0.3 | 89 ± 7 | 3.9 ± 0.2 | 129 ± 12 |

TABLE 56
Toxicological indicators on 30 day (before treatment) - the scheme A

| | Sub- | | | | Toxicological parameters M ± SEM | | |
|---|---|---|---|---|---|---|---|
| Group | group | Dose of active | | | AST, | ALT, | CPK, |
| [symbol] o | [symbol] o | ingredient (mg) | Group description | N | U/l | U/l | U/l |
| 2 | 2A | 0 | Control | 6 | 121 ± 11 | 92 ± 8 | 515 ± 14 |
| 5 | 3A | 20 + 200 | Treatment with | 6 | 123 ± 5 | 90 ± 6 | 517 ± 13 |
| 7 | 4A | 20 + 100 | rosuvastatin + AGA | 6 | 125 ± 12 | 87 ± 9 | 521 ± 32 |
| 9 | 5A | 20 + 50 | (the present invention) | 6 | 119 ± 11 | 89 ± 6 | 530 ± 42 |
| 11 | 6A | 20 | Comparison- treatment | 6 | 120 ± 8 | 90 ± 9 | 542 ± 55 |
| 13 | 6A2 | 40 | with rosuvastatin | 6 | 127 ± 12 | 89 ± 5 | 531 ± 18 |
| 14 | 7A | 20 + 200 | Treatment with | 6 | 127 ± 10 | 87 ± 8 | 520 ± 11 |
| 16 | 8A | 20 + 100 | atorvastatin + AGA | 6 | 119 ± 11 | 92 ± 4 | 514 ± 25 |
| 18 | 9A | 20 + 50 | (the present invention) | 6 | 121 ± 12 | 90 ± 8 | 529 ± 32 |
| 20 | 10A | 20 | Comparison- treatment | 6 | 119 ± 10 | 89 ± 6 | 534 ± 39 |
| 22 | 10A2 | 40 | with atorvastatin | 6 | 122 ± 11 | 87 ± 7 | 519 ± 29 |
| 23 | 11A | 108 | Comparison- treatment | 6 | 123 ± 10 | 89 ± 8 | 521 ± 32 |
| | | | with AGA | | | | |

| | Toxicological parameters M ± SEM | | | | | |
|---|---|---|---|---|---|---|
| Group | TB, | DB, | B.ind., | Glucose, | K+, | Na+, |
| [symbol] o | μmol/l | μmol/l | μmol/l | mmol/l | mmol/l | mmol/l |
| 2 | 3.6 ± 0.3 | 1.5 ± 0.1 | 2.1 ± 0.3 | 108 ± 10 | 3.8 ± 0.3 | 132 ± 10 |
| 5 | 3.5 ± 0.2 | 1.6 ± 0.1 | 1.9 ± 0.2 | 107 ± 5 | 4.0 ± 0.1 | 135 ± 11 |
| 7 | 3.5 ± 0.4 | 1.5 ± 0.1 | 2.0 ± 0.4 | 108 ± 5 | 3.8 ± 0.3 | 128 ± 7 |
| 9 | 3.7 ± 0.4 | 1.5 ± 0.1 | 2.4 ± 0.4 | 109 ± 10 | 3.9 ± 0.2 | 131 ± 14 |
| 11 | 3.6 ± 0.2 | 1.6 ± 0.1 | 2.0 ± 0.3 | 108 ± 11 | 3.9 ± 0.3 | 129 ± 10 |
| 13 | 3.7 ± 0.3 | 1.5 ± 0.1 | 2.2 ± 0.3 | 109 ± 10 | 3.9 ± 0.2 | 123 ± 10 |
| 14 | 3.5 ± 0.3 | 1.4 ± 0.2 | 2.1 ± 0.4 | 105 ± 4 | 3.8 ± 0.3 | 133 ± 10 |
| 16 | 3.5 ± 0.3 | 1.5 ± 0.1 | 2.0 ± 0.3 | 109 ± 7 | 3.9 ± 0.3 | 132 ± 12 |
| 18 | 3.6 ± 0.4 | 1.5 ± 0.1 | 2.1 ± 0.4 | 106 ± 8 | 3.7 ± 0.4 | 130 ± 11 |
| 20 | 3.5 ± 0.2 | 1.6 ± 0.1 | 1.9 ± 0.3 | 108 ± 10 | 3.8 ± 0.3 | 131 ± 12 |
| 22 | 3.4 ± 0.3 | 1.5 ± 0.2 | 1.9 ± 0.2 | 107 ± 10 | 3.9 ± 0.2 | 134 ± 14 |
| 23 | 3.5 ± 0.1 | 1.5 ± 0.1 | 2.0 ± 0.1 | 111 ± 12 | 3.8 ± 0.4 | 135 ± 12 |

TABLE 57
Toxicological indicators after 15 days of treatment (45 days of research) - the scheme A

| | Sub- | | | | Toxicological parameters M ± SEM, M ± SEM | | |
|---|---|---|---|---|---|---|---|
| Group | group | Dose of active | | | AST, | ALT, | CPK, |
| [symbol] o | [symbol] o | ingredient, mg | Group description | N | U/l | U/l | U/l |
| 2 | 2A | 0 | Control | 6 | 138 ± 10 | 106 ± 14 | 569 ± 22 |
| 5 | 3A | 20 + 200 | Treatment with | 6 | 124 ± 10 | 100 ± 7 | 501 ± 15 |
| 7 | 4A | 20 + 100 | rosuvastatin + AGA | 6 | 124 ± 11 | 105 ± 8 | 509 ± 22 |
| 9 | 5A | 20 + 50 | (the present invention) | 6 | 121 ± 14 | 101 ± 8 | 525 ± 32 |
| 11 | 6A | 20 | Comparison- treatment | 6 | 118 ± 5 | 113 ± 8 | 522 ± 42 |
| 13 | 6A2 | 40 | with rosuvastatin | 6 | 111 ± 10 | 100 ± 10 | 524 ± 12 |
| 14 | 7A | 20 + 200 | Treatment with | 6 | 121 ± 15 | 107 ± 9 | 505 ± 12 |
| 16 | 8A | 20 + 100 | atorvastatin + AGA | 6 | 118 ± 14 | 104 ± 10 | 508 ± 32 |
| 18 | 9A | 20 + 50 | (the present invention) | 6 | 119 ± 15 | 112 ± 7 | 518 ± 22 |
| 20 | 10A | 20 | Comparison- treatment | 6 | 100 ± 11 | 114 ± 9 | 530 ± 41 |
| 22 | 10A2 | 40 | with atorvastatin | 6 | 116 ± 14 | 119 ± 14 | 532 ± 22 |
| 23 | 11A | 100 | Comparison- treatment | 6 | 131 ± 11 | 104 ± 11 | 545 ± 31 |
| | | | with AGA | | | | |

| | Toxicological parameters M ± SEM, M ± SEM | | | | | |
|---|---|---|---|---|---|---|
| Group | TB, | DB, | B.ind., | Glucose, | K+, | Na+, |
| [symbol] o | μmol/l | μmol/l | μmol/l | mmol/l | mmol/l | mmol/l |
| 2 | 3.7 ± 0.2 | 1.6 ± 0.1 | 2.1 ± 0.3 | 119 ± 11 | 3.9 ± 0.3 | 128 ± 7 |
| 5 | 3.6 ± 0.3 | 1.7 ± 0.1 | 2.0 ± 0.3 | 114 ± 9 | 4.0 ± 0.4 | 131 ± 10 |
| 7 | 3.6 ± 0.4 | 1.6 ± 0.1 | 2.0 ± 0.4 | 112 ± 9 | 3.9 ± 0.4 | 129 ± 8 |
| 9 | 3.7 ± 0.3 | 1.6 ± 0.1 | 2.1 ± 0.3 | 112 ± 8 | 3.9 ± 0.3 | 127 ± 8 |
| 11 | 3.7 ± 0.1 | 1.6 ± 0.1 | 2.1 ± 0.2 | 117 ± 10 | 4.0 ± 0.2 | 122 ± 10 |
| 13 | 3.6 ± 0.2 | 1.5 ± 0.1 | 2.1 ± 0.2 | 110 ± 12 | 3.8 ± 0.3 | 123 ± 11 |
| 14 | 3.6 ± 0.3 | 1.5 ± 0.1 | 2.1 ± 0.3 | 109 ± 11 | 3.9 ± 0.4 | 128 ± 12 |
| 16 | 3.6 ± 0.2 | 1.6 ± 0.2 | 2.0 ± 0.4 | 111 ± 6 | 3.8 ± 0.2 | 125 ± 8 |
| 18 | 3.7 ± 0.3 | 1.6 ± 0.1 | 2.1 ± 0.2 | 109 ± 11 | 3.9 ± 0.2 | 127 ± 12 |
| 20 | 3.6 ± 0.1 | 1.5 ± 0.1 | 2.1 ± 1.2 | 112 ± 11 | 3.9 ± 0.1 | 130 ± 10 |
| 22 | 3.5 ± 0.2 | 1.6 ± 0.1 | 1.9 ± 0.1 | 109 ± 12 | 3.8 ± 0.3 | 131 ± 11 |
| 23 | 3.6 ± 0.2 | 1.6 ± 0.1 | 2.0 ± 0.3 | 112 ± 10 | 3.9 ± 0.2 | 132 ± 10 |

TABLE 58
Toxicity on 30 day of treatment (60 day of study) - Scheme A

| | | Active | | | Toxicity, M ± SEM | | |
|---|---|---|---|---|---|---|---|
| [symbol] o | [symbol] o | substance | | | AST, | ALT, | CPK, |
| gr | subgroup | dose, mg | Group description | N | U/l | U/l | U/l |
| 2 | 2A | 0 | Control | 6 | 152 ± 11 | 105 ± 11 | 557 ± 30 |
| 5 | 3A | 20 + 200 | Present invention - | 6 | 120 ± 16 | 90 ± 8 | 503 ± 27 |
| 7 | 4A | 20 + 100 | rosuvastatin + AGA | 6 | 130 ± 12 | 98 ± 9 | 501 ± 21 |
| 9 | 5A | 20 + 50 | treatment | 6 | 132 ± 5 | 106 ± 10 | 524 ± 18 |
| 11 | 6A | 20 | Comparison - | 6 | 117 ± 11 | 127 ± 6 | 538 ± 16 |
| 13 | 6A2 | 40 | rosuvastatin treatment | 6 | 125 ± 14 | 129 ± 11 | 552 ± 24 |
| 14 | 7A | 20 + 200 | Present invention- | 6 | 111 ± 10 | 96 ± 4 | 490 ± 25 |
| 16 | 8A | 20 + 100 | atorvastatin + AGA | 6 | 121 ± 10 | 100 ± 7 | 500 ± 43 |
| 18 | 9A | 20 + 50 | treatment | 6 | 129 ± 11 | 105 ± 9 | 526 ± 14 |
| 20 | 10A | 20 | Comparison - | 6 | 122 ± 11 | 125 ± 11 | 532 ± 35 |
| 22 | 10A2 | 40 | atorvastatin treatment | 6 | 125 ± 10 | 129 ± 10 | 550 ± 14 |
| 23 | 11A | 100 | Comparison - | 6 | 141 ± 15 | 98 ± 3 | 529 ± 51 |
| | | | AGA treatment | | | | |

| | Toxicity, M ± SEM | | | | | |
|---|---|---|---|---|---|---|
| [symbol] o | TB, | DB, | B.ind., | Glucose, | K+, | Na+, |
| gr | μmol/l | μmol/l | μmol/l | mmol/l | mmol/l | mmol/l |
| 2 | 3.9 ± 0.2 | 1.7 ± 0.1 | 2.2 ± 0.2 | 119 ± 12 | 3.9 ± 0.1 | 131 ± 10 |
| 5 | 3.8 ± 0.1 | 1.6 ± 0.1 | 2.2 ± 0.1 | 109 ± 11 | 3.9 ± 0.3 | 129 ± 12 |
| 7 | 3.9 ± 0.1 | 1.7 ± 0.3 | 2.2 ± 0.2 | 111 ± 10 | 3.8 ± 0.1 | 127 ± 11 |
| 9 | 3.9 ± 0.1 | 1.6 ± 0.2 | 2.3 ± 0.2 | 112 ± 14 | 3.9 ± 0.3 | 132 ± 12 |
| 11 | 3.8 ± 0.2 | 1.6 ± 0.1 | 2.2 ± 0.2 | 110 ± 11 | 3.8 ± 0.2 | 128 ± 11 |
| 13 | 3.8 ± 0.1 | 1.6 ± 0.1 | 2.2 ± 0.1 | 112 ± 10 | 3.8 ± 0.1 | 129 ± 10 |
| 14 | 3.8 ± 0.1 | 1.6 ± 0.1 | 2.2 ± 0.1 | 111 ± 4 | 3.9 ± 0.2 | 127 ± 12 |
| 16 | 3.9 ± 0.1 | 1.7 ± 0.1 | 2.2 ± 0.2 | 110 ± 6 | 3.9 ± 0.2 | 131 ± 11 |
| 18 | 3.9 ± 0.2 | 1.7 ± 0.1 | 2.2 ± 0.2 | 108 ± 11 | 3.8 ± 0.4 | 130 ± 10 |
| 20 | 3.8 ± 0.3 | 1.6 ± 0.1 | 2.2 ± 0.3 | 109 ± 6 | 3.9 ± 0.3 | 135 ± 14 |
| 22 | 3.8 ± 0.2 | 1.6 ± 0.1 | 2.2 ± 0.3 | 105 ± 9 | 4.0 ± 0.4 | 128 ± 11 |
| 23 | 3.8 ± 0.3 | 1.7 ± 0.1 | 2.1 ± 0.3 | 111 ± 10 | 4.0 ± 0.3 | 132 ± 14 |

TABLE 59
Toxicity on 45 day of treatment (75 day of study) - scheme A

| | | Active | | | Toxicity, M ± SEM | | |
|---|---|---|---|---|---|---|---|
| [symbol] o | [symbol] o | substance | | | AST, | ALT, | CPK, |
| gr | subgroup | dose, mg | Group description | N | U/l | U/l | U/l |
| 2 | 2A | 0 | Control | 6 | 181 ± 14 | 129 ± 11 | 615 ± 45 |
| 5 | 3A | 20 + 200 | Present invention - | 6 | 108 ± 12* | 91 ± 9 | 532 ± 29* |
| 7 | 4A | 20 + 100 | rosuvastatin + AGA | 6 | 105 ± 10* | 102 ± 7 | 549 ± 14 |
| 9 | 5A | 20 + 50 | treatment | 6 | 131 ± 15* | 104 ± 8 | 555 ± 23 |
| 11 | 6A | 20 | Comparison - | 6 | 118 ± 13* | 119 ± 7 | 568 ± 26 |
| 13 | 6A2 | 40 | rosuvastatin treatment | 6 | 129 ± 12* | 130 ± 15 | 502 ± 21 |
| 14 | 7A | 20 + 200 | Present invention - | 6 | 110 ± 11* | 85 ± 8 | 510 ± 48* |
| 16 | 8A | 20 + 100 | atorvastatin + AGA | 6 | 108 ± 12* | 90 ± 8 | 518 ± 32* |
| 18 | 9A | 20 + 50 | treatment | 6 | 121 ± 12* | 110 ± 8 | 549 ± 23 |
| 20 | 10A | 20 | Comparison - | 6 | 114 ± 9* | 121 ± 10 | 550 ± 31 |
| 22 | 10A2 | 40 | atorvastatin treatment | 6 | 124 ± 8* | 131 ± 14 | 571 ± 10 |
| 23 | 11A | 100 | Comparison - | 6 | 151 ± 14 | 108 ± 10 | 509 ± 21 |
| | | | AGA treatment | | | | |

| | Toxicity, M ± SEM | | | | | |
|---|---|---|---|---|---|---|
| [symbol] o | TB, | DB, | B.ind., | Glucose, | K+, | Na+, |
| gr | μmol/l | μmol/l | μmol/l | mmol/l | mmol/l | mmol/l |
| 2 | 4.1 ± 0.4 | 1.9 ± 0.1 | 2.2 ± 0.5 | 127 ± 9 | 3.9 ± 0.3 | 128 ± 10 |
| 5 | 3.9 ± 0.3 | 1.8 ± 0.1 | 2.1 ± 0.3 | 108 ± 6 | 4.0 ± 0.3 | 131 ± 11 |
| 7 | 3.8 ± 0.2 | 1.8 ± 0.1 | 2.0 ± 0.2 | 105 ± 10 | 3.9 ± 0.2 | 129 ± 10 |
| 9 | 3.9 ± 0.2 | 1.7 ± 0.1 | 2.2 ± 0.3 | 109 ± 12 | 3.8 ± 0.2 | 126 ± 8 |
| 11 | 3.8 ± 0.3 | 1.7 ± 0.1 | 2.1 ± 0.4 | 108 ± 10 | 3.9 ± 0.1 | 132 ± 10 |
| 13 | 3.8 ± 0.2 | 1.6 ± 0.1 | 2.2 ± 0.2 | 111 ± 9 | 3.8 ± 0.3 | 135 ± 12 |
| 14 | 3.9 ± 0.2 | 1.7 ± 0.1 | 2.2 ± 0.3 | 102 ± 9 | 3.9 ± 0.1 | 128 ± 11 |
| 16 | 3.8 ± 0.1 | 1.6 ± 0.1 | 2.2 ± 0.1 | 114 ± 6 | 3.8 ± 0.3 | 129 ± 12 |
| 18 | 3.8 ± 0.2 | 1.6 ± 0.1 | 2.2 ± 0.3 | 102 ± 9 | 3.9 ± 0.2 | 125 ± 13 |
| 20 | 3.8 ± 0.1 | 1.6 ± 0.1 | 2.2 ± 0.1 | 107 ± 9 | 3.8 ± 0.1 | 128 ± 11 |
| 22 | 3.8 ± 0.1 | 1.6 ± 0.1 | 2.2 ± 0.1 | 108 ± 5 | 3.8 ± 0.3 | 119 ± 12 |
| 23 | 3.9 ± 0.3 | 1.8 ± 0.1 | 2.1 ± 0.3 | 104 ± 10 | 4.0 ± 0.1 | 128 ± 12 |

Note:
*p < 0.05 - significant difference from control group (Newman-Keuls test);

TABLE 60
Toxicity 60 day of treatment (90 day of study) - scheme A

| | | Active | | | Toxicity, M ± SEM | | |
|---|---|---|---|---|---|---|---|
| [symbol] o | [symbol] o | substance | | | AST, | ALT, | CPK, |
| gr | subgroup | dose, mg | Group description | N | U/l | U/l | U/l |
| 2 | 2A | 0 | Control | 6 | 289 ± 12 | 109 ± 11 | 469 ± 41 |
| 5 | 3A | 20 + 200 | Present invention - | 6 | 84 ± 4* | 78 ± 6 | 400 ± 15* |
| 7 | 4A | 20 + 100 | rosuvastatin + AGA | 6 | 85 ± 4* | 88 ± 7 | 410 ± 10 |
| 9 | 5A | 20 + 50 | treatment | 6 | 90 ± 3* | 91 ± 8 | 415 ± 12 |
| 11 | 6A | 20 | Comparison - | 6 | 89 ± 7* | 111 ± 5 | 449 ± 14 |
| 13 | 6A2 | 40 | rosuvastatin treatment | 6 | 98 ± 5* | 116 ± 8 | 478 ± 12 |
| 14 | 7A | 20 + 200 | Present invention - | 6 | 75 ± 9* | 75 ± 7 | 380 ± 18* |
| 16 | 8A | 20 + 100 | atorvastatin + AGA | 6 | 80 ± 7* | 90 ± 9 | 390 ± 15* |
| 18 | 9A | 20 + 50 | treatment | 6 | 91 ± 8* | 97 ± 9 | 411 ± 10 |
| 20 | 10A | 20 | Comparison - | 6 | 87 ± 5* | 110 ± 5 | 438 ± 12 |
| 22 | 10A2 | 40 | atorvastatin treatment | 6 | 88 ± 4* | 118 ± 7 | 455 ± 23 |
| 23 | 11A | 100 | Comparison - | 6 | 145 ± 11* | 98 ± 5 | 425 ± 18 |
| | | | AGA treatment | | | | |

| | Toxicity, M ± SEM | | | | | |
|---|---|---|---|---|---|---|
| [symbol] o | TB, | DB, | B.ind., | Glucose, | K+, | Na+, |
| gr | μmol/l | μmol/l | μmol/l | mmol/l | mmol/l | mmol/l |
| 2 | 4.0 ± 0.3 | 1.9 ± 0.1 | 2.1 ± 0.3 | 129 ± 11 | 3.9 ± 0.2 | 121 ± 8 |
| 5 | 3.5 ± 0.2 | 1.5 ± 0.1 | 2.0 ± 0.2 | 89 ± 4* | 3.9 ± 0.1 | 125 ± 10 |
| 7 | 3.6 ± 0.1 | 1.6 ± 0.1 | 2.0 ± 0.1 | 95 ± 7* | 3.9 ± 0.2 | 121 ± 9 |
| 9 | 3.6 ± 0.3 | 1.5 ± 0.1 | 2.1 ± 0.4 | 97 ± 5* | 3.8 ± 0.2 | 124 ± 12 |
| 11 | 3.6 ± 0.2 | 1.6 ± 0.1 | 2.0 ± 0.3 | 99 ± 7* | 3.8 ± 0.3 | 121 ± 11 |
| 13 | 3.5 ± 0.3 | 1.6 ± 0.1 | 1.9 ± 0.3 | 95 ± 5* | 3.8 ± 0.1 | 120 ± 7 |
| 14 | 3.6 ± 0.1 | 1.5 ± 0.1 | 2.1 ± 0.2 | 90 ± 7* | 3.9 ± 0.2 | 125 ± 9 |
| 16 | 3.7 ± 0.3 | 1.6 ± 0.1 | 2.1 ± 0.3 | 92 ± 8* | 3.8 ± 0.1 | 127 ± 8 |
| 18 | 3.5 ± 0.2 | 1.6 ± 0.1 | 2.0 ± 0.1 | 98 ± 6* | 3.9 ± 0.2 | 124 ± 9 |
| 20 | 3.5 ± 0.3 | 1.5 ± 0.1 | 2.0 ± 0.3 | 95 ± 4* | 3.8 ± 0.1 | 119 ± 8 |
| 22 | 3.6 ± 0.2 | 1.6 ± 0.1 | 2.0 ± 0.2 | 96 ± 5* | 3.9 ± 0.2 | 120 ± 9 |
| 23 | 3.8 ± 0.3 | 1.7 ± 0.1 | 2.1 ± 0.3 | 95 ± 9* | 3.9 ± 0.3 | 121 ± 4 |

Note:
*p < 0.05 - significant difference from control group(Newman-Keuls test);

TABLE 61
Toxicity before study (background level) - Scheme B

| | | | | | Toxicity, M ± SEM | | |
|---|---|---|---|---|---|---|---|
| [symbol] o | [symbol] o | | | | AST, | ALT, | CPK, |
| gr | subgroup | Dose, mg | Group description | N | U/l | U/l | U/l |
| 1 | 1 | 0 | Intact (without pathology, | 6 | 79 ± 4 | 73 ± 3 | 407 ± 11 |
| | | | without treatment) | | | | |
| 3 | 2B | 0 | Control with pathology, without | 6 | 82 ± 5 | 75 ± 5 | 399 ± 25 |
| | | | treatment | | | | |
| 4 | 2B1 | 0 | Control -with pathology, | 6 | 81 ± 3 | 72 ± 8 | 421 ± 20 |
| | | | without treatment | | | | |
| 6 | 3B | 20 + 200 | Test - with pathology + | 6 | 78 ± 4 | 74 ± 5 | 415 ± 12 |
| 8 | 4B | 20 + 100 | treatment with the present | 6 | 79 ± 5 | 70 ± 7 | 402 ± 25 |
| 10 | 5B | 20 + 50 | invention (rosuvastatin + AGA) | 6 | 83 ± 7 | 71 ± 9 | 411 ± 22 |
| 12 | 6B | 20 | Animals with pathology + | 6 | 82 ± 8 | 72 ± 5 | 407 ± 25 |
| | | | rosuvastatin treatment | | | | |
| 15 | 7B | 20 + 200 | | 6 | 78 ± 5 | 71 ± 2 | 409 ± 32 |
| 17 | 8B | 20 + 100 | Test - with pathology + | 6 | 80 ± 8 | 75 ± 7 | 415 ± 25 |
| 19 | 9B | 20 + 50 | treatment with the present | 6 | 81 ± 8 | 71 ± 4 | 425 ± 31 |
| | | | invention (atorvastatin + AGA) | | | | |
| 21 | 10B | 20 | Animals with pathology + | 6 | 78 ± 5 | 72 ± 5 | 422 ± 40 |
| | | | atorvastatin treatment | | | | |
| 24 | 11B | 108 | Animals with pathology + AGA | 6 | 80 ± 4 | 73 ± 2 | 427 ± 42 |
| | | | treatment | | | | |

| | Toxicity, M ± SEM | | | | | |
|---|---|---|---|---|---|---|
| [symbol] o | TB, | DB, | B.ind., | Glucose, | K+, | Na+, |
| gr | μmol/l | μmol/l | μmol/l | mmol/l | mmol/l | mmol/l |
| 1 | 3.1 ±0.2 | 0.8 ±0.1 | 2.3 ± 0.2 | 85 ±6 | 3.8 ± 0.3 | 123 ± 4 |
| 3 | 3.0 ± 0.1 | 1.0 ± 0.1 | 2.0 ± 0.1 | 91 ± 7 | 3.6 ± 0.2 | 121 ± 7 |
| 4 | 2.8 ± 0.3 | 0.9 ± 0.1 | 1.9 ± 0.3 | 90 ± 8 | 3.9 ± 0.3 | 119 ± 12 |
| 6 | 2.9 ± 0.2 | 1.1 ± 0.1 | 1.8 ± 0.3 | 85 ± 7 | 3.8 ± 0.2 | 122 ± 10 |
| 8 | 2.9 ± 0.1 | 1.0 ± 0.1 | 1.9 ± 0.2 | 84 ± 8 | 3.9 ± 0.1 | 125 ± 11 |
| 10 | 3.0 ± 0.2 | 1.1 ± 0.1 | 2.0 ± 0.1 | 92 ± 7 | 4.0 ± 0.2 | 123 ± 12 |
| 12 | 2.9 ± 0.1 | 0.9 ± 0.1 | 1.8 ± 0.2 | 90 ± 5 | 3.9 ± 0.2 | 125 ± 11 |
| 15 | 2.8 ± 0.3 | 1.0 ± 0.1 | 1.8 ± 0.2 | 91 ± 9 | 3.9 ± 0.3 | 124 ± 12 |
| 17 | 2.9 ± 0.2 | 1.1 ± 0.1 | 1.8 ± 0.2 | 92 ± 8 | 3.8 ± 0.3 | 125 ± 11 |
| 19 | 3.0 ± 0.3 | 0.9 ± 0.1 | 2.1 ± 0.3 | 93 ± 5 | 3.9 ± 0.2 | 124 ± 12 |
| 21 | 2.8 ± 0.3 | 0.9 ± 0.1 | 1.9 ± 0.3 | 89 ± 4 | 4.0 ± 0.3 | 125 ± 11 |
| 24 | 2.9 ± 0.2 | 0.9 ± 0.1 | 2.0 ± 0.1 | 90 ± 6 | 3.9 ± 0.3 | 122 ± 12 |

TABLE 62
Toxicity on 60 day (before treatment) - scheme B

| | | | | | Toxicity, M ± SEM | | |
|---|---|---|---|---|---|---|---|
| [symbol] o | [symbol] o | | | | AST, | ALT, | CPK, |
| Gr | subgroup | Dose, mg | Group description | N | U/l | U/l | U/l |
| 1 | 1 | 0 | Intact(without pathology, without | 6 | 85 ± 5 | 71 ± 5 | 425 ± 16 |
| | | | treatment) | | | | |
| 3 | 2B | 0 | Control - with pathology, without | 6 | 134 ± 5¹ | 95 ± 5 | 535 ± 12¹ |
| | | | treatment | | | | |
| 4 | 2B1 | 0 | Control - with pathology, without | 6 | 141 ± 4 | 93 ± 7 | 541 ± 22¹ |
| | | | treatment | | | | |
| 6 | 3B | 20 + 200 | Test- with pathology + treatment | 6 | 145 ± 10¹ | 96 ± 9 | 555 ± 25¹ |
| 8 | 4B | 20 + 100 | with the present invention | 6 | 139 ± 18¹ | 97 ± 8 | 544 ± 30¹ |
| 10 | 5B | 20 + 50 | (rosuvastatin + AGA) | 6 | 140 ± 15¹ | 96 ± 9 | 551 ± 23¹ |
| 12 | 6B | 20 | Comparison - animals with | 6 | 137 ± 13¹ | 99 ± 8 | 554 ± 25¹ |
| | | | pathology + treatment rosuvastatin | | | | |
| 15 | 7B | 20 + 200 | Test- with pathology + treatment | 6 | 147 ± 18 | 98 ± 8 | 545 ± 17¹ |
| 17 | 8B | 20 + 100 | with the present invention | 6 | 139 ± 17¹ | 99 ± 9 | 552 ± 39¹ |
| 19 | 9B | 20 + 50 | (atorvastatin + AGA) | 6 | 141 ± 14¹ | 95 ± 7 | 552 ± 25¹ |
| 21 | 10B | 20 | Animals with pathology + | 6 | 139 ± 15¹ | 92 ± 8 | 545 ± 32¹ |
| | | | treatment atorvastatin | | | | |
| 24 | 11B | 108 | Animals with pathology + | 6 | 142 ± 16 | 95 ± 9 | 549 ± 30¹ |
| | | | treatment AGA | | | | |

| | Toxicity, M ± SEM | | | | | |
|---|---|---|---|---|---|---|
| [symbol] o | TB, | DB, | B.ind., | Glucose, | K+, | Na+, |
| Gr | μmol/l | μmol/l | μmol/l | mmol/l | mmol/l | mmol/l |
| 1 | 2.9 ± 0.2 | 0.9 ± 0.1 | 2.0 ± 0.2 | 88 ± 6 | 3.9 ± 0.3 | 125 ± 4 |
| 3 | 3.9 ± 0.3 | 1.7 ± 0.1¹ | 2.2 ± 0.2 | 112 ± 2 | 4.0 ± 0.3 | 135 ± 6 |
| 4 | 3.8 ± 0.3 | 1.6 ± 0.2¹ | 2.2 ± 0.4 | 100 ± 11 | 4.0 ± 0.2 | 141 ± 11 |
| 6 | 3.9 ± 0.3 | 1.7 ± 0.1¹ | 2.6 ± 0.3 | 118 ± 15 | 3.9 ± 0.2 | 132 ± 10 |
| 8 | 4.0 ± 0.4 | 1.5 ± 0.2¹ | 2.5 ± 0.5 | 121 ± 15 | 3.9 ± 0.1 | 135 ± 15 |
| 10 | 3.8 ± 0.2 | 1.7 ± 0.1¹ | 2.1 ± 0.3 | 100 ± 16 | 4.0 ± 0.2 | 137 ± 14 |
| 12 | 3.9 ± 0.3 | 1.7 ± 0.1¹ | 2.2 ± 0.3 | 122 ± 15 | 3.9 ± 0.1 | 139 ± 15 |
| 15 | 3.8 ± 0.2¹ | 1.7 ± 0.2¹ | 2.1 ± 0.3 | 100 ± 11 | 4.0 ± 0.2 | 142 ± 17 |
| 17 | 3.9 ± 0.1 | 1.6 ± 0.1¹ | 2.3 ± 0.1 | 114 ± 8 | 4.0 ± 0.1 | 145 ± 11 |
| 19 | 3.8 ± 0.3 | 1.7 ± 0.1¹ | 2.1 ± 0.3 | 116 ± 7 | 3.8 ± 0.3 | 139 ± 14 |
| 21 | 3.9 ± 0.4 | 1.6 ± 0.1¹ | 2.3 ± 0.4 | 118 ± 14 | 3.9 ± 0.2 | 140 ± 15 |
| 24 | 4.0 ± 0.3 | 1.7 ± 0.2¹ | 2.3 ± 0.3 | 117 ± 12 | 3.9 ± 0.1 | 144 ± 12 |

Note:
¹p < 0.05 - significant difference from intact group (Newman-Keuls test).

TABLE 63
Toxicity on 15 days of treatment (75 day of study) - Scheme B

| | | | | | Toxicity, M ± SEM | | |
|---|---|---|---|---|---|---|---|
| [symbol] o | [symbol] o | | | | AST, | ALT, | CPK, |
| gr | subgroup | Dose, mg | Group description | N | U/l | U/l | U/l |
| 1 | 1 | 0 | Intact (without pathology, without | 6 | 70 ± 7 | 62 ± 5 | 411 ± 22 |
| | | | treatment) | | | | |
| 4 | 2B1 | 0 | Control - with pathology, without | 6 | 189 ± 11¹ | 193 ± 25¹ | 621 ± 23¹ |
| | | | treatment | | | | |
| 6 | 3B | 20 + 200 | Test- with pathology + treatment | 6 | 119 ± 14^{1,2} | 92 ± 7² | 542 ± 27¹ |
| 8 | 4B | 20 + 100 | with the present invention | 6 | 147 ± 5¹ | 125 ± 11¹² | 552 ± 21¹ |
| 10 | 5B | 20 + 50 | (rosuvastatin + AGA) | 6 | 152 ± 12¹ | 149 ± 14¹ | 575 ± 32¹ |
| 12 | 6B | 20 | Comparison - animals with | 6 | 155 ± 12¹ | 172 ± 9¹ | 599 ± 23¹ |
| | | | pathology + treatment rosuvastatin | | | | |
| 15 | 7B | 20 + 200 | Test- with pathology + treatment | 6 | 121 ± 19^{1,2} | 101 ± 14² | 531 ± 12¹ |
| 17 | 8B | 20 + 100 | with the present invention | 6 | 139 ± 12¹ | 127 ± 11^{1,2} | 547 ± 21¹ |
| 19 | 9B | 20 + 50 | (atorvastatin + AGA) | 6 | 145 ± 10¹ | 143 ± 5¹ | 564 ± 32¹ |
| 21 | 10B | 20 | Comparison - animals with | 6 | 160 ± 18¹ | 173 ± 15¹ | 501 ± 24¹ |
| | | | pathology + treatment atorvastatin | | | | |
| 24 | 11B | 100 | Comparison - animals with | 6 | 141 ± 12¹ | 118 ± 10¹² | 550 ± 20¹ |
| | | | pathology + treatment AGA | | | | |

| | Toxicity, M ± SEM | | | | | |
|---|---|---|---|---|---|---|
| [symbol] o | TB, | DB, | B.ind., | Glucose, | K+, | Na+, |
| gr | μmol/l | μmol/l | μmol/l | mmol/l | mmol/l | mmol/l |
| 1 | 3.0 ± 0.3 | 1.0 ± 0.1 | 2.0 ± 0.3 | 82 ± 5 | 3.9 ± 0.4 | 123 ± 5 |
| 4 | 4.2 ± 0.4 | 1.9 ± 0.2¹ | 2.3 ± 0.6 | 123 ± 12 | 4.0 ± 0.1 | 132 ± 8 |
| 6 | 3.3 ± 0.2 | 1.4 ± 0.1 | 1.9 ± 0.1 | 103 ± 8 | 4.3 ± 0.4 | 131 ± 7 |
| 8 | 3.5 ± 0.1 | 1.5 ± 0.2 | 2.0 ± 0.2 | 112 ± 10 | 4.3 ± 0.2 | 121 ± 10 |
| 10 | 3.4 ± 0.3 | 1.6 ± 0.2 | 1.8 ± 0.4 | 121 ± 16 | 4.0 ± 0.2 | 117 ± 14 |
| 12 | 3.6 ± 0.2 | 1.8 ± 0.2¹ | 1.8 ± 0.3 | 123 ± 10 | 3.9 ± 0.3 | 118 ± 10 |
| 15 | 3.5 ± 0.2 | 1.4 ± 0.2 | 2.1 ± 0.2 | 110 ± 9 | 4.2 ± 0.4 | 128 ± 12 |
| 17 | 3.6 ± 0.2 | 1.5 ± 0.1 | 2.1 ± 0.2 | 114 ± 9 | 4.1 ± 0.3 | 125 ± 8 |
| 19 | 3.7 ± 0.2 | 1.7 ± 0.2 | 2.0 ± 0.3 | 119 ± 9 | 3.9 ± 0.4 | 119 ± 12 |
| 21 | 3.6 ± 0.4 | 1.6 ± 0.1 | 2.0 ± 0.4 | 118 ± 14 | 3.9 ± 0.3 | 120 ± 11 |
| 24 | 3.9 ± 0.3 | 1.8 ± 0.1¹ | 2.0 ± 0.3 | 100 ± 17 | 4.0 ± 0.2 | 127 ± 14 |

Note:
¹p < 0.05 - significant difference from intact group (Newman-Keuls test);
²p < 0.05 - significant difference from control group (Newman-Keuls test).

TABLE 64
Toxicity after 30 days of treatment (90 day of study) - Scheme B

| | | | | | Toxicity, M ± SEM | | |
|---|---|---|---|---|---|---|---|
| [symbol] o | [symbol] o | | | | AST, | ALT, | CPK, |
| gr | subgroup | Dose, mg | Group description | N | U/l | U/l | U/l |
| 1 | 1 | 0 | Intact(without pathology, without | 6 | 71 ± 5 | 65 ± 4 | 392 ± 37 |
| | | | treatment) | | | | |
| 4 | 2B1 | 0 | Control - with pathology, | 6 | 267 ± 14¹ | 111 ± 10¹ | 475 ± 44 |
| | | | without treatment | | | | |
| 6 | 3B | 20 + 200 | Test- with pathology + treatment | 6 | 77 ± 5² | 90 ± 8¹ | 408 ± 31 |
| 8 | 4B | 20 + 100 | with the present invention | 6 | 80 ± 8² | 98 ± 9¹ | 419 ± 11 |
| 10 | 5B | 20 + 50 | (rosuvastatin + AGA) | 6 | 85 ± 2² | 102 ± 10¹ | 425 ± 19 |
| 12 | 6B | 20 | Comparison -animals with | 6 | 88 ± 4² | 105 ± 8¹ | 478 ± 31 |
| | | | pathology + treatment rosuvastatin | | | | |
| 15 | 7B | 20 + 200 | Test- with pathology + treatment | 6 | 71 ± 7² | 81 ± 5¹ | 398 ± 25² |
| 17 | 8B | 20 + 100 | with the present invention | 6 | 80 ± 8² | 90 ± 4¹ | 405 ± 32 |
| 19 | 9B | 20 + 50 | (atorvastatin + AGA) | 6 | 87 ± 6² | 104 ± 9¹ | 434 ± 35 |
| 21 | 10B | 20 | Comparison- animals with | 6 | 101 ± 6² | 100 ± 5¹ | 481 ± 35 |
| | | | pathology + treatment atorvastatin | | | | |
| 24 | 11B | 100 | Comparison -animals with | 6 | 142 ± 14¹² | 109 ± 6¹ | 453 ± 37 |
| | | | pathology + treatment AGA | | | | |

| | Toxicity, M ± SEM | | | | | |
|---|---|---|---|---|---|---|
| [symbol] o | TB, | DB, | B.ind., | Glucose, | K+, | Na+, |
| gr | μmol/l | μmol/l | μmol/l | mmol/l | mmol/l | mmol/l |
| 1 | 2.9 ± 0.2 | 1.0 ± 0.1 | 1.9 ± 0.2 | 78 ± 6 | 3.7 ± 0.2 | 118 ± 11 |
| 4 | 3.9 ± 0.2 | 1.9 ± 0.1¹ | 2.0 ± 0.3 | 128 ± 10¹ | 3.9 ± 0.3 | 124 ± 9 |
| 6 | 3.4 ± 0.3 | 1.4 ± 0. 1² | 2.0 ± 0.3 | 105 ± 9 | 4.0 ± 0.4 | 120 ± 11 |
| 8 | 3.5 ± 0.4 | 1.5 ± 0.1¹² | 2.0 ± 0.5 | 111 ± 11 | 3.9 ± 0.3 | 119 ± 8 |
| 10 | 3.7 ± 0.4 | 1.7 ± 0.1¹ | 2.0 ± 0.4 | 114 ± 10 | 3.8 ± 0.1 | 120 ± 11 |
| 12 | 3.5 ± 0.3 | 1.4 ± 0.1² | 2.1 ± 0.4 | 112 ± 9 | 3.7 ± 0.4 | 120 ± 7 |
| 15 | 3.3 ± 0.1 | 1.3 ± 0.1² | 2.0 ± 0.2 | 100 ± 8 | 4.0 ± 0.3 | 124 ± 10 |
| 17 | 3.5 ± 0.1 | 1.5 ± 0.2¹ | 2.0 ± 0.2 | 109 ± 5 | 4.0 ± 0.2 | 121 ± 10 |
| 19 | 3.5 ± 0.3 | 1.4 ± 0.1¹² | 2.1 ± 0.3 | 112 ± 7 | 3.8 ± 0.3 | 119 ± 15 |
| 21 | 3.6 ± 0.2 | 1.5 ± 0.1¹ | 2.1 ± 0.2 | 108 ± 12 | 3.7 ± 0.3 | 120 ± 8 |
| 24 | 3.5 ± 0.1 | 1.4 ± 0.1² | 2.1 ± 0.1 | 109 ± 8 | 3.9 ± 0.3 | 123 ± 12 |

Note:
¹p < 0.05 - significant difference from intact group (Newman-Keuls test);
²p < 0.05 - significant difference from control group(Newman-Keuls test).

TABLE 65
Toxicity at 45 day of treatment (105 day of study) - Scheme B

| | | | | | Toxicity, M ± SEM | | |
|---|---|---|---|---|---|---|---|
| [symbol] o | [symbol] o | | | | AST, | ALT, | CPK, |
| gr | subgroup | Dose, mg | Group description | N | U/l | U/l | U/l |
| 1 | 1 | 0 | Intact (without pathology, without | 6 | 69 ± 4 | 70 ± 7 | 385 ± 28 |
| | | | treatment) | | | | |
| 4 | 2B1 | 0 | Control - with pathology, | 6 | 279 ± 12¹ | 100 ± 11¹ | 432 ± 14 |
| | | | without treatment | | | | |
| 6 | 3B | 20 + 200 | Test- with pathology + treatment | 6 | 78 ± 9² | 81 ± 7 | 403 ± 22 |
| 8 | 4B | 20 + 100 | with the present invention | 6 | 79 ± 5² | 95 ± 8 | 415 ± 11 |
| 10 | 5B | 20 + 50 | (rosuvastatin + AGA) | 6 | 81 ± 8² | 98 ± 4 | 419 ± 28 |
| 12 | 6B | 20 | Comparison - Animals with | 6 | 86 ± 8² | 100 ± 7 | 488 ± 21 |
| | | | pathology + treatment rosuvastatin | | | | |
| 15 | 7B | 20 + 200 | Test- with pathology + treatment | 6 | 70 ± 6² | 70 ± 9 | 395 ± 21² |
| 17 | 8B | 20 + 100 | with the present invention | 6 | 82 ± 2² | 85 ± 7 | 402 ± 16 |
| 19 | 9B | 20 + 50 | (atorvastatin + AGA) | 6 | 84 ± 8² | 93 ± 8 | 429 ± 15 |
| 21 | 10B | 20 | Comparison - Animals with | 6 | 105 ± 5² | 95 ± 8 | 490 ± 32 |
| | | | pathology + treatment atorvastatin | | | | |
| 24 | 11B | 100 | Comparison - Animals with | 6 | 127 ± 11¹² | 99 ± 8 | 384 ± 32 |
| | | | pathology + treatment AGA | | | | |

| | Toxicity, M ± SEM | | | | | |
|---|---|---|---|---|---|---|
| [symbol] o | TB, | DB, | B.ind., | Glucose, | K+, | Na+, |
| gr | μmol/l | μmol/l | μmol/l | mmol/l | mmol/l | mmol/l |
| 1 | 3.0 ± 0.3 | 1.0 ±0.1 | 2.0 ± 0.2 | 80 ± 7 | 3.9 ± 0.1 | 125 ± 10 |
| 4 | 3.8 ± 0.3 | 1.8 ± 0.1¹ | 2.0 ± 0.4 | 132 ± 11¹ | 4.0 ± 0.3 | 124 ± 5 |
| 6 | 3.5 ± 0.2 | 1.5 ± 0.2¹ | 2.0 ± 0.2 | 92 ± 8² | 3.9 ± 0. | 119 ± 10 |
| 8 | 3.5 ± 0.4 | 1.5 ± 0.1 | 2.0 ± 0.4 | 101 ± 11 | 3.9 ± 0.3 | 126 ± 8 |
| 10 | 3.6 ± 0.3 | 1.6 ± 0.1¹ | 2.0 ± 0.4 | 95 ± 9 | 3.9 ± 0.2 | 123 ± 12 |
| 12 | 3.6 ± 0.3 | 1.6 ± 0.1¹ | 2.0 ± 0.2 | 102 ± 8² | 3.8 ± 0.4 | 121 ± 10 |
| 15 | 3.4 ± 0.2 | 1.3 ± 0.1 | 2.1 ± 0.1 | 90 ± 7² | 3.8 ± 0.3 | 119 ± 12 |
| 17 | 3.5 ± 0.3 | 1.5 ± 0.1¹ | 2.0 ± 0.4 | 102 ± 10² | 4.0 ± 0.3 | 121 ± 8 |
| 19 | 3.4 ± 0.3 | 1.4 ± 0.1 | 2.0 ± 0.3 | 100 ± 8 | 3.8 ± 0.4 | 122 ± 12 |
| 21 | 3.4 ± 0.2 | 1.3 ± 0.1 | 2.1 ± 0.2 | 88 ± 6² | 3.7 ± 0.4 | 119 ± 10 |
| 24 | 3.6 ± 0.3 | 1.3 ± 0.1 | 2.3 ± 0.3 | 99 ± 8 | 4.0 ± 0.4 | 126 ± 11 |

Note:
¹p < 0.05 - significant difference from intact group (Newman-Keuls test);
²p < 0.05 - significant difference from control group(Newman-Keuls test).

TABLE 66
Toxicity at 60 days of treatment (120 day of study) - Scheme B

| | | | | | Toxicity, M ± SEM | | |
|---|---|---|---|---|---|---|---|
| [symbol] o | [symbol] o | | | | AST, | ALT, | CPK, |
| gr | subgroup | Dose, mg | Group description | N | U/l | U/l | U/l |
| 1 | 1 | 0 | Intact (without pathology, without | 6 | 78 ± 5 | 75 ± 6 | 323 ± 28 |
| | | | treatment) | | | | |
| 4 | 2B1 | 0 | Control - with pathology, without | 6 | 271 ± 14¹ | 118 ± 10¹ | 444 ± 12¹ |
| | | | treatment | | | | |
| 6 | 3B | 20 + 200 | Test - with pathology + treatment | 6 | 80 ± 8² | 76 ± 4² | 341 ± 28² |
| 8 | 4B | 20 + 100 | with the present invention | 6 | 82 ± 6² | 85 ± 6² | 365 ± 31 |
| 10 | 5B | 20 + 50 | (rosuvastatin + AGA) | 6 | 99 ± 5² | 91 ± 4² | 425 ± 10¹ |
| 12 | 6B | 20 | Comparison - animals with | 6 | 103 ± 5² | 112 ± 3¹ | 466 ± 34¹ |
| | | | pathology + treatment rosuvastatin | | | | |
| 15 | 7B | 20 + 200 | Test - with pathology + treatment | 6 | 77 ± 5² | 74 ± 4² | 321 ± 15² |
| 17 | 8B | 20 + 100 | with the present invention | 6 | 81 ± 5² | 80 ± 2² | 335 ± 12² |
| 19 | 9B | 20 + 50 | (atorvastatin + AGA) | 6 | 86 ± 7² | 94 ± 6² | 415 ± 18¹ |
| 21 | 10B | 20 | Comparison - Animals with | 6 | 99 ± 6² | 105 ± 4¹ | 475 ± 11¹ |
| | | | pathology + treatment atorvastatin | | | | |
| 24 | 11B | 100 | Comparison - Animals with | 6 | 132 ± 12¹² | 98 ± 5² | 322 ± 30² |
| | | | pathology + treatment AGA | | | | |

| | Toxicity, M ± SEM | | | | | |
|---|---|---|---|---|---|---|
| [symbol] o | TB, | DB, | B.ind., | Glucose, | K+, | Na+, |
| gr | μmol/l | μmol/l | μmol/l | mmol/l | mmol/l | mmol/l |
| 1 | 2.9 ± 0.3 | 1.0 ± 0.1 | 1.9 ± 0.2 | 84 ± 6 | 4.0 ± 0.3 | 122 ± 11 |
| 4 | 4.2 ± 0.3¹ | 1.9 ± 0. l¹ | 2.3 ± 0.2 | 144 ± 10¹ | 3.9 ± 0.2 | 120 ± 11 |
| 6 | 3.1 ± 0.1 | 1.3 ± 0.1² | 1.8 ± 0.1 | 84 ± 5² | 3.9 ± 0.3 | 122 ± 8 |
| 8 | 3.5 ± 0.2 | 1.4 ± 0.1² | 2.1 ± 03 | 92 ± 5² | 4.0 ± 0.1 | 120 ± 7 |
| 10 | 3.5 ± 0.3 | 1.4 ± 0.1² | 2.1 ± 0.4 | 99 ± 7² | 3.9 ± 0.3 | 120 ± 10 |
| 12 | 3.5 ± 0.2 | 1.5 ± 0.1¹² | 2.0 ± 0.3 | 92 ± 8² | 3.9 ± 0.3 | 122 ± 7 |
| 15 | 3.1 ± 0.3 | 1.2 ± 0.1² | 1.9 ± 0.4 | 88 ± 5² | 3.9 ± 0.4 | 124 ± 12 |
| 17 | 3.3 ± 0.2 | 1.3 ± 0.1² | 2.0 ± 0.2 | 92 ± 9² | 4.0 ± 0.2 | 123 ± 7 |
| 19 | 3.3 ± 0.3 | 1.3 ± 0.1² | 2.0 ± 0.3 | 97 ± 6² | 3.9 ± 0.2 | 121 ± 9 |
| 21 | 3.2 ± 0.2 | 1.3 ± 0.1² | 1.9 ± 0.2 | 89 ± 7² | 3.9 ± 0.4 | 118 ± 11 |
| 24 | 3.7 ± 0.4 | 1.7 ± 0.1¹ | 2.0 ± 0.3 | 97 ± 5² | 4.0 ± 0.2 | 127 ± 12 |

Note:
¹p < 0.05 - significant difference from intact group (Newman-Keuls test);
²p < 0.05 - significant difference from control group (Newman-Keuls test).

Changes in Blood Coagulation Before and after the Treatment

### Scheme A

Data on blood coagulation of scheme A animals before treatment are represented in table 67.

TABLE 67
Blood coagulation 0 day (before treatment) - scheme A

| | Sub- | Active | | | | | |
|---|---|---|---|---|---|---|---|
| Group | group | ingredient | | | M ± SEM | | |
| [symbol] o | [symbol] o | dose, mg | Group description | N | PT | APTT | Platelets |
| 2 | 2A | 0 | Control | 6 | 18.0 ± 1.2 | 15.2 ± 0.2 | 303 ± 7 |
| 5 | 3A | 20 + 200 | Present invention - | 6 | 17.5 ± 0.4 | 16.2 ± 1.0 | 302 ± 4 |
| 7 | 4A | 20 + 100 | rosuvastatin + AGA | 6 | 18.0 ± 0.5 | 16.2 ± 1.2 | 312 ± 11 |
| 9 | 5A | 20 + 50 | treatment | 6 | 18.0 ± 1.0 | 15.8 ± 0.3 | 314 ± 10 |
| 11 | 6A | 20 | Comparison - | 6 | 18.5 ± 1.1 | 16.2 ± 0.5 | 311 ± 15 |
| 13 | 6A2 | 40 | rosuvastatin treatment | 6 | 17.8 ± 1.2 | 16.2 ± 0.8 | 321 ± 15 |
| 14 | 7A | 20 + 200 | Present invention - | 6 | 18.2 ± 1.2 | 15.8 ± 0.8 | 314 ± 12 |
| 16 | 8A | 20 + 100 | atorvastatin + AGA | 6 | 17.8 ± 1.4 | 15.8 ± 0.3 | 316 ± 18 |
| 18 | 9A | 20 + 50 | treatment | 6 | 17.7 ± 1.0 | 15.5 ± 0.8 | 321 ± 19 |
| 20 | 10A | 20 | Comparison - | 6 | 18.5 ± 1.4 | 16.0 ± 0.7 | 314 ± 18 |
| 22 | 10A2 | 40 | atorvastatin treatment | 6 | 18.7 ± 1.6 | 16.1 ± 0.7 | 320 ± 19 |
| 23 | 11A | 108 | Comparison - | 6 | 19.0 ± 1.7 | 16.3 ± 1.0 | 317 ± 18 |
| | | | AGA treatment | | | | |

Results of dispersion analysis before treatment demonstrated that the influence of group factor is being characterized by values F3;33=0.17, p=1.00. Under Newman-Keuls test, there was also no statistically significant difference of blood coagulation between studied groups before the experiment.

Data on blood coagulation of scheme A animals at 30 day are presented in table 68.

TABLE 68
Blood coagulation 30 day (before treatment) - scheme A

| | Sub- | Active | | | | | |
|---|---|---|---|---|---|---|---|
| Group | group | ingredient | | | M ± SEM | | |
| [symbol] o | [symbol] o | dose, mg | Group description | N | PT | APTT | Platelets |
| 2 | 2A | 0 | Control | 6 | 8.5 ± 0.6 | 15.0 ± 0.4 | 361 ± 22 |
| 5 | 3A | 20 + 200 | Present invention - | 6 | 8.5 ± 0.2 | 15.2 ± 0.7 | 350 ± 25 |
| 7 | 4A | 20 + 100 | rosuvastatin + AGA | 6 | 8.7 ± 0.7 | 14.7 ± 0.3 | 354 ± 30 |
| 9 | 5A | 20 + 50 | treatment | 6 | 8.5 ± 0.8 | 14.5 ± 0.8 | 367 ± 29 |
| 11 | 6A | 20 | Comparison - | 6 | 8.5 ± 0.7 | 14.3 ± 0.6 | 372 ± 32 |
| 13 | 6A2 | 40 | rosuvastatin treatment | 6 | 8.3 ± 0.8 | 14.5 ± 0.8 | 365 ± 28 |
| 14 | 7A | 20 + 200 | Present invention - | 6 | 8.5 ± 0.9 | 14.8 ± 0.6 | 361 ± 30 |
| 16 | 8A | 20 + 100 | atorvastatin + AGA | 6 | 8.0 ± 0.8 | 16.0 ± 0.9 | 350 ± 29 |
| 18 | 9A | 20 + 50 | treatment | 6 | 8.5 ± 0.8 | 15.2 ± 0.7 | 362 ± 35 |
| 20 | 10A | 20 | Comparison - | 6 | 8.3 ± 0.8 | 15.3 ± 0.3 | 371 ± 35 |
| 22 | 10A2 | 40 | atorvastatin treatment | 6 | 8.7 ± 0.6 | 15.2 ± 0.2 | 364 ± 28 |
| 23 | 11A | 108 | Comparison - | 6 | 8.5 ± 0.7 | 15.0 ± 0.4 | 360 ± 15 |
| | | | AGA treatment | | | | |

Results of dispersion analysis while modeling a pathology demonstrated that the influence of group factor is being characterized by values F3;33=0.22, p=1.00. Under Newman-Keuls test, there was also no statistically significant difference of blood coagulation between studied groups before the experiment.

At 30 day of pathology modeling in animals the significant decrease of PT and increase of platelets amount was demonstrated compare to baseline value. Such symptoms correspond to the development of atherosclerosis with high thrombosis risk and usually occur in clinical picture.

### Blood Coagulation Parameters on Treatment

Data on blood coagulation of scheme A animals at 45 day of study are presented in table 69.

TABLE 69
Blood coagulation 15 day of treatment (45 day of study) - scheme A

| | Sub- | Active | | | | | |
|---|---|---|---|---|---|---|---|
| Group | group | ingredient | | | M ± SEM | | |
| [symbol] o | [symbol] o | dose, mg | Group description | N | PT | APTT | Platelets |
| 2 | 2A | 0 | Control | 6 | 7.5 ± 0.6 | 13.8 ± 0.9 | 368 ± 32 |
| 5 | 3A | 20 + 200 | Present invention - | 6 | 8.5 ± 0.5 | 14.1 ± 1.0 | 332 ± 25 |
| 7 | 4A | 20 + 100 | rosuvastatin + AGA | 6 | 8.6 ± 0.4 | 13.9 ± 0.8 | 327 ± 15 |
| 9 | 5A | 20 + 50 | treatment | 6 | 8.5 ± 0.2 | 14.0 ± 0.5 | 332 ± 19 |
| 11 | 6A | 20 | Comparison - | 6 | 8.4 ± 0.8 | 14.0 ± 0.5 | 327 ± 24 |
| 13 | 6A2 | 40 | rosuvastatin treatment | 6 | 8.7 ± 0.6 | 14.2 ± 0.3 | 315 ± 19 |
| 14 | 7A | 20 + 200 | Present invention - | 6 | 9.0 ± 0.3 | 14.0 ± 0.5 | 320 ± 12 |
| 16 | 8A | 20 + 100 | atorvastatin + AGA | 6 | 9.1 ± 0.5 | 13.9 ± 0.7 | 332 ± 25 |
| 18 | 9A | 20 + 50 | treatment | 6 | 9.0 ± 0.9 | 14.1 ± 0.5 | 329 ± 32 |
| 20 | 10A | 20 | Comparison - | 6 | 9.0 ± 0.5 | 14.0 ± 0.5 | 331 ± 32 |
| 22 | 10A2 | 40 | atorvastatin treatment | 6 | 9.2 ± 0.8 | 14.1 ± 0.3 | 337 ± 14 |
| 23 | 11A | 108 | Comparison - | 6 | 7.8 ± 0.6 | 14.0 ± 0.3 | 349 ± 12 |
| | | | AGA treatment | | | | |

Results of dispersion analysis while 15 days of scheme A treatment demonstrated that the influence of group factor is being characterized by values F3;33=0.43, p=1.00. Under Newman-Keuls test, there was also no statistically significant difference of blood coagulation between studied groups at 45 day of experiment. But on 15 day of scheme A treatment a tendency to increase of PT occurred in groups receiving studied drug and reference drugs, that may evidence a decrease of atherothrombosis risk while pathology modeling using studied drugs. Total score showed equipotency of administration of all studied combinations and reference drugs with statins at 15 day of scheme A treatment.

### Scheme A

Data on blood coagulation of scheme A animals at 60 day of the study are presented in table 70.

TABLE 70
Blood coagulation 30 day of treatment (60 day of study)—scheme A

| | Sub- | Active | | | | | |
|---|---|---|---|---|---|---|---|
| Group | group | ingredient | | | M ± SEM | | |
| [symbol] ^{o} | [symbol] ^{o} | dose, mg | Group description | N | PT | APTT | Platelets |
| 2 | 2A | 0 | Control | 6 | 5.7 ± 0.5 | 13.7 ± 0.5 | 381 ± 29 |
| 5 | 3A | 20 + 200 | Present invention— | 6 | 12.8 ± 0.3* | 15.0 ± 1.1 | 311 ± 18 |
| 7 | 4A | 20 + 100 | rosuvastatin + AGA | 6 | 11.9 ± 0.7* | 14.8 ± 1.0 | 319 ± 11 |
| 9 | 5A | 20 + 50 | treatment | 6 | 11.6 ± 0.8* | 14.7 ± 0.4 | 325 ± 28 |
| 11 | 6A | 20 | Comparison | 6 | 11.2 ± 0.9* | 14.9 ± 0.3 | 318 ± 25 |
| 13 | 6A2 | 40 | rosuvastatin treatment | 6 | 11.5 ± 0.8* | 14.9 ± 0.7 | 320 ± 23 |
| 14 | 7A | 20 + 200 | Present invention— | 6 | 12.2 ± 0.7* | 15.4 ± 1.2 | 311 ± 27 |
| 16 | 8A | 20 + 100 | atorvastatin + AGA | 6 | 11.9 ± 0.8* | 15.1 ± 0.6 | 315 ± 19 |
| 18 | 9A | 20 + 50 | treatment | 6 | 11.0 ± 0.8* | 14.9 ± 0.8 | 319 ± 21 |
| 20 | 10A | 20 | | 6 | 10.9 ± 0.9* | 14.5 ± 0.3 | 321 ± 30 |
| 22 | 10A2 | 40 | Comparison— | 6 | 11.6 ± 0.9* | 14.9 ± 0.9 | 315 ± 12 |
| | | | atorvastatin treatment | | | | |
| 23 | 11A | 100 | Comparison—AGA | 6 | 7.0 ± 0.9 | 14.1 ± 0.5 | 369 ± 11 |
| | | | treatment | | | | |

Note:
*p < 0.05—significant difference from control group (Newman-Keuls test).

Results of dispersion analysis while 30 days of scheme A treatment demonstrated that the influence of group factor is being characterized by values F3;33=1.725, p=0.01. Under Newman-Keuls test, there was the only statistically significant difference of blood coagulation between studied groups—PT parameter. Statistically significant increase of PT was demonstrated in all groups compared to control group except group with AGA 100 mg. This group nevertheless demonstrated tendency to recovery of PT parameter values. There is a tendency to have dose-dependent relation with AGA.

Data on blood coagulation of scheme A animals at 75 day of the study are presented in table 71.

TABLE 71
Blood coagulation 45 day of treatment (75 day of study)—scheme A

| Group | Sub-group | Active ingredient | Group | | M ± SEM | | |
|---|---|---|---|---|---|---|---|
| [symbol] ^{o} | [symbol] ^{o} | dose, mg | description | N | PT | APTT | Platelets |
| 2 | 2A | 0 | Control | 6 | 5.8 ± 0.6 | 13.1 ± 0.9 | 395 ± 32 |
| 5 | 3A | 20 + 200 | Present | 6 | 13.9 ± 0.9* | 15.1 ± 0.8 | 335 ± 24 |
| 7 | 4A | 20 + 100 | invention— | 6 | 12.8 ± 0.7* | 14.9 ± 1.2 | 321 ± 25 |
| 9 | 5A | 20 + 50 | rosuvastatin + | 6 | 11.9 ± 0.6* | 14.3 ± 1.3 | 329 ± 18 |
| | | | AGA | | | | |
| | | | treatment | | | | |
| 11 | 6A | 20 | Comparison— | 6 | 11.7 ± 0.4* | 14.2 ± 0.5 | 335 ± 15 |
| 13 | 6A2 | 40 | rosuvastatin | | | | |
| | | | treatment | 6 | 12.6 ± 0.9* | 14.0 ± 0.8 | 320 ± 11 |
| 14 | 7A | 20 + 200 | Present | 6 | 13.0 ± 0.5* | 15.9 ± 0.3 | 309 ± 15 |
| 16 | 8A | 20 + 100 | invention— | 6 | 12.9 ± 1.2* | 15.3 ± 0.9 | 315 ± 18 |
| 18 | 9A | 20 + 50 | atorvastatin + | 6 | 12.5 ± 1.2* | 14.3 ± 0.9 | 315 ± 14 |
| | | | AGA | | | | |
| | | | treatment | | | | |
| 20 | 10A | 20 | Comparison— | 6 | 11.9 ± 0.7* | 14.1 ± 0.7 | 319 ± 11 |
| 22 | 10A2 | 40 | atorvastatin | 6 | 12.9 ± 0.5* | 14.6 ± 0.8 | 310 ± 10 |
| | | | treatment | | | | |
| 23 | 11A | 108 | Comparison | 6 | 8.1 ± 0.8* | 14.0 ± 0.6 | 365 ± 38 |
| | | | AGA | | | | |
| | | | treatment | | | | |

Note:
*p < 0.05—significant difference from control group(Newman-Keuls test).

Results of dispersion analysis after 45 days of scheme A treatment demonstrated that the influence of drug administration factor is being characterized by values F3;33=2.491, p=0.00008. Under Newman-Keuls test, there was the only statistically significant difference of blood coagulation parameters between studied groups—PT parameter. Statistically significant increase of PT was demonstrated in all groups compare to control group. Groups administered the combination therapy of the present invention demonstrated tendency to faster recovery of PT parameter.

Data on blood coagulation of scheme A animals at 90 day of the study are represented in table 72.

TABLE 72
Blood coagulation at 60 day of treatment (90 day of study) —scheme A

| | Sub- | Active | | | | | |
|---|---|---|---|---|---|---|---|
| Group | group | ingredient | | | M ± SEM | | |
| [symbol] ^{o} | [symbol] ^{o} | dose, mg | Group description | N | PT | APTT | Platelets |
| 2 | 2A | 0 | Control | 6 | 6.2 ± 0.4 | 13.5 ± 0.4 | 398 ± 22 |
| 5 | 3A | 20 + 200 | Present invention— | 6 | 15.1 ± 0.6* | 15.2 ± 0.7 | 309 ± 14 |
| 7 | 4A | 20 + 100 | rosuvastatin + AGA | 6 | 14.5 ± 0.6* | 15.1 ± 1.0 | 322 ± 21* |
| 9 | 5A | 20 + 50 | treatment | 6 | 14.5 ± 0.7* | 15.0 ± 1.2 | 321 ± 19* |
| 11 | 6A | 20 | Comparison— | 6 | 14.5 ± 0.8* | 15.3 ± 0.9 | 323 ± 13* |
| 13 | 6A2 | 40 | rosuvastatin treatment | 6 | 15.6 ± 0.8* | 15.7 ± 0.4 | 315 ± 10* |
| 14 | 7A | 20 + 200 | Present invention— | 6 | 17.1 ± 0.7* | 15.5 ± 0.6 | 310 ± 12 |
| 16 | 8A | 20 + 100 | atorvastatin + AGA | 6 | 16.3 ± 0.2* | 15.2 ± 0.5 | 312 ± 14* |
| 18 | 9A | 20 + 50 | treatment | 6 | 16.0 ± 1.2* | 14.9 ± 0.5 | 318 ± 12 |
| 20 | 10A | 20 | Comparison | 6 | 15.8 ± 0.4* | 14.9 ± 0.5 | 320 ± 22* |
| 22 | 10A2 | 40 | atorvastatin treatment | 6 | 16.5 ± 0.7* | 15.7 ± 0.3 | 308 ± 9* |
| 23 | 11A | 108 | Comparison—AGA | 6 | 9.3 ± 0.7* | 14.1 ± 0.5 | 353 ± 34 |
| | | | treatment | | | | |

Note:
*p < 0.05 —significant difference from control group (Newman-Keuls test).

Results of dispersion analysis after 60 days of scheme A treatment demonstrated that the influence of drug administration factor is being characterized by values F3;33=3.811, p<0.000001. Under Newman-Keuls test, statistically significant difference of blood coagulation parameters between studied groups was in PT parameter and platelets amount. Statistically significant increase of PT was demonstrated in all groups compare to control group while using studied drugs.

More expressed normalization in groups of statin therapy with AGA in a dose of 200 mg.

The amount of platelets decreased was statistically significant in groups of monotherapy with atorvastatin and rosuvastatin in a dose of 20 and 40 mg and combinations with rosuvastatin+AGA in a dose of 20+200 mg, atorvastatin+AGA in a dose of 20+100 mg, 20+200 mg. However, integral assessment of the influence of studied drugs on blood coagulation parameters revealed the most effectiveness of administration during 60 days by scheme A atorvastatin in a dose of 40 mg, combination of atorvastatin+AGA in a dose of 20+200 mg and 20+100 mg and combination of rosuvastatin+AGA in a dose of 20+200 mg.

According to total score of effectiveness of studied drugs, after 60 days of treatment by scheme A, combinations of atorvastatin+AGA according to the present invention in a dose of 20+200 mg demonstrated the most effectiveness. The effectiveness of the combination of atorvastatin+AGA in that dose was comparable to such of reference drug rosuvastatin in a double dose of 40 mg and exceeded the efficacy of atorvastatin monotherapy in a dose of 20 mg and efficacy of combination of rosuvastatin+AGA.

### Scheme B

Data on blood coagulation of scheme B animals before the treatment are presented in table 73.

TABLE 73
Blood coagulation 0 day (before treatment)—scheme B

| | Sub- | Active | | | | | |
|---|---|---|---|---|---|---|---|
| Group | group | ingredient | | | M ± SEM | | |
| [symbol] ^{o} | [symbol] ^{o} | dose, mg | Group description | N | PT | APTT | Platelets |
| 1 | 1 | 0 | Intact | 6 | 18.5 ± 0.6 | 15.5 ± 0.3 | 305 ± 11 |
| 3 | 2B | 0 | Control | 6 | 17.8 ± 1.0 | 15.5 ± 0.2 | 302 ± 5 |
| 4 | 2B1 | 0 | Control | 6 | 17.8 ± 0.5 | 16.2 ± 1.0 | 299 ± 7 |
| 6 | 3B | 20 + 200 | Present invention: | 6 | 17.5 ± 0.8 | 16.3 ± 1.3 | 314 ± 21 |
| 8 | 4B | 20 + 100 | treatment rosuvastatin + | 6 | 18.8 ± 1.2 | 15.7 ± 0.3 | 310 ± 20 |
| 10 | 5B | 20 + 50 | AGA | 6 | 18.7 ± 1.5 | 15.7 ± 0.4 | 309 ± 11 |
| 12 | 6B | 20 | Comparison: treatment | 6 | 18.3 ± 1.7 | 16.3 ± 0.6 | 305 ± 17 |
| | | | rosuvastatin | | | | |
| 15 | 7B | 20 + 200 | Present invention: | 6 | 17.7 ± 0.6 | 16.8 ± 1.3 | 314 ± 10 |
| 17 | 8B | 20 + 100 | treatment atorvastatin + | 6 | 18.0 ± 0.7 | 16.2 ± 0.9 | 321 ± 33 |
| 19 | 9B | 20 + 50 | AGA | 6 | 18.0 ± 0.6 | 16.5 ± 0.7 | 307 ± 12 |
| 21 | 10B | 20 | Comparison: treatment | 6 | 18.4 ± 0.9 | 16.0 ± 1.2 | 311 ± 25 |
| | | | atorvastatin | | | | |
| 24 | 11B | 100 | Comparison: treatment | 6 | 18.3 ± 0.7 | 16.2 ± 1.4 | 310 ± 12 |
| | | | AGA | | | | |

Results of dispersion analysis of blood coagulation in scheme B animals before treatment demonstrated that the influence of group factor is being characterized by values F3;33=0.17, p=1.00. Under Newman-Keuls test, there was also no statistically significant difference of blood coagulation between studied groups before the experiment.

Data on blood coagulation of scheme B animals at 60 day of study is represented in table 74.

TABLE 74
Blood coagulation 60 day (before treatment)—scheme B

| | Sub- | Active | | | | | |
|---|---|---|---|---|---|---|---|
| Group | group | ingredient | | | M ± SEM | | |
| [symbol] ^{o} | [symbol] ^{o} | dose, mg | Group description | N | PT | APTT | Platelets |
| 1 | 1 | 0 | Intact | 6 | 18.0 ± 0.6 | 15.3 ± 0.6 | 311 ± 8 |
| 3 | 2B | 0 | Control | 6 | 6.2 ± 0.5* | 14.1 ± 0.8 | 379 ± 30* |
| 4 | 2B1 | 0 | Control | 6 | 6.0 ± 0.4* | 13.8 ± 0.7 | 385 ± 32 |
| 6 | 3B | 20 + 200 | Present invention: | 6 | 5.8 ± 0.5* | 14.3 ± 0.9 | 380 ± 15* |
| 8 | 4B | 20 + 100 | treatment | 6 | 6.3 ± 0.6* | 14.3 ± 0.7 | 377 ± 12* |
| 10 | 5B | 20 + 50 | rosuvastatin + AGA | 6 | 6.0 ± 0.5* | 14.8 ± 0.7 | 384 ± 14 |
| 12 | 6B | 20 | Comparison: | 6 | 5.8 ± 0.5* | 14.2 ± 0.9 | 389 ± 11 |
| | | | treatment rosuvastatin | 6 | 5.8 ± 0.6* | 14.7 ± 0.2 | 388 ± 15 |
| 15 | 7B | 20 + 200 | Present invention | 6 | 6.0 ± 0.5* | 14.7 ± 0.3 | 382 ± 12 |
| 17 | 8B | 20 + 100 | treatment | | | | |
| 19 | 9B | 20 + 50 | atorvastatin + AGA | 6 | 6.0 ± 0.5* | 14.5 ± 0.4 | 387 ± 14 |
| 21 | 10B | 20 | Comparison: treatment | 6 | 6.2 ± 0.5* | 14.2 ± 0.3 | 389 ± 12 |
| | | | atorvastatin | | | | |
| 24 | 11B | 108 | Comparison: treatment | 6 | 6.0 ± 0.4* | 15.0 ± 0.5 | 380 ± 15 |
| | | | AGA | | | | |

Note—
*p < 0.05—significant difference from intact group (Newman-Keuls test).

Results of dispersion analysis of blood coagulation in scheme B animals at 60 day of pathology modeling demonstrated that the influence of group factor is being characterized by values F3;33=6.189, p<0.000001.

Under Newman-Keuls test, statistically significant difference of blood coagulation parameters between studied groups was in PT and platelets amount between groups with modeled pathology and intact one.

Such symptoms correlate to the development of atherosclerosis with high risk of thrombosis and usually occur in clinical picture.

### Parameters of Blood Coagulation Under the Treatment

Data on blood coagulation of scheme B animals at 75 day of study is represented in table 75.

TABLE 75
Blood coagulation at 15 day of treatment (75 day of study)—scheme B

| | Sub- | Active | | | | | |
|---|---|---|---|---|---|---|---|
| Group | group | ingredient | | | M ± SEM | | |
| [symbol] ^{o} | [symbol] ^{o} | dose, mg | Group description | N | PT | APTT | Platelets |
| 1 | 1 | 0 | Intact | 6 | 17.8 ± 1.3 | 15.9 ± 1.2 | 309 ± 25 |
| 4 | 2B1 | 0 | Control | 6 | 6.2 ± 0.3* | 13.2 ± 0.4 | 392 ± 30 |
| 6 | 3B | 20 + 200 | Present invention: | 6 | 7.0 ± 0.5* | 13.5 ± 1.1 | 373 ± 14 |
| 8 | 4B | 20 + 100 | treatment rosuvastatin | 6 | 7.5 ± 0.4* | 13.9 ± 0.9 | 375 ± 22 |
| 10 | 5B | 20 + 50 | + AGA | 6 | 7.2 ± 0.6* | 14.2 ± 0.8 | 371 ± 11 |
| 12 | 6B | 20 | Comparison: | 6 | 7.5 ± 0.3* | 14.5 ± 0.7 | 372 ± 10 |
| | | | treatment rosuvastatin | | | | |
| 15 | 7B | 20 + 200 | Present invention | 6 | 7.4 ± 0.7* | 14.0 ± 1.2 | 374 ± 23 |
| 17 | 8B | 20 + 100 | treatment | 6 | 7.0 ± 0.6* | 14.2 ± 0.9 | 371 ± 11 |
| 19 | 9B | 20 + 50 | atorvastatin + AGA | 6 | 7.5 ± 0.6* | 14.0 ± 1.3 | 374 ± 15 |
| 21 | 10B | 20 | Comparison: | 6 | 7.2 ± 0.6* | 13.9 ± 1.2 | 362 ± 25 |
| | | | treatment atorvastatin | | | | |
| 24 | 11B | 100 | Comparison: | 6 | 6.5 ± 0.6* | 13.5 ± 0.6 | 389 ± 23 |
| | | | treatment AGA | | | | |

Note—
*p < 0.05—significant difference from intact group (Newman-Keuls test).

Results of dispersion analysis after 75 days of scheme A treatment demonstrated that the influence of drug administration factor is being characterized by values F3;30=4.438, p<0.000001. Under Newman-Keuls test, at 75 day of pathology there was a statistically significant decrease of PT in all groups with modeled pathology compare to intact one.

There was no statistically significant difference during 15 days of treatment with drugs intake in blood coagulation parameters from control group. But accordingly to total score of efficacy there was shown a tendency to an increase of PT while 15-day administration of studied combinations of rosuvastatin+AGA in a dose of 20+100 mg and atorvastatin+AGA in a dose of 20+50 mg.

Data on blood coagulation of scheme B animals at 90 day of study is represented in table 76.

TABLE 76
Blood coagulation 30 day of treatment (90 day of study)—scheme B

| | Sub- | Active | | | | | |
|---|---|---|---|---|---|---|---|
| Group | group | ingredient | | | M ± SEM | | |
| [symbol] ^{o} | [symbol] ^{o} | dose, mg | Group description | N | PT | APTT | Platelets |
| 1 | 1 | 0 | Intact | 6 | 18.3 ± 1.0 | 15.3 ± 0.7 | 298 ± 22 |
| 4 | 2B1 | 0 | Control | 6 | 5.7 ± 0.6¹ | 13.2 ± 1.1 | 389 ± 25 |
| 6 | 3B | 20 + 200 | Present invention: | 6 | 10.1 ± 1.0¹² | 14.2 ± 1.4 | 339 ± 25 |
| 8 | 4B | 20 + 100 | treatment | 6 | 10.5 ± 0.8¹² | 14.7 ± 1.5 | 341 ± 30 |
| 10 | 5B | 20 + 50 | rosuvastatin + AGA | 6 | 9.9 ± 0.8¹² | 14.5 ± 1.3 | 350 ± 14 |
| 12 | 6B | 20 | Comparison: | 6 | 10.8 ± 0.7¹² | 14.6 ± 1.2 | 341 ± 12 |
| | | | treatment rosuvastatin | | | | |
| 15 | 7B | 20 + 200 | Present invention: | 6 | 9.7 ± 1.0¹² | 14.7 ± 1.2 | 330 ± 35 |
| 17 | 8B | 20 + 100 | treatment | 6 | 10.1 ± 0.7¹² | 14.5 ± 1.0 | 332 ± 12 |
| 19 | 9B | 20 + 50 | atorvastatin + AGA | 6 | 10.7 ± 0.4¹² | 14.5 ± 1.2 | 339 ± 14 |
| 21 | 10B | 20 | Comparison: | 6 | 10.9 ± 1.0¹² | 14.7 ± 0.3 | 333 ± 31 |
| | | | atorvastatin | | | | |
| 24 | 11B | 100 | Comparison: | 6 | 6.9 ± 0.8¹ | 13.6 ± 0.4 | 376 ± 30 |
| | | | treatment AGA | | | | |

Note:
¹p < 0.05—significant difference from intact group (Newman-Keuls test);
²p < 0.05—significant difference from control group (Newman-Keuls test).

Results of dispersion analysis of blood coagulation in scheme B animals at 90 day of pathology modeling demonstrated that the influence of group factor is being characterized by values F3;30=2.989, p<0.000001. Under Newman-Keuls test, at 90 day of pathology there was a statistically significant decrease of PT in all groups with modeled pathology compare to intact one, confirming further development of the pathology.

But statistically significant difference of blood coagulation from control group was demonstrated after drugs administration of 30 days: PT was statistically significant less, than in control group, in groups of both combination in all doses as well as monotherapy in all doses too. Dose-relation reveals from AGA amount.

Data on blood coagulation of scheme B animals at 105 day of study is presented in table 77.

TABLE 77
Blood coagulation 45 day of treatment (105 day of study)—scheme B

| | Sub- | Active | | | | | |
|---|---|---|---|---|---|---|---|
| Group | group | ingredient | | | M ± SEM | | |
| [symbol] ^{o} | [symbol] ^{o} | dose, mg | Group description | N | PT | APTT | Platelets |
| 1 | 1 | 0 | Intact | 6 | 17.9 ± 0.5 | 14.9 ± 1.0 | 301 ± 25 |
| 4 | 2B1 | 0 | Control | 6 | 5.9 ± 0.3¹ | 12.5 ± 1.0 | 392 ± 34 |
| 6 | 3B | 20 + 200 | Present invention: | 6 | 13.3 ± 1.11¹² | 15.0 ± 0.6 | 327 ± 20 |
| 8 | 4B | 20 + 100 | treatment | 6 | 13.4 ± 1.2¹² | 14.8 ± 0.8 | 325 ± 20 |
| 10 | 5B | 20 + 50 | rosuvastatin + AGA | 6 | 12.5 ± 0.9¹² | 14.7 ± 1.0 | 317 ± 15 |
| 12 | 6B | 20 | Comparison: | 6 | 12.9 ± 0.9¹² | 14.8 ± 1.1 | 320 ± 24 |
| | | | treatment | | | | |
| | | | rosuvastatin | | | | |
| 15 | 7B | 20 + 200 | Present invention: | 6 | 13.7 ± 1.2¹² | 14.7 ± 1.1 | 314 ± 22 |
| 17 | 8B | 20 + 100 | treatment | 6 | 13.1 ± 1.4¹² | 14.6 ± 0.5 | 311 ± 10 |
| 19 | 9B | 20 + 50 | atorvastatin + AGA | 6 | 12.6 ± 1.1¹² | 14.7 ± 0.9 | 309 ± 24 |
| 21 | 10B | 20 | Comparison: | 6 | 12.8 ± 0.9¹² | 14.6 ± 0.6 | 312 ± 24 |
| | | | treatment atorvastatin | | | | |
| 24 | 11B | 100 | Comparison: | 6 | 6.7 ± 0.5¹ | 13.5 ± 0.8 | 380 ± 32 |
| | | | treatment AGA | | | | |

Note:
¹p < 0.05—significant difference from intact group (Newman-Keuls test);
²p < 0.05—significant difference from control group (Newman-Keuls test).

Results of dispersion analysis of blood coagulation in scheme B animals at 105 day of study demonstrated that the influence of group factor is being characterized by values F3;30=2.712, p=0.000035. Under Newman-Keuls test, from all blood coagulation′ parameters, there was a statistically significant difference of PT parameter in all groups with pathology compare to intact one. The administration of studied objects was accompanied by significant

increase of PT in all groups but AGA in a dose of 50 mg group, compare to control group. But even this group demonstrated the tendency to increasing PT. There was no statistically significant difference between therapy groups. But accordingly to total score of treatment effectiveness, there was a significant tendency for efficacy of combination of Atorvastatin+AGA in all doses to override such of combination of rosuvastatin+AGA and monotherapy.

Data on blood coagulation of scheme B animals at 120 day of study are presented in table 78.

TABLE 78
Blood coagulation 60 day of treatment (120 day of study)—scheme B

| | Sub- | Active | | | | | |
|---|---|---|---|---|---|---|---|
| Group | group | ingredient | | | M ± SEM | | |
| [symbol] ^{o} | [symbol] ^{o} | dose, mg | Group description | N | PT | APTT | Platelets |
| 1 | 1 | 0 | Intact | 6 | 18.3 ± 0.4 | 15.0 ± 1.2 | 309 ± 22 |
| 4 | 2B1 | 0 | Control | 6 | 6.4 ± 0.5¹ | 13.1 ± 1.1 | 384 ± 25 |
| 6 | 3B | 20 + 200 | Present invention: | 6 | 14.5 ± 1.2¹² | 15.2 ± 0.9 | 315 ± 18 |
| 8 | 4B | 20 + 100 | treatment | 6 | 13.9 ± 1.4¹² | 15.0 ± 0.5 | 319 ± 23 |
| 10 | 5B | 20 + 50 | rosuvastatin + AGA | 6 | 14.0 ± 0.7¹² | 15.1 ± 1.2 | 318 ± 14 |
| 12 | 6B | 20 | Comparison: | 6 | 14.0 ± 1.2² | 15.1 ± 1.2 | 312 ± 20 |
| | | | treatment rosuvastatin | | | | |
| 15 | 7B | 20 + 200 | Present invention: | 6 | 15.7 ± 1.5¹² | 15.3 ± 1.2 | 314 ± 16 |
| 17 | 8B | 20 + 100 | treatment | 6 | 14.9 ± 1.2² | 15.2 ± 0.8 | 310 ± 14 |
| 19 | 9B | 20 + 50 | atorvastatin + AGA | 6 | 14.8 ± 1.3¹² | 15.0 ± 0.6 | 310 ± 25 |
| 21 | 10B | 20 | Comparison: | 6 | 14.9 ± 0.7² | 15.1 ± 0.4 | 312 ± 14 |
| | | | treatment atorvastatin | | | | |
| 24 | 11B | 100 | Comparison: treatment | 6 | 7.2 ± 0.3¹ | 13.8 ± 0.5 | 374 ± 21 |
| | | | AGA | | | | |

Note:
¹p < 0.05—significant difference from intact group (Newman-Keuls test);
²p < 0.05—significant difference from control group (Newman-Keuls test).

Under Newman-Keuls test, statistically significant difference on PT parameter at 120 day of study between study groups was demonstrated. Pathology was associated with a statistically significant decrease of PT parameter compare to intact group. The administration of studied objects was accompanied by significant increase of PT in all groups but AGA compare to control group. But even this group demonstrated the tendency to recover PT, confirming coagulation study data on 60 day of treatment and meaning possible therapeutic benefit against atherosclerosis development with atherothrombosis risk.

The normalization of platelets amount occurred, more expressed in statins groups with AGA 200 mg. There was no significant difference between combinations of atorvastatin+AGA and rosuvastatin+AGA.

Total score of efficacy showed equipotency of administration of studied drugs administration regarding blood coagulation parameters at 60 day of scheme B treatment.

Total score of efficacy of scheme B administration of studied drugs demonstrated tendency of the combination of Atorvastatin+AGA according to the present invention to exhibit excellent efficacy regarding blood coagulation. The Efficacy of such combination exceeded such of Atorvastatin in a dose of 20 mg monotherapy and exceed the efficacy of Rosuvastatin+AGA combination.

### Morphometry Results of Aortal Atherosclerotic Plaque

Percentage of atherosclerotic plaques in scheme A animals is presented in table 79.

TABLE 79
Percentage of atherosclerotic plaques in animals—scheme A

| | | | | | Percentage of |
|---|---|---|---|---|---|
| | | | | | atherosclerotic |
| | | | | | plaque from |
| | Sub- | Active | | | all aortal |
| Group | group | ingredient | | | segment square, |
| [symbol] o | [symbol] o | dose, mg | Group description | N | M ± SEM |
| 2 | 2A | 0 | Control | 6 | 83 ± 5 |
| 5 | 3A | 20 + 200 | Present invention: | 6 | 27 ± 6* |
| 7 | 4A | 20 + 100 | rosuvastatin + AGA | 6 | 41 ± 5* |
| 9 | 5A | 20 + 50 | treatment | 6 | 51 ± 10* |
| 11 | 6A | 20 | Comparison: | 6 | 46 ± 9* |
| 13 | 6A2 | 40 | rosuvastatin | 6 | 26 ± 2* |
| | | | treatment | | |
| 14 | 7A | 20 + 200 | Present invention: | 6 | 27 ± 3* |
| 16 | 8A | 20 + 100 | atorvastatin + AGA | 6 | 40 ± 10* |
| 18 | 9A | 20 + 50 | treatment | 6 | 67 ± 5 |
| 20 | 10A | 20 | Comparison: | 6 | 50 ± 12* |
| 22 | 10A2 | 40 | atorvastatin | 6 | 24 ± 5* |
| | | | treatment | | |
| 23 | 11A | 100 | Comparison: AGA | 6 | 75 ± 8 |
| | | | treatment | | |

Note:
*p < 0.05—significant difference from control group (Newman-Keuls test).

Dispersion analysis demonstrated that the influence of scheme A drug administration factor on atherosclerotic plaque percentage is being characterized by values F11;60=7.786, p<0.000001. Under Newman-Keuls test, statistically significant difference of atherosclerotic plaque percentage from control group was demonstrated in all treatment groups but those who received combination of Rosuvastatin and AGA in a dose of 20+50 mg and AGA monotherapy. But these groups also demonstrated significant tendency to reduce the square of atherosclerotic plaque.

The administration of combination of atorvastatin and AGA in a dose of 20+200 mg by scheme A according to the present invention was most effective regarding the percentage of atherosclerotic disease of aorta. The efficacy of such combination exceeded twice the amount of each drug as monotherapy and was comparable to an effect of double dose of atorvastatin 40 mg.

The tendency of combination of atorvastatin+AGA in a dose of 20+200 mg according to the present invention to exhibit efficacy exceeding that of combination of rosuvastatin+AGA should also be mentioned. In addition, the atherosclerotic plaque square in group of such dose of the atorvastatin+AGA combination of the present invention statistically differs from such of monotherapy group.

Percentage of atherosclerotic plaques in scheme B animals is presented in table 80.

TABLE 80
Percentage of atherosclerotic plaques in animals—scheme B

| | | | | | Percentage of |
|---|---|---|---|---|---|
| | | | | | atherosclerotic |
| | | | | | plaque from |
| | Sub- | Active | | | all aortal |
| Group | group | ingredient | | | segment square, |
| [symbol] o | [symbol] o | dose, mg | Group description | N | M ± SEM |
| 3 | 2B | 0 | Control* | 6 | 87 ± 4 |
| 4 | 2B1 | 0 | Control | 6 | 92 ± 3 |
| 6 | 3B | 20 + 200 | Present invention: | 6 | 65 ± 7¹ |
| 8 | 4B | 20 + 100 | rosuvastatin + AGA | 6 | 70 ± 9 |
| 10 | 5B | 20 + 50 | treatment | 6 | 72 ± 6 |
| 12 | 6B | 20 | Comparison: | 6 | 77 ± 6 |
| | | | rosuvastatin | | |
| | | | treatment | | |
| 15 | 7B | 20 + 200 | Present invention: | 6 | 50 ± 31 |
| 17 | 8B | 20 + 100 | atorvastatin + AGA | 6 | 56 ± 81 |
| 19 | 9B | 20 + 50 | treatment | 6 | 51 ± 71 |
| 21 | 10B | 20 | Comparison: | 6 | 56 ± 51 |
| | | | atorvastatin treatment | | |
| 24 | 11B | 100 | Comparison: AGA | 6 | 73 ± 5 |
| | | | treatment | | |

Note:
¹p < 0.05—significant difference from control group 2B1 (Newman-Keuls test);
*Control group 2B was euthanized due to control plaque development on 61 day of study.

Dispersion analysis demonstrated that the influence of scheme B′ drug administration factor on atherosclerotic plaque percentage is being characterized by values F10;55=6.020, p=0.000004. Under Newman-Keuls test, statistically significant difference of atherosclerotic plaque percentage from control group was demonstrated in groups who received atorvastatin monotherapy, combination of Rosuvastatin and AGA in a dose of 20+200 mg according to the present invention and combination of atorvastatin+AGA according to the present invention in all studied doses.

Administration of combination of atorvastatin+AGA according to the present invention in all studied doses by scheme B was most effective concerning atherosclerotic disease of aorta percentage.

### Results of Histologic Examination of Aorta

Microscopic examination assessed stage of atherosclerotic plaques formation. Standard classification points out four microscopic stages of atherosclerosis: prelipid, lipidosis, liposclerosis and complications stage (atheromatous, thrombosis, calcinosis). To reveal early stages of atherosclerosis-prelipid, toluidine blue was used, by metachromasia reaction of which allows visualizing dystrophic changes of conjunctive tissue. Severity of aortal atherosclerotic damage scored accordingly atherogenesis stages. The pathological process velocity and intensity (duy to a large amount of exogenous cholesterol, vessel wall damage and additional intake of D3 vitamin) allowed forth stage (calcification) to develop, but there was not enough time for full formation of conjunctive tissue, so liposclerosis was expressed not enough and was excluded from analysis.

FIGS. 2-13 show photomicrography of aorta on different stages of atherosclerotic damage.

Intact aorta (FIGS. 2-4): Aortal wall is presented by three coats: inner (intima), mid (media) and external (adventicia). Intima is formatted by endothelium and subendothelial layer from loose conjunctive tissue. The nucleus of endothelial cells—flattened. Media has visible thin strands of smooth muscle cells separated by thick elastic membranes. Adventicia consists of loose conjunctive tissue penetrated with vasa vasorum.

### Stages of Atherogenesis

Prelipid stage (FIGS. 5-7): Microscopic study on this stage reveals initial manifestations of conjunctive tissue disorganization by way of collagen fibres mucoid degeneration along with accumulation of acid glycosaminoglycans, staining by toluidine blue results in lilac coloring. Damaging of endothelium also took place, as well as its swelling, proliferation of smooth muscle cells.

Lipidosis (FIGS. 8-10): Lipidosis stage is characterized by focal infiltration of intima with lipids, lipoproteins, resulting in formation of fat (lipid) stains and lines. Such fat stains look macroscopically like yellow areas which may intermix. Using fat stain on these areas (sudan III, red oil O) reveals lipids, accumulated in smooth muscle cells and macrophages called foam cells or xanthome cells. Multiple lipid vacuoles are revealed in endothelium.

Calcinosis (FIGS. 11-13): Areas of petrification form in atherosclerotic plaques with significant reactive sclerosis.

Efficacy of studied drugs on atherosclerotic damage was scored in each group.

The score system was accepted concerning stages of atherosclerotic process: prelipid stage—1 score, lipidosis—2 scores, calcinosis—3 scores. Not only maximal stages were taken into account, but also background processes (for example, calcinosis—3 scores in the course of significant lipidosis—2 scores, in total 5 scores). Each group of animals was scored. The results are presented in tables 81 and 82.

TABLE 81
Aortal atherosclerosis intensity in scheme A animals

| | | | | | Aortal |
|---|---|---|---|---|---|
| | Sub- | Active | | | damage |
| Group | group | ingredient | | | intensity, |
| [symbol] o | [symbol] o | dose, mg | Group description | N | total score |
| 2 | 2A | 0 | Control | 6 | 27 |
| 5 | 3A | 20 + 200 | Present invention— | 6 | 11 |
| 7 | 4A | 20 + 100 | rosuvastatin + AGA | 6 | 12 |
| 9 | 5A | 20 + 50 | treatment | 6 | 14 |
| 11 | 6A | 20 | Comparison— | 6 | 20 |
| 13 | 6A2 | 40 | rosuvastatm treatment | 6 | 13 |
| 14 | 7A | 20 + 200 | Present invention | 6 | 9 |
| 16 | 8A | 20 + 100 | atorvastatin + AGA | 6 | 8 |
| 18 | 9A | 20 + 50 | treatment | 6 | 9 |
| 20 | 10A | 20 | Comparison— | 6 | 14 |
| 22 | 10A2 | 40 | atorvastatin treatment | 6 | 10 |
| 23 | 11A | 100 | Comparison—AGA | 6 | 24 |
| | | | treatment | | |

The administration of studied combination by scheme A was effective concerning intensity of aortal atherosclerotic damage. As is clear from the table 84, most effective was administration of combination of atorvastatin+AGA. Nevertheless, the efficacy of the combinations of the present invention in all doses was comparable to atorvastatin and rosuvastatin double dose (40 mg) efficacy.

TABLE 82
Aortal atherosclerosis intensity in scheme B animals

| | | | | | Aortal |
|---|---|---|---|---|---|
| | Sub- | Active | | | damage |
| Group | group | ingredient | | | intensity, |
| [symbol] o | [symbol] o | dose, mg | Group description | N | total score |
| 1 | 1 | 0 | Intact | 6 | 0 |
| 4 | 2B1 | 0 | Control | 6 | 30 |
| 6 | 3B | 20 + 200 | Present invention: | 6 | 15 |
| 8 | 4B | 20 + 100 | rosuvastatin + AGA | 6 | 15 |
| 10 | 5B | 20 + 50 | treatment | 6 | 18 |
| 12 | 6B | 20 | Comparison— | 6 | 18 |
| | | | rosuvastatm treatment | | |
| 15 | 7B | 20 + 200 | Present invention: | 6 | 13 |
| 17 | 8B | 20 + 100 | atorvastatin + AGA | 6 | 16 |
| 19 | 9B | 20 + 50 | treatment | 6 | 19 |
| 21 | 10B | 20 | Comparison— | 6 | 19 |
| | | | atorvastatin treatment | | |
| 24 | 11B | 100 | Comparison—AGA | 6 | 24 |
| | | | treatment | | |

The condition of studied animals' aortas was worse by scheme B (treatment) than by administration of scheme A (prophylactic). This corresponds to significant positive influence of statins for prophylaxis of atherosclerotic damages and more difficult process of their regression that also took place under scheme B treatment. The administration of combination of atorvastatin and AGA was found to be most effective concerning histological structure of aorta.

### Histological Findings of Hepar

Liver damages concordant to non-alcoholic steatohepatitis by histological picture was formed in animals with modeled pathology as a result of a study. Thereby for hepar damage scoring were used histological criteria of non-alcoholic steatohepatitis, offered by Professor Brunt in 2002 (Brunt E M, Kleiner D E, Wilson L A, Unalp A, Behling C E, Lavine J E, et al. Portal chronic inflammation in nonalcoholic fatty liver disease (NAFLD): a histologic marker of advanced NAFLD-clinicopathologic correlations from the nonalcoholic steatohepatitis clinical research network. Hepatology. 2009; 49: 809-820).

Those criteria were slightly modified in a study, but common principles remained unchanged. Each morphological sign of hepar damage was totally scored for each group. Criteria selection and their scores are represented in table 83.

TABLE 83
Histological criteria of liver damage score.

| Sign | Grade | Description | Score |
|---|---|---|---|
| Steatosis | O | No adipose degeneration; | 0 |
| | I | Less than 33% hepatocytes underwent | 1 |
| | | adipose degeneration | |
| | II | 33-66% hepatocytes underwent adipose | 2 |
| | | degeneration | |
| | II | More than 66% hepatocytes underwent | 3 |
| | | adipose degeneration | |
| Balloon | O | No damage | 0 |
| dystrophy | I | Steatosis 1-2 grade, minimal balloon | 1 |
| of | | dystrophy in 3 zone of acinus | |
| hepatocytes | II | Steatosis of any grade, moderate balloon | 2 |
| | | dystrophy in 3 zone of acinus | |
| | II | Panacinar steatosis, severe balloon | 3 |
| | | dystrophy | |

Normal hepar (FIG. 14): Hepar had an ordered girder structure, was moderately full-blooded, without sinusoidal dilation, hepatocytes without dystrophy or damage signs, some with significant granulosity caused by glycogen. Portal tracts had typical histological structure, were not dilated, there were hepar triads in the stroma of tracts, submitted by interlobular artery, vein, and bile duct.

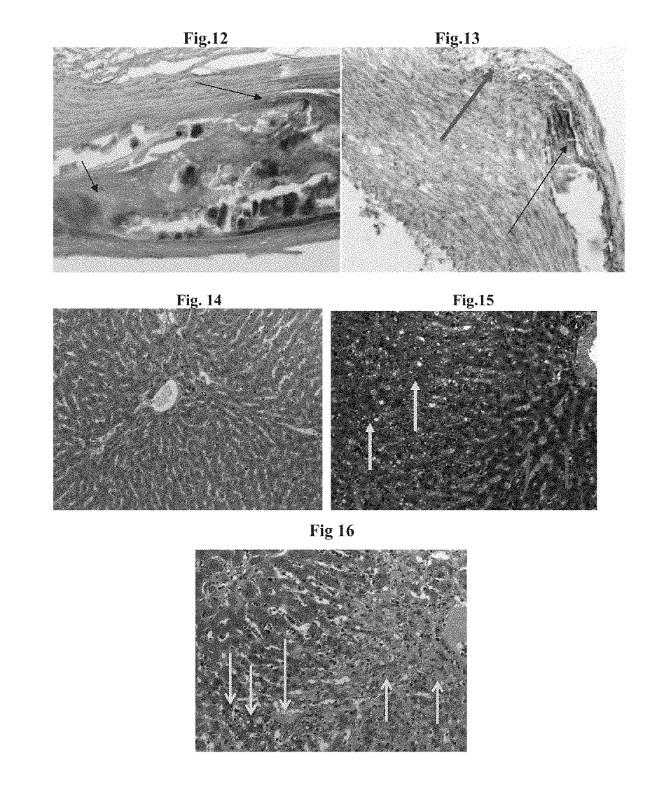

Balloon dystrophy of hepatocytes I gr (FIG. 15-16): Precentral zone hepatocytes (3 zone of acinus) with mild dystrophy on different stages of progression—from mostly granulose (FIG. 15) to hydropic (balloon) (FIG. 16), in addition there is a drop-size adipose degeneration less than ⅓ of cells. Portal tracts without degenerative changes, no signs of inflammation and fibrosis.

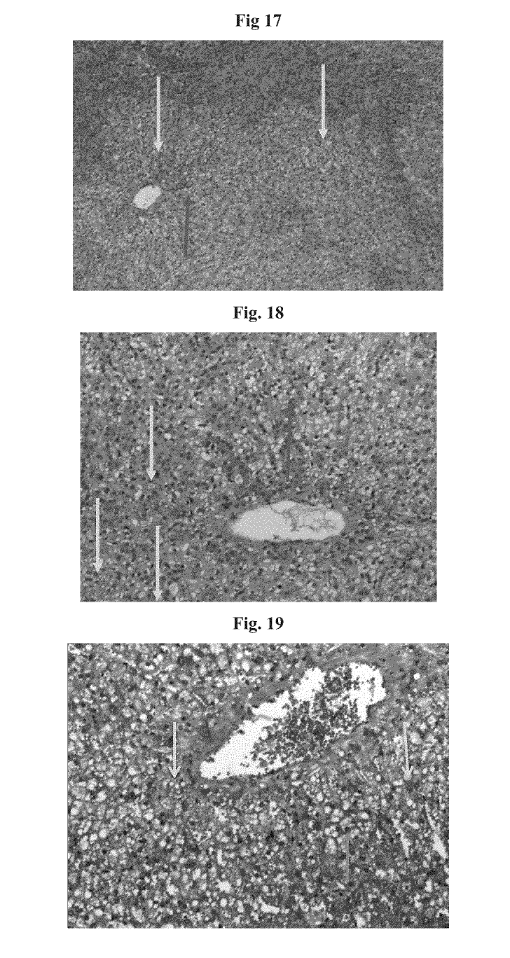

Balloon dystrophy of hepatocytes II gr (FIGS. 17-18): Precentral zone hepatocytes (3 zone of acinus) with moderate balloon dystrophy, same time there is small and large drop adipose degeneration ⅔ of cells. Portal tracts without degenerative changes, no signs of inflammation and fibrosis.

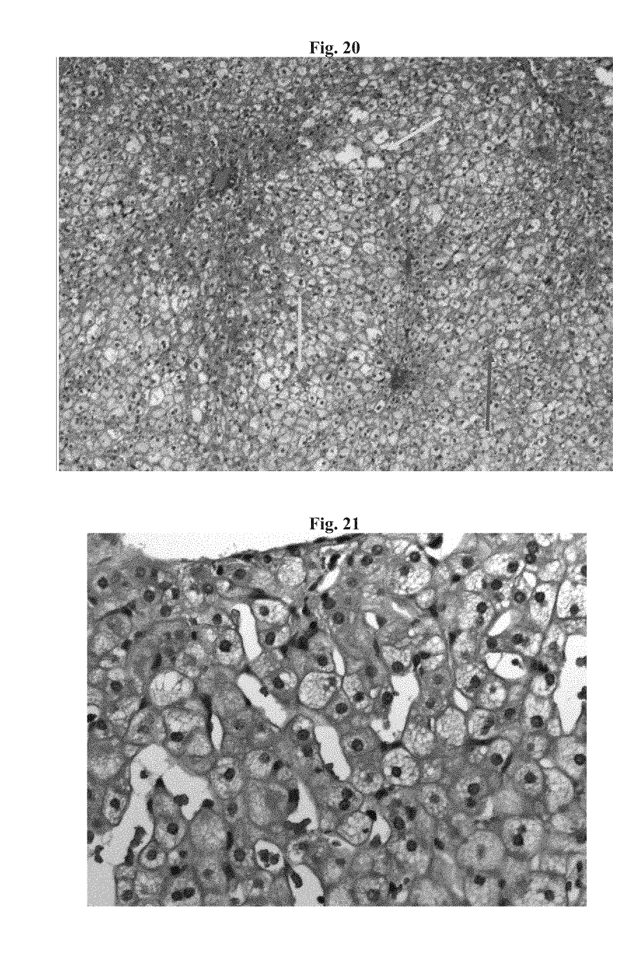

Balloon dystrophy of hepatocytes III gr (FIGS. 19-22): Girder structure of hepar is disturbed, acinar hepatocytes are with significant (panacinar) balloon dystrophy, hepatocytes with small and large drop steatosis are present in all parts of acinus. In most animals portal tracts have no degenerative changes, there are no signs of inflammation and fibrosis. In singular animals there are initial presentations of portal tracts fibrosis (FIG. 22).

Data received are presented in tables 84 and 85.

TABLE 84
Grade of hepar involvement in scheme A animals

| | | | | | Grade of | |
|---|---|---|---|---|---|---|
| | Sub- | Active | | | hepar | Total |
| Group | group | substance | Group | | involvement, | score of |
| [symbol] ^{o} | [symbol] ^{o} | dose, mg | description | N | total score | efficacy |
| 2 | 2A | 0 | Control | 6 | 24 | 5 |
| 5 | 3A | 20 + 200 | Present | 6 | 14 | 9 |
| 7 | 4A | 20 + 100 | invention— | 6 | 19 | 7 |
| 9 | 5A | 20 + 50 | rosuvastatin + | 6 | 20 | 6 |
| | | | AGA treatment | | | |
| 11 | 6A | 20 | Comparison— | 6 | 28 | 3 |
| 13 | 6A2 | 40 | rosuvastatin | 6 | 31 | 1 |
| | | | treatment | | | |
| 14 | 7A | 20 + 200 | Present | 6 | 13 | 10 |
| 16 | 8A | 20 + 100 | invention— | 6 | 14 | 9 |
| 18 | 9A | 20 + 50 | atorvastatin + | 6 | 15 | 8 |
| | | | AGA treatment | | | |
| 20 | 10A | 20 | Comparison— | 6 | 25 | 4 |
| 22 | 10A2 | 40 | atorvastatin | 6 | 29 | 2 |
| | | | treatment | | | |
| 23 | 11A | 100 | Comparison— | 6 | 20 | 6 |
| | | | AGA treatment | | | |

TABLE 85
Grade of hepar involvement in scheme B animals

| | | | | | Grade of | |
|---|---|---|---|---|---|---|
| | Sub- | Active | | | aortal | Total |
| Group | group | ingredient | Group | | involvement, | score of |
| [symbol] ^{o} | [symbol] ^{o} | dose, mg | description | N | total score | efficacy |
| 1 | 1 | 0 | Intact | 6 | 0 | 9 |
| 4 | 2B1 | 0 | Control | 6 | 24 | 4 |
| 6 | 3B | 20 + 200 | Present | 6 | 12 | 8 |
| 8 | 4B | 20 + 100 | invention— | 6 | 19 | 5 |
| 10 | 5B | 20 + 50 | rosuvastatin + | 6 | 25 | 3 |
| | | | AGA treatment | | | |
| 12 | 6B | 20 | Comparison— | 6 | 30 | 2 |
| | | | rosuvastatin | | | |
| | | | treatment | | | |
| 15 | 7B | 20 + 200 | Present | 6 | 14 | 7 |
| 17 | 8B | 20 + 100 | invention— | 6 | 19 | 5 |
| 19 | 9B | 20 + 50 | atorvastatin + | 6 | 25 | 3 |
| | | | AGA treatment | | | |
| 21 | 10B | 20 | Comparison— | 6 | 32 | 1 |
| | | | atorvastatin | | | |
| | | | treatment | | | |
| 24 | 11B | 100 | Comparison— | 6 | 18 | 6 |
| | | | AGA treatment | | | |

All studied groups of animals demonstrated large and small falls in steatosis of hepatocytes on some severity. Hepar involvement was more significant in case of administration high dose of statins as monotherapy than when simultaneous administration of statins and AGA.

As tables 84 and 85 show, administration of both scheme of treatment resulted in more significant histological findings of hepar damage in groups with statins as monotherapy compare to control group. However, administration of rosuvastatin+AGA and atorvastatin+AGA combinations according to the present invention resulted in less grade of hepar involvement than in statins' monotherapy. Also the administration of atorvastatin+AGA combination of the present invention in a dose of 20+200 mg was found to be most effective.

### Histological Findings of Pancreas

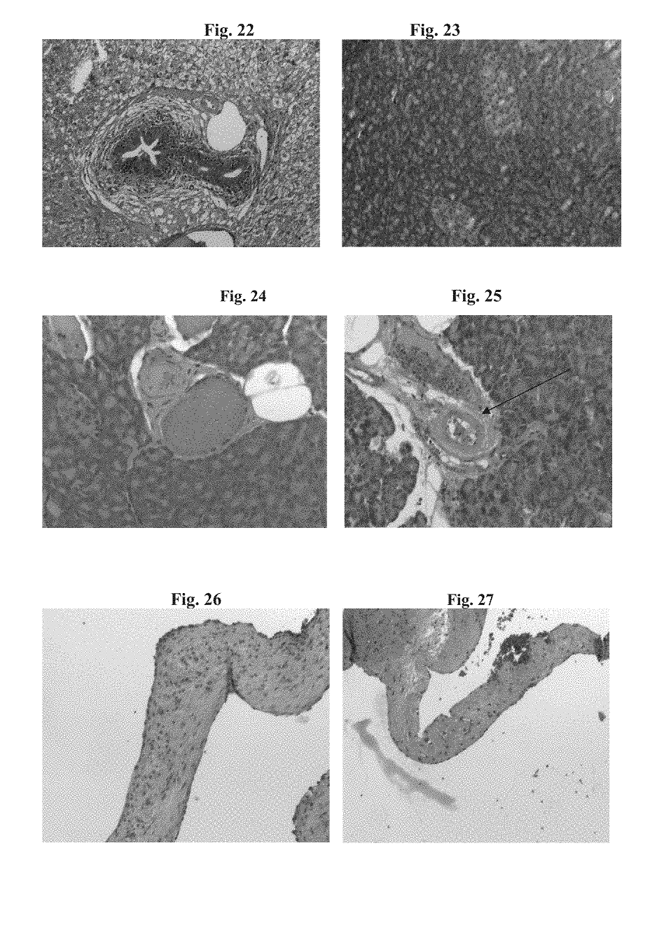

Pancreas had a lobular structure with interlayers of conjunctive and fat tissue between lobules, last presented with exocrine part made of pancreatocytes, forming acinuses opening in pancreatic ducts of a gland. Large pancreatic islet (islet of Langerhans), formed by insulocytes, surrounded by thin net of fenestrated capillary, sited in a diffuse way between acinuses. (FIGS. 23, 24)

In some animals slight gliosis of pancreatis vessels' wall was revealed—wall was thickened (FIG. 25). There were no other pathological processes in pancreas.

### Histological Findings of Heart Valves

Grade of atherosclerotic damage of heart valves was scored on the same criteria as aortal damage. But only lipidosis (2 scores) and calcinosis (3 scores) were estimated. Data received are presented in tables 86 and 87.

TABLE 86
Grade of heart valves damage in scheme A animals

| | | | | | Grade of | |
|---|---|---|---|---|---|---|
| | Sub- | Active | | | heart valves1 | Total |
| Group | group | ingredient | Group | | involvement, | score of |
| [symbol] ^{o} | [symbol] ^{o} | dose, mg | description | N | total score | efficacy |
| 2 | 2A | 0 | Control | 6 | 14 | 1 |
| 5 | 3A | 20 + 200 | Present | 6 | 2 | 5 |
| 7 | 4A | 20 + 100 | invention: | 6 | 2 | 6 |
| 9 | 5A | 20 + 50 | rosuvastatin + | 6 | 4 | 5 |
| | | | AGA treatment | | | |
| 11 | 6A | 20 | Comparison | 6 | 9 | 3 |
| 13 | 6A2 | 40 | rosuvastatin | 6 | 4 | 5 |
| | | | treatment | | | |
| 14 | 7A | 20 + 200 | Present | 6 | 2 | 6 |
| 16 | 8A | 20 + 100 | invention: | 6 | 2 | 4 |
| 18 | 9A | 20 + 50 | atorvastatin + | 6 | 6 | 6 |
| | | | AGA treatment | | | |
| 20 | 10A | 20 | Comparison: | 6 | 9 | 3 |
| 22 | 10A2 | 40 | atorvastatin | 6 | 2 | 6 |
| | | | treatment | | | |
| 23 | 11A | 100 | Comparison— | 6 | 12 | 2 |
| | | | AGA treatment | | | |

TABLE 87
Grade of heart valves damage in scheme B animals

| | | | | | Grade of | |
|---|---|---|---|---|---|---|
| | Sub- | Active | | | heart valves1 | Total |
| Group | group | ingredient | | | involvement, | score of |
| [symbol] ^{o} | [symbol] ^{o} | dose, mg | Group description | N | total score | efficacy |
| 1 | 1 | 0 | Intact | 6 | 0 | 9 |
| 4 | 2B1 | 0 | Control | 6 | 16 | 1 |
| 6 | 3B | 20 + 200 | Present invention: | 6 | 4 | 8 |
| 8 | 4B | 20 + 100 | rosuvastatin + | 6 | 9 | 4 |
| 10 | 5B | 20 + 50 | AGA treatment | 6 | 7 | 6 |
| 12 | 6B | 20 | Comparison— | 6 | 8 | 5 |
| | | | rosuvastatin | | | |
| | | | treatment | | | |
| 15 | 7B | 20 + 200 | Present invention: | 6 | 6 | 7 |
| 17 | 8B | 20 + 100 | atorvastatin + | 6 | 4 | 8 |
| 19 | 9B | 20 + 50 | AGA treatment | 6 | 11 | 3 |
| 21 | 10B | 20 | Comparison— | 6 | 12 | 2 |
| | | | atorvastatin | | | |
| | | | treatment | | | |
| 24 | 11B | 100 | Comparison— | 6 | 12 | 2 |
| | | | AGA treatment | | | |

As for severity of aortal atherosclerosis, a tendency of dependence of process severity from time of pathological process development, treatment scheme and dose of combination administered remains in histological picture of valves damage. Thus, prophylactic scheme of drug administration showed more favorable effect on pathogenesis. However, treatment with the combination of the present invention also made a significant influence: the damage level of heart valves was twice less expressed on the combined therapy of the present invention than monotherapy, showing AGA input on antiatherosclerotic activity of combination.

Three layers were expressed in valve leaflet: inner, middle and outer. Inner layer, faced to heart ventricle, is an extension of endocardium and contains many elastic fibres. Middle layer consisted from loose areolar connective tissue. Outer layer, faced to aorta, contained except endothelium much amount of collagen fibres (FIGS. 26 and 27)

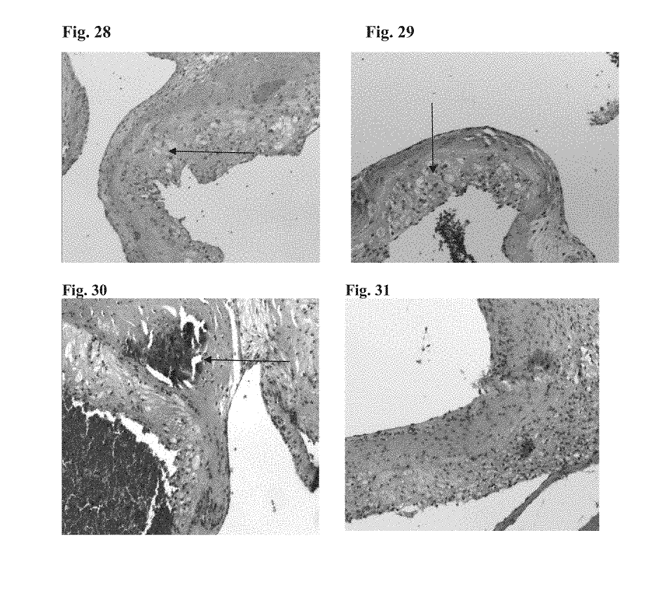

Lipidosis stage: optically empty foam cells and lipids deposits are located under endothelium (FIGS. 28 and 29).

Calcinosis stage: under lipidosis petrification focuses appear in muscle fibres mostly at valves basis (FIGS. 30 and 31)

### Conclusions

As a result of atherogenic factors influence during all study in experimental animals' dyslipidemia was formed, manifested in persistent increase of CS, LDL and TG. Dyslipidemia developed after 15 day of modeling, getting maximum of severity at 75-120 days of study. Animals' dyslipidemia was characterized by high atherogenesis risk from the 15 day of modeling, confirmed by statistically much more high values of atherogenic index compare to intact group. Atherosclerotic lesions of animal's aorta were observed on the 90th day of the study, and to the 120—initial signs of atherocalcinosis. Modeled pathology was also accompanied by changes in biochemical parameters of blood: pathology was accompanied by rising level of activity of transaminases, CPK, concentrations of bilirubin and glucose, PT and APTT decreased and platelet count increased.

Using studied combinations by Scheme B affected the body weight of animals. 120 days of pathology resulted in an excessive weight gain of animals, which manifested in statistically significant higher body weight of animals with modeled pathology than that of intact animals. Increased body weight is one of the most often clinical symptoms associated with atherosclerosis.

Treatment of animals with the combination of rosuvastatin+AGA according to the present invention in a dose of 20 mg+200 mg and with combination of atorvastatin+AGA according to the present invention in a dose of 20 mg+100 mg, as well as monotherapy AGA in a dose of 100 mg, was accompanied by significantly lower body weight of animals was than in the control group of animals. In this case, the use of rosuvastatin and atorvastatin monotherapies and combinations with the lowest AGA (Rosuvastatin+AGA 20+50 and 20 Atorvastatin+AGA+50 mg) did not lead to a significant decrease in body weight of animals relative to the control group. Thus, treatment scheme of administration of studied combinations with the highest content of AGA could contribute to the normalization of the body weight dynamics in atherosclerosis.

Modeled pathology tended to increase mass ratios of the hepar. A significant trend of an increase of mass ratios of the hepar also accompanied the administration of the comparison monotherapies rosuvastatin and Atorvastatin. Mass ratios of the hepar were lower in combinations of statins and AGA according to the present invention than in the control group and significantly lower than in the groups treated with atorvastatin and rosuvastatin as monotherapy, which may indirectly indicate a possible hepatoprotective action of AGA component of combination. It should be noted that despite the lack of statistical significance, mass ratios of the hepar in the group receiving AGA monotherapy, were less than those in the control group.

The observed trends are consistent with the finding of Example 1.

Mass coefficients of control animals' pancreas were statistically significant more than those in the intact group. The group receiving rosuvastatin monotherapy in a dose of 40 mg, showed a statistically significant increase in pancreatic mass ratios relative to the control group. During the Scheme A AGA monotherapy in a dose of 100 mg, a statistically significant decrease in pancreatic mass ratios relative to the control group was observed. The most effective against pancreatic mass ratios was the Scheme B administration of combination of atorvastatin+AGA according to the present invention in a dose of 20 mg+200 mg. In the group treated with this combination, values of mass ratios of pancreas were identical to those of the intact group. The observed trends are consistent with the findings of Example 1.

Regarding to lipid spectrum, therapy with studied combinations was effective under both schemes treatment. The most effective was the use of a combination of atorvastatin+AGA according to the present invention in a dose of 20 mg+200 mg according to the scheme A. Administration of this combination resulted in the set of antiatherogenic effect already within 15 days of treatment, manifested in a statistically significant reduction in atherogenic index relative to that in the control group of animals.

Dyslipidemia progressed in control groups during all study, indicating the progression of pathology. However, the administration of studied combinations and drugs according to scheme A prevented the development of a pathology, as confirmed by a statistically significant reduce of cholesterol, LDL and atherogenic index in treatment groups compare to those in the control group of animals.

It is noteworthy that the effectiveness of studied combinations regarding lipid profile parameters was characterized by a direct dose-dependence from AGA dose in combinations, suggesting the contribution of AGA into the antiatherosclerotic effect of the combinations of the present invention. On the 90th day of Scheme A study, most effective was the use of combination of atorvastatin+AGA in a dose of 20+200 mg. The effectiveness of this combination exceeded such of atorvastatin monotherapy in a dose of 20 mg and was equal to atorvastatin efficiency in a dose of 40 mg.

Treatment with studied combinations by scheme B was also effective against lipid spectrum. Despite the fact that as a result of prolonged pathology dyslipidemia in animals was stronger than for the study on Scheme A, the use of studied combinations also was highly effective against all the tested parameters. Particular efficacy of the studied combinations of the present invention in relation to atherogenic index is noteworthy. By day 60 of treatment by Scheme B in groups with treatment, this parameter was significantly lower than in the control group 3-9 times. Thus, all study drugs, including AGA monotherapy, had a significant antiatherogenic effect being applied during 60 days. A significant tendency to increase efficacy dependent of AGA dose in combination is noteworthy in the treatment by scheme B. Treatment with a combination of Atorvastatin and AGA in a dose of 20+200 mg was most effective for Scheme A as for Scheme B treatment.

Modeled pathology was accompanied by an increase of activity of transaminases, CPK, bilirubin and glucose concentrations in both schemes of the study. There were statistically significant differences from control group on AST activity parameter in all groups with the treatment according to the scheme A. Activity of AST and CPK in the groups treated by scheme B with combination of atorvastatin+AGA and rosuvastatin+AGA according to the present invention was lower than in the control group and lower than in the groups treated with statin monotherapy by the end of the study. The efficacy of the studied combinations on the concentration of glucose and bilirubin was revealed.

Modeled pathology development was also accompanied by a statistically significant decrease in PT and increase in platelet count relative to intact group. Administration of studied objects showed a statistically significant increase of PT in all groups of treatment either scheme A or B relative to the control group. Platelet count decreased significantly in groups treated with monodrugs atorvastatin and rosuvastatin in a dose of 20 and 40 mg, and combinations with Rosuvastatin+AGA and atorvastatin+AGA according to the present invention.

In the study of percentage of atherosclerotic aortal damage by morphometry method, all groups treated by scheme A statistically significant differed from control group of atherosclerotic plaque, except those received a combination of rosuvastatin and AGA in a dose of 20+50 mg and AGA monotherapy. However, these groups demonstrated a tendency to reduce the square of atherosclerotic plaque. The effectiveness of the combination of atorvastatin and rosuvastatin with AGA according to the present invention in a dose of 200 mg exceeded that of monotherapies in 2 times and was comparable to the effect of atorvastatin and rosuvastatin administration in a double dose of 40 mg. The use of combination of atorvastatin and AGA according to the present invention in a dose of 20+200 mg by scheme A was found to be most effective on the percentage of atherosclerotic aortal damage.

Statistically significant difference from control group of an atherosclerotic plaque was showed in groups of scheme B treatment receiving atorvastatin monotherapy, a combination of rosuvastatin and AGA in a dose of 20+200 mg and a combination of atorvastatin+AGA in all investigated doses. Efficacy of combination of atorvastatin and rosuvastatin with a AGA dose of 200 mg according to the present invention exceeded that of monotherapies in 2 times and was comparable to the effect of the use of atorvastatin and rosuvastatin in a double dose of 40 mg. The use of scheme B combination of atorvastatin and AGA according to the present invention in all investigated doses was found to be most effective on the percentage of atherosclerotic aortal damage.

Results of aortal morphometry were mostly confirmed by histological examination of the aorta. Use of studied combinations by scheme A was effective on the severity of atherosclerotic aortal damage. The most effective was the use of a combination of atorvastatin+AGA according to the present invention. The efficacy of this combination in all doses was comparable to atorvastatin in a double dose (40 mg).

The state of animal's aortas after treatment by scheme B was worse than when using preventive scheme A. The most effective in respect of the histological structure of the aorta was also the use a combination of atorvastatin and AGA according to the present invention.

Histological findings of heart valves changes also showed the benefit of the efficacy of the combinations of the present invention compared with the efficacy of statin monotherapy.

Histological examination of the hepar showed significant pathological changes in hepar structure as a result of modeled pathology as well as of long-term use of statins. Administration of both treatment regimens resulted in more significant histological hepar damage in statin monotherapy groups than in the control group. Administration of a combination of rosuvastatin+AGA and atorvastatin+AGA according to the present invention showed less significant hepar damage than in statin monotherapy. The use of a combination of atorvastatin+AGA in a dose of 20+200 mg was found to be most effective.

Thus, the study showed the efficacy of rosuvastatin+AGA and atorvastatin+AGA combinations according to the present invention in all investigated doses. In most cases, the efficacy of combinations was characterized by a direct dose-dependence and exceeded the efficacy of rosuvastatin and atorvastatin monotherapy in appropriate doses. The use of combinations by studied scheme A was found to be most effective.

The study of atherosclerotic lesions of the aorta, aortal histology, histology findings of heart valves in the treatment scheme showed that the severity of damages almost was twice lower with combination therapy than with statin monotherapy, demonstrating the contribution of AGA in antiatherosclerotic activity of the combination by this scheme and confirming the antiatherosclerotic efficacy of AGA. In most cases, the efficacy of atorvastatin+AGA combination exceeded thereof combination of rosuvastatin+AGA.

The total score of studied drugs efficacy is presented in Table 88 (scheme A) and Table 89 (scheme B).

According to the total score of studied drugs administration by Scheme A, represented in Table 88, the use of studied combinations was effective concerning all studied parameters. The efficacy of both studied combinations was characterized by a direct dose-dependence from AGA and exceeded that of statin monotherapy at equivalent dose. Total score of each of the combinations was deducted with total score of monotherapy in equivalent dose for comparison of the total efficacy of the combination itself.

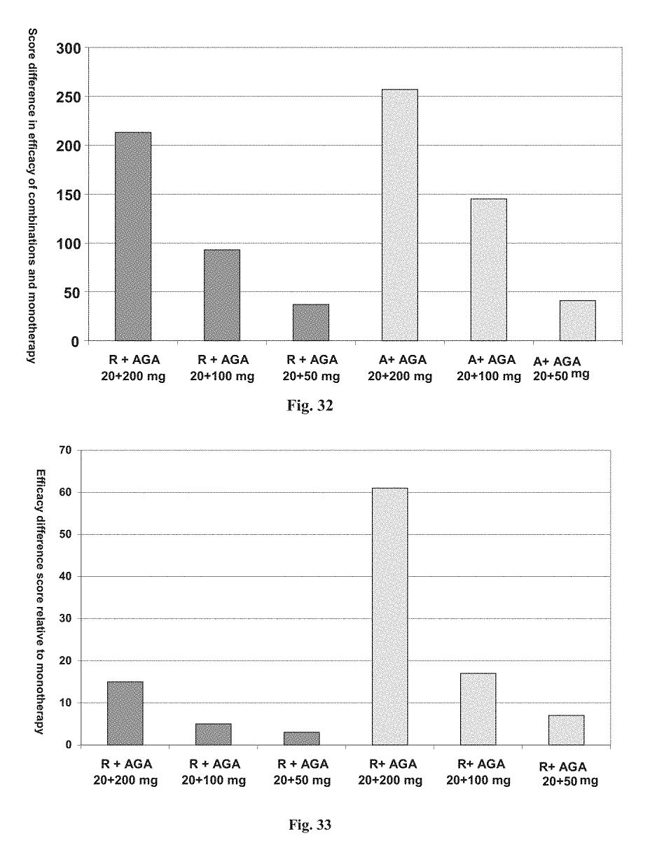

The results of total score in order to select the most promising combination are represented on FIG. 32. As can be seen from FIG. 32, the administration of combination of atorvastatin+AGA by scheme A was characterized by a higher total score than rosuvastatin+AGA combination.

The use of combination of atorvastatin+AGA according to the present invention in a dose of 20+200 mg by studied scheme A was found to be most effective concerning modeled pathology.

According to the total score of studied drugs administration by Scheme B, represented in Table 90, the use of studied combinations was effective concerning all studied parameters. The efficacy of both studied combinations was characterized by a direct dose-dependence from AGA and exceeded that of statin monotherapy at equivalent dose. Total score of each of the combinations was deducted with total score of monotherapy in equivalent dose for comparison of the total efficacy of the combination itself. The results of total score in order to select the most promising combination are represented on FIG. 33.

As is seen from the FIG. 33, the administration of combination of atorvastatin+AGA by scheme B was characterized by a higher total score than rosuvastatin+AGA combination.

The use of combination of atorvastatin+AGA in a dose of 20+200 mg by studied scheme B was found to be most effective concerning modeled pathology. This tendency may be a basis for choosing atorvastatin for the combination with AGA as a safe and effective antiatherosclerotic drug of choice.

TABLE 88
Total score of studied drugs efficacy—Scheme A

| | | | | | Total score in parameters | | | | | | | | |
|---|---|---|---|---|---|---|---|---|---|---|---|---|---|
| | | | | | Mass | | | | | | | | |
| | | | | | ratio of | | | | Morphometry | | | | |
| | Sub- | | | | hepar | Lipid | | | of | Aortal | Hepatic | Heart | |
| Group | group | Dose, | Group | | and | spectrum | Toxicity | Blood | atherosclerotic | his- | his- | valves | Total |
| [symbol] ^{o} | [symbol] ^{o} | mg | description | N | pancreas | (×10)* | (×10)* | coagulation | plaque | tology | tology | histology | score |
| 2 | 2A | 0 | Control | 6 | 4 | 200 | 380 | 12 | 10 | 1 | 5 | 1 | 613 |
| 5 | 3A | 20 + 200 | Present | 6 | 6 | 600 | 550 | 24 | 40 | 4 | 9 | 5 | 1238 |
| 7 | 4A | 20 + 100 | invention— | 6 | 6 | 530 | 510 | 23 | 30 | 6 | 7 | 6 | 1118 |
| 9 | 5A | 20 + 50 | rosuvastatm + | 6 | 4 | 520 | 480 | 23 | 20 | 4 | 6 | 5 | 1062 |
| | | | AGA | | | | | | | | | | |
| | | | treatment | | | | | | | | | | |
| 11 | 6A | 20 | Comparison— | 6 | 3 | 520 | 440 | 23 | 30 | 3 | 3 | 3 | 1025 |
| 13 | 6A2 | 40 | rosuvastatin | 6 | 2 | 670 | 460 | 23 | 40 | 5 | 1 | 5 | 1206 |
| | | | treatment | | | | | | | | | | |
| 14 | 7A | 20 + 200 | Present | 6 | 7 | 650 | 550 | 24 | 40 | 7 | 10 | 6 | 1294 |
| 16 | 8A | 20 + 100 | invention— | 6 | 6 | 500 | 520 | 24 | 30 | 9 | 9 | 4 | 1182 |
| 18 | 9A | 20 + 50 | atorvastatin + | 6 | 3 | 540 | 470 | 23 | 20 | 8 | 8 | 6 | 1078 |
| | | | AGA | | | | | | | | | | |
| | | | treatment | | | | | | | | | | |
| 20 | 10A | 20 | Comparison— | 6 | 3 | 540 | 440 | 23 | 20 | 4 | 4 | 3 | 1037 |
| 22 | 10A2 | 40 | atorvastatin | 6 | 2 | 720 | 450 | 25 | 40 | 7 | 2 | 6 | 1252 |
| | | | treatment | | | | | | | | | | |
| 23 | 11A | 100 | Comparison— | 6 | 7 | 380 | 440 | 15 | 10 | 2 | 6 | 2 | 862 |
| | | | AGA | | | | | | | | | | |
| | | | treatment | | | | | | | | | | |

Note—
*efficacy score of “lipid profile” and “Toxicity” parameters was multiplied by 10, as these are key parameters to assess the effectiveness and safety of studied drugs and are characterized by greatest significance.

TABLE 89
Total score of studied drugs efficacy—Scheme B

| | | | | | Total score in parameters | | | | | | | | | |
|---|---|---|---|---|---|---|---|---|---|---|---|---|---|---|
| | | | | | Mass | | Mass | | Mass | | Mass | | Mass | |
| | | | | | ratio of | | ratio | | ratio of | | ratio of | | ratio of | |
| | Sub- | | | | hepar | Lipid | of hepar | Lipid | hepar | Lipid | hepar | Lipid | hepar | |
| Group | group | Dose, | Group | | and | spectrum | and | spectrum | and | spectrum | and | spectrum | and | Total |
| [symbol] ^{o} | [symbol] ^{o} | mg | description | N | pancreas | (×10)* | pancreas | (×10)* | pancreas | (×10)* | pancreas | (×10)* | pancreas | score |
| 1 | 1 | 0 | Intact | 6 | 2 | 7 | 620 | 620 | 28 | 10 | 7 | 9 | 9 | 1312 |
| 4 | 2B1 | 0 | Control | 6 | 1 | 3 | 200 | 200 | 12 | 10 | 1 | 4 | 1 | 432 |
| 6 | 3B | 20 + 200 | Present | 6 | 3 | 5 | 530 | 530 | 20 | 20 | 5 | 8 | 8 | 1129 |
| 8 | 4B | 20 + 100 | invention— | 6 | 2 | 4 | 530 | 530 | 21 | 20 | 3 | 5 | 4 | 1119 |
| 10 | 5B | 20 + 50 | rosuvastatin + | 6 | 1 | 3 | 530 | 530 | 20 | 20 | 4 | 3 | 6 | 1117 |
| | | | AGA treatment | | | | | | | | | | | |
| 12 | 6B | 20 | Comparison— | 6 | 1 | 2 | 530 | 530 | 20 | 20 | 4 | 2 | 5 | 1114 |
| | | | rosuvastatin | | | | | | | | | | | |
| | | | treatment | | | | | | | | | | | |
| 15 | 7B | 20 + 200 | Present | 6 | 2 | 7 | 550 | 550 | 21 | 30 | 6 | 7 | 7 | 1180 |
| 17 | 8B | 20 + 100 | invention— | 6 | 2 | 6 | 530 | 530 | 21 | 30 | 4 | 5 | 8 | 1136 |
| 19 | 9B | 20 + 50 | atorvastatin + | 6 | 1 | 4 | 530 | 530 | 22 | 30 | 3 | 3 | 3 | 1126 |
| | | | AGA treatment | | | | | | | | | | | |
| 21 | 10B | 20 | Comparison— | 6 | 1 | 2 | 530 | 530 | 20 | 30 | 3 | 1 | 2 | 1119 |
| | | | atorvastatin | | | | | | | | | | | |
| | | | treatment | | | | | | | | | | | |
| 24 | 11B | 100 | Comparison— | 6 | 2 | 6 | 360 | 360 | 15 | 20 | 2 | 6 | 2 | 773 |
| | | | AGA treatment | | | | | | | | | | | |

Note—
*efficacy score of “lipid profile” and “Toxicity” parameters was multiplied by 10, as these are key parameters to assess the effectiveness and safety of studied drugs and are characterized by greatest significance.

### Conclusions

The study of specific pharmacological activity and safety of the fixed combinations of atorvastatin+monoammonium glycyrrhizinate, rosuvastatin+monoammonium glycyrrhizinate, as examples of the combination therapy of the present invention, compared with atorvastatin and rosuvastatin monotherapies in a model of hypercholesterolemia, dyslipidemia and atherosclerosis in rabbits caused by exposure to atherogenic factors, demonstrated high efficacy of the studied combinations of rosuvastatin+AGA and atorvastatin+AGA. The effectiveness of combinations in most cases was characterized by a direct dose-dependence from AGA and exceeded that of monotherapies.

1. The study established the most effective and safe dose of monoammonium glycyrrhizinate in fixed combinations, which amounted to 200 mg as glycyrrhizic acid equivalent.
2. The margins of fixed combinations safety with the perspective of selecting the optimum range of dosage for use in clinical practice was determined: the use of both combinations in a dose of 20 mg statin+100 mg glycyrrhizinate and 20 mg statin+200 mg glycyrrhizinate provided the most significant effect of combinations and their safety.
3. Significant hypercholesterolemic effect of the fixed combinations of the present invention was showed.
4. High antiatherosclerotic efficacy of the fixed combinations of the present invention was showed, manifested in significant atherogenic index decrease, reducing the area of aortal lesion and severity of histological findings in aorta and heart valves.
5. Safety profile of the fixed combinations of the present invention compare to rosuvastatin and atorvastatin monotherapies was studied. The intake of monoammonium glycyrrhizinate in hepatoprotective and myoprotective activity of combinations was demonstrated.
6. The absence of pathology influence on concentration of sodium and potassium ions was shown, that may be an indirect proof of the absence of steroid-like effect of combinations.
7. The stabilization of fluctuations of glucose level along with treatment with studied combinations was shown.
8. According to the results of biometric, biochemical and pathomorphological studies, we can conclude the advantages of efficacy and safety profile of rosuvastatin+AGA and atorvastatin+AGA combinations on the efficacy of statin monotherapy.

The above-mentioned effects of the tested combinations would also be expected to be achieved for combination of other statins (especially atorvastatin, lovastatin, pravastatin, pitavastatin, rosuvastatin, simvastatin and fluvastatin) and/or other glycyrrhizin derivatives (especially glycyrrhizic acid, glycyrrhetic acid or a pharmaceutically acceptable salt, solvate or hydrate of either thereof), particularly in the dose ranges set out in the present invention.

All publications mentioned in the above specification are herein incorporated by reference. Various modifications and variations of the described methods of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in chemistry, biology, medicine or related fields are intended to be within the scope of the following claims.

1. A combination comprising:
(a) a glycyrrhizin derivative; and
(b) a hypolipidemic drug;
provided that:
wherein the hypolipidemic drug is atorvastatin, the combination does not contain a molecular complex of atorvastatin and glycyrrhizic acid; and
wherein the hypolipidemic drug is simvastatin, the combination does not contain a molecular complex of simvastatin and glycyrrhizic acid.

2. A pharmaceutical composition comprising:
(a) a glycyrrhizin derivative; and
(b) a hypolipidemic drug;
provided that:
wherein the hypolipidemic drug is atorvastatin, the composition does not contain a molecular complex of atorvastatin and glycyrrhizic acid; and
wherein the hypolipidemic drug is simvastatin, the composition does not contain a molecular complex of simvastatin and glycyrrhizic acid.

3. A pharmaceutical composition comprising:
(a) a glycyrrhizin derivative; and
(b) a hypolipidemic drug;
wherein the pharmaceutical composition is a solid pharmaceutical composition.

4. A pharmaceutical composition according to claim 3, which is a solid mixture of the glycyrrhizin derivative and the hypolipidemic drug.

5. A kit comprising:
(a) a therapeutically effective amount of a glycyrrhizin derivative, and optionally a pharmaceutically acceptable carrier or diluent in a first unit dosage form;
(b) a therapeutically effective amount of a hypolipidemic drug, and optionally a pharmaceutically acceptable carrier or diluent in a second unit dosage form; and
(c) container means for containing said first and second dosage forms.

6. A method of preparing the pharmaceutical composition of claim 3 or claim 4, the method comprising mixing a solid form of the glycyrrhizin and a solid form of the hypolipidemic drug.

7. The combination of claim 1, the pharmaceutical composition of any of claims 2 to 4, or the kit of claim 5, wherein the hypolipidemic drug is a statin.

8. The combination, pharmaceutical composition, or kit of claim 7, wherein the statin is selected from the group consisting of atorvastatin, lovastatin, pravastatin, pitavastatin, rosuvastatin, simvastatin and fluvastatin, or a pharmaceutically acceptable salt, solvate or hydrate thereof, or a mixtures of any thereof.

9. The combination of claim 1, the pharmaceutical composition of any of claims 2 to 4, or the kit of claim 5, wherein the glycyrrhizin derivative is a compound of the formula: 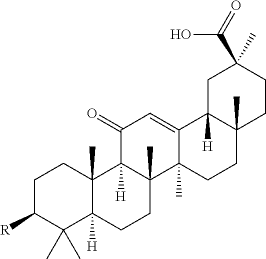
wherein R is selected from the group consisting of:
hydrogen;
a monosaccharide, disaccharide or oligosaccharide moiety, said moiety being optionally oxidised, reduced, deoxy, etherified and/or esterified;
hydroxy;
amino;
halo;
C₁₋₁₀ alkoxy optionally substituted by one or more substituents selected from halo, hydroxy, C₁₋₁₀ alkoxy, carboxy, (C₁₋₁₀ alkoxy)carbonyl, or a monosaccharide, disaccharide or oligosaccharide moiety, said moiety being optionally oxidised, reduced, deoxy, etherified and/or esterified;
C₁₋₁₀ alkyl optionally substituted by one or more substituents selected from halo, hydroxy, C₁₋₁₀ alkoxy, carboxy, (C₁₋₁₀ alkoxy)carbonyl, or a monosaccharide, disaccharide or oligosaccharide moiety; said moiety being optionally oxidised, reduced, deoxy, etherified and/or esterified;
or a deoxy derivative thereof;
or a pharmaceutically acceptable salt, solvate or hydrate thereof.

10. The combination, pharmaceutical composition, or kit of claim 9, wherein R is hydroxy or a monosaccharide or disaccharide moiety, said moiety being optionally oxidised, reduced, deoxy, etherified and/or esterified.

11. The combination, pharmaceutical composition, or kit of claim 9, wherein R is hydroxy or a disaccharide moiety, said moiety being optionally oxidised.

12. The combination, pharmaceutical composition, or kit of claim 9, wherein the glycyrrhizin derivative is glycyrrhizic acid or a pharmaceutically acceptable salt, solvate or hydrate thereof.

13. The combination, pharmaceutical composition, or kit of claim 9, wherein the glycyrrhizin derivative is glycyrrhetic acid or a pharmaceutically acceptable salt, solvate or hydrate thereof.

14. The combination of claim 1, the pharmaceutical composition of any of claims 2 to 4, or the kit of claim 5 wherein the glycyrrhizin derivative and the hypolipidemic drug are present in a ratio by mass (glycyrrhizin derivative:hypolipidemic drug) of from 1:0.03 to 1:5.

15. The combination of claim 1, the pharmaceutical composition of any of claims 2 to 4, or the kit of claim 5 wherein the glycyrrhizin derivative and the hypolipidemic drug are present in a ratio by mass (glycyrrhizin derivative:hypolipidemic drug) of from 1:0.03 to 1:2.

16. The combination of claim 1, the pharmaceutical composition of any of claims 2 to 4, or the kit of claim 5 wherein the hypolipidemic drug is a statin and the glycyrrhizin derivative and the statin are present in a ratio by mass (glycyrrhizin derivative:statin) of from 1:0.05 to 1:1.

17. The combination of claim 1, the pharmaceutical composition of any of claims 2 to 4, or the kit of claim 5, wherein the statin is simvastatin or a pharmaceutically acceptable salt, solvate or hydrate thereof, the glycyrrhizin derivative is glycyrrhizic acid or a pharmaceutically acceptable salt, solvate or hydrate thereof, the simvastatin or pharmaceutically acceptable salt, solvate or hydrate thereof is dosed at 0.1 to 200 mg/day, preferably 1 to 100 mg/day, more preferably 5 to 50 mg/day, even more preferably 10 to 30 mg/day, still more preferably 15 to 25 mg/day, and most preferably 20 mg/day, and the glycyrrhizic acid or pharmaceutically acceptable salt, solvate or hydrate thereof is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day and most preferably 80 to 120 mg/day.

18. The combination of claim 1, the pharmaceutical composition of any of claims 2 to 4, or the kit of claim 5, wherein the statin is atorvastatin or a pharmaceutically acceptable salt, solvate or hydrate thereof, the glycyrrhizin derivative is glycyrrhizic acid or a pharmaceutically acceptable salt, solvate or hydrate thereof, the atorvastatin or pharmaceutically acceptable salt, solvate or hydrate thereof is dosed at 0.05 to 100 mg/day, preferably 0.5 to 50 mg/day, more preferably 1 to 40 mg/day, even more preferably 2 to 20 mg/day, still more preferably 5 to 15 mg/day, and most preferably 10 mg/day, and the glycyrrhizic acid or pharmaceutically acceptable salt, solvate or hydrate thereof is dosed at 0.55 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day and most preferably 80 to 120 mg/day.

19. The combination of claim 1, the pharmaceutical composition of any of claims 2 to 4, or the kit of claim 5, wherein the statin is lovastatin or a pharmaceutically acceptable salt, solvate or hydrate thereof, the glycyrrhizin derivative is glycyrrhizic acid or a pharmaceutically acceptable salt, solvate or hydrate thereof, the lovastatin or a pharmaceutically acceptable salt, solvate or hydrate thereof is dosed at 0.1 to 200 mg/day, preferably 1 to 100 mg/day, more preferably 5 to 50 mg/day, even more preferably 10 to 30 mg/day, still more preferably 15 to 25 mg/day, and most preferably 20 mg/day, and the glycyrrhizic acid or a pharmaceutically acceptable salt, solvate or hydrate thereof is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day and most preferably 80 to 120 mg/day.

20. The combination of claim 1, the pharmaceutical composition of any of claims 2 to 4, or the kit of claim 5, wherein the statin is pravastatin or a pharmaceutically acceptable salt, solvate or hydrate thereof, the glycyrrhizin derivative is glycyrrhetic acid or a pharmaceutically acceptable salt or solvate thereof, the pravastatin or pharmaceutically acceptable salt, solvate or hydrate thereof is dosed at 0.02 to 400 mg/day, preferably 1 to 200 mg/day, more preferably 2 to 100 mg/day, even more preferably 5 to 50 mg/day, still more preferably 20 to 30 mg/day, and most preferably 40 mg/day, and the glycyrrhetic acid or pharmaceutically acceptable salt, solvate or hydrate thereof is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day and most preferably 80 to 120 mg/day.

21. The combination of claim 1, the pharmaceutical composition of any of claims 2 to 4, or the kit of claim 5, wherein the statin is rosuvastatin or a pharmaceutically acceptable salt, solvate or hydrate thereof, and the glycyrrhizin derivative is glycyrrhizic acid or a pharmaceutically acceptable salt, solvate or hydrate thereof, the rosuvastatin is dosed at 0.05 to 100 mg/day, preferably 0.5 to 50 mg/day, more preferably 1 to 40 mg/day, even more preferably 2 to 20 mg/day, still more preferably 5 to 15 mg/day, and most preferably 10 mg/day, and the glycyrrhizic acid or a pharmaceutically acceptable salt, solvate or hydrate thereof is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day and most preferably 80 to 120 mg/day.

22. The combination of claim 1, the pharmaceutical composition of any of claims 2 to 4, or the kit of claim 5, wherein the statin is fluvastatin or a pharmaceutically acceptable salt, solvate or hydrate thereof, and the glycyrrhizin derivative is glycyrrhizic acid or a pharmaceutically acceptable salt, solvate or hydrate thereof, the fluvastatin or pharmaceutically acceptable salt, solvate or hydrate thereof is dosed at 0.1 to 800 mg/day, preferably 1 to 400 mg/day, more preferably 20 to 200 mg/day, even more preferably 40 to 120 mg/day, still more preferably 60 to 100 mg/day, and most preferably 80 mg/day and the glycyrrhizic acid or a pharmaceutically acceptable salt, solvate or hydrate thereof is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day and most preferably 80 to 120 mg/day.

23. The combination of claim 1, the pharmaceutical composition of any of claims 2 to 4, or the kit of claim 5, wherein the statin is pitavastatin or a pharmaceutically acceptable salt, solvate or hydrate thereof, and the glycyrrhizin derivative is glycyrrhizic acid or a pharmaceutically acceptable salt, solvate or hydrate thereof, the pitavastatin or pharmaceutically acceptable salt, solvate or hydrate thereof is dosed at 0.2 to 800 mg/day, preferably 0.5 to 200 mg/day, more preferably 2 to 100 mg/day, even more preferably 5 to 50 mg/day, still more preferably 10 to 30 mg/day, and most preferably 40 mg/day and the glycyrrhizic acid or a pharmaceutically acceptable salt, solvate or hydrate thereof is dosed at 0.5 to 1000 mg/day, preferably 1 to 500 mg/day, more preferably 5 to 400 mg/day, even more preferably 10 to 300 mg/day, still more preferably 20 to 250 mg/day, yet more preferably 50 to 200 mg/day and most preferably 80 to 120 mg/day.

24. The combination of claim 1, the pharmaceutical composition of any of claims 2 to 4, or the kit of claim 5, for use as a medicament.

25. The combination of claim 1, the pharmaceutical composition of any of claims 2 to 4, or the kit of claim 5, for use in treating a disease selected from the group consisting of hyperlipidemia, hypercholesterolemia and triglyceridemia.

26. The combination of claim 1, the pharmaceutical composition of any of claims 2 to 4, or the kit of claim 5, for use in treating cardiovascular disease.

27. The combination, pharmaceutical composition or kit for use of claim 22, wherein the cardiovascular disease is selected from the group consisting of ischemic heart disease, myocardial infarction, angina, stroke, atherosclerotic vascular disease, coronary heart disease, coronary artery disease, peripheral vascular disease, peripheral arterial disease, and intermittent claudication.

28. The combination of claim 1, the pharmaceutical composition of any of claims 2 to 4, or the kit of claim 5, for use in treating atherosclerosis.
